(19) United States
(12) Patent Application Publication
(10) Pub. No.: US 2023/0115274 A1
(43) Pub. Date: Apr. 13, 2023

(54) COMPOUNDS AND COMPOSITIONS FOR TREATING CONDITIONS ASSOCIATED WITH STING ACTIVITY

(71) Applicant: IFM Due, Inc., Boston, MA (US)

(72) Inventors: William R. Roush, Boston, MA (US); Hans Martin Seidel, Concord, MA (US); Shankar Venkatraman, Lansdale, PA (US); Jason Katz, Newton, MA (US)

(21) Appl. No.: 17/789,623

(22) PCT Filed: Dec. 30, 2020
(86) PCT No.: PCT/US2020/067463

§ 371 Date: Jun. 28, 2022

Related U.S. Application Data
(60) Provisional application No. 62/955,853, filed on Dec. 31, 2019.
(60) Provisional application No. 63/090,547, filed on Oct. 12, 2020.

(51) Int. Cl. C07D 471/04; C07D 519/00

(52) U.S. Cl. CPC C07D 471/04; C07D 519/00

(57) ABSTRACT

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

### CROSS REFERENCE TO RELATED APPLICATIONS

This application claims the benefit of U.S. Provisional Application Ser. No. 62/955,853, filed on Dec. 31, 2019; and U.S. Provisional Application Ser. No. 63/090,547, filed on Oct. 12, 2020; each of which is incorporated herein by reference in its entirety.

### TECHNICAL FIELD

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

### BACKGROUND

STING, also known as transmembrane protein 173 (TMEM173) and MPYS/MITA/ERIS, is a protein that in humans is encoded by the TMEM173 gene. STING has been shown to play a role in innate immunity. STING induces type I interferon production when cells are infected with intracellular pathogens, such as viruses, mycobacteria and intracellular parasites. Type I interferon, mediated by STING, protects infected cells and nearby cells from local infection in an autocrine and paracrine manner.

The STING pathway is pivotal in mediating the recognition of cytosolic DNA. In this context, STING, a transmembrane protein localized to the endoplasmic reticulum (ER), acts as a second messenger receptor for 2′, 3′ cyclic GMP-AMP (hereafter cGAMP), which is produced by cGAS after dsDNA binding. In addition, STING can also function as a primary pattern recognition receptor for bacterial cyclic dinucleotides (CDNs) and small molecule agonists. The recognition of endogenous or prokaryotic CDNs proceeds through the carboxy-terminal domain of STING, which faces into the cytosol and creates a V-shaped binding pocket formed by a STING homodimer. Ligand-induced activation of STING triggers its re-localization to the Golgi, a process essential to promote the interaction of STING with TBK1. This protein complex, in turn, signals through the transcription factors IRF-3 to induce type I interferons (IFNs) and other co-regulated antiviral factors. In addition, STING was shown to trigger NF-κB and MAP kinase activation. Following the initiation of signal transduction, STING is rapidly degraded, a step considered important in terminating the inflammatory response.

Excessive activation of STING is associated with a subset of monogenic autoinflammatory conditions, the so-called type I interferonopathies. Examples of these diseases include a clinical syndrome referred to as STING-associated vasculopathy with onset in infancy (SAVI), which is caused by gain-of-function mutations in TMEM173 (the gene name of STING). Moreover, STING is implicated in the pathogenesis of Aicardi-Goutieres Syndrome (AGS) and genetic forms of lupus. As opposed to SAVI, it is the dysregulation of nucleic acid metabolism that underlies continuous innate immune activation in AGS. Apart from these genetic disorders, emerging evidence points to a more general pathogenic role for STING in a range of inflammation-associated disorders such as systemic lupus erythematosus, rheumatoid arthritis and cancer. Thus, small molecule-based pharmacological interventions into the STING signaling pathway hold significant potential for the treatment of a wide spectrum of diseases

### SUMMARY

This disclosure features chemical entities (e.g., a compound or a pharmaceutically acceptable salt, and/or hydrate, and/or cocrystal, and/or prodrug, and/or tautomer, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

An “antagonist” of STING includes compounds that, at the protein level, directly bind or modify STING such that an activity of STING is decreased, e.g., by inhibition, blocking or dampening agonist-mediated responses, altered distribution, or otherwise.

STING antagonists include chemical entities, which interfere or inhibit STING signaling.

In one aspect, compounds of Formula (I), or a pharmaceutically acceptable salt thereof, are featured:

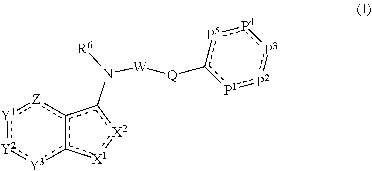

in which Z, Y¹, Y², Y³, X, X², R⁶, W, Q, P¹, P², P³, P⁴, and P⁵ can be as defined anywhere herein.

In one aspect, compounds of Formula (I), or a pharmaceutically acceptable salt thereof, or a prodrug thereof, or a tautomer thereof, or any combination of the foregoing, are featured “Prodrug” is meant to indicate a compound that may be converted under physiological conditions or by solvolysis to a biologically active compound described herein (e.g., compound of Formula (I)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for inhibiting (e.g., antagonizing) STING activity are featured that include contacting STING with a chemical entity described herein (e.g., a compound described generically or specifically herein or a pharmaceutically acceptable salt thereof or compositions containing the same). Methods include in vitro methods, e.g., contacting a sample that includes one or more cells comprising STING (e.g., innate immune cells, e.g., mast cells, macrophages, dendritic cells (DCs), and natural killer cells) with the chemical entity. Methods can also include in vivo methods; e.g., administering the chemical entity to a subject (e.g., a human) having a disease in which increased (e.g., excessive) STING signaling contributes to the pathology and/or symptoms and/or progression of the disease.

In one aspect, methods of treating a condition, disease or disorder ameliorated by antagonizing STING are featured, e.g., treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). The methods include administering to a subject in need of such treatment an effective amount of a chemical entity described herein (e.g., a compound described generically or specifically herein or a pharmaceutically acceptable salt thereof or compositions containing the same).

In another aspect, methods of treating cancer are featured that include administering to a subject in need of such treatment an effective amount of a chemical entity described herein (e.g., a compound described generically or specifically herein or a pharmaceutically acceptable salt thereof or compositions containing the same).

In a further aspect, methods of treating other STING-associated conditions are featured, e.g., type I interferonopathies (e.g., STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis. The methods include administering to a subject in need of such treatment an effective amount of a chemical entity described herein (e.g., a compound described generically or specifically herein or a pharmaceutically acceptable salt thereof or compositions containing the same).

In another aspect, methods of suppressing STING-dependent type I interferon production in a subject in need thereof are featured that include administering to the subject an effective amount of a chemical entity described herein (e.g., a compound described generically or specifically herein or a pharmaceutically acceptable salt thereof or compositions containing the same).

In a further aspect, methods of treating a disease in which increased (e.g., excessive) STING activation (e.g., STING signaling) contributes to the pathology and/or symptoms and/or progression of the disease are featured. The methods include administering to a subject in need of such treatment an effective amount of a chemical entity described herein (e.g., a compound described generically or specifically herein or a pharmaceutically acceptable salt thereof or compositions containing the same).

In another aspect, methods of treatment are featured that include administering an effective amount of a chemical entity described herein (e.g., a compound described generically or specifically herein or a pharmaceutically acceptable salt thereof or compositions containing the same) to a subject; wherein the subject has (or is predisposed to have) a disease in which increased (e.g., excessive) STING activation (e.g., STING signaling) contributes to the pathology and/or symptoms and/or progression of the disease.

In a further aspect, methods of treatment that include administering to a subject a chemical entity described herein (e.g., a compound described generically or specifically herein or a pharmaceutically acceptable salt thereof or compositions containing the same), wherein the chemical entity is administered in an amount effective to treat a disease in which increased (e.g., excessive) STING activation (e.g., STING signaling) contributes to the pathology and/or symptoms and/or progression of the disease, thereby treating the disease.

In another aspect, is a compound, or a pharmaceutically acceptable salt or tautomer thereof, described herein, for use in the treatment of a disease, condition or disorder modulated by STING inhibition.

In another aspect, is a compound, or a pharmaceutically acceptable salt or tautomer thereof, described herein for use in the treatment of a condition, disease or disorder associated with increased (e.g., excessive) STING activation.

In another aspect, is a compound, or a pharmaceutically acceptable salt or tautomer thereof, described herein for use in the treatment of cancer.

In another aspect, is a compound, or a pharmaceutically acceptable salt or tautomer thereof, described herein for use in the treatment of cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

In another aspect, is a compound, or a pharmaceutically acceptable salt or tautomer thereof, described herein for use in the treatment of type I interferonopathies.

In another aspect, is a compound, or a pharmaceutically acceptable salt or tautomer thereof, described herein for use in the treatment of type I interferonopathies selected from STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis.

In another aspect, is the use of a compound, or a pharmaceutically acceptable salt or tautomer thereof, described herein for use in the manufacture of a medicament for the treatment of a condition, disease or disorder associated with increased (e.g., excessive) STING activation.

In another aspect, is the use of a compound, or a pharmaceutically acceptable salt or tautomer thereof, described herein for use in the manufacture of a medicament for the treatment of cancer.

In another aspect, is the use of a compound, or a pharmaceutically acceptable salt or tautomer thereof, described herein for use in the manufacture of a medicament for the treatment of cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

In another aspect, is the use of a compound, or a pharmaceutically acceptable salt or tautomer thereof, described herein for use in the manufacture of a medicament for the treatment of type I interferonopathies.

In another aspect, is the use of a compound, or a pharmaceutically acceptable salt or tautomer thereof, described herein for use in the manufacture of a medicament for the treatment of type I interferonopathies selected from STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis.

In another aspect, is the use of a compound, or a pharmaceutically acceptable salt or tautomer thereof, described herein, for the treatment of a disease, condition or disorder modulated by STING inhibition.

In another aspect, is the use of a compound, or a pharmaceutically acceptable salt or tautomer thereof, described herein for the treatment of a condition, disease or disorder associated with increased (e.g., excessive) STING activation.

In another aspect, is the use of a compound, or a pharmaceutically acceptable salt or tautomer thereof, described herein for the treatment of cancer.

In another aspect, is the use of a compound, or a pharmaceutically acceptable salt or tautomer thereof, described herein for the treatment of cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

In another aspect, is the use of a compound, or a pharmaceutically acceptable salt or tautomer thereof, described herein for the treatment of type I interferonopathies.

In another aspect, is the use of a compound, or a pharmaceutically acceptable salt or tautomer thereof, described herein for the treatment of type I interferonopathies selected from STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis.

Embodiments can include one or more of the following features.

The chemical entity can be administered in combination with one or more additional therapeutic agents and/or regimens. For examples, methods can further include administering one or more (e.g., two, three, four, five, six, or more) additional agents.

The chemical entity can be administered in combination with one or more additional therapeutic agents and/or regimens that are useful for treating other STING-associated conditions, e.g., type I interferonopathies (e.g., STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis.

The chemical entity can be administered in combination with one or more additional cancer therapies (e.g., surgery, radiotherapy, chemotherapy, toxin therapy, immunotherapy, cryotherapy or gene therapy, or a combination thereof, e.g., chemotherapy that includes administering one or more (e.g., two, three, four, five, six, or more) additional chemotherapeutic agents. Non-limiting examples of additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

The subject can have cancer; e.g., the subject has undergone and/or is undergoing and/or will undergo one or more cancer therapies.

Non-limiting examples of cancer include melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma. In certain embodiments, the cancer can be a refractory cancer.

The chemical entity can be administered intratumorally.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “STING” is meant to include, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The term “halo” refers to fluoro (F), chloro (Cl), bromo (Br), or iodo (I).

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents.

Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—).

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, dihydro-1H-indenyl and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butanyl, bicyclo[2.1.0]pentanyl, bicyclo[1.1.1]pentanyl, bicyclo[3.1.0]hexanyl, bicyclo[2.1.1]hexanyl, bicyclo[3.2.0]heptanyl, bicyclo[4.1.0]heptanyl, bicyclo[2.2.1]heptanyl, bicyclo[3.1.1]heptanyl, bicyclo[4.2.0]octanyl, bicyclo[3.2.1]octanyl, bicyclo[2.2.2]octanyl, and the like. Cycloalkyl also includes spirocyclic rings (e.g., spirocyclic bicycle wherein two rings are connected through just one atom). Non-limiting examples of spirocyclic cycloalkyls include spiro[2.2]pentanyl, spiro[2.5]octanyl, spiro[3.5]nonanyl, spiro[3.5]nonanyl, spiro[3.5]nonanyl, spiro[4.4]nonanyl, spiro[2.6]nonanyl, spiro[4.5]decanyl, spiro[3.6]decanyl, spiro[5.5]undecanyl, and the like. The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic, and at least one ring in the system contains one or more heteroatoms independently selected from the group consisting of N, O, and S (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, benzo[d]thiazolyl, 2,3-dihydrobenzofuranyl, tetrahydroquinolinyl, 2,3-dihydrobenzo[b][1,4]oxathiinyl, indolinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl.

The term “heterocyclyl” refers to a mon-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butanyl, 2-azabicyclo[2.1.0]pentanyl, 2-azabicyclo[1.1.1]pentanyl, 3-azabicyclo[3.1.0]hexanyl, 5-azabicyclo[2.1.1]hexanyl, 3-azabicyclo[3.2.0]heptanyl, octahydrocyclopenta[c]pyrrolyl, 3-azabicyclo[4.1.0]heptanyl, 7-azabicyclo[2.2.1]heptanyl, 6-azabicyclo[3.1.1]heptanyl, 7-azabicyclo[4.2.0]octanyl, 2-azabicyclo[2.2.2]octanyl, 3-azabicyclo[3.2.1]octanyl, 2-oxabicyclo[1.1.0]butanyl, 2-oxabicyclo[2.1.0]pentanyl, 2-oxabicyclo[1.1.1]pentanyl, 3-oxabicyclo[3.1.0]hexanyl, 5-oxabicyclo[2.1.1]hexanyl, 3-oxabicyclo[3.2.0]heptanyl, 3-oxabicyclo[4.1.0]heptanyl, 7-oxabicyclo[2.2.1]heptanyl, 6-oxabicyclo[3.1.1]heptanyl, 7-oxabicyclo[4.2.0]octanyl, 2-oxabicyclo[2.2.2]octanyl, 3-oxabicyclo[3.2.1]octanyl, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentanyl, 4-azaspiro[2.5]octanyl, 1-azaspiro[3.5]nonanyl, 2-azaspiro[3.5]nonanyl, 7-azaspiro[3.5]nonanyl, 2-azaspiro[4.4]nonanyl, 6-azaspiro[2.6]nonanyl, 1,7-diazaspiro[4.5]decanyl, 7-azaspiro[4.5]decanyl, 2,5-diazaspiro[3.6]decanyl, 3-azaspiro[5.5]undecanyl, 2-oxaspiro[2.2]pentanyl, 4-oxaspiro[2.5]octanyl, 1-oxaspiro[3.5]nonanyl, 2-oxaspiro[3.5]nonanyl, 7-oxaspiro[3.5]nonanyl, 2-oxaspiro[4.4]nonanyl, 6-oxaspiro[2.6]nonanyl, 1,7-dioxaspiro[4.5]decanyl, 2,5-dioxaspiro[3.6]decanyl, 1-oxaspiro[5.5]undecanyl, 3-oxaspiro[5.5]undecanyl, 3-oxa-9-azaspiro[5.5]undecanyl and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl. As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings.

As used herein, when a ring is described as being “aromatic”, it means said ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

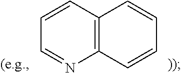

(ii) a single ring atom (spiro-fused ring systems)

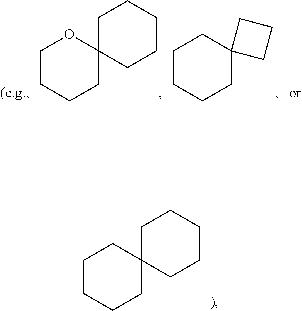

or (iii) a contiguous array of ring atoms (bridged ring systems having all bridge lengths >0)

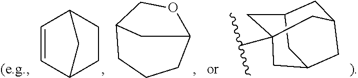

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

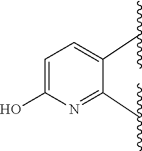 [SMILES: *c1ccc(O)nc1*]

encompasses the tautomeric form containing the moiety:

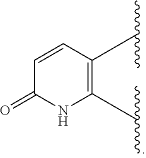 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The details of one or more embodiments of the invention are set forth in the accompanying drawings and the description below. Other features and advantages of the invention will be apparent from the description and drawings, and from the claims.

### DETAILED DESCRIPTION

This disclosure features chemical entities (e.g., a compound or a pharmaceutically acceptable salt, and/or hydrate, and/or cocrystal, and/or prodrug, and/or tautomer, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

Formula I Compounds

In one aspect, this disclosure features compounds of Formula (I):

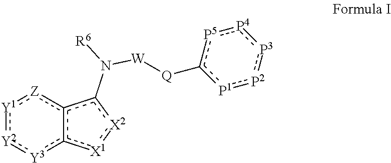

or a pharmaceutically acceptable salt thereof or a tautomer thereof, wherein:

each of Z, Y¹, Y², and Y³ is independently selected from the group consisting of CR¹, N, and NR², provided that 1-3 of Z, Y¹, Y², and Y³ is an independently selected N or NR²;

X¹ is selected from the group consisting of O, S, N, NR², and CR¹;

X² is selected from the group consisting of O, S, N, NR⁴, and CR⁵;

each [symbol] is independently a single bond or a double bond, provided that the five-membered ring comprising X¹ and X² is heteroaryl; the six-membered ring comprising Z, Y¹, Y², and Y³ is heteroaryl; and the ring comprising P¹, P², P³, P⁴, and P⁵ is aromatic;

W is selected from the group consisting of: (i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);

Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents the point of attachment to W;

P¹, P², P³, P⁴, and P⁵ are defined according to (AA) or (BB):
(AA)

each of P¹, P², P³, P⁴, and P⁵ is independently selected from the group consisting of: N, CH, CR⁷, and CR^{c}, provided that: 1-2 of P¹, P², P³, P⁴, and P⁵ is an independently selected CR⁷; or
(BB)

P¹ is absent, thereby providing a 5-membered ring, each of P², P³, P⁴, and P⁵ is independently selected from the group consisting of O, S, N, NH, NR^{d}, NR⁷, CH, CR⁷, and CR^{c};

provided that 1-3 of P², P³, P⁴, and P⁵ is O, S, N, NH, NR^{d}, or NR⁷; and

1-2 of P², P³, P⁴, and P⁵ is an independently selected NR⁷ or CR⁷;

each R⁷ is independently selected from the group consisting of: —R⁸ and -L³-R⁹

—R⁸ is selected from the group consisting of:

(a) C₃₋₁₂ cycloalkyl or C₃₋₁₂ cycloalkenyl, each of which is substituted with 1-4 independently selected R^{7′};

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′};

(c) C₃₋₆ cycloalkyl or C₃₋₆ cycloalkenyl, each of which is optionally substituted with 1-4 independently selected C₁₋₄ alkyl;

(d) C₇₋₁₂ cycloalkyl or C₇₋₁₂ cycloalkenyl, each of which is optionally substituted with 1-4 independently selected C₁₋₄ alkyl;

(e) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected C₁₋₄ alkyl;

(f) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(g) C₆₋₁₀ aryl optionally substituted with 1-4 independently selected R^{7′};

-L³ is selected from the group consisting of —O—, —S—, —NH—, S(O)₁₋₂, —CH₂—, C(═O)NH, NHC(═O), C(═O)O, OC(═O), C(═O), NHS(O)₂, and S(O)₂NH;

—R⁹ is selected from the group consisting of:

(a) C₃₋₁₂ cycloalkyl or C₃₋₁₂ cycloalkenyl, each of which is optionally substituted with 1-4 independently selected R^{7′},

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(d) C₆₋₁₀ aryl optionally substituted with 1-4 independently selected R^{7′};

each occurrence of R^{7′} is independently selected from the group consisting of:

halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),

provided that when R⁷ is R⁸; and R⁸ is cycloalkyl, cycloalkenyl, heterocyclyl, or heterocycloalkenyl and substituted with 1-4 R^{7′}, then:

R⁸ cannot be monosubstituted with C₁₋₄ alkyl, and

when R⁸ is substituted with 2-4 R^{7′}, then at least one R^{7′} must be a substituent other than C₁₋₄ alkyl;

each occurrence of R¹ is independently selected from the group consisting of:

H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy optionally substituted with —OH, C₁₋₄ alkoxy, C₁₋₄ haloalkoxy, or —NR^{e}R^{f}; C₁₋₄ haloalkoxy; -L¹-L²-R^{h}; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);

each occurrence of R² is independently selected from the group consisting of:

(i) H;

(ii) C₁₋₆ alkyl optionally substituted with 1-3 independently selected R^{a};

(iii) —C(O)(C₁₋₆ alkyl) optionally substituted with 1-3 independently selected R^{a};

(iv) —C(O)O(C₁₋₄ alkyl) optionally substituted with 1-3 independently selected R^{a};

(v) —CON(R′)(R″);

(vi) —S(O)₁₋₂(NR′R″);

(vii) —S(O)₁₋₂(C₁₋₄ alkyl) optionally substituted with 1-3 independently selected R^{a};

(viii) —OH;

(ix) C₁₋₄ alkoxy; and

(x) -L⁴-L⁵-R;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂, wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and

C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH;

oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

halo; cyano; C₁₋₁₀ alkyl optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};

R^{d} is selected from the group consisting of: C₁₋₆ alkyl optionally substituted with 1-3 substituents each independently selected from the group consisting of halo, C₁₋₄ alkoxy, and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or

R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl;

and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene; -L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and

C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};

-L⁵- is selected from the group consisting of a bond and C₁₋₄ alkylene;

Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;

each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH2, NH(C₁₋₄ alkyl), N(C₁₋₄ alkyl)₂, C₁₋₄ alkyl, and C₁₋₄ haloalkyl; or

R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In one aspect, this disclosure features compounds of Formula (I):

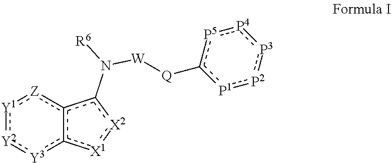

or a pharmaceutically acceptable salt thereof or a tautomer thereof, wherein:

each of Z, Y¹, Y², and Y³ is independently selected from the group consisting of CR¹, N, and NR², provided that 1-3 of Z, Y¹, Y², and Y³ is an independently selected N or NR²;

X¹ is selected from the group consisting of O, S, N, NR², and CR¹;

X² is selected from the group consisting of O, S, N, NR⁴, and CR⁵;

each [symbol] is independently a single bond or a double bond, provided that the five-membered ring comprising X¹ and X² is heteroaryl; the six-membered ring comprising Z, Y¹, Y², and Y³ is heteroaryl; and the ring comprising P¹, P², P³, P⁴, and P⁵ is aromatic;

W is selected from the group consisting of: (i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);

Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents the point of attachment to W;

P¹, P², P³, P⁴, and P⁵ are defined according to (AA) or (BB):
(AA)

each of P¹, P², P³, P⁴, and P⁵ is independently selected from the group consisting of: N, CH, CR⁷, and CR^{c}, provided that: 1-2 of P¹, P², P³, P⁴, and P⁵ is an independently selected CR⁷; or
(BB)

P¹ is absent, thereby providing a 5-membered ring,

each of P², P³, P⁴, and P⁵ is independently selected from the group consisting of O, S, N, NH, NR^{d}, NR⁷, CH, CR⁷, and CR^{e};

provided that 1-3 of P², P³, P⁴, and P⁵ is O, S, N, NH, NR^{d}, or NR⁷; and

1-2 of P², P³, P⁴, and P⁵ is an independently selected NR⁷ or CR⁷;

each R⁷ is independently selected from the group consisting of: —R⁸ and -L³-R⁹;

—R⁸ is selected from the group consisting of:

(a) C₃₋₁₂ cycloalkyl or C₃₋₁₂ cycloalkenyl, each of which is substituted with 1-4 independently selected R^{7′};

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′} (c) C₃ cycloalkyl, C3 cycloalkenyl, C5 cycloalkyl, or C5 cycloalkenyl, each of which is optionally substituted with 1-4 independently selected C₁₋₄ alkyl;

(d) C₇₋₁₂ cycloalkyl or C₇₋₁₂ cycloalkenyl, each of which is optionally substituted with 1-4 independently selected C₁₋₄ alkyl;

(e) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl or heterocycloalkenyl ring is optionally substituted with 1-4 independently selected C₁₋₄ alkyl;

(f) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(g) C₆₋₁₀ aryl optionally substituted with 1-4 independently selected R^{7′};

-L³ is selected from the group consisting of —O—, —S—, —NH—, S(O)₁₋₂, —CH₂—, C(═O)NH, NHC(═O), C(═O)O, OC(═O), C(═O), NHS(O)₂, and S(O)₂NH;

—R⁹ is selected from the group consisting of:

(a) C₃₋₁₂ cycloalkyl or C₃₋₁₂ cycloalkenyl, each of which is optionally substituted with 1-4 independently selected R^{7′},

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(d) C₆₋₁₀ aryl optionally substituted with 1-4 independently selected R^{7′};

each occurrence of R^{7′} is independently selected from the group consisting of:

halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),

provided that when R⁷ is R⁸; and R⁸ is cycloalkyl, cycloalkenyl, heterocyclyl, or heterocycloalkenyl and substituted with 1-4 R^{7′}, then:

R⁸ cannot be monosubstituted with C₁₋₄ alkyl, and

when R⁸ is substituted with 2-4 R^{7′}, then at least one R^{7′} must be a substituent other than C₁₋₄ alkyl;

each occurrence of R¹ is independently selected from the group consisting of:

H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L¹-L²-R^{h}; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂;

—C(═O)(C₁₋₄ alkyl); —C(═O)O(C₁₋₄ alkyl); —C(═O)OH; and —C(═O)N(R′)(R″);

each occurrence of R² is independently selected from the group consisting of:

(i) H;

(ii) C₁₋₆ alkyl optionally substituted with 1-3 independently selected R^{a};

(iii) —C(O)(C₁₋₆ alkyl) optionally substituted with 1-3 independently selected R^{a};

(iv) —C(O)O(C₁₋₄ alkyl) optionally substituted with 1-3 independently selected R^{a};

(v) —CON(R′)(R″);

(vi) —S(O)₁₋₂(NR′R″);

(vii) —S(O)₁₋₂(C₁₋₄ alkyl) optionally substituted with 1-3 independently selected R^{a};

(viii) —OH;

(ix) C₁₋₄ alkoxy; and

(x) -L⁴-L⁵-R;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂, wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and

C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH;

oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and

-L¹-L²-R^{h};

each occurrence of R^{c} is independently selected from the group consisting of:

halo; cyano; C₁₋₁₀ alkyl optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};

R^{d} is selected from the group consisting of: C₁₋₆ alkyl optionally substituted with 1-3 substituents each independently selected from the group consisting of halo, C₁₋₄ alkoxy, and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or

R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene; -L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};

-L⁵- is selected from the group consisting of a bond and C₁₋₄ alkylene;

Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy; and

each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH2, NH(C₁₋₄ alkyl), N(C₁₋₄ alkyl)₂, C₁₋₄ alkyl, and C₁₋₄ haloalkyl; or

R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In one aspect, this disclosure features compounds of Formula (I):

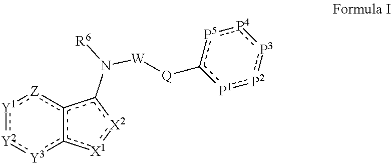

or a pharmaceutically acceptable salt thereof or a tautomer thereof, wherein:

each of Z, Y¹, Y², and Y³ is selected from the group consisting of CR¹, N, and NR², provided that 1-3 of Z, Y¹, Y², and Y³ is an independently selected N or NR²;

X¹ is selected from the group consisting of O, S, N, NR², and CR¹;

X² is selected from the group consisting of O, S, N, NR⁴, and CR⁵;

each [symbol] is independently a single bond or a double bond, provided that the five-membered ring comprising X¹ and X² is heteroaryl; the six-membered ring comprising Z, Y¹, Y², and Y³ is heteroaryl; and the ring including P¹, P², P³, P⁴, and P⁵ is aromatic;

W is selected from the group consisting of:

(i) C(═O); (ii) C(═S); (iii) S(O)₁₋₂; (iv) C(═NR^{d}) or C(═N—CN); (v) C(═NH); (vi) C(═C—NO₂); (vii) S(O)N(R^{d}); and (viii) S(O)NH;

Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents point of attachment to W;

P¹, P², P³, P⁴, and P⁵ are defined according to (AA) or (BB):
(AA)

each of P¹, P², P³, P⁴, and P⁵ is independently selected from the group consisting of: N, CH, CR⁷, and CR^{c}, provided that:

1-2 of P¹, P², P³, P⁴, and P⁵ is an independently selected CR⁷; or
(BB)

P¹ is absent (thereby providing a 5-membered ring), each of P², P³, P⁴, and P⁵ is independently selected from the group consisting of O, S, N, NH, NR^{d}, NR⁷, CH, CR⁷, and CR^{c};

provided that 1-3 of P², P³, P⁴, and P⁵ is O, S, N, NH, NR^{d}, or NR⁷; and

1-2 of P², P³, P⁴, and P⁵ is an independently selected NR⁷ or CR⁷ each R⁷ is independently selected from the group consisting of: —R⁸ and -L³-R⁹

—R⁸ is selected from the group consisting of:

(a) C₃₋₁₂ cycloalkyl or C₃₋₁₂ cycloalkenyl, each of which is substituted with 1-4 independently selected R^{7′};

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′};

(c) C₃ cycloalkyl, C3 cycloalkenyl, C5 cycloalkyl, or C5 cycloalkenyl, each of which is optionally substituted with 1-4 independently selected C₁₋₄ alkyl;

(d) C₇₋₁₂ cycloalkyl or C₇₋₁₂ cycloalkenyl, each of which is optionally substituted with 1-4 independently selected C₁₋₄ alkyl;

(e) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl or heterocycloalkenyl ring is optionally substituted with 1-4 independently selected C₁₋₄ alkyl;

(f) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(g) C₆₋₁₀ aryl optionally substituted with 1-4 independently selected R^{7′};

-L³ is selected from the group consisting of —O—, —S—, —NH—, S(O)₁₋₂, —CH₂—, C(═O)NH, NHC(═O), C(═O)O, OC(═O), C(═O), NHS(O)₂, and S(O)₂NH;

—R⁹ is selected from the group consisting of:

(a) C₃₋₁₂ cycloalkyl or C₃₋₁₂ cycloalkenyl, each of which is optionally substituted with 1-4 independently selected R^{7′},

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(d) C₆₋₁₀ aryl optionally substituted with 1-4 independently selected R^{7′};

each occurrence of R^{7′} is independently selected from the group consisting of:

halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),

provided that when R⁷ is R⁸; and R⁸ is cycloalkyl, cycloalkenyl, heterocyclyl, or heterocycloalkenyl, then one or more occurrence of R^{7′} is other than —C₁₋₄ alkyl;

each occurrence of R¹ is independently selected from the group consisting of

H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L¹-L²-R^{h}; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);

each occurrence of R² is independently selected from the group consisting of:

(i) H;

(ii) C₁₋₆ alkyl, which is optionally substituted with 1-3 independently selected R^{a};

(iii) —C(O)(C₁₋₆ alkyl) optionally substituted with 1-3 independently selected R^{a};

(iv) —C(O)O(C₁₋₄ alkyl) optionally substituted with 1-3 independently selected R^{a};

(v) —CON(R′)(R″);

(vi) —S(O)₁₋₂(NR′R″);

(vii) —S(O)₁₋₂(C₁₋₄ alkyl) optionally substituted with 1-3 independently selected R^{a};

(viii) —OH;

(ix) C₁₋₄ alkoxy; and

(x) -L⁴-L⁵-Rⁱ;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₀ alkyl which is optionally substituted with 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₁₀ alkyl); (p) —C(═O)O(C₁₋₄ alkyl); (q) —C(═O)OH; (r) —C(═O)N(R′)(R″); and (s) -L¹-L²-R^{h};

R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene; -L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};

-L⁵- is selected from the group consisting of a bond and C₁₋₄ alkylene;

Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy; and

each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH2, NH(C₁₋₄ alkyl), N(C₁₋₄ alkyl)₂, C₁₋₄ alkyl, and C₁₋₄ haloalkyl;

or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

### Compound Provisions

In some embodiments, it is provided that the compound of Formula (I) is other than:

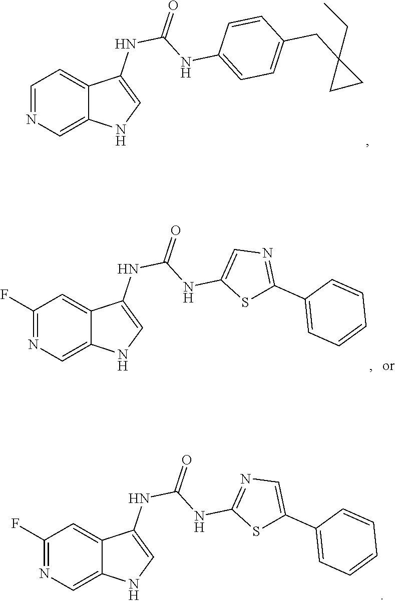 [SMILES: CCC1(Cc2ccc(NC(=O)Nc3c[nH]c4cnccc34)cc2)CC1 ; O=C(Nc1cnc(-c2ccccc2)s1)Nc1c[nH]c2cnc(F)cc12 ; O=C(Nc1ncc(-c2ccccc2)s1)Nc1c[nH]c2cnc(F)cc12]

In some embodiments, when R⁸ is (e) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected C₁₋₄ alkyl, then the heterocyclyl is other than tetrahydropyranyl (e.g., unsubstituted tetrahydropyranyl); and

when R⁸ is (c) C₃₋₆ cycloalkyl or C₃₋₆ cycloalkenyl, each of which is optionally substituted with 1-4 independently selected C₁₋₄ alkyl, then R⁸ is C₃ cycloalkyl, C₃ cycloalkenyl, C₅ cycloalkyl, or C₅ cycloalkenyl, each of which is optionally substituted with 1-4 independently selected C₁₋₄ alkyl.

In some embodiments, when P¹, P², P³, P⁴, and P⁵ are as defined according to (BB); one of P² and P⁵ is S; one of P³ and P⁴ is CR⁷, then R⁷ is other than unsubstituted phenyl.

In some embodiments, when P¹, P², P³, P⁴, and P⁵ are as defined according to (BB), and one of P² and P⁵ is S, then R⁷ is other than unsubstituted phenyl.

In some embodiments, when P¹, P², P³, P⁴, and P⁵ are as defined according to (AA); P³ is CR⁷, wherein R⁷ is L³-R⁹; and each of P¹, P², P⁴, and P⁵ is independently selected from the group consisting of CH and CR^{c}, then R⁹ is other than C₃ cycloalkyl substituted with 1-4 C₁₋₄ alkyl.

In some embodiments, when P¹, P², P³, P⁴, and P⁵ are as defined according to (AA); P³ is CR⁷, wherein R⁷ is L³-R⁹, then R⁹ is other than C₃ cycloalkyl substituted with 1-4 C₁₋₄ alkyl.

In some embodiments, when R⁷ is R⁸, then R⁸ is selected from the group consisting of:

(a) C₄₋₁₂ cycloalkyl or C₄₋₁₂ cycloalkenyl, each of which is substituted with 1-4 independently selected R^{7′};

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′};

(c) C₅ cycloalkyl or C5 cycloalkenyl, each of which is optionally substituted with 1-4 independently selected C₁₋₄ alkyl;

(d) C₇₋₁₂ cycloalkyl or C₇₋₁₂ cycloalkenyl, each of which is optionally substituted with 1-4 independently selected C₁₋₄ alkyl;

(e) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl or heterocycloalkenyl ring is optionally substituted with 1-4 independently selected C₁₋₄ alkyl;

(f) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(g) C₆₋₁₀ aryl optionally substituted with 1-4 independently selected R^{7′}.

In some embodiments, when R⁷ is L³-R⁹, then R⁹ is selected from the group consisting of:

(a) C₃₋₁₂ cycloalkyl or C₃₋₁₂ cycloalkenyl, each of which is optionally substituted with 1-4 independently selected R^{7′},

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′};

(d) C₆ aryl substituted with 1-4 independently selected R^{7′}; and

(e) C₈₋₁₀ aryl optionally substituted with 1-4 independently selected R^{7′}.

In some embodiments, the compound of Formula (I) is other than the compounds disclosed in International Patent Application No. PCT/US2019/040317 (published as WO 2020/010092), filed on Jul. 2, 2019, which is incorporated herein by reference in its entirety.

In some embodiments, the compound of Formula (I) is other than the compounds disclosed in U.S. patent application Ser. No. 16/460,381 (published as US 2020/0172534), filed on Jul. 2, 2019, which is incorporated herein by reference in its entirety.

In certain of these embodiments, the compound of Formula (I) is other than a compound selected from the group consisting of the following:

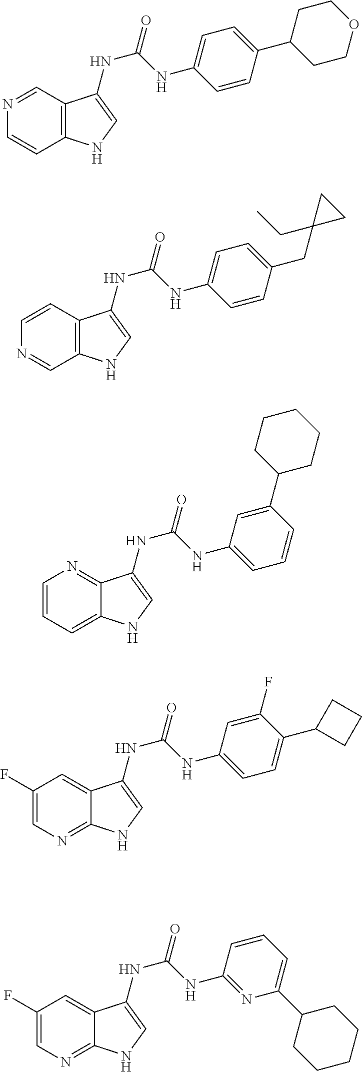 [SMILES: O=C(Nc1ccc(C2CCOCC2)cc1)Nc1c[nH]c2ccncc12 ; CCC1(Cc2ccc(NC(=O)Nc3c[nH]c4cnccc34)cc2)CC1 ; O=C(Nc1cccc(C2CCCCC2)c1)Nc1c[nH]c2cccnc12 ; O=C(Nc1ccc(C2CCC2)c(F)c1)Nc1c[nH]c2ncc(F)cc12 ; O=C(Nc1cccc(C2CCCCC2)n1)Nc1c[nH]c2ncc(F)cc12]

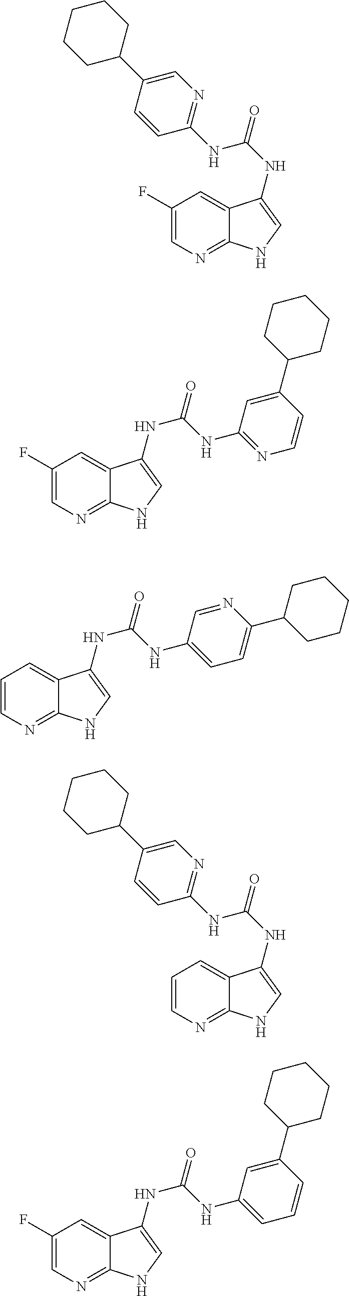 [SMILES: O=C(Nc1ccc(C2CCCCC2)cn1)Nc1c[nH]c2ncc(F)cc12 ; O=C(Nc1cc(C2CCCCC2)ccn1)Nc1c[nH]c2ncc(F)cc12 ; O=C(Nc1ccc(C2CCCCC2)nc1)Nc1c[nH]c2ncccc12 ; O=C(Nc1ccc(C2CCCCC2)cn1)Nc1c[nH]c2ncccc12 ; O=C(Nc1cccc(C2CCCCC2)c1)Nc1c[nH]c2ncc(F)cc12]

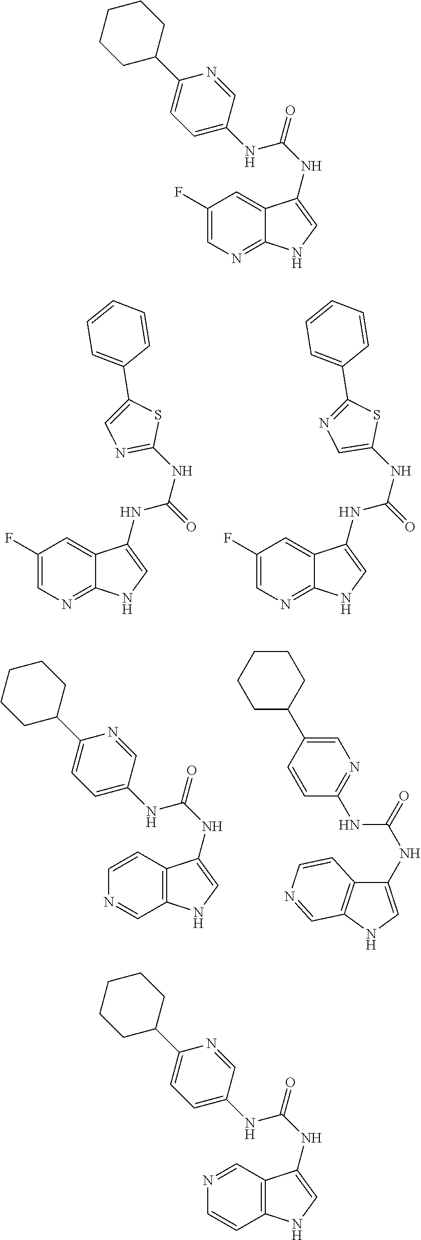

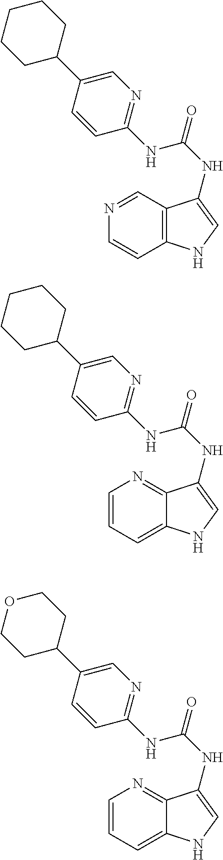 [SMILES: O=C(Nc1ccc(C2CCCCC2)cn1)Nc1c[nH]c2ccncc12 ; O=C(Nc1ccc(C2CCCCC2)cn1)Nc1c[nH]c2cccnc12 ; O=C(Nc1ccc(C2CCOCC2)cn1)Nc1c[nH]c2cccnc12]

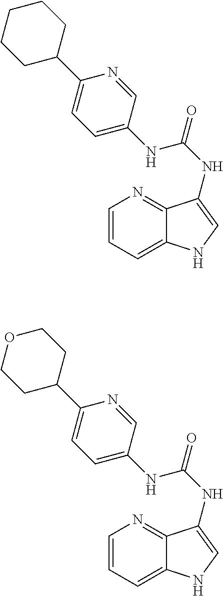 [SMILES: O=C(Nc1ccc(C2CCCCC2)nc1)Nc1c[nH]c2cccnc12 ; O=C(Nc1ccc(C2CCOCC2)nc1)Nc1c[nH]c2cccnc12]

In certain embodiments, the compound is other than a compound having a CAS Registry number selected from the group consisting of: 2408407-73-6; 2408407-81-6; 2408408-01-3; 2408409-07-2; 2408409-17-4; 2408409-18-5; 2408409-19-6; 2408409-20-9; 2408409-21-0; 2408409-28-7; 2408409-29-8; 2408409-49-2; 2408409-51-6; 2408409-67-4; 2408409-68-5; 2408409-70-9; 2408409-71-0; 2408409-81-2; 2408409-82-3; 2408409-86-7; and 2408409-88-9.

Embodiments can include any one or more of the features delineated below and/or in the claims.

The Variables P¹, P², P³, P⁴, and P⁵
Embodiments when P¹, P², P³, P⁴, and P⁵ are as Defined According to (AA)

In some embodiments, P¹, P², P³, P⁴, and P⁵ are as defined according to (AA).

In some embodiments, one or more (e.g., one) of P¹, P², P³, P⁴, and P⁵ is N. In certain embodiments, one of P¹, P², P³, P⁴, and P⁵ is N. In certain embodiments, two of P¹, P², P³, P⁴, and P⁵ are N.

In some embodiments, each one of P¹, P², P³, P⁴, and P⁵ is independently selected from the group consisting of CH, CR⁷, and, CR^{c}.

In some embodiments, one of P¹, P², P³, P⁴, and P⁵ is CR⁷.

In some embodiments, P³ is CR⁷. In certain embodiments (when one of P¹, P², P³, P⁴, and P⁵ is CR⁷), P³ is CR⁷.

In some embodiments, P⁴ is N. In certain embodiments (when one of P¹, P², P³, P⁴, and P⁵ is CR⁷; and P³ is CR⁷), P⁴ is N.

In some embodiments, each of P¹, P², and P⁵ is independently selected from the group consisting of CH and CR. In certain embodiments, 0-1 of P¹, P², and P⁵ is CR^{c}; and each remaining of P¹, P², and P⁵ is CH. As a non-limiting example, P² is CR^{c}; and P¹ and P⁵ are CH. As another non-limiting example, each of P¹, P² and P⁵ is CH.

In certain embodiments, P³ is CR⁷; P⁴ is N; and each of P¹, P², and P⁵ is independently selected from the group consisting of CH and CR^{c}. In certain of these embodiments, 0-1 of P¹, P², and P⁵ is CR^{c}; and each remaining of P¹, P², and P⁵ is CH. As a non-limiting example, P² is CR^{c}; and P¹ and P⁵ are CH. As another non-limiting example, each of P¹, P² and P⁵ is CH.

In certain embodiments, P³ is CR⁷; P⁴ is N; one of P¹, P², and P⁵ is N; and each remaining of P¹, P², and P⁵ is independently selected from the group consisting of CH and CR^{c}.

As a non-limiting example, P³ is CR⁷; P⁴ is N; P¹ is N; and each of P² and P⁵ is independently selected from the group consisting of CH and CR^{c}. As another non-limiting example, P³ is CR⁷; P⁴ is N; P⁵ is N; and each of P² and P¹ is independently selected from the group consisting of CH and CR^{c}.

In certain other embodiments, P³ is CR⁷; and each of P¹, P², P⁴ and P⁵ is independently selected from the group consisting of CH and CR^{c}. In certain of these embodiments, 0-1 of P¹, P², P⁴ and P⁵ is CR^{c}; and each remaining of P¹, P², P⁴ and P⁵ is CH.

In some embodiments, P¹ is N. In certain of these embodiments, P³ is CR⁷; and each of P², P⁴, and P⁵ is independently selected from the group consisting of CH and CR^{c}In certain other embodiments, P³ is CR⁷; one of P², P⁴, and P⁵ is N; and each remaining of P², P⁴, and P⁵ is independently selected from the group consisting of CH and CR^{c}.

In certain embodiments, P³ is CR⁷; P¹ is N; and each of P², P⁴, and P⁵ is independently selected from the group consisting of CH and CR^{c}.

In some embodiments, P⁴ is CR⁷. In certain embodiments (when P⁴ is CR⁷), each of P¹, P², P³, and P⁵ is independently selected from the group consisting of N, CH, and CR^{c}. In certain of these embodiments, each of P¹, P², P³, and P⁵ is independently selected from the group consisting of CH and CR^{c}. In certain other embodiments (when P⁴ is CR⁷) one of P¹, P², P³, and P⁵ is N; and each remaining of P¹, P², P³, and P⁵ is independently selected from the group consisting of CH and CR^{c}.

Embodiments when P¹, P², P³, P⁴, and P⁵ are as Defined According to (BB)

In some embodiments, P¹, P², P³, P⁴, and P⁵ are as defined according to (BB).

In some embodiments, one of P², P³, P⁴, and P⁵ is CR⁷ or NR⁷. For example, P³ is CR⁷ or NR⁷. In certain of these embodiments, each remaining P², P³, P⁴, and P⁵ is independently selected from the group consisting of: CH, CR^{c}, S, N, NH, and NR^{d}, provided that 1-3 of P², P³, P⁴, and P⁵ is S, N, NH, or NR^{d}.

In certain embodiments, P³ is CR⁷; and each of P², P⁴, and P⁵ is independently selected from the group consisting of: CH, CR^{c}, S, N, NH, and NR^{d}, provided that 1-2 (e.g., 2) of P², P⁴, and P⁵ is S, N, NH, or NR^{d}.

In certain embodiments, P³ is CR⁷; P² is NH, NR^{d}, or S (e.g., S); P⁵ is N; and P⁴ is CH or CR^{c} (e.g., CH).

In certain embodiments, P³ is CR⁷; P² is NH, NR^{d}, or S (e.g., S); P⁵ is CH or CR^{c}, and P⁴ is N.

In certain embodiments, P³ is NR⁷; P² is CH or CR^{c} (e.g., CH); P⁴ is N; and P⁵ is CH or CR^{c} (e.g., CH).

Non-Limiting Combinations of P¹, P², P³, P⁴, and P⁵

In some embodiments, the

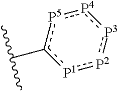

moiety has the formula:

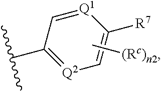

wherein Q¹ is N or CH; Q² is N or CH; and n2 is 0, 1, or 2.

In some embodiments,

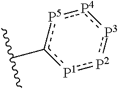

the moiety has the formula:

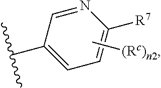

wherein n2 is 0, 1, or 2. In certain of these embodiments, the

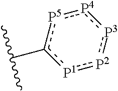

moiety has the formula:

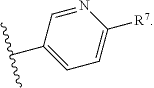

In certain other embodiments,

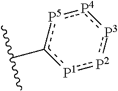

moiety has the formula:

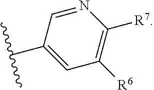

In some embodiments, the

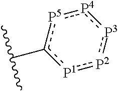

moiety has the formula:

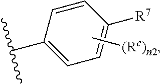

wherein n2 is 0, 1, or 2. In certain of these embodiments, the

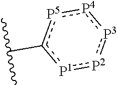

moiety has the formula:

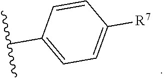

In certain other embodiments, the

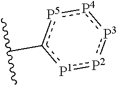

moiety has the formula:

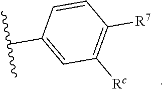

In some embodiments, the

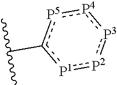

moiety has the formula:

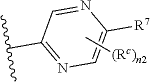

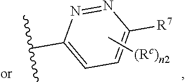

wherein n2 is 0, 1, or 2.

In some embodiments, the

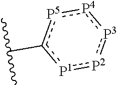

moiety has the formula:

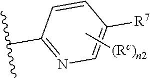

wherein n2 is 0, 1, or 2. In certain embodiments, the

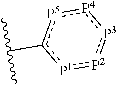

moiety has the formula:

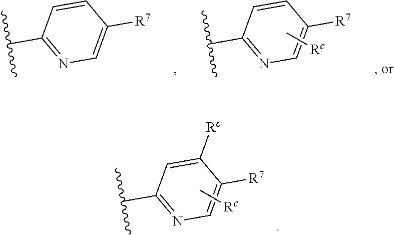

In some embodiments, the

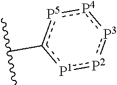

moiety has the formula:

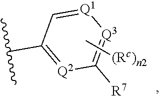

wherein each of Q¹, Q², and Q³ is independently N or CH; and n2 is 0, 1, or 2

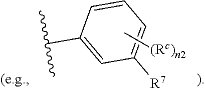

In some embodiments, the

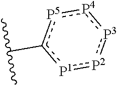

moiety has the formula:

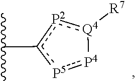

P⁵, wherein Q⁴ is N or C.

In some embodiments, the

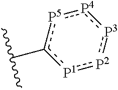

moiety has the formula:

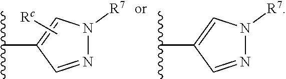

### The Variable R⁷

In some embodiments, R⁷ is R⁸. In certain embodiments P³ is CR⁷; and R⁷ is R⁸.

In certain embodiments (when R⁷ is R⁸), R⁸ is selected from the group consisting of:

(a) C₃₋₁₂ cycloalkyl or C₃₋₁₂ cycloalkenyl, each of which is substituted with 1-4 independently selected R^{7′}; and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

In certain of these embodiments, R⁸ is C₃₋₁₂ cycloalkyl or C₃₋₁₂ cycloalkenyl, each of which is substituted with 1-4 independently selected R^{7′}. In certain embodiments, R⁸ is C₄₋₁₀ cycloalkyl or C₄₋₁₀ cycloalkenyl, each of which is substituted with 1-4 independently selected R^{7′}. In certain embodiments, R⁸ is C₄₋₈ cycloalkyl or C₄₋₈ cycloalkenyl, each of which is substituted with 1-4 independently selected R^{7′}. In certain embodiments, R⁸ is C₄₋₈ cycloalkyl which is substituted with 1-4 independently selected R^{7′}, such as R⁸ is C₄₋₈ cycloalkyl which is substituted with 2-4 independently selected R^{7′}.

In certain embodiments, R⁸ is cyclohexyl which is substituted with 2-4 independently selected R^{7′}.

As a non-limiting example of the foregoing embodiments, R⁸ can be

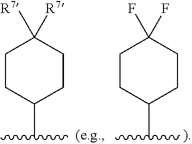

In certain embodiments, R⁸ is cyclobutyl which is substituted with 2-4 independently selected R^{7′}.

As a non-limiting example of the foregoing embodiments, R⁸ can be

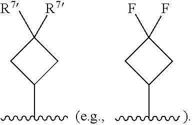

In certain embodiments, R⁸ is C₄₋₈ cycloalkyl which is substituted with 1-3 independently selected R^{7′}, such as R⁸ is C₄₋₈ cycloalkyl which is substituted with 1-2 (e.g., 1) independently selected R^{7′}.

In certain embodiments, R⁸ is cyclohexyl which is substituted with 1-2 (e.g., 1) independently selected R^{7′}. As a non-limiting example, R⁸ can be:

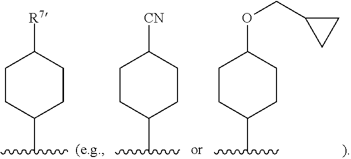

In certain embodiments, R⁸ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

In certain of these embodiments, R⁸ is heterocyclyl or heterocycloalkenyl of 4-10 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

In certain embodiments, R⁸ is heterocyclyl or heterocycloalkenyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

In certain of these embodiments, R⁸ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

In certain embodiments, R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 2-4 independently selected R^{7′}.

In certain of these embodiments, R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 2-4 (e.g., 2) independently selected R^{7′}.

In certain embodiments, R⁸ is selected from the group consisting of azetidinyl, pyrrolidinyl, and piperidinyl, each of which is substituted with 2-4 (e.g., 2) independently selected R^{7′}.

As a non-limiting example of the foregoing embodiments, R⁸ can be selected from the group consisting of:

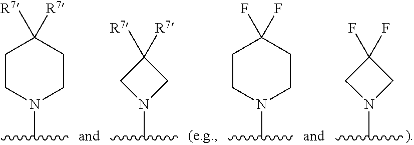

In certain embodiments, R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 1-3 (e.g., 1-2, e.g., 1) independently selected R^{7′}.

In certain of these embodiments, R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 1-3 (e.g., 1-2, e.g., 1) independently selected R^{7′}.

In certain embodiments, R⁸ is selected from the group consisting of azetidinyl, pyrrolidinyl, and piperidinyl, each of which is substituted with 1-3 (e.g., 1-2, e.g., 1) independently selected R^{7′}.

As a non-limiting example, R⁸ can be:

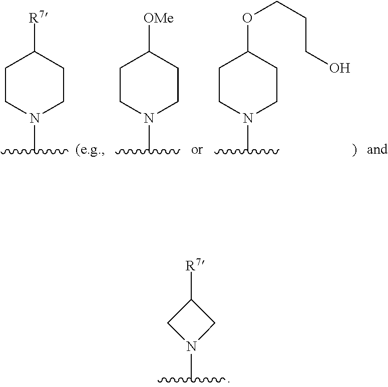

In certain embodiments, R⁸ is spirocyclic heterocyclyl of 6-12 (e.g., 6-10 (e.g., 7-10)) ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

As a non-limiting example of the foregoing embodiments, R⁸ can be 2-azaspiro[3.3]heptane, substituted with 1-4 independently selected R^{7′} at the ring carbon atoms. For example, R⁸ can be:

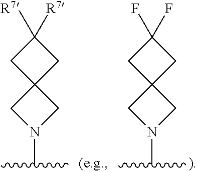

As a further non-limiting example, R⁸ can be 7-azaspiro[3.5]nonanyl which is substituted with 1-4 independently selected R^{7′} at the ring carbon atoms. For example, R⁸ can be

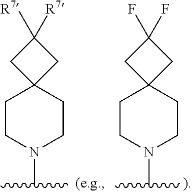

In certain embodiments, R⁸ is selected from the group consisting of:

(c) C₃ cycloalkyl, C3 cycloalkenyl, C₅ cycloalkyl, or C₅ cycloalkenyl, each of which is optionally substituted with 1-4 independently selected C₁₋₄ alkyl; and

(d) C₇₋₁₂ cycloalkyl or C₇₋₁₂ cycloalkenyl, each of which is optionally substituted with 1-4 independently selected C₁₋₄ alkyl.

In certain embodiments, R⁸ is selected from the group consisting of:

(c) C₃ cycloalkyl, or C₅ cycloalkyl, each of which is optionally substituted with 1-4 independently selected C₁₋₄ alkyl; and

(d) C₇₋₁₂ cycloalkyl which is optionally substituted with 1-4 independently selected C₁₋₄ alkyl.

In certain embodiments, R⁸ is C₃ cycloalkyl, or C₅ cycloalkyl, each of which is optionally substituted with 1-4 independently selected C₁₋₄ alkyl, such as unsubstituted C₃ or C₅ cycloalkyl. As a non-limiting example, R⁸ can be cyclopentyl.

In certain embodiments, R⁸ is C3 cycloalkyl, or C₅ cycloalkyl, each of which is substituted with 1-4 independently selected C₁₋₄ alkyl.

In some embodiments, R⁸ is C₇₋₁₂ bicyclic cycloalkyl which is unsubstituted.

In certain embodiments, R⁸ is C₇₋₁₂ spirobicyclic cycloalkyl which is unsubstituted. As a non-limiting example, R⁸ can be: R⁸ is

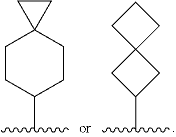 [SMILES: *C1CCC2(CC1)CC2 ; *C1CC2(CCC2)C1]

In some embodiments, R⁸ is C₇₋₁₂ bridged bicyclic cycloalkyl which is unsubstituted. As a non-limiting example, R⁸ can be R⁸ is

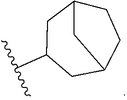 [SMILES: *C1CC2CCC(C1)C2]

In certain embodiments, R⁸ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl or heterocycloalkenyl ring is optionally substituted with 1-4 independently selected C₁₋₄ alkyl.

In certain embodiments, R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl ring is optionally substituted with 1-4 independently selected C₁₋₄ alkyl. In certain of these embodiments, R⁸ contains a ring N(R^{d}) group; and R^{d} is C₁₋₆ alkyl optionally substituted with 1-3 substituents each independently selected from the group consisting of halo, C₁₋₄ alkoxy, and OH. For example, R^{d} can be C₁₋₆ alkyl substituted with 1-3 independently selected halo.

In certain embodiments, R⁸ is selected from the group consisting of: azetidinyl, oxetanyl, pyrrolidinyl, tetrahydrofuranyl, piperidinyl, piperazinyl, morpholinyl, and azepinyl, each of which is optionally substituted with 1-4 independently selected C₁₋₄ alkyl.

In certain embodiments, R⁸ is azetidinyl, pyrrolidinyl, piperidinyl, piperazinyl, and morpholinyl, each of which is optionally substituted with 1-2 independently selected C₁₋₄ alkyl. For example, R⁸ is morpholinyl.

As anonther non-limiting example, R⁸ can be piperidinyl

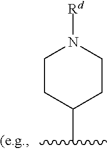

such as

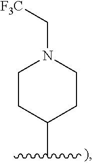 [SMILES: *C1CCN(CC(F)(F)F)CC1]

or oxepanyl, wherein the ring nitrogen atom of the piperidinyl is optionally substituted with R^{d}.

In certain embodiments, R⁸ is bicyclic or polycyclic heterocyclyl or heterocycloalkenyl of 7-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected C₁₋₄ alkyl.

In certain embodiments, R⁸ is bicyclic or polycyclic heterocyclyl of 7-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected C₁₋₄ alkyl. As a non-limiting example, R⁸ can be

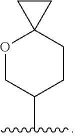 [SMILES: *C1CCC2(CC2)OC1]

In certain embodiments, R⁸ is spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected C₁₋₄ alkyl. As a non-limiting example of the foregoing embodiments, R⁸ can be

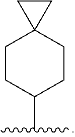 [SMILES: *C1CCC2(CC1)CC2]

In certain embodiments, R⁸ is heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}.

In certain embodiments, R⁸ is heteroaryl of 5-6 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R^{7′}.

In certain embodiments, R⁸ is bicyclic heteroaryl of 7-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R^{7′}. As a non-limiting example, R⁸ is

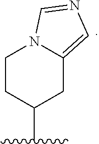 [SMILES: *C1CCn2cncc2C1]

In some embodiments, R⁷ is -L³-R⁹.

In certain embodiments, -L³ is —O—, —NH—, or —S—.

In certain embodiments, -L³ is —O—.

In certain embodiments, -L³ is —NH—.

In certain embodiments, -L³ is —S— or S(O)₁₋₂.

In certain embodiments, -L³ is selected from the group consisting of: C(═O)NH, NHC(═O), C(═O)O, OC(═O), C(═O), NHS(O)₂, and S(O)₂NH.

In certain embodiments, R⁹ is selected from the group consisting of:

(a) C₃₋₁₂ cycloalkyl or C₃₋₁₂ cycloalkenyl, each of which is optionally substituted with 1-4 independently selected R^{7′}, and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′}.

In certain embodiments, R⁹ is C₃₋₁₂ cycloalkyl or C₃₋₁₂ cycloalkenyl, each of which is optionally substituted with 1-4 independently selected R^{7′}.

In certain of these embodiments, R⁹ is C₄₋₈ cycloalkyl which is optionally substituted with 1-2 independently selected R^{7′}. As a non-limiting example of the foregoing embodiments, R⁹ is cyclobutyl, cyclopentyl, or cyclohexyl, each of which is optionally substituted with 1-2 independently selected R^{7′} (e.g., unsubstituted).

In certain embodiments, R⁹ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′}.

In certain embodiments, R⁹ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}. As a non-limiting example of the foregoing embodiments, R⁹ can be selected from the group consisting of azetidinyl, oxetanyl, pyrrolidinyl, tetrahydrofuranyl, piperidinyl, piperazinyl, morpholinyl, and azepinyl, each of which is optionally substituted with 1-2 independently selected R^{7′} (e.g., unsubstituted).

In certain embodiments, R⁷ is L³-R⁹; L³ is —O— or —NH—; and R⁹ is selected from the group consisting:

C₄₋₈ cycloalkyl which is optionally substituted with 1-2 independently selected R^{7′}; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}. In certain of these embodiments, L³ is —O—. In certain other embodiments, L³ is —NH—.

In certain of these embodiments, R⁷ is L³-R⁹; L³ is —O— or —NH—; and R⁹ is selected from the group consisting of cyclobutyl, cyclopentyl, cyclohexyl, and oxetanyl, each of which is optionally substituted with 1-2 independently selected R^{7′} (e.g., unsubstituted).

In certain of these embodiments, L³ is —O—. In certain other embodiments, L³ is —NH—.

When R⁷ is L³-R⁹, non-limiting examples of R⁷ can include:

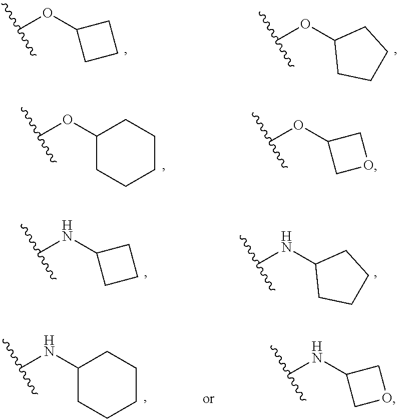

Further non-limiting examples of R⁷ can include:

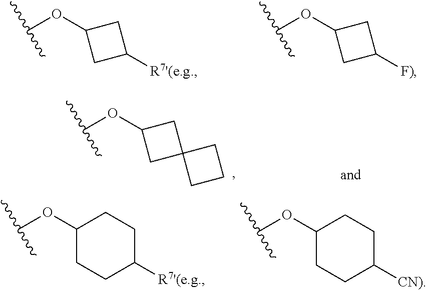

In certain embodiments, the

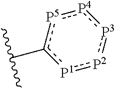

moiety has the formula:

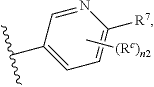

wherein n2 is 0, 1, or 2; and R⁷ is R⁸, wherein R⁸ is selected from the group consisting of:

C₄₋₈ cycloalkyl which is substituted with 1-4 independently selected R^{7′}; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

In certain of these embodiments, n2 is 0.

In certain other embodiments, n2 is 1. In certain of these embodiments, R^{c} is located ortho to R⁷.

In certain embodiments (when the

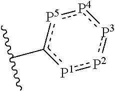

moiety has the formula:

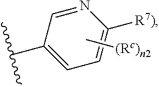

R⁷ is R⁸; and R⁸ is C₄₋₈ cycloalkyl which is substituted with 2-4 independently selected R^{7′}. As a non-limiting example of the foregoing embodiments, R⁸ can be cyclohexyl which is substituted with 2-4 independently selected R^{7′}, such as

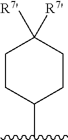

In certain embodiments (when the

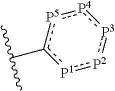

moiety has the formula:

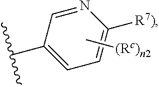

R⁷ is R⁸; and R⁸ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

In certain of these embodiments, R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 2-4 independently selected R^{7′}.

In certain of the foregoing embodiments, R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 2-4 (e.g., 2) independently selected R^{7′}.

As a non-limiting example of the foregoing embodiments, R⁸ can be selected from the group consisting of azetidinyl, pyrrolidinyl, and piperidinyl, each of which is substituted with 2-4 (e.g., 2) independently selected R^{7′}, such as

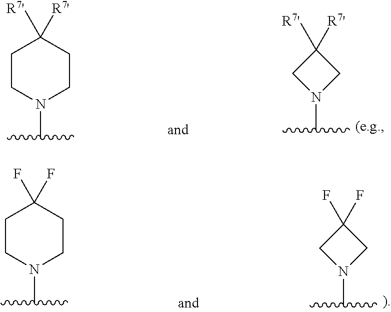

In certain embodiments (when R⁷ is R⁸), R⁷ is

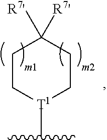

wherein m1 and m2 are independently 0, 1, or 2, and T¹ is CH or N. For example, m1 can be 1; and m2 can be 1. As another non-limiting example, m1 can be 0; and m2 can be 0. In certain embodiments, each R^{7′} is an independently selected halo, such as —F.

In certain embodiments (when R⁷ is R⁸), R⁷ is

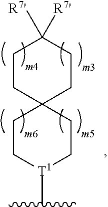

wherein m3, m4, m5, and m6 are independently 0 or 1; and T¹ is CH or N. For example, m1, m2, m3, and m4 can each be 0. As a non-limiting example, R⁷ can be

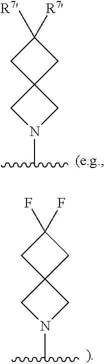

As another non-limiting example, R⁷ can be

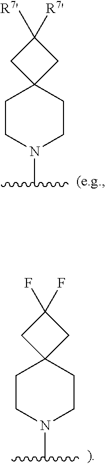

In certain embodiments, each R^{7′} is an independently selected halo, such as —F.

In certain embodiments, the

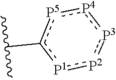

moiety has the formula:

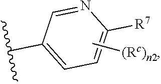

wherein n2 is 0, 1, or 2; and R⁷ is -L³-R⁹, wherein:

L³ is —NH— or —O—; and R⁹ is selected from the group consisting:

C₄₋₈ cycloalkyl which is optionally substituted with 1-2 independently selected R^{7′}; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

In certain of these embodiments, R⁷ is L³-R⁹; L³ is —O— or —NH—; and R⁹ is selected from the group consisting of cyclobutyl, cyclopentyl, cyclohexyl, and oxetanyl, each of which is optionally substituted with 1-2 independently selected R^{7′} (e.g., unsubstituted). In certain of the foregoing embodiments, L³ is —O—. In certain other embodiments, L³ is —NH—. By way of non-limiting examples, R⁷ can be

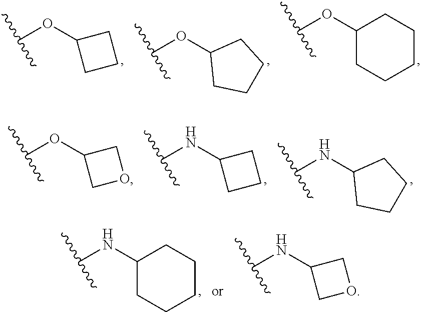

The Variable R^{7′}

In certain embodiments, one occurrence of R^{7′} is selected from the group consisting of: halo, —CN, —NO₂, —OH, —C₂₋₄ alkenyl, —C₂₋₄ alkynyl, —C₁₋₄ haloalkyl, —C₁₋₆ alkoxy, —C₁₋₆ haloalkoxy, S(O)₁₋₂(C₁₋₄ alkyl), —NR′R″, oxo, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″); and each remaining R^{7′} is independently selected from the group consisting of: halo, —CN, —NO₂, —OH, —C₁₋₄ alkyl, —C₂₋₄ alkenyl, —C₂₋₄ alkynyl, —C₁₋₄ haloalkyl, —C₁₋₆ alkoxy, —C₁₋₆ haloalkoxy, S(O)₁₋₂(C₁₋₄ alkyl), —NR′R″, oxo, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″), In certain embodiments, each R^{7′} when present is independently selected from the group consisting of: halo, —CN, —OH, —C₁₋₄ alkyl, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″), provided that when R⁷ is R⁸; and R⁸ is cycloalkyl, cycloalkenyl, heterocyclyl, or heterocycloalkenyl, then one or more occurrence of R^{7′} is other than —C₁₋₄ alkyl.

In certain embodiments, each R^{7′} when present is independently selected from the group consisting of: halo, —CN, —C₁₋₄ alkyl, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″), provided that when R⁷ is R⁸; and R⁸ is cycloalkyl, cycloalkenyl, heterocyclyl, or heterocycloalkenyl, then one or more occurrence of R^{7′} is other than —C₁₋₄ alkyl.

In certain embodiments, each R^{7′} when present is independently halo. As a non-limiting example, each R^{7′} when present can be —F.

In certain embodiments, each R^{c} when present is independently selected from the group consisting of: halo; cyano; C₁₋₁₀ alkyl which is optionally substituted with 1-6 independently selected R^{a}; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; and —C(═O)N(R′)(R″).

In certain embodiments, each R^{c} when present is independently selected from the group consisting of: halo; cyano; C₁₋₁₀ alkyl optionally substituted with 1-6 independently selected halo; C₁₋₄ alkoxy; C₁₋₄ haloalkoxy; —S(O)₁₋₂(C₁₋₄ alkyl); and —C(═O)(C₁₋₁₀ alkyl), such as wherein each R^{c} is independently halo (e.g., —F), C₁₋₄ alkyl (e.g., CH₃), or CF₃.

In certain embodiments, each R^{c} when present is halo (e.g., —F or —Cl). In certain embodiments, each R^{c} when present is CN. In certain embodiments, each R^{c} when present is C₁₋₄ alkoxy or C₁₋₄ haloalkoxy. In certain embodiments, each R^{c} when present is C₁₋₄ alkyl (e.g., CH₃).

The Variables Q and W

In some embodiments, Q is NH. In some embodiments, Q is N(C₁₋₃ alkyl).

In some embodiments, Q is *—NH—(C₁₋₃ alkylene)-, wherein the asterisk represents point of attachment to W.

In some embodiments, W is C(═O). In some embodiments, W is S(O)₁₋₂ (e.g., S(O)₂). In some embodiments, W is C(═S). In some embodiments, W is C(═NR^{d}) (e.g., C(═NH)). In some embodiments, W is C(═C—NO₂). In some embodiments, W is C(═N—CN).

In certain embodiments, Q is NH; and W is C(═O).

The Variables X, X², Z, Y¹, Y², and Y³

In some embodiments, X¹ is NR². In certain of these embodiments, X¹ is NH.

In some embodiments, X² is CR⁵. In certain of these embodiments, X² is CH.

In some embodiments, X¹ is NR²; and X² is CR⁵. In certain of these embodiments, X¹ is NH; and X² is CH.

In some embodiments, Z is CR¹.

In certain embodiments, 1-2 of Y¹, Y², and Y³ is independently N or NR² (e.g., N); and each of the remaining of Y¹, Y², and Y³ is an independently selected CR¹.

In certain embodiments, one of Y¹, Y², and Y³ is independently N or NR²; and each of the remaining of Y¹, Y², and Y³ is an independently selected CR¹.

In certain embodiments, one of Y¹, Y², and Y³ is independently N; and each of the remaining of Y¹, Y², and Y³ is an independently selected CR¹.

In certain embodiments, the

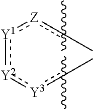

moiety is R

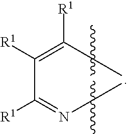

In certain embodiments, the

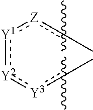

moiety is

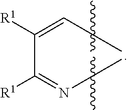

In certain embodiments, the

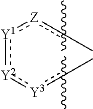

moiety is

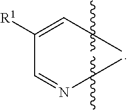

In certain embodiments, the

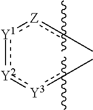

moiety is

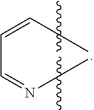 [SMILES: */C=C\C=N/*]

In certain embodiments, the

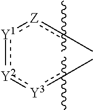

moiety is

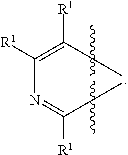

In certain embodiments, the

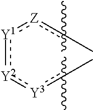

moiety is

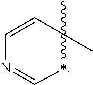

In certain embodiments, the

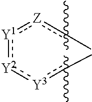

moiety is

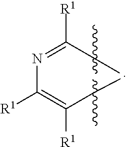

In certain embodiments, the

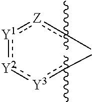

moiety is

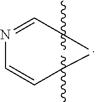 [SMILES: */C=C\N=C/*]

In some embodiments, Z is N.

In certain of these embodiments, each of Y¹, Y², and Y³ is an independently selected CR¹.

In certain embodiments, the

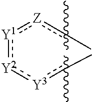

moiety is

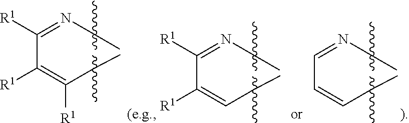

In some embodiments, the compound is selected from a compound of the following formulae:

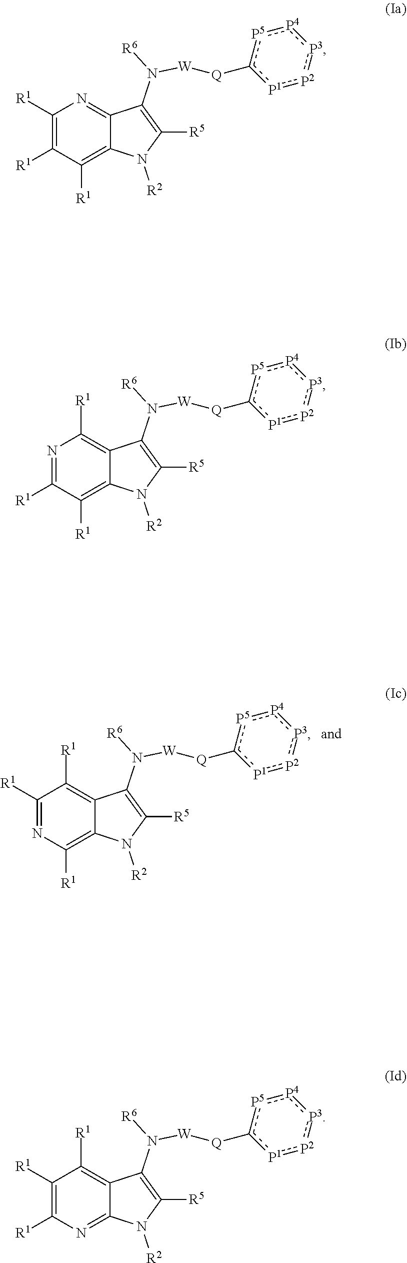

In certain embodiments, compound has formula (Ia):

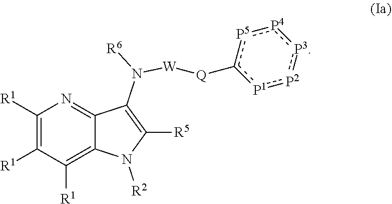

In certain embodiments, the compound has formula (Ia-1):

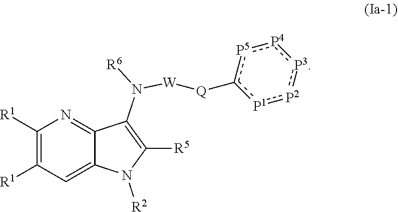

In certain embodiments, the compound has formula (Ia-2):

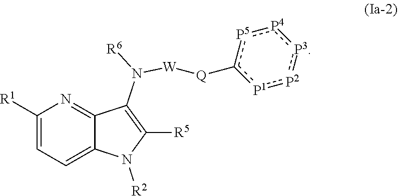

In certain embodiments, the compound has formula (Ia-3):

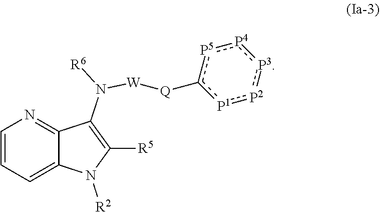

In certain embodiments, the compound has formula (I-b):

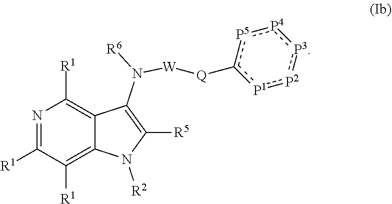

In certain embodiments, the compound has formula (Ib-1):

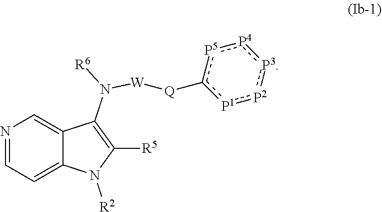

In certain embodiments, the compound has formula (I-c):

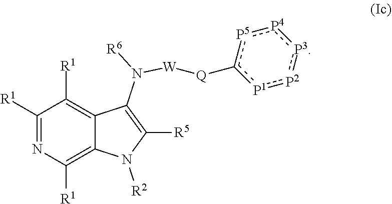

In certain embodiments, the compound has formula (Ic-1):

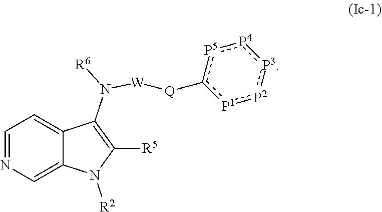

In certain embodiments, the compound has formula (I-d):

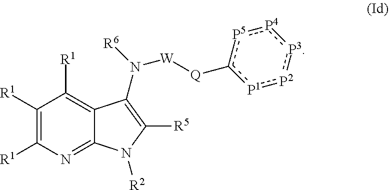

In certain embodiments, the compound has formula (Id-1):

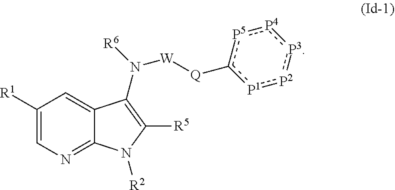

In certain embodiments, the compound has formula (Id-2):

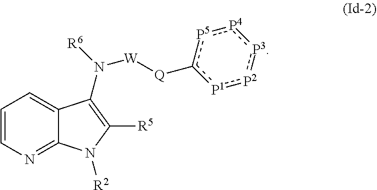

In certain embodiments, the compound has formula (Id-3):

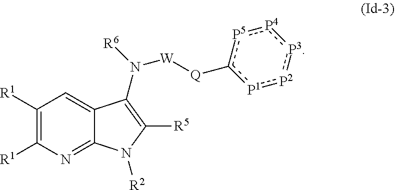

### The Variable R′

In some embodiments, each occurrence of R′ is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L¹-L²-R^{h}; —S(O)₁₋₂(C₁₋₄ alkyl); —S(O)(═NH)(C₁₋₄ alkyl); SF₅; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

In some embodiments, 0-2 (e.g., 0, 1, or 2) occurrences of R′ is other than H; and each of the remaining occurrences of R′ is H.

In certain embodiments, each occurrence of R¹ is H.

In certain other embodiments, 1-2 occurrences of R¹ is other than H. In certain of these embodiments, one occurrence of R¹ is other than H.

In certain embodiments, one occurrence of R¹ is selected from the group consisting of: halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)₁₋₂(NR′R″); —NO₂; -L-L²-R^{h}; —C(═O)(C₁₋₄ alkyl); —C(═O)O(C₁₋₄ alkyl); —C(═O)OH; and —C(═O)N(R′)(R″).

In certain embodiments, one occurrence of R¹ is halo (e.g., F or Cl (e.g., F)). In certain of these embodiments, each remaining R¹ is H. In certain other embodiments, one of the remaining R¹ is other than H (e.g., one of the remaining R¹ is halo (e.g., F)); and each of the other remaining R¹ is H.

In certain embodiments, one occurrence of R¹ is C₁₋₆ alkyl optionally substituted with 1-2 R^{a} (e.g., C₁₋₃ alkyl optionally substituted with 1-2 R^{a} (e.g., C₁₋₃ alkyl, such as methyl). In certain of these embodiments, each remaining R¹ is H. In certain other embodiments, one of the remaining R¹ is other than H (e.g., one of the remaining R¹ is halo (e.g., F)); and each of the other remaining R¹ is H.

In some embodiments, one occurrence of R¹ is selected from the group consisting of C₁₋₄ haloalkoxy (e.g., C₂₋₄ haloalkoxy); and C₁₋₄ alkoxy which is optionally substituted with —OH, C₁₋₄ alkoxy, C₁₋₄ haloalkoxy, or —NR^{e}R^{f}. For example, R¹ can be C₁₋₄ haloalkoxy (e.g., C₂₋₄ haloalkoxy). As another non-limiting example, R¹ can be C₁₋₄ alkoxy substituted with C₁₋₄ alkoxy (e.g., C₂₋₄ alkoxy substituted with C₁₋₄ alkoxy). As a further non-limiting example, R¹ can be C₁₋₄ alkoxy.

In certain embodiments, one occurrence of R¹ is -L¹-L²-R^{h}. In certain of these embodiments, -L¹ is a bond. In certain embodiments, R¹ is -L¹-L²-R^{h}; and -L⁴ is a bond. In certain embodiments, R¹ is -L¹-L²-R^{h}, L¹ is a bond; and L² is a bond.

In certain embodiments, R¹ is -L¹-L²-R^{h}, wherein —R^{h} is selected from the group consisting of:
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

In certain embodiments, —R^{h} is selected from the group consisting of:
heteroaryl of 5-6 ring atoms (e.g., pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy.

In certain embodiments, one of R′ is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy

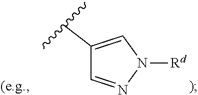

and
phenyl which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy

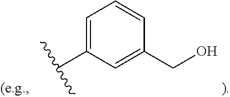 [SMILES: *c1cccc(CO)c1]

In certain of these embodiments, each remaining R¹ is H.

The Variable R²

In some embodiments, R² is H.

In some embodiments, R² is selected from the group consisting of:

(iii) —C(O)(C₁₋₆ alkyl) optionally substituted with 1-3 independently selected R^{a};

(iv) —C(O)O(C₁₋₄ alkyl) optionally substituted with 1-3 independently selected R^{a};

(v) —CON(R′)(R″);

(vi) —S(O)₁₋₂(NR′R″); and

(vii) —S(O)₁₋₂(C₁₋₄ alkyl) optionally substituted with 1-3 independently selected R^{a} In certain embodiments, R² is —C(O)(C₁₋₆ alkyl) optionally substituted with 1-3 independently selected R^{a}. In certain of these embodiments, each R^{a} substituent of R² is independently —F, —Cl, —OH, or —NR^{e}R^{f}.

As a non-limiting example, R² can be selected from the group consisting of: C(═O)Me,

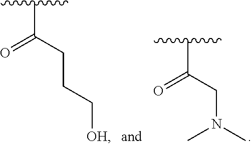

In certain embodiments, R² is —S(O)₁₋₂(C₁₋₄ alkyl) optionally substituted with 1-3 independently selected R^{a} (e.g., S(O)₂Me).

In some embodiments, R² is -L⁴-L⁵-Rⁱ. In certain of these embodiments, -L⁴ is a bond. In certain embodiments, -L⁴ is C(═O). In certain embodiments, R² is -L⁴-L⁵-Rⁱ, wherein -L⁴ is S(O)₂. In certain embodiments, R² is -L⁴-L⁵-Rⁱ, wherein -L⁵ is a bond. In certain other embodiments, -L⁵ is C₁₋₄ alkylene (e.g., C₁₋₂ alkylene).

In certain embodiments, R² is -L⁴-L⁵-Rⁱ, wherein Rⁱ is C₃₋₈ cycloalkyl, optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is

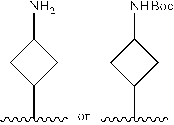 [SMILES: *C1CC(N)C1 ; *C1CC(NC(=O)OC(C)(C)C)C1]

wherein “Boc” represents tert-butoxycarbonyl).

In certain embodiments, R² is -L⁴-L⁵-Rⁱ, wherein Rⁱ is heterocyclyl, wherein the heterocyclyl has 3-8 ring atoms, wherein 1-3 ring atoms are heteroatoms, each independently selected from the group consisting of N, N(H), N(R^{d}), O, and S(O)₀₋₂, wherein the heterocyclyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., Rⁱ is

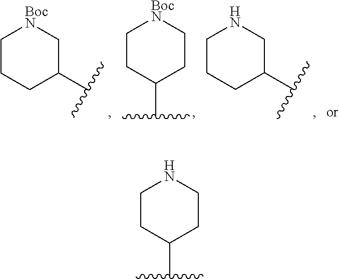

or wherein “Boc” represents tert-butoxycarbonyl).

In certain embodiments, R² is -L⁴-L⁵-Rⁱ, wherein Rⁱ is heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy).

In certain embodiments, R² is -L⁴-L⁵-Rⁱ, wherein Rⁱ is C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

In certain embodiments, R² is -L⁴-L⁵-Rⁱ; L⁴ is a bond; L⁵ is a bond or C₁₋₄ alkylene; and Rⁱ is C₃₋₈ cycloalkyl, optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g.,

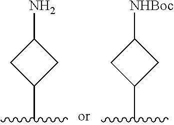 [SMILES: *C1CC(N)C1 ; *C1CC(NC(=O)OC(C)(C)C)C1]

wherein “Boc” represents tert-butoxycarbonyl).

In certain embodiments, R² is -L⁴-L⁵-Rⁱ; L⁴ is a bond; L⁵ is a bond or C₁₋₄ alkylene; and Rⁱ is heterocyclyl, wherein the heterocyclyl has 3-8 ring atoms, wherein 1-3 ring atoms are heteroatoms, each independently selected from the group consisting of N, N(H), N(R^{d}), O, and S(O)₀₋₂, wherein the heterocyclyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

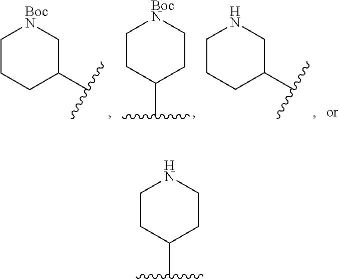

wherein “Boc” represents tert-butoxycarbonyl).

In certain embodiments, R² is -L⁴-L⁵-R; L⁴ is a bond; L⁵ is a bond or C₁₋₄ alkylene; and Rⁱ is heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy).

In certain embodiments, R² is -L⁴-L⁵-Rⁱ; L⁴ is a bond; L⁵ is a bond or C₁₋₄ alkylene; and Rⁱ is C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

In certain embodiments, R² is -L⁴-L⁵-R; L⁴ is C(═O) or S(O)₂; L⁵ is a bond or C₁₋₄ alkylene; and Rⁱ is heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy).

In certain embodiments, R² is -L⁴-L⁵-Rⁱ; L⁴ is C(═O) or S(O)₂; L⁵ is a bond or C₁₋₄ alkylene; and Rⁱ is C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄haloalkoxy).

As non-limiting examples, R² can be selected from the group consisting of:

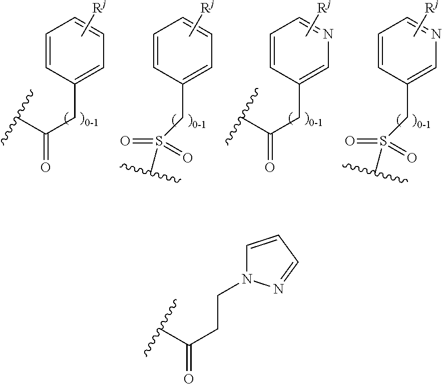

wherein R^{j} is H; halo; C₁₋₄ alkyl; C₁₋₄ haloalkyl; cyano; C₁₋₄ alkoxy; or C₁₋₄ haloalkoxy.

The Variable R⁵

In some embodiments, R⁵ is H.

The Variable R⁶

In some embodiments, R⁶ is H. In some embodiments, R⁶ is C₁₋₃ alkyl.

Non-Limiting Combinations

In some embodiments, the compound is a compound of Formula (I-1a):

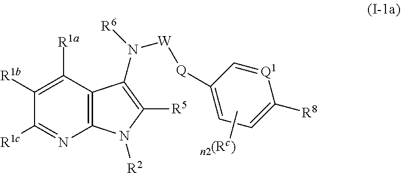

or a pharmaceutically acceptable salt thereof, wherein

each of R^{1a}, R^{1b}, and R^{1c} is an independently selected R¹;

### Q¹ is N or CH; and n2 is 0, 1, or 2.

In some embodiments, wherein the compound is a compound of Formula (I-1b):

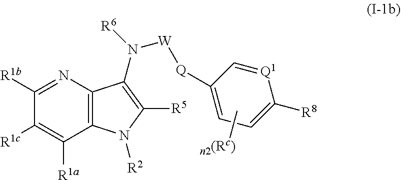

or a pharmaceutically acceptable salt thereof, wherein

each of R^{1a}, R^{1b}, and R^{1c} is an independently selected R¹;

Q¹ is N or CH; and n2 is 0, 1, or 2.

In certain embodiments of Formula (I-1a) or (I-1b), R⁸ is C₃₋₁₂ cycloalkyl or C₃₋₁₂ cycloalkenyl, each of which is substituted with 1-4 independently selected R^{7′}.

In certain embodiments of Formula (I-1a) or (I-1b), R⁸ is C₄₋₈ cycloalkyl which is substituted with 2-4 independently selected R^{7′}. In certain of these embodiments, R⁸ is cyclohexyl which is substituted with 2-4 independently selected R^{7′}. As a non-limiting example of the foregoing embodiments, R⁸ is

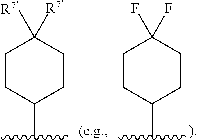

In certain embodiments of Formula (I-1a) or (I-1b), R⁸ is heterocyclyl or heterocycloalkenyl of 4-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

In certain of these embodiments, R⁸ is heterocyclyl of 4-8 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 2-4 independently selected R^{7′}.

In certain embodiments of Formula (I-1a) or (I-1b), R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 2-4 (e.g., 2) independently selected R^{7′}. In certain of these embodiments, R⁸ is selected from the group consisting of azetidinyl, pyrrolidinyl, and piperidinyl, each of which is substituted with 2-4 (e.g., 2) independently selected R^{7′}.

As non-limiting examples of the foregoing embodiments, R⁸ is selected from the group consisting of:

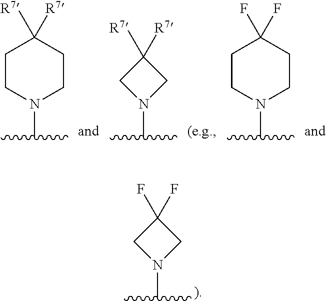

In certain embodiments of Formula (I-1a) or (I-1b), R⁸ is spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

As non-limiting examples of the foregoing embodiments, R⁸ can be selected from the group consisting of:

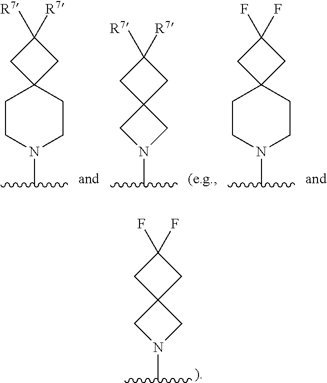

In certain embodiments of Formula (I-1a) or (I-1b), R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl ring is optionally substituted with 1-4 independently selected C₁₋₄ alkyl.

In certain embodiments of Formula (I-1a) or (I-1b), R⁸ is azetidinyl, oxetanyl, pyrrolidinyl, tetrahydrofuranyl, piperidinyl, piperazinyl, morpholinyl, and azepinyl, each of which is optionally substituted with 1-4 independently selected C₁₋₄ alkyl, such as R⁸ is morpholinyl.

In certain embodiments of Formula (I-1a) or (I-1b), R⁸ is selected from the group consisting of:

(c) C₃ cycloalkyl, or C₅ cycloalkyl, each of which is optionally substituted with 1-4 independently selected C₁₋₄ alkyl; and

(d) C₇₋₁₂ cycloalkyl which is optionally substituted with 1-4 independently selected C₁₋₄ alkyl.

As a non-limiting example, R⁸ can be unsubstituted C₃, C₅, or C₇₋₁₂ cycloalkyl (such as cyclopentyl).

In certain embodiments of Formula (I-1a) or (I-1b), R⁸ is selected from the group consisting of:

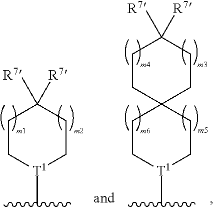

wherein:

T¹ is N or CH;

m1 and m2 are independently 0, 1, or 2; and

m3, m4, m5, and m6 are independently 0 or 1.

In certain of these embodiments, each R^{7′} is an independently selected halo (e.g., —F).

In certain embodiments of Formula (I-1a) or (I-1b) (when R⁸ is

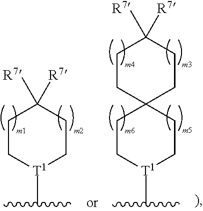

R⁸ is selected from the group consisting of:

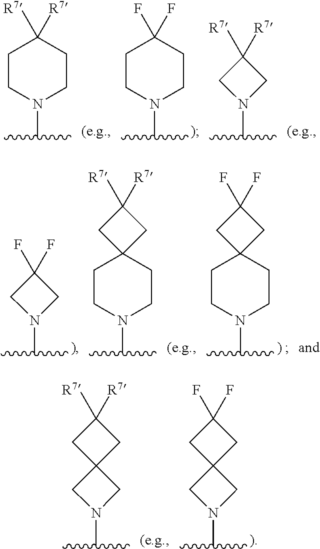

In some embodiments, the compound has Formula (I-2):

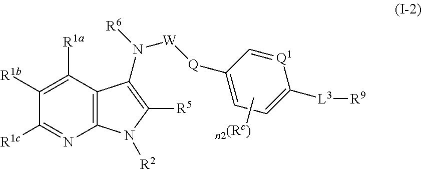

wherein each of R^{1a}, R^{1b}, and R^{1c} is an independently selected R¹;

Q¹ is N or CH; and n2 is 0, 1, or 2.

In certain embodiments of Formula (I-2), L³ is —O—.

In certain other embodiments, L³ is —NH—.

In certain embodiments of Formula (I-2), L³ is selected from the group consisting of: —S—, —S(O)₂—, C(═O)NH, NHC(═O), C(═O)O, OC(═O), C(═O), NHS(O)₂, and S(O)₂NH.

In certain embodiments of Formula (I-2), R⁹ is C₃₋₁₂ cycloalkyl or C₃₋₁₂ cycloalkenyl, each of which is optionally substituted with 1-4 independently selected R^{7′}.

In certain embodiments of Formula (I-2), R⁹ is C₄₋₈ cycloalkyl which is optionally substituted with 1-2 independently selected R^{7′}.

In certain embodiments of Formula (I-2), R⁹ is cyclobutyl, cyclopentyl, or cyclohexyl, each of which is optionally substituted with 1-2 independently selected R^{7′} (e.g., unsubstituted).

In certain embodiments of Formula (I-2), R⁹ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

In certain embodiments of Formula (I-2), R⁹ is selected from the group consisting of azetidinyl, oxetanyl, pyrrolidinyl, tetrahydrofuranyl, piperidinyl, piperazinyl, morpholinyl, and azepinyl, each of which is optionally substituted with 1-2 independently selected R^{7′} (e.g., unsubstituted).

As non-limiting examples, -L³-R⁹ in Formula (I-2) can be selected from the group consisting of:

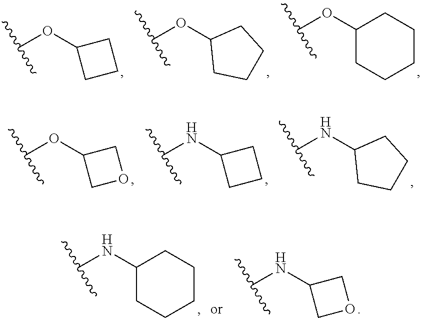

In certain embodiments of Formula (I-1a), (I-1b), or (I-2), each R^{7′} when present is independently selected from the group consisting of: halo, —CN, —C₁₋₄ alkyl, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″), provided that when R^{7′} is a substituent of R⁸; and R⁸ is cycloalkyl, cycloalkenyl, heterocyclyl, or heterocycloalkenyl, then one or more occurrence of R^{7′} is other than —C₁₋₄ alkyl.

In certain embodiments of Formula (I-1a), (I-1b), or (I-2), each R^{7′} when present is independently selected from the group consisting of: halo, —CN, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″), provided that when R^{7′} is a substituent of R⁸; and R⁸ is cycloalkyl, cycloalkenyl, heterocyclyl, or heterocycloalkenyl, then one or more occurrence of R^{7′} is other than —C₁₋₄ alkyl.

In certain embodiments of Formula (I-1a), (I-1b), or (I-2), each R^{7′} when present is independently selected from the group consisting of halo, —CN, —C₁₋₄ alkyl, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″).

In certain embodiments of Formula (I-1a), (I-1b), or (I-2), each R^{7′} when present is independently halo. As a non-limiting example, each R^{7′} when present can be —F.

In certain embodiments of Formula (I-1a), (I-1b), or (I-2), n2 is 0.

In certain embodiments of Formula (I-1a), (I-1b), or (I-2), n2 is 1.

In certain of these embodiments, R^{c} when present is independently selected from the group consisting of: (a) halo; (b) cyano; (c) C₁₋₁₀ alkyl; (g) C₁₋₄ alkoxy; (h) C₁₋₄ haloalkoxy; (i) —S(O)₁₋₂(C₁₋₄ alkyl); and —C(═O)(C₁₋₁₀ alkyl).

In certain embodiments of Formula (I-1a), (I-1b), or (I-2), each R^{c} when present is halo (e.g., —F, —Br, or —Cl) or cyano.

In certain embodiments of Formula (I-1a), (I-1b), or (I-2), each R^{c} when present is C₁₋₃ alkyl (e.g., methyl or ethyl).

In certain embodiments of Formula (I-1a), (I-1b), or (I-2), Q¹ is N.

In certain embodiments of Formula (I-1a), (I-1b), or (I-2), Q¹ is CH.

In certain embodiments of Formula (I-1a), (I-1b), or (I-2), Q is NH.

In certain embodiments of Formula (I-1a), (I-1b), or (I-2), W is C(═O).

In certain embodiments of Formula (I-1a), (I-1b), or (I-2), Q is NH; and W is C(═O).

In certain embodiments of Formula (I-1a), (I-1b), or (I-2), W is S(O)₂, C(═S), or C(═NR^{d}).

In certain embodiments of Formula (I-1a), (I-1b), or (I-2), each one of R¹, Rⁱ, and R^{1c} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy optionally substituted with C₁₋₄ alkoxy; C₁₋₄ haloalkoxy; -L¹-L²-R^{h}; —S(O)₁₋₂(C₁₋₄ alkyl); —S(O)(═NH)(C₁₋₄ alkyl); SF₅; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

In certain embodiments of Formula (I-1a), (I-1b), or (I-2), each one of R^{1a}, R^{1b}, and R^{1c} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L¹-L²-R^{h}; —S(O)₁₋₂(C₁₋₄ alkyl); —S(O)(═NH)(C₁₋₄ alkyl); SF₅; —S(O)₁₋₂(NR′R′); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

In certain embodiments of Formula (I-1a), (I-1b), or (I-2), R^{1a} is H.

In certain embodiments of Formula (I-1a), (I-1b), or (I-2), R^{1c} is H.

In certain embodiments of Formula (I-1a), (I-1b), or (I-2), R^{1b} is H.

In certain embodiments of Formula (I-1a), (I-1b), or (I-2), R^{1b} is other than H. In certain of these embodiments, R^{1b} is halo. As a non-limiting example of the foregoing embodiments, R^{1b} can be —F. As another non-limiting example of the foregoing embodiments, R^{1b} can be —Br or —Cl. In certain embodiments of Formula (I-1a), (I-1b), or (I-2), R^{1b} is C₁₋₃ alkoxy such as methoxy. In certain embodiments of Formula (I-1a), (I-1b), or (I-2), R^{1b} is C₁₋₄ alkoxy (e.g., C₂₋₄ alkoxy) substituted with C₁₋₄ alkoxy. In certain embodiments of Formula (I-1a), (I-1b), or (I-2), R^{1b} is C₁₋₄ haloalkoxy (e.g., C₂₋₄ haloalkoxy).

In certain embodiments of Formula (I-1a), (I-1b), or (I-2), R^{1b} is L¹-L²-R^{h}. In certain of these embodiments, -L¹ is a bond. In certain embodiments of Formula (I-1a), (I-1b), or (I-2), R^{1b} is L¹-L²-R^{h}, wherein -L² is a bond.

In certain embodiments of Formula (I-1a), (I-1b), or (I-2), R^{1b} is L¹-L²-R^{h}, wherein —R^{h} is selected from the group consisting of:
heteroaryl of 5-6 ring atoms (e.g., pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
phenyl which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

In certain embodiments of Formula (I-1a), (I-1b), or (I-2), R^{1b} is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy

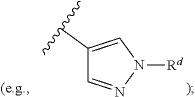

and
phenyl which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy

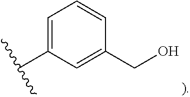 [SMILES: *c1cccc(CO)c1]

In certain embodiments of Formula (I-1a), (I-1b), or (I-2), R⁵ is H.

In certain embodiments of Formula (I-1a), (I-1b), or (I-2), R² is H.

In certain embodiments of Formula (I-1a), (I-1b), or (I-2), R² is —C(O)(C₁₋₆ alkyl) optionally substituted with 1-3 independently selected R^{a}; or —S(O)₁₋₂(C₁₋₄ alkyl) optionally substituted with 1-3 independently selected R^{a} (e.g., S(O)₂Me).

As a non-limiting example, R² can be selected from the group consisting of:

C(═O)Me, S(O)₂Me,

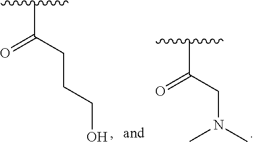

In certain embodiments of Formula (I-1a), (I-1b), or (I-2), R² is -L⁴-L⁵-R; L⁴ is a bond; L⁵ is a bond or C₁₋₄ alkylene (e.g., CH₂); and Rⁱ is selected from the group consisting of:

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

In certain embodiments of Formula (I-1a), (I-1b), or (I-2), R² is -L⁴-L⁵-Rⁱ; L⁴ is C(═O) or S(O)₂; L⁵ is a bond or C₁₋₄ alkylene; and Rⁱ is selected from the group consisting of:

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

As non-limiting examples, R² in Formula (I-1a), (I-1b), or (I-2) can be selected from the group consisting of:

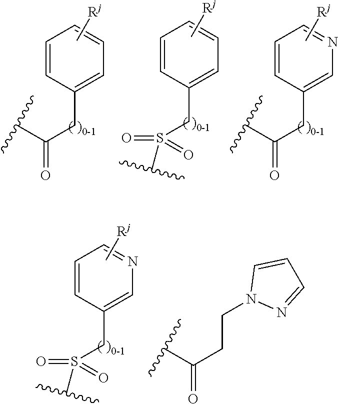

wherein R^{j} is H; halo; C₁₋₄ alkyl; C₁₋₄ haloalkyl; cyano; C₁₋₄ alkoxy; or C₁₋₄ haloalkoxy.

In certain embodiments of Formula (I-1a), (I-1b), or (I-2), R⁶ is hydrogen.

In some embodiments, the compound of Formula (I) is a compound of Formula (I-3a):

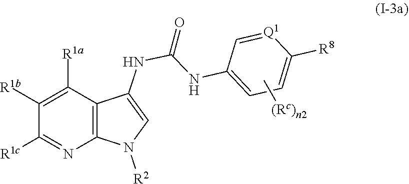

or a pharmaceutically acceptable salt thereof, wherein:

each of R^{1a}, R^{1b}, and R^{1c} is independently selected from the group consisting of: H; halo; cyano; C₁₋₆ alkyl optionally substituted with 1-2 R^{a}; C₁₋₄ haloalkyl; C₁₋₄ alkoxy optionally substituted with C₁₋₄ alkoxy (e.g., C₁₋₄ alkoxy); and C₁₋₄ haloalkoxy;

Q¹ is N or CH;

R⁸ is selected from the group consisting of:

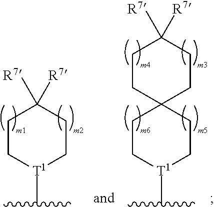

n2 is 0, 1, or 2;

each R^{c} when present is independently selected from the group consisting of: halo, cyano, C₁₋₃ alkyl, and C₁₋₃ alkoxy;

m1 and m2 are independently 0, 1, or 2; m3, m4, m5, and m6 are independently 0 or 1; and

T¹ is CH or N.

In some embodiments, the compound of Formula (I) is a compound of Formula (I-3b):

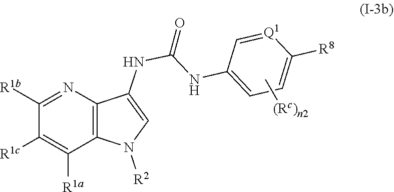

or a pharmaceutically acceptable salt thereof, wherein:

each of R^{a}, R^{1b}, and R^{1c} is independently selected from the group consisting of:

H; halo; cyano; C₁₋₆ alkyl optionally substituted with 1-2 R^{a}; C₁₋₄ haloalkyl; C₁₋₄ alkoxy optionally substituted with C₁₋₄ alkoxy (e.g., C₁₋₄ alkoxy); and C₁₋₄ haloalkoxy;

Q¹ is N or CH;

R⁸ is selected from the group consisting of:

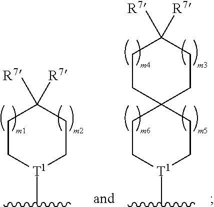

n2 is 0, 1, or 2;

each R^{c} when present is independently selected from the group consisting of: halo, cyano, C₁₋₃ alkyl, and C₁₋₃ alkoxy;

m1 and m2 are independently 0, 1, or 2; m3, m4, m5, and m6 are independently 0 or 1; and

T¹ is CH or N.

In certain embodiments of Formula (I-3a) or (I-3b), R² is H.

In certain embodiments of Formula (I-3a) or (I-3b), R^{1a} and R^{1c} are H.

In certain embodiments of Formula (I-3a) or (I-3b), R^{1b} is H.

In certain other embodiments, R^{1b} is other than H. For example, R^{1b} can be halo such as F. As another non-limiting example, R^{1b} can be C₁₋₃ alkoxy, such as methoxy. As another non-limiting example, R^{1b} can be C₁₋₄ haloalkoxy (e.g., C₂₋₄ haloalkoxy). As a further non-limiting example, R^{1b} can be C₁₋₄ alkoxy substituted with C₁₋₄ alkoxy (e.g., C₂₋₄ alkoxy substituted with C₁₋₄ alkoxy).

In certain embodiments of Formula (I-3a) or (I-3b), each R^{7′} is halo such as —F.

In certain embodiments of Formula (I-3a) or (I-3b), R⁸ is

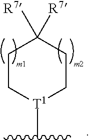

In certain of these embodiments, m1=m2=1. In certain other embodiments, m1=m2=0.

In certain embodiments of Formula (I-3a) or (I-3b), T¹ is N. In certain other embodiments, T¹ is CH.

In certain embodiments of Formula (I-3a) or (I-3b), n2 is 1. In certain of these embodiments, R^{c} is ortho to R⁸. In certain of the foregoing embodiments, R^{c} is halo, such as —Cl. In certain embodiments, R^{c} is C₁₋₃ alkyl, such as methyl or ethyl. In certain embodiments, R^{c} is cyano.

In certain embodiments of Formula (I-3a) or (I-3b), Q¹ is N. In certain other embodiments, Q¹ is CH.

In certain embodiments of Formula (I-3a) or (I-3b), each R^{7′} when present is independently halo.

Non-Limiting Exemplary Formula I Compounds In certain embodiments, the compound is selected from the group consisting of the compound delineated in Table C1, or a pharmaceutically acceptable salt thereof.

TABLE C1

| Com- | |
|---|---|
| pound | |
| No. | Structure |
| 101 | 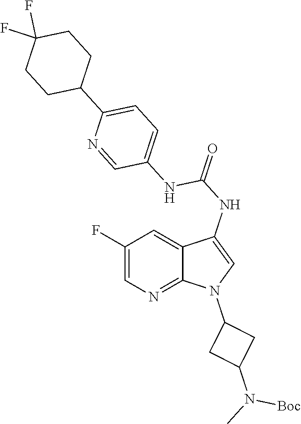 [SMILES: CN(C(=O)OC(C)(C)C)C1CC(n2cc(NC(=O)Nc3ccc(C4CCC(F)(F)CC4)nc3)c3cc(F)cnc32)C1] |
| 102 | 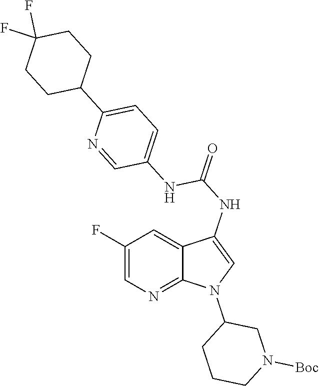 [SMILES: CC(C)(C)OC(=O)N1CCCC(n2cc(NC(=O)Nc3ccc(C4CCC(F)(F)CC4)nc3)c3cc(F)cnc32)C1] |
| 103 | 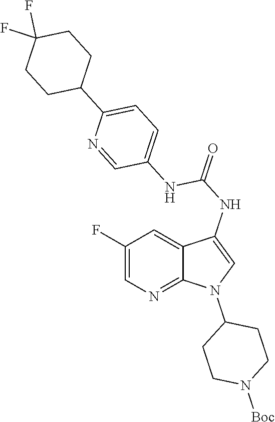 [SMILES: CC(C)(C)OC(=O)N1CCC(n2cc(NC(=O)Nc3ccc(C4CCC(F)(F)CC4)nc3)c3cc(F)cnc32)CC1] |
| 104 | 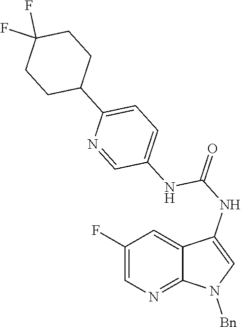 [SMILES: O=C(Nc1ccc(C2CCC(F)(F)CC2)nc1)Nc1cn(Cc2ccccc2)c2ncc(F)cc12] |
| 105 | 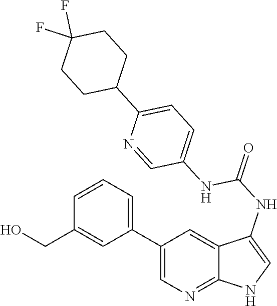 [SMILES: O=C(Nc1ccc(C2CCC(F)(F)CC2)nc1)Nc1c[nH]c2ncc(-c3cccc(CO)c3)cc12] |
| 106 | 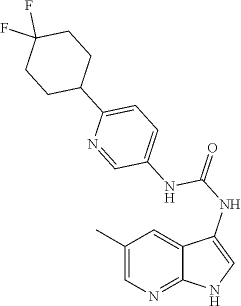 [SMILES: Cc1cnc2[nH]cc(NC(=O)Nc3ccc(C4CCC(F)(F)CC4)nc3)c2c1] |
| 107 | 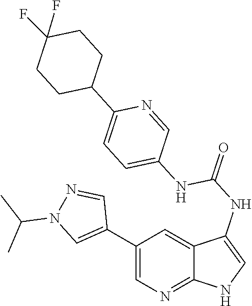 [SMILES: CC(C)n1cc(-c2cnc3[nH]cc(NC(=O)Nc4ccc(C5CCC(F)(F)CC5)nc4)c3c2)cn1] |
| 108 | 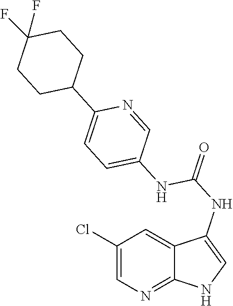 [SMILES: O=C(Nc1ccc(C2CCC(F)(F)CC2)nc1)Nc1c[nH]c2ncc(Cl)cc12] |
| 109 | 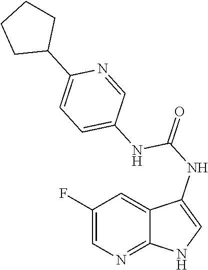 [SMILES: O=C(Nc1ccc(C2CCCC2)nc1)Nc1c[nH]c2ncc(F)cc12] |
| 110 | 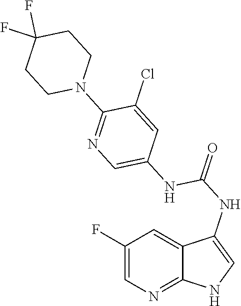 [SMILES: O=C(Nc1cnc(N2CCC(F)(F)CC2)c(Cl)c1)Nc1c[nH]c2ncc(F)cc12] |
| 111 | 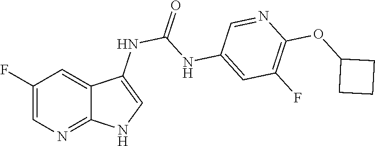 [SMILES: O=C(Nc1cnc(OC2CCC2)c(F)c1)Nc1c[nH]c2ncc(F)cc12] |
| 112 | 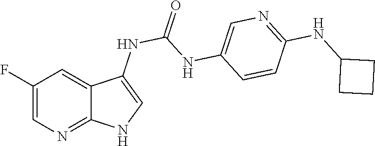 [SMILES: O=C(Nc1ccc(NC2CCC2)nc1)Nc1c[nH]c2ncc(F)cc12] |
| 113 | 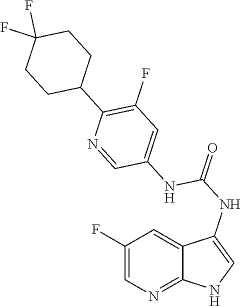 [SMILES: O=C(Nc1cnc(C2CCC(F)(F)CC2)c(F)c1)Nc1c[nH]c2ncc(F)cc12] |
| 114 | 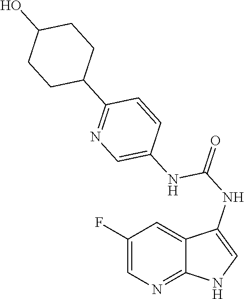 [SMILES: O=C(Nc1ccc(C2CCC(O)CC2)nc1)Nc1c[nH]c2ncc(F)cc12] |
| 115 | 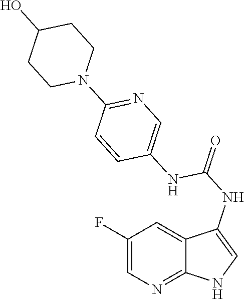 [SMILES: O=C(Nc1ccc(N2CCC(O)CC2)nc1)Nc1c[nH]c2ncc(F)cc12] |
| 116 | 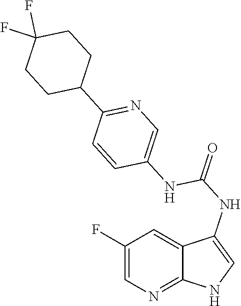 [SMILES: O=C(Nc1ccc(C2CCC(F)(F)CC2)nc1)Nc1c[nH]c2ncc(F)cc12] |
| 117 | 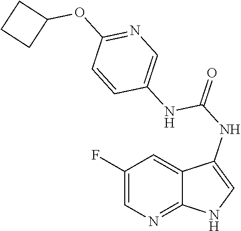 [SMILES: O=C(Nc1ccc(OC2CCC2)nc1)Nc1c[nH]c2ncc(F)cc12] |
| 118 | 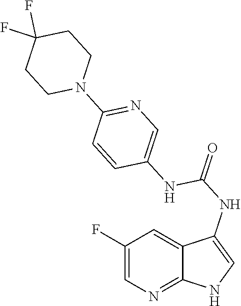 [SMILES: O=C(Nc1ccc(N2CCC(F)(F)CC2)nc1)Nc1c[nH]c2ncc(F)cc12] |
| 119 | 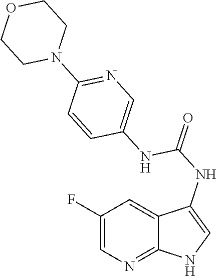 [SMILES: O=C(Nc1ccc(N2CCOCC2)nc1)Nc1c[nH]c2ncc(F)cc12] |
| 120 | 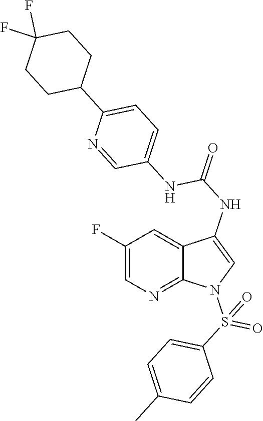 [SMILES: Cc1ccc(S(=O)(=O)n2cc(NC(=O)Nc3ccc(C4CCC(F)(F)CC4)nc3)c3cc(F)cnc32)cc1] |
| 121 | 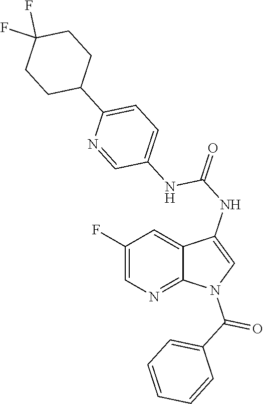 [SMILES: O=C(Nc1ccc(C2CCC(F)(F)CC2)nc1)Nc1cn(C(=O)c2ccccc2)c2ncc(F)cc12] |
| 122 | 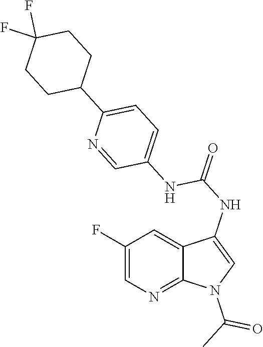 [SMILES: CC(=O)n1cc(NC(=O)Nc2ccc(C3CCC(F)(F)CC3)nc2)c2cc(F)cnc21] |
| 123 | 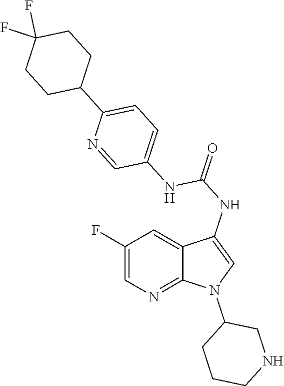 [SMILES: O=C(Nc1ccc(C2CCC(F)(F)CC2)nc1)Nc1cn(C2CCCNC2)c2ncc(F)cc12] |
| 124 | 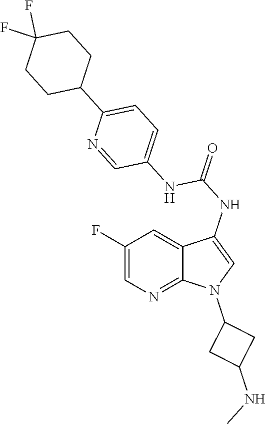 [SMILES: CNC1CC(n2cc(NC(=O)Nc3ccc(C4CCC(F)(F)CC4)nc3)c3cc(F)cnc32)C1] |
| 125 | 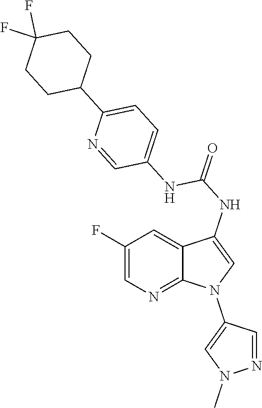 [SMILES: Cn1cc(-n2cc(NC(=O)Nc3ccc(C4CCC(F)(F)CC4)nc3)c3cc(F)cnc32)cn1] |
| 127 | 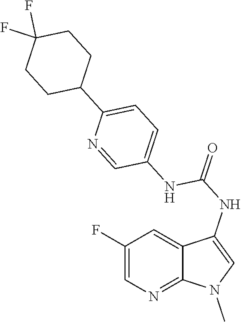 [SMILES: Cn1cc(NC(=O)Nc2ccc(C3CCC(F)(F)CC3)nc2)c2cc(F)cnc21] |
| 128 | 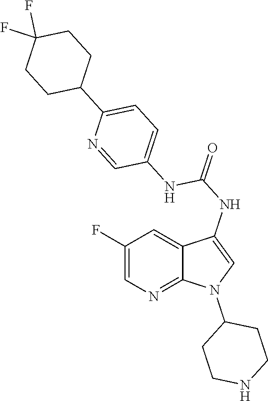 [SMILES: O=C(Nc1ccc(C2CCC(F)(F)CC2)nc1)Nc1cn(C2CCNCC2)c2ncc(F)cc12] |
| 129 | 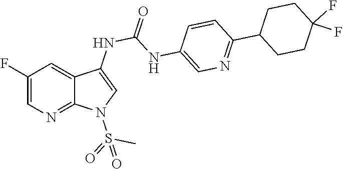 [SMILES: CS(=O)(=O)n1cc(NC(=O)Nc2ccc(C3CCC(F)(F)CC3)nc2)c2cc(F)cnc21] |
| 130 | 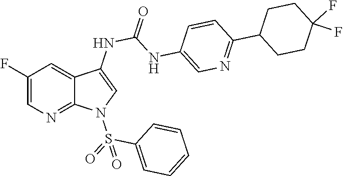 [SMILES: O=C(Nc1ccc(C2CCC(F)(F)CC2)nc1)Nc1cn(S(=O)(=O)c2ccccc2)c2ncc(F)cc12] |
| 131 | 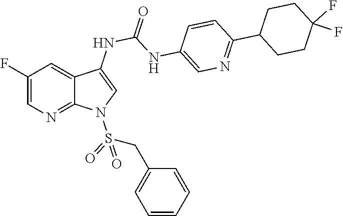 [SMILES: O=C(Nc1ccc(C2CCC(F)(F)CC2)nc1)Nc1cn(S(=O)(=O)Cc2ccccc2)c2ncc(F)cc12] |
| 132 | 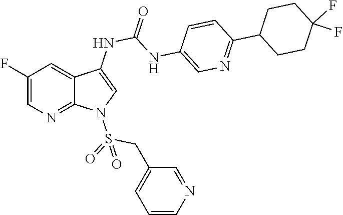 [SMILES: O=C(Nc1ccc(C2CCC(F)(F)CC2)nc1)Nc1cn(S(=O)(=O)Cc2cccnc2)c2ncc(F)cc12] |
| 133 | 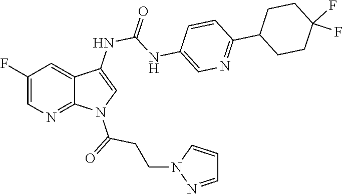 [SMILES: O=C(Nc1ccc(C2CCC(F)(F)CC2)nc1)Nc1cn(C(=O)CCn2cccn2)c2ncc(F)cc12] |
| 134 | 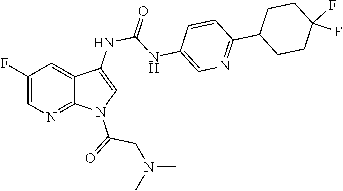 [SMILES: CN(C)CC(=O)n1cc(NC(=O)Nc2ccc(C3CCC(F)(F)CC3)nc2)c2cc(F)cnc21] |
| 135 | 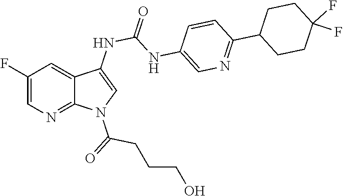 [SMILES: O=C(Nc1ccc(C2CCC(F)(F)CC2)nc1)Nc1cn(C(=O)CCCO)c2ncc(F)cc12] |
| 136 | 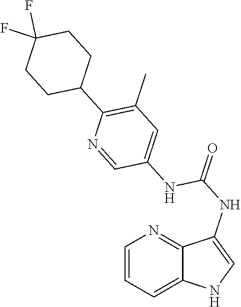 [SMILES: Cc1cc(NC(=O)Nc2c[nH]c3cccnc23)cnc1C1CCC(F)(F)CC1] |
| 137 | 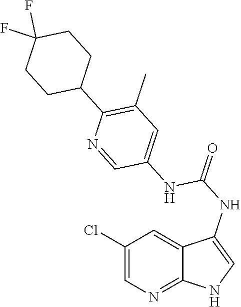 [SMILES: Cc1cc(NC(=O)Nc2c[nH]c3ncc(Cl)cc23)cnc1C1CCC(F)(F)CC1] |
| 138 | 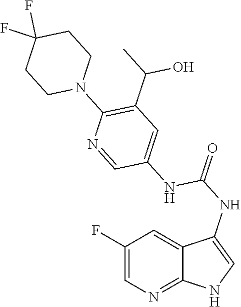 [SMILES: CC(O)c1cc(NC(=O)Nc2c[nH]c3ncc(F)cc23)cnc1N1CCC(F)(F)CC1] |
| 139 | 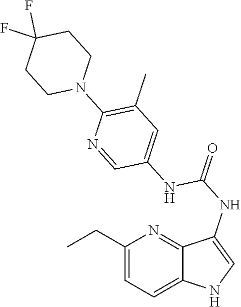 [SMILES: CCc1ccc2[nH]cc(NC(=O)Nc3cnc(N4CCC(F)(F)CC4)c(C)c3)c2n1] |
| 140 | 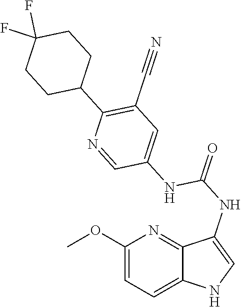 [SMILES: COc1ccc2[nH]cc(NC(=O)Nc3cnc(C4CCC(F)(F)CC4)c(C#N)c3)c2n1] |
| 141 | 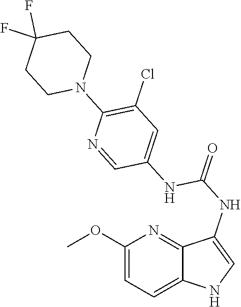 [SMILES: COc1ccc2[nH]cc(NC(=O)Nc3cnc(N4CCC(F)(F)CC4)c(Cl)c3)c2n1] |
| 142 | 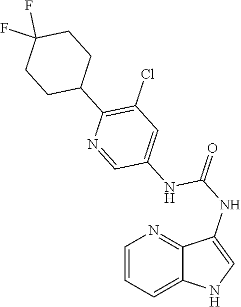 [SMILES: O=C(Nc1cnc(C2CCC(F)(F)CC2)c(Cl)c1)Nc1c[nH]c2cccnc12] |
| 143 | 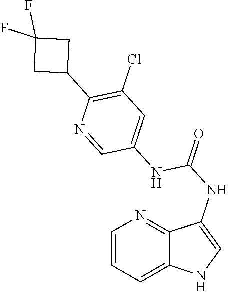 [SMILES: O=C(Nc1cnc(C2CC(F)(F)C2)c(Cl)c1)Nc1c[nH]c2cccnc12] |
| 144 | 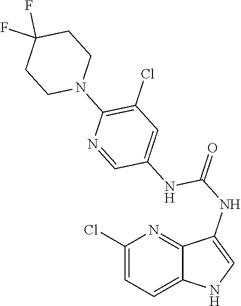 [SMILES: O=C(Nc1cnc(N2CCC(F)(F)CC2)c(Cl)c1)Nc1c[nH]c2ccc(Cl)nc12] |
| 145 | 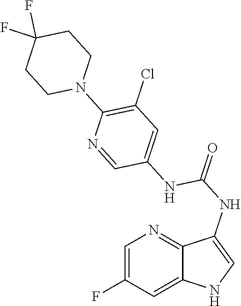 [SMILES: O=C(Nc1cnc(N2CCC(F)(F)CC2)c(Cl)c1)Nc1c[nH]c2cc(F)cnc12] |
| 146 | 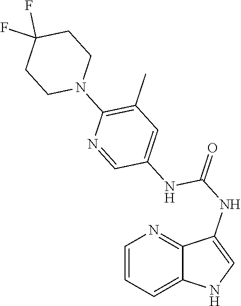 [SMILES: Cc1cc(NC(=O)Nc2c[nH]c3cccnc23)cnc1N1CCC(F)(F)CC1] |
| 147 | 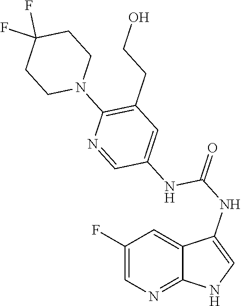 [SMILES: O=C(Nc1cnc(N2CCC(F)(F)CC2)c(CCO)c1)Nc1c[nH]c2ncc(F)cc12] |
| 148 | 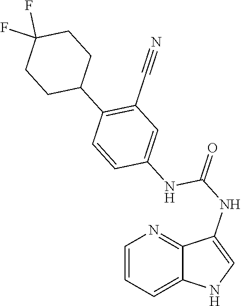 [SMILES: N#Cc1cc(NC(=O)Nc2c[nH]c3cccnc23)ccc1C1CCC(F)(F)CC1] |
| 149 | 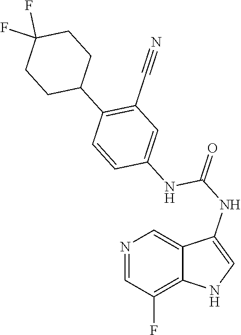 [SMILES: N#Cc1cc(NC(=O)Nc2c[nH]c3c(F)cncc23)ccc1C1CCC(F)(F)CC1] |
| 150 | 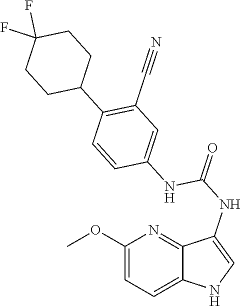 [SMILES: COc1ccc2[nH]cc(NC(=O)Nc3ccc(C4CCC(F)(F)CC4)c(C#N)c3)c2n1] |
| 151 | 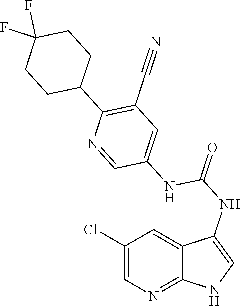 [SMILES: N#Cc1cc(NC(=O)Nc2c[nH]c3ncc(Cl)cc23)cnc1C1CCC(F)(F)CC1] |
| 152 | 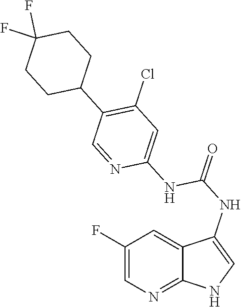 [SMILES: O=C(Nc1cc(Cl)c(C2CCC(F)(F)CC2)cn1)Nc1c[nH]c2ncc(F)cc12] |
| 154 | 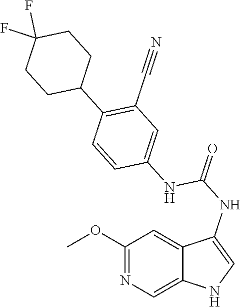 [SMILES: COc1cc2c(NC(=O)Nc3ccc(C4CCC(F)(F)CC4)c(C#N)c3)c[nH]c2cn1] |
| 155 | 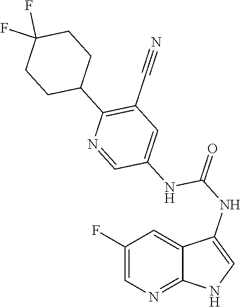 [SMILES: N#Cc1cc(NC(=O)Nc2c[nH]c3ncc(F)cc23)cnc1C1CCC(F)(F)CC1] |
| 156 | 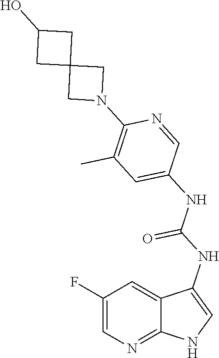 [SMILES: Cc1cc(NC(=O)Nc2c[nH]c3ncc(F)cc23)cnc1N1CC2(CC(O)C2)C1] |
| 157 | 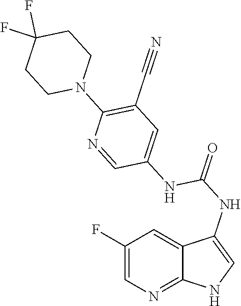 [SMILES: N#Cc1cc(NC(=O)Nc2c[nH]c3ncc(F)cc23)cnc1N1CCC(F)(F)CC1] |
| 158 | 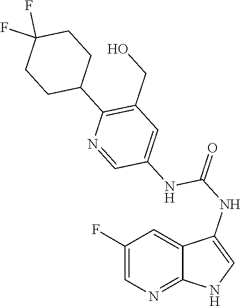 [SMILES: O=C(Nc1cnc(C2CCC(F)(F)CC2)c(CO)c1)Nc1c[nH]c2ncc(F)cc12] |
| 159 | 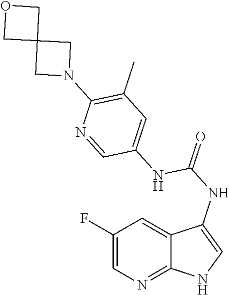 [SMILES: Cc1cc(NC(=O)Nc2c[nH]c3ncc(F)cc23)cnc1N1CC2(COC2)C1] |
| 160 | 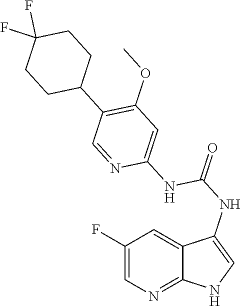 [SMILES: COc1cc(NC(=O)Nc2c[nH]c3ncc(F)cc23)ncc1C1CCC(F)(F)CC1] |
| 161 | 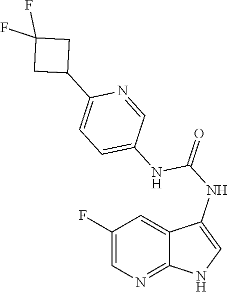 [SMILES: O=C(Nc1ccc(C2CC(F)(F)C2)nc1)Nc1c[nH]c2ncc(F)cc12] |
| 162 | 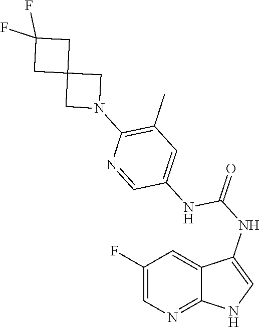 [SMILES: Cc1cc(NC(=O)Nc2c[nH]c3ncc(F)cc23)cnc1N1CC2(C1)CC(F)(F)C2] |
| 163 | 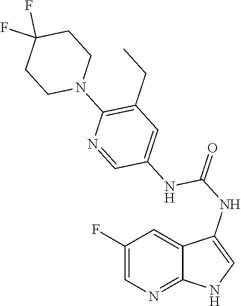 [SMILES: CCc1cc(NC(=O)Nc2c[nH]c3ncc(F)cc23)cnc1N1CCC(F)(F)CC1] |
| 164 | 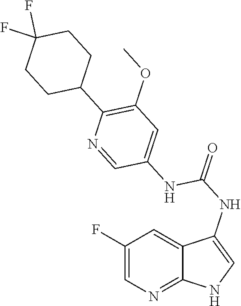 [SMILES: COc1cc(NC(=O)Nc2c[nH]c3ncc(F)cc23)cnc1C1CCC(F)(F)CC1] |
| 165 | 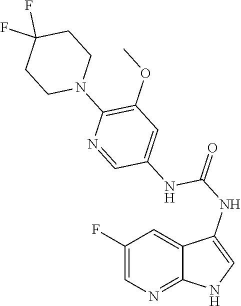 [SMILES: COc1cc(NC(=O)Nc2c[nH]c3ncc(F)cc23)cnc1N1CCC(F)(F)CC1] |
| 166 | 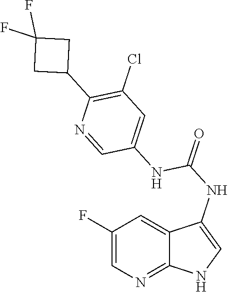 [SMILES: O=C(Nc1cnc(C2CC(F)(F)C2)c(Cl)c1)Nc1c[nH]c2ncc(F)cc12] |
| 167 | 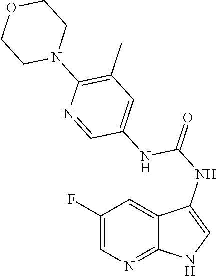 [SMILES: Cc1cc(NC(=O)Nc2c[nH]c3ncc(F)cc23)cnc1N1CCOCC1] |
| 168 | 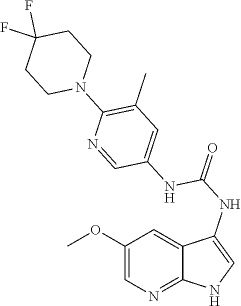 [SMILES: COc1cnc2[nH]cc(NC(=O)Nc3cnc(N4CCC(F)(F)CC4)c(C)c3)c2c1] |
| 169 | 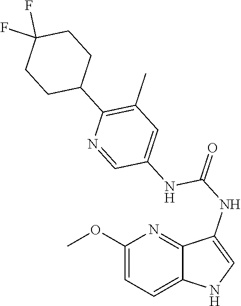 [SMILES: COc1ccc2[nH]cc(NC(=O)Nc3cnc(C4CCC(F)(F)CC4)c(C)c3)c2n1] |
| 170 | 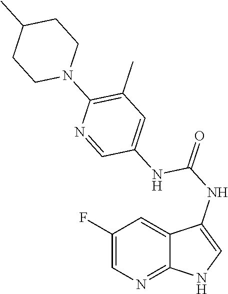 [SMILES: Cc1cc(NC(=O)Nc2c[nH]c3ncc(F)cc23)cnc1N1CCC(C)CC1] |
| 171 | 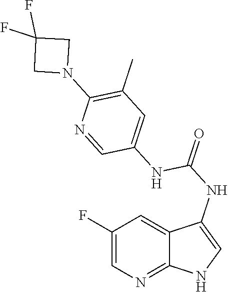 [SMILES: Cc1cc(NC(=O)Nc2c[nH]c3ncc(F)cc23)cnc1N1CC(F)(F)C1] |
| 172 | 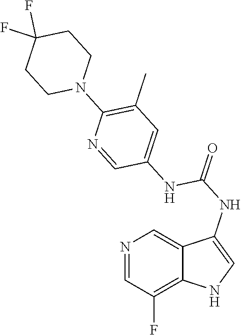 [SMILES: Cc1cc(NC(=O)Nc2c[nH]c3c(F)cncc23)cnc1N1CCC(F)(F)CC1] |
| 173 | 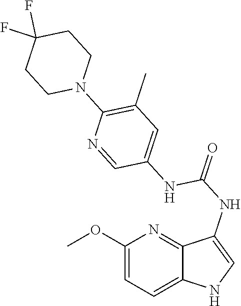 [SMILES: COc1ccc2[nH]cc(NC(=O)Nc3cnc(N4CCC(F)(F)CC4)c(C)c3)c2n1] |
| 174 | 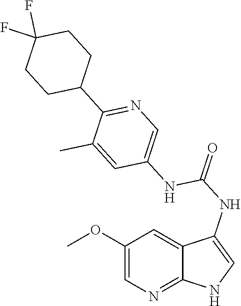 [SMILES: COc1cnc2[nH]cc(NC(=O)Nc3cnc(C4CCC(F)(F)CC4)c(C)c3)c2c1] |
| 175 | 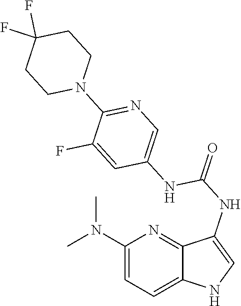 [SMILES: CN(C)c1ccc2[nH]cc(NC(=O)Nc3cnc(N4CCC(F)(F)CC4)c(F)c3)c2n1] |
| 177 | 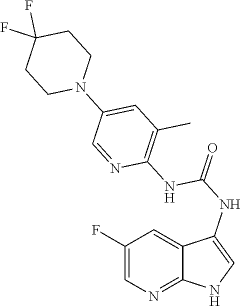 [SMILES: Cc1cc(N2CCC(F)(F)CC2)cnc1NC(=O)Nc1c[nH]c2ncc(F)cc12] |
| 178 | 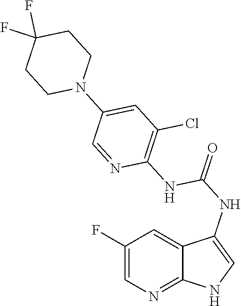 [SMILES: O=C(Nc1ncc(N2CCC(F)(F)CC2)cc1Cl)Nc1c[nH]c2ncc(F)cc12] |
| 179 | 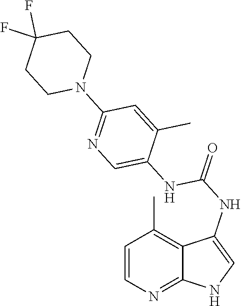 [SMILES: Cc1cc(N2CCC(F)(F)CC2)ncc1NC(=O)Nc1c[nH]c2nccc(C)c12] |
| 180 | 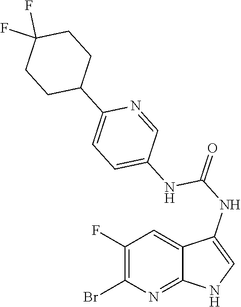 [SMILES: O=C(Nc1ccc(C2CCC(F)(F)CC2)nc1)Nc1c[nH]c2nc(Br)c(F)cc12] |
| 181 | 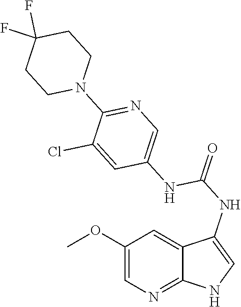 [SMILES: COc1cnc2[nH]cc(NC(=O)Nc3cnc(N4CCC(F)(F)CC4)c(Cl)c3)c2c1] |
| 182 | 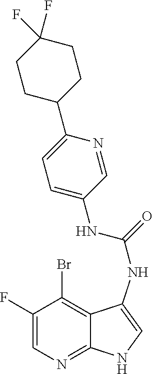 [SMILES: O=C(Nc1ccc(C2CCC(F)(F)CC2)nc1)Nc1c[nH]c2ncc(F)c(Br)c12] |
| 183 | 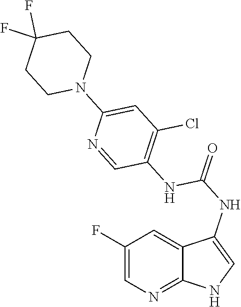 [SMILES: O=C(Nc1cnc(N2CCC(F)(F)CC2)cc1Cl)Nc1c[nH]c2ncc(F)cc12] |
| 184 | 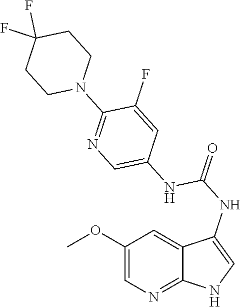 [SMILES: COc1cnc2[nH]cc(NC(=O)Nc3cnc(N4CCC(F)(F)CC4)c(F)c3)c2c1] |
| 185 | 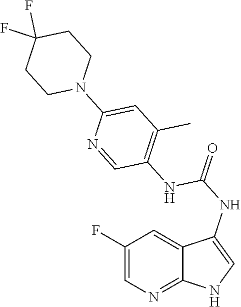 [SMILES: Cc1cc(N2CCC(F)(F)CC2)ncc1NC(=O)Nc1c[nH]c2ncc(F)cc12] |
| 186 | 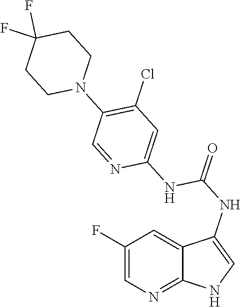 [SMILES: O=C(Nc1cc(Cl)c(N2CCC(F)(F)CC2)cn1)Nc1c[nH]c2ncc(F)cc12] |
| 187 | 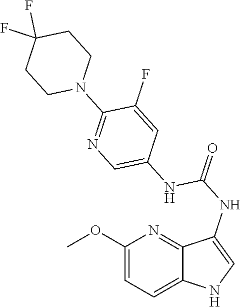 [SMILES: COc1ccc2[nH]cc(NC(=O)Nc3cnc(N4CCC(F)(F)CC4)c(F)c3)c2n1] |
| 188 | 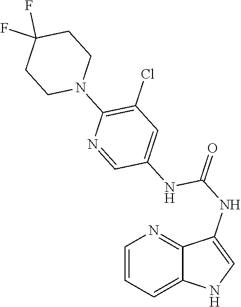 [SMILES: O=C(Nc1cnc(N2CCC(F)(F)CC2)c(Cl)c1)Nc1c[nH]c2cccnc12] |
| 189 | 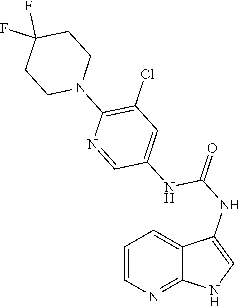 [SMILES: O=C(Nc1cnc(N2CCC(F)(F)CC2)c(Cl)c1)Nc1c[nH]c2ncccc12] |
| 190 | 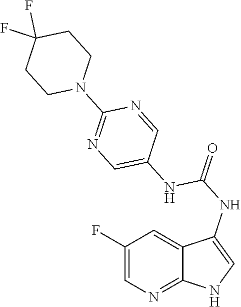 [SMILES: O=C(Nc1cnc(N2CCC(F)(F)CC2)nc1)Nc1c[nH]c2ncc(F)cc12] |
| 191 | 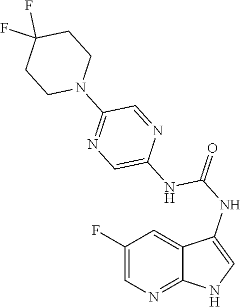 [SMILES: O=C(Nc1cnc(N2CCC(F)(F)CC2)cn1)Nc1c[nH]c2ncc(F)cc12] |
| 192 | 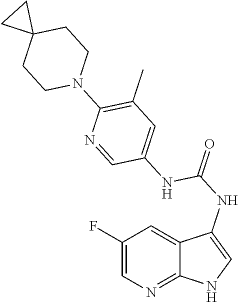 [SMILES: Cc1cc(NC(=O)Nc2c[nH]c3ncc(F)cc23)cnc1N1CCC2(CC1)CC2] |
| 193 | 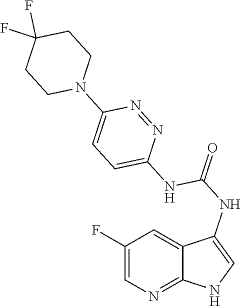 [SMILES: O=C(Nc1ccc(N2CCC(F)(F)CC2)nn1)Nc1c[nH]c2ncc(F)cc12] |
| 194 | 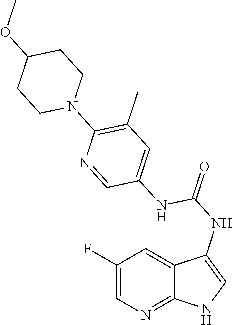 [SMILES: COC1CCN(c2ncc(NC(=O)Nc3c[nH]c4ncc(F)cc34)cc2C)CC1] |
| 195 | 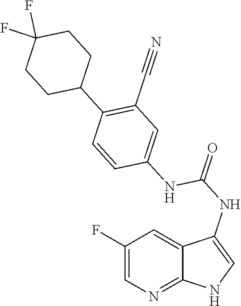 [SMILES: N#Cc1cc(NC(=O)Nc2c[nH]c3ncc(F)cc23)ccc1C1CCC(F)(F)CC1] |
| 196 | 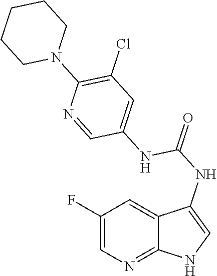 [SMILES: O=C(Nc1cnc(N2CCCCC2)c(Cl)c1)Nc1c[nH]c2ncc(F)cc12] |
| 197 | 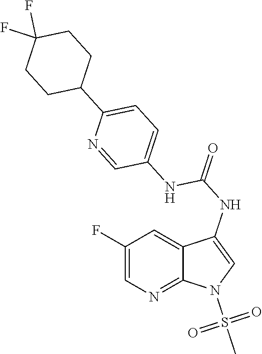 [SMILES: CS(=O)(=O)n1cc(NC(=O)Nc2ccc(C3CCC(F)(F)CC3)nc2)c2cc(F)cnc21] |
| 198 | 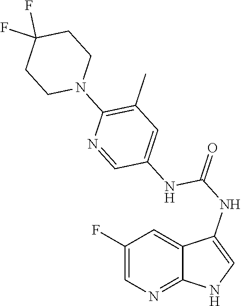 [SMILES: Cc1cc(NC(=O)Nc2c[nH]c3ncc(F)cc23)cnc1N1CCC(F)(F)CC1] |
| 199 | 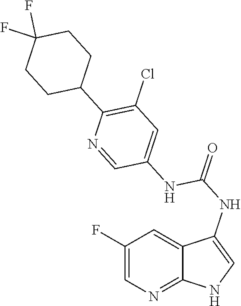 [SMILES: O=C(Nc1cnc(C2CCC(F)(F)CC2)c(Cl)c1)Nc1c[nH]c2ncc(F)cc12] |
| 200 | 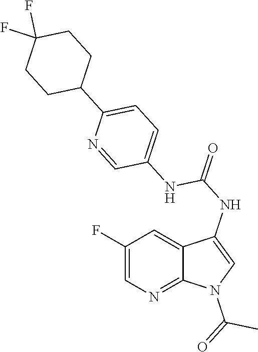 [SMILES: CC(=O)n1cc(NC(=O)Nc2ccc(C3CCC(F)(F)CC3)nc2)c2cc(F)cnc21] |
| 201 | 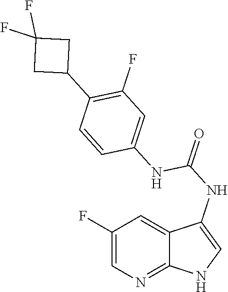 [SMILES: O=C(Nc1ccc(C2CC(F)(F)C2)c(F)c1)Nc1c[nH]c2ncc(F)cc12] |
| 202 | 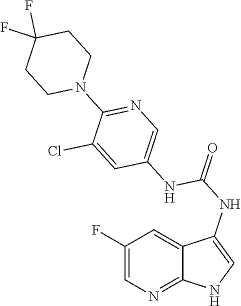 [SMILES: O=C(Nc1cnc(N2CCC(F)(F)CC2)c(Cl)c1)Nc1c[nH]c2ncc(F)cc12] |
| 203 | 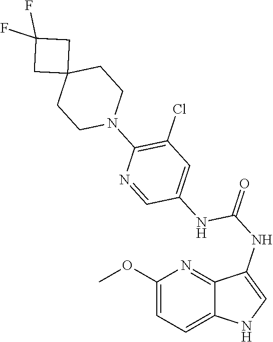 [SMILES: COc1ccc2[nH]cc(NC(=O)Nc3cnc(N4CCC5(CC4)CC(F)(F)C5)c(Cl)c3)c2n1] |
| 204 | 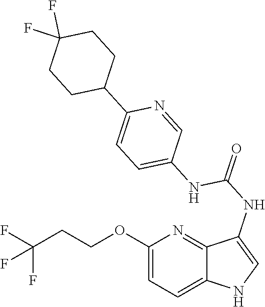 [SMILES: O=C(Nc1ccc(C2CCC(F)(F)CC2)nc1)Nc1c[nH]c2ccc(OCCC(F)(F)F)nc12] |
| 205 | 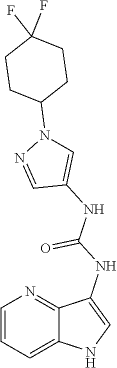 [SMILES: O=C(Nc1cnn(C2CCC(F)(F)CC2)c1)Nc1c[nH]c2cccnc12] |
| 206 | 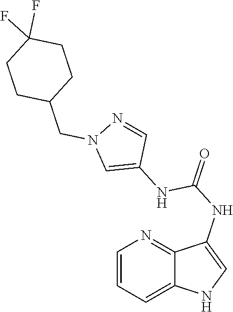 [SMILES: O=C(Nc1cnn(CC2CCC(F)(F)CC2)c1)Nc1c[nH]c2cccnc12] |
| 207 | 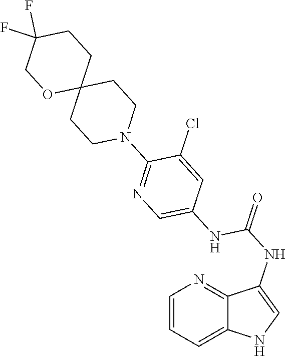 [SMILES: O=C(Nc1cnc(N2CCC3(CC2)CCC(F)(F)CO3)c(Cl)c1)Nc1c[nH]c2cccnc12] |
| 208 | 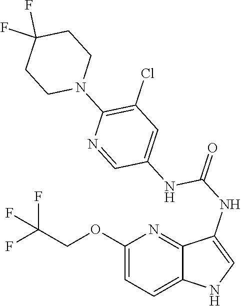 [SMILES: O=C(Nc1cnc(N2CCC(F)(F)CC2)c(Cl)c1)Nc1c[nH]c2ccc(OCC(F)(F)F)nc12] |
| 209 | 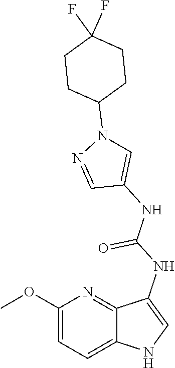 [SMILES: COc1ccc2[nH]cc(NC(=O)Nc3cnn(C4CCC(F)(F)CC4)c3)c2n1] |
| 210 | 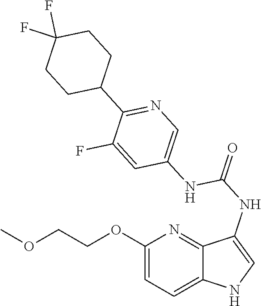 [SMILES: COCCOc1ccc2[nH]cc(NC(=O)Nc3cnc(C4CCC(F)(F)CC4)c(F)c3)c2n1] |
| 211 | 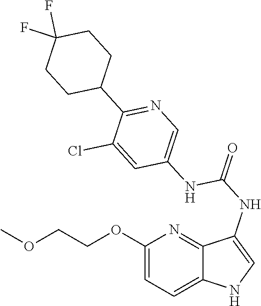 [SMILES: COCCOc1ccc2[nH]cc(NC(=O)Nc3cnc(C4CCC(F)(F)CC4)c(Cl)c3)c2n1] |
| 212 | 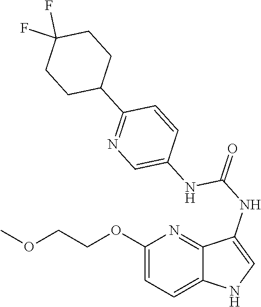 [SMILES: COCCOc1ccc2[nH]cc(NC(=O)Nc3ccc(C4CCC(F)(F)CC4)nc3)c2n1] |
| 213 | 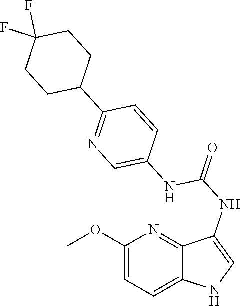 [SMILES: COc1ccc2[nH]cc(NC(=O)Nc3ccc(C4CCC(F)(F)CC4)nc3)c2n1] |
| 214 | 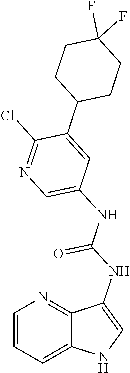 [SMILES: O=C(Nc1cnc(Cl)c(C2CCC(F)(F)CC2)c1)Nc1c[nH]c2cccnc12] |
| 215 | 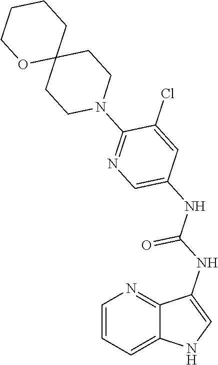 [SMILES: O=C(Nc1cnc(N2CCC3(CCCCO3)CC2)c(Cl)c1)Nc1c[nH]c2cccnc12] |
| 216 | 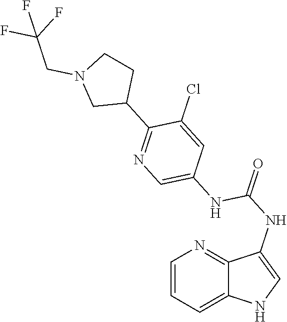 [SMILES: O=C(Nc1cnc(C2CCN(CC(F)(F)F)C2)c(Cl)c1)Nc1c[nH]c2cccnc12] |
| 217 | 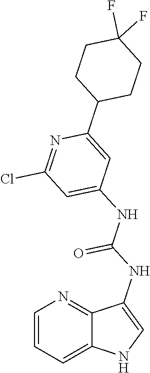 [SMILES: O=C(Nc1cc(Cl)nc(C2CCC(F)(F)CC2)c1)Nc1c[nH]c2cccnc12] |
| 218 | 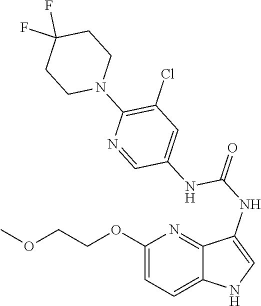 [SMILES: COCCOc1ccc2[nH]cc(NC(=O)Nc3cnc(N4CCC(F)(F)CC4)c(Cl)c3)c2n1] |
| 219 | 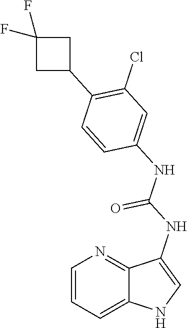 [SMILES: O=C(Nc1ccc(C2CC(F)(F)C2)c(Cl)c1)Nc1c[nH]c2cccnc12] |
| 220 | 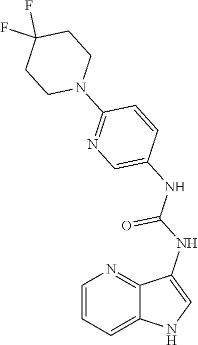 [SMILES: O=C(Nc1ccc(N2CCC(F)(F)CC2)nc1)Nc1c[nH]c2cccnc12] |
| 221 | 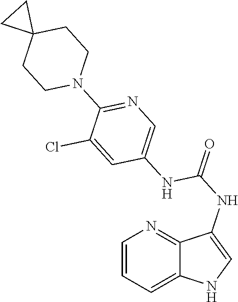 [SMILES: O=C(Nc1cnc(N2CCC3(CC2)CC3)c(Cl)c1)Nc1c[nH]c2cccnc12] |
| 222 | 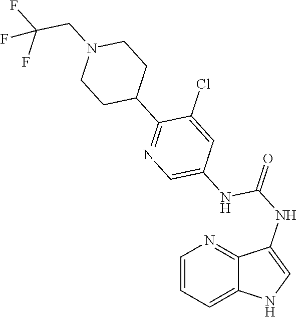 [SMILES: O=C(Nc1cnc(C2CCN(CC(F)(F)F)CC2)c(Cl)c1)Nc1c[nH]c2cccnc12] |
| 223 | 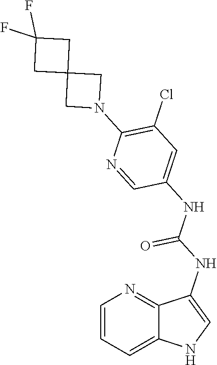 [SMILES: O=C(Nc1cnc(N2CC3(C2)CC(F)(F)C3)c(Cl)c1)Nc1c[nH]c2cccnc12] |
| 224 | 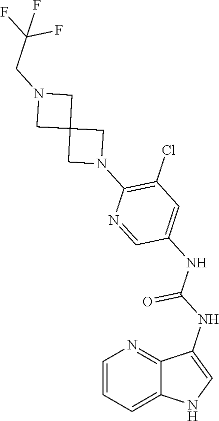 [SMILES: O=C(Nc1cnc(N2CC3(CN(CC(F)(F)F)C3)C2)c(Cl)c1)Nc1c[nH]c2cccnc12] |
| 225 | 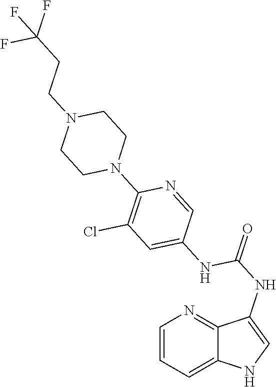 [SMILES: O=C(Nc1cnc(N2CCN(CCC(F)(F)F)CC2)c(Cl)c1)Nc1c[nH]c2cccnc12] |
| 226 | 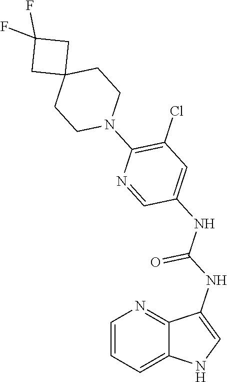 [SMILES: O=C(Nc1cnc(N2CCC3(CC2)CC(F)(F)C3)c(Cl)c1)Nc1c[nH]c2cccnc12] |
| 227 | 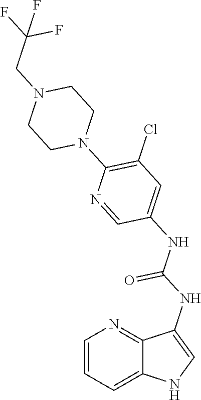 [SMILES: O=C(Nc1cnc(N2CCN(CC(F)(F)F)CC2)c(Cl)c1)Nc1c[nH]c2cccnc12] |
| 228 | 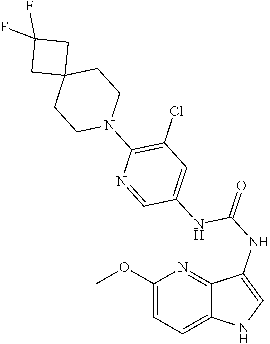 [SMILES: COc1ccc2[nH]cc(NC(=O)Nc3cnc(N4CCC5(CC4)CC(F)(F)C5)c(Cl)c3)c2n1] |
| 229 | 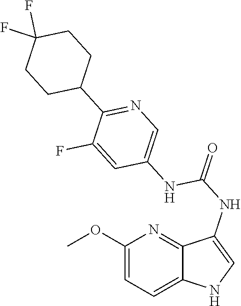 [SMILES: COc1ccc2[nH]cc(NC(=O)Nc3cnc(C4CCC(F)(F)CC4)c(F)c3)c2n1] |
| 230 | 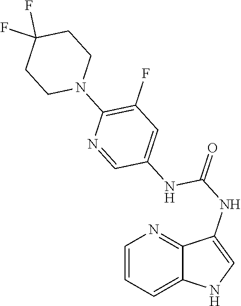 [SMILES: O=C(Nc1cnc(N2CCC(F)(F)CC2)c(F)c1)Nc1c[nH]c2cccnc12] |
| 231 | 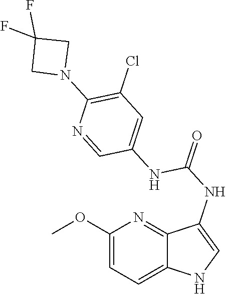 [SMILES: COc1ccc2[nH]cc(NC(=O)Nc3cnc(N4CC(F)(F)C4)c(Cl)c3)c2n1] |
| 232 | 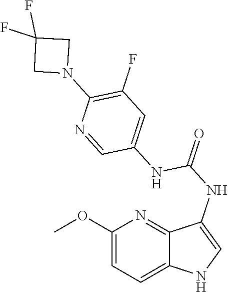 [SMILES: COc1ccc2[nH]cc(NC(=O)Nc3cnc(N4CC(F)(F)C4)c(F)c3)c2n1] |
| 233 | 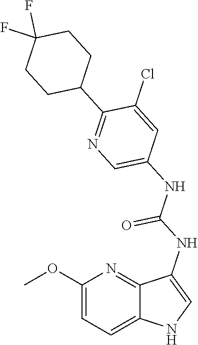 [SMILES: COc1ccc2[nH]cc(NC(=O)Nc3cnc(C4CCC(F)(F)CC4)c(Cl)c3)c2n1] |
| 234 | 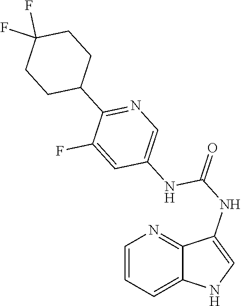 [SMILES: O=C(Nc1cnc(C2CCC(F)(F)CC2)c(F)c1)Nc1c[nH]c2cccnc12] |
| 235 | 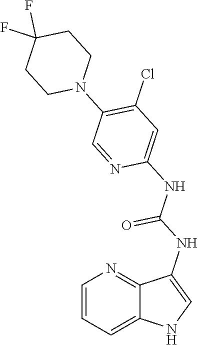 [SMILES: O=C(Nc1cc(Cl)c(N2CCC(F)(F)CC2)cn1)Nc1c[nH]c2cccnc12] |
| 236 | 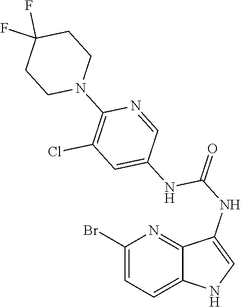 [SMILES: O=C(Nc1cnc(N2CCC(F)(F)CC2)c(Cl)c1)Nc1c[nH]c2ccc(Br)nc12] |
| 237 | 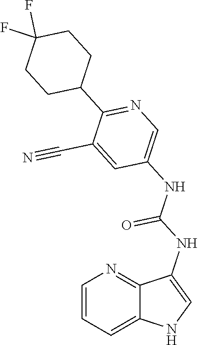 [SMILES: N#Cc1cc(NC(=O)Nc2c[nH]c3cccnc23)cnc1C1CCC(F)(F)CC1] |
| 238 | 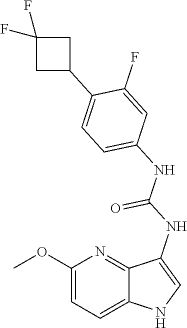 [SMILES: COc1ccc2[nH]cc(NC(=O)Nc3ccc(C4CC(F)(F)C4)c(F)c3)c2n1] |
| 239 | 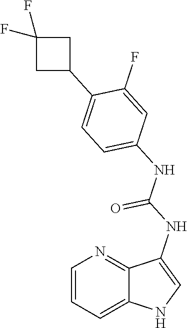 [SMILES: O=C(Nc1ccc(C2CC(F)(F)C2)c(F)c1)Nc1c[nH]c2cccnc12] |
| 240 | 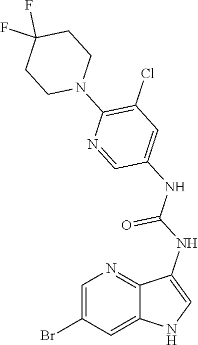 [SMILES: O=C(Nc1cnc(N2CCC(F)(F)CC2)c(Cl)c1)Nc1c[nH]c2cc(Br)cnc12] |
| 241 | 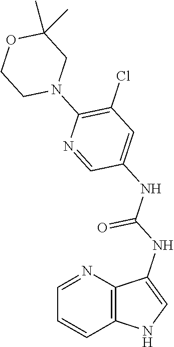 [SMILES: CC1(C)CN(c2ncc(NC(=O)Nc3c[nH]c4cccnc34)cc2Cl)CCO1] |
| 242 | 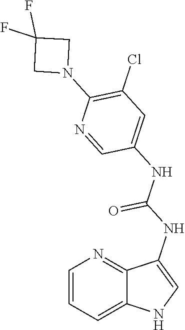 [SMILES: O=C(Nc1cnc(N2CC(F)(F)C2)c(Cl)c1)Nc1c[nH]c2cccnc12] |
| 243 | 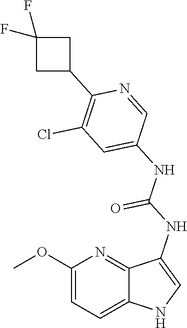 [SMILES: COc1ccc2[nH]cc(NC(=O)Nc3cnc(C4CC(F)(F)C4)c(Cl)c3)c2n1] |
| 244 | 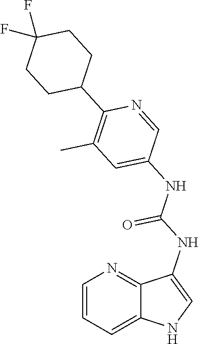 [SMILES: Cc1cc(NC(=O)Nc2c[nH]c3cccnc23)cnc1C1CCC(F)(F)CC1] |
| 245 | 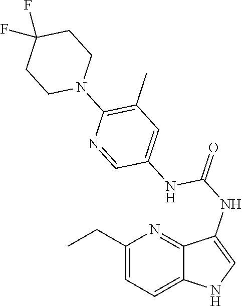 [SMILES: CCc1ccc2[nH]cc(NC(=O)Nc3cnc(N4CCC(F)(F)CC4)c(C)c3)c2n1] |
| 246 | 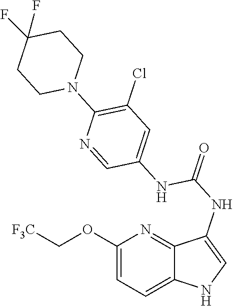 [SMILES: O=C(Nc1cnc(N2CCC(F)(F)CC2)c(Cl)c1)Nc1c[nH]c2ccc(OCC(F)(F)F)nc12] |

Pharmaceutical Compositions and Administration

General

In some embodiments, a chemical entity (e.g., a compound that inhibits (e.g., antagonizes) STING, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%.

Actual methods of preparing such dosage forms are known, or will be apparent, to those skilled in this art; for example, see Remington: The Science and Practice of Pharmacy, 22^{nd} Edition (Pharmaceutical Press, London, U K. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, weeks, 6 weeks, 7 weeks, 8 weeks, 9 weeks, 10 weeks, 11 weeks, 12 weeks, 4 months, months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which increased (e.g., excessive) STING activity (e.g., e.g., STING signaling) contributes to the pathology and/or symptoms and/or progression of the condition, disease or disorder (e.g., immune disorders, cancer) are provided.

Indications

In some embodiments, the condition, disease or disorder is cancer. Non-limiting examples of cancer include melanoma, carcinoma, lymphoma, blastoma, sarcoma, and leukemia or lymphoid malignancies. More particular examples of such cancers include breast cancer, colon cancer, rectal cancer, colorectal cancer, kidney or renal cancer, clear cell cancer lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, prostatic neoplasms, liver cancer, bladder cancer, cancer of the peritoneum, hepatocellular cancer, gastric or stomach cancer including gastrointestinal cancer, gastrointestinal stromal tumor, pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma, myelodysplasia disorders, myeloproliferative disorders, chronic myelogenous leukemia, and acute hematologic malignancies, endometrial or uterine carcinoma, endometriosis, endometrial stromal sarcoma, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, mast cell sarcoma, ovarian sarcoma, uterine sarcoma, melanoma, malignant mesothelioma, skin carcinomas, Schwannoma, oligodendroglioma, neuroblastomas, neuroectodermal tumor, rhabdomyosarcoma, osteogenic sarcoma, leiomyosarcomas, Ewing Sarcoma, peripheral primitive neuroectodermal tumor, urinary tract carcinomas, thyroid carcinomas, Wilm's tumor, as well as abnormal vascular proliferation associated with phakomatoses, edema (such as that associated with brain tumors), and Meigs' syndrome. In some cases, the cancer is melanoma.

In some embodiments, the condition, disease or disorder is a neurological disorder, which includes disorders that involve the central nervous system (brain, brainstem and cerebellum), the peripheral nervous system (including cranial nerves), and the autonomic nervous system (parts of which are located in both central and peripheral nervous system). Non-limiting examples of such neurological disorders include acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wildon's disease; amyotrophe lateral sclerosis and Zellweger syndrome.

In some embodiments, the condition, disease or disorder is STING-associated conditions, e.g., type I interferonopathies (e.g., STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis. In certain embodiments, the condition, disease or disorder is an autoimmune disease (e.g., a cytosolic DNA-triggered autoinflammatory disease). Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, modulation of the immune system by STING provides for the treatment of diseases, including diseases caused by foreign agents. Exemplary infections by foreign agents which may be treated and/or prevented by the method of the present invention include an infection by a bacterium (e.g., a Gram-positive or Gram-negative bacterium), an infection by a fungus, an infection by a parasite, and an infection by a virus. In one embodiment of the present invention, the infection is a bacterial infection (e.g., infection by E. coli, Klebsiella pneumoniae, Pseudomonas aeruginosa, Salmonella spp., Staphylococcus aureus, Streptococcus spp., or vancomycin-resistant enterococcus), or sepsis. In another embodiment, the infection is a fungal infection (e.g. infection by a mould, a yeast, or a higher fungus). In still another embodiment, the infection is a parasitic infection (e.g., infection by a single-celled or multicellular parasite, including Giardia duodenalis, Cryptosporidium parvum, Cyclospora cayetanensis, and Toxoplasma gondiz). In yet another embodiment, the infection is a viral infection (e.g., infection by a virus associated with AIDS, avian flu, chickenpox, cold sores, common cold, gastroenteritis, glandular fever, influenza, measles, mumps, pharyngitis, pneumonia, rubella, SARS, and lower or upper respiratory tract infection (e.g., respiratory syncytial virus)).

In some embodiments, the condition, disease or disorder is hepatitis B (see, e.g., WO 2015/061294).

In some embodiments, the condition, disease or disorder is selected from cardiovascular diseases (including e.g., myocardial infarction).

In some embodiments, the condition, disease or disorder is age-related macular degeneration.

In some embodiments, the condition, disease or disorder is mucositis, also known as stomatitis, which can occur as a result of chemotherapy or radiation therapy, either alone or in combination as well as damage caused by exposure to radiation outside of the context of radiation therapy.

In some embodiments, the condition, disease or disorder is uveitis, which is inflammation of the uvea (e.g., anterior uveitis, e.g., iridocyclitis or iritis; intermediate uveitis (also known as pars planitis); posterior uveitis; or chorioretinitis, e.g., pan-uveitis).

In some embodiments, the condition, disease or disorder is selected from the group consisting of a cancer, a neurological disorder, an autoimmune disease, hepatitis B, uvetitis, a cardiovascular disease, age-related macular degeneration, and mucositis.

Still other examples can include those indications discussed herein and below in contemplated combination therapy regimens.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the methods described herein can further include administering one or more additional cancer therapies.

The one or more additional cancer therapies can include, without limitation, surgery, radiotherapy, chemotherapy, toxin therapy, immunotherapy, cryotherapy, cancer vaccines (e.g., HPV vaccine, hepatitis B vaccine, Oncophage, Provenge) and gene therapy, as well as combinations thereof. Immunotherapy, including, without limitation, adoptive cell therapy, the derivation of stem cells and/or dendritic cells, blood transfusions, lavages, and/or other treatments, including, without limitation, freezing a tumor.

In some embodiments, the one or more additional cancer therapies is chemotherapy, which can include administering one or more additional chemotherapeutic agents.

In certain embodiments, the additional chemotherapeutic agent is an immunomodulatory moiety, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

In certain embodiments, the additional chemotherapeutic agent is an alkylating agent. Alkylating agents are so named because of their ability to alkylate many nucleophilic functional groups under conditions present in cells, including, but not limited to cancer cells. In a further embodiment, an alkylating agent includes, but is not limited to, Cisplatin, carboplatin, mechlorethamine, cyclophosphamide, chlorambucil, ifosfamide and/or oxaliplatin. In an embodiment, alkylating agents can function by impairing cell function by forming covalent bonds with the amino, carboxyl, sulfhydryl, and phosphate groups in biologically important molecules or they can work by modifying a cell's DNA. In a further embodiment an alkylating agent is a synthetic, semisynthetic or derivative.

In certain embodiments, the additional chemotherapeutic agent is an anti-metabolite. Anti-metabolites masquerade as purines or pyrimidines, the building-blocks of DNA and in general, prevent these substances from becoming incorporated in to DNA during the “S” phase (of the cell cycle), stopping normal development and division. Anti-metabolites can also affect RNA synthesis. In an embodiment, an antimetabolite includes, but is not limited to azathioprine and/or mercaptopurine. In a further embodiment an anti-metabolite is a synthetic, semisynthetic or derivative.

In certain embodiments, the additional chemotherapeutic agent is a plant alkaloid and/or terpenoid. These alkaloids are derived from plants and block cell division by, in general, preventing microtubule function. In an embodiment, a plant alkaloid and/or terpenoid is a vinca alkaloid, a podophyllotoxin and/or a taxane. Vinca alkaloids, in general, bind to specific sites on tubulin, inhibiting the assembly of tubulin into microtubules, generally during the M phase of the cell cycle. In an embodiment, a vinca alkaloid is derived, without limitation, from the Madagascar periwinkle, Catharanthus roseus (formerly known as Vinca rosea). In an embodiment, a vinca alkaloid includes, without limitation, Vincristine, Vinblastine, Vinorelbine and/or Vindesine. In an embodiment, a taxane includes, but is not limited, to Taxol, Paclitaxel and/or Docetaxel. In a further embodiment a plant alkaloid or terpernoid is a synthetic, semisynthetic or derivative. In a further embodiment, a podophyllotoxin is, without limitation, an etoposide and/or teniposide. In an embodiment, a taxane is, without limitation, docetaxel and/or ortataxel. [021] In an embodiment, a cancer therapeutic is a topoisomerase. Topoisomerases are essential enzymes that maintain the topology of DNA. Inhibition of type I or type II topoisomerases interferes with both transcription and replication of DNA by upsetting proper DNA supercoiling. In a further embodiment, a topoisomerase is, without limitation, a type I topoisomerase inhibitor or a type II topoisomerase inhibitor. In an embodiment a type I topoisomerase inhibitor is, without limitation, a camptothecin. In another embodiment, a camptothecin is, without limitation, exatecan, irinotecan, lurtotecan, topotecan, BNP 1350, CKD 602, DB 67 (AR67) and/or ST 1481. In an embodiment, a type II topoisomerase inhibitor is, without limitation, epipodophyllotoxin. In a further embodiment an epipodophyllotoxin is, without limitation, an amsacrine, etoposid, etoposide phosphate and/or teniposide. In a further embodiment a topoisomerase is a synthetic, semisynthetic or derivative, including those found in nature such as, without limitation, epipodophyllotoxins, substances naturally occurring in the root of American Mayapple (Podophyllum peltatum).

In certain embodiments, the additional chemotherapeutic agent is a stilbenoid. In a further embodiment, a stilbenoid includes, but is not limited to, Resveratrol, Piceatannol, Pinosylvin, Pterostilbene, Alpha-Viniferin, Ampelopsin A, Ampelopsin E, Diptoindonesin C, Diptoindonesin F, Epsilon-Vinferin, Flexuosol A, Gnetin H, Hemsleyanol D, Hopeaphenol, Trans-Diptoindonesin B, Astringin, Piceid and Diptoindonesin A. In a further embodiment a stilbenoid is a synthetic, semisynthetic or derivative.

In certain embodiments, the additional chemotherapeutic agent is a cytotoxic antibiotic. In an embodiment, a cytotoxic antibiotic is, without limitation, an actinomycin, an anthracenedione, an anthracycline, thalidomide, dichloroacetic acid, nicotinic acid, 2-deoxyglucose and/or chlofazimine. In an embodiment, an actinomycin is, without limitation, actinomycin D, bacitracin, colistin (polymyxin E) and/or polymyxin B. In another embodiment, an antracenedione is, without limitation, mitoxantrone and/or pixantrone. In a further embodiment, an anthracycline is, without limitation, bleomycin, doxorubicin (Adriamycin), daunorubicin (daunomycin), epirubicin, idarubicin, mitomycin, plicamycin and/or valrubicin. In a further embodiment a cytotoxic antibiotic is a synthetic, semisynthetic or derivative.

In certain embodiments, the additional chemotherapeutic agent is selected from endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, signal transduction inhibitors, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-3), vasculostatin, vasostatin (calreticulin fragment) and the like.

In certain embodiments, the additional chemotherapeutic agent is selected from abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In certain embodiments, the additional chemotherapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In still other embodiments, the additional chemotherapeutic agent can be selected from those delineated in U.S. Pat. No. 7,927,613, which is incorporated herein by reference in its entirety.

In some embodiments, the additional therapeutic agent and/or regimen are those that can be used for treating other STING-associated conditions, e.g., type I interferonopathies (e.g., STING-associated vasculopathy with onset in infancy (SAVI)), Aicardi-Goutieres Syndrome (AGS), genetic forms of lupus, and inflammation-associated disorders such as systemic lupus erythematosus, and rheumatoid arthritis and the like.

Non-limiting examples of additional therapeutic agents and/or regimens for treating rheumatoid arthritis include non-steroidal anti-inflammatory drugs (NSAIDs; e.g., ibuprofen and naproxen), corticosteroids (e.g, prednisone), disease-modifying antirheumatic drugs (DMARDs; e.g., methotrexate (Trexall®, Otrexup®, Rasuvo®, Rheumatrex®), leflunomide (Arava®), hydroxychloroquine (Plaquenil), PF-06650833, iguratimod, tofacitinib (Xeljanz®), ABBV-599, evobrutinib, and sulfasalazine (Azulfidine®)), and biologics (e.g., abatacept (Orencia®), adalimumab (Humira®), anakinra (Kineret®), certolizumab (Cimzia®), etanercept (Enbrel®), golimumab (Simponi®), infliximab (Remicade®), rituximab (Rituxan®), tocilizumab (Actemra®), vobarilizumab, sarilumab (Kevzara®), secukinumab, ABP 501, CHS-0214, ABC-3373, and tocilizumab (ACTEMRA®)).

Non-limiting examples of additional therapeutic agents and/or regimens for treating lupus include steroids, topical immunomodulators (e.g., tacrolimus ointment (Protopic®) and pimecrolimus cream (Elidel®)), thalidomide (Thalomid®), non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., evobrutinib, iberdomide, voclosporin, cenerimod, azathioprine (Imuran®), cyclophosphamide (Cytoxan®, Neosar®, Endoxan®), and cyclosporine (Neoral, Sandimmune®, Gengraf®), and mycophenolate mofetil) baricitinb, iguratimod, filogotinib, GS-9876, rapamycin, and PF-06650833), and biologics (e.g., belimumab (Benlysta®), anifrolumab, prezalumab, MEDIO700, obinutuzumab, vobarilizumab, lulizumab, atacicept, PF-06823859, and lupizor, rituximab, BT063, BI655064, BIIB059, aldesleukin (Proleukin®), dapirolizumab, edratide, IFN-α-kinoid, OMS721, RC18, RSLV-132, theralizumab, XmAb5871, and ustekinumab (Stelara®)). For example, non-limiting treatments for systemic lupus erythematosus include non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., iberdomide, voclosporin, azathioprine (Imuran®), cyclophosphamide (Cytoxan®, Neosar®, Endoxan®), and cyclosporine (Neoral, Sandimmune®, Gengraf®), and mycophenolate mofetil, baricitinb, filogotinib, and PF-06650833), and biologics (e.g., belimumab (Benlysta®), anifrolumab, prezalumab, MEDIO700, vobarilizumab, lulizumab, atacicept, PF-06823859, lupizor, rituximab, BT063, BI655064, BIIB059, aldesleukin (Proleukin®), dapirolizumab, edratide, IFN-α-kinoid, RC18, RSLV-132, theralizumab, XmAb5871, and ustekinumab (Stelara®)). As another example, non-limiting examples of treatments for cutaneous lupus include steroids, immunomodulators (e.g., tacrolimus ointment (Protopic®) and pimecrolimus cream (Elidel®)), GS-9876, filogotinib, and thalidomide (Thalomid®). Agents and regimens for treating drug-induced and/or neonatal lupus can also be administered.

Non-limiting examples of additional therapeutic agents and/or regimens for treating STING-associated vasculopathy with onset in infancy (SAVI) include JAK inhibitors (e.g., tofacitinib, ruxolitinib, filgotinib, and baricitinib).

Non-limiting examples of additional therapeutic agents and/or regimens for treating Aicardi-Goutieres Syndrome (AGS) include physiotherapy, treatment for respiratory complications, anticonvulsant therapies for seizures, tube-feeding, nucleoside reverse transcriptase inhibitors (e.g., emtricitabine (e.g., Emtriva®), tenofovir (e.g., Viread®), emtricitabine/tenofovir (e.g., Truvada®), zidovudine, lamivudine, and abacavir), and JAK inhibitors (e.g., tofacitinib, ruxolitinib, filgotinib, and baricitinib).

Non-limiting examples of additional therapeutic agents and/or regimens for treating IBDs include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (IL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

Non-limiting examples of additional therapeutic agents and/or regimens for treating irritable bowel syndrome include alosetron, bile acid sequesterants (e.g., cholestyramine, colestipol, colesevelam), chloride channel activators (e.g., lubiprostone), coated peppermint oil capsules, desipramine, dicyclomine, ebastine, eluxadoline, farnesoid X receptor agonist (e.g., obeticholic acid), fecal microbiota transplantation, fluoxetine, gabapentin, guanylate cyclase-C agonists (e.g., linaclotide, plecanatide), ibodutant, imipramine, JCM-16021, loperamide, lubiprostone, nortriptyline, ondansetron, opioids, paroxetine, pinaverium, polyethylene glycol, pregabalin, probiotics, ramosetron, rifaximin, and tanpanor.

Non-limiting examples of additional therapeutic agents and/or regimens for treating scleroderma include non-steroidal anti-inflammatory drugs (NSAIDs; e.g., ibuprofen and naproxen), corticosteroids (e.g, prednisone), immunomodulators (e.g., azathioprine, methotrexate (Trexall®, Otrexup®, Rasuvo®, Rheumatrex®), cyclophosphamide (Cytoxan®, Neosar®, Endoxan®), and cyclosporine (Neoral®, Sandimmune®, Gengraf®), antithymocyte globulin, mycophenolate mofetil, intravenous immunoglobulin, rituximab, sirolimus, and alefacept), calcium channel blockers (e.g., nifedipine), alpha blockers, serotonin receptor antagonists, angiotensin II receptor inhibitors, statins, local nitrates, iloprost, phosphodiesterase 5 inhibitors (e.g., sildenafil), bosentan, tetracycline antibiotics, endothelin receptor antagonists, prostanoids, and tyrosine kinase inhibitors (e.g., imatinib, nilotinib and dasatinib).

Non-limiting examples of additional therapeutic agents and/or regimens for treating Crohn's Disease (CD) include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, figlotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

Non-limiting examples of additional therapeutic agents and/or regimens for treating UC include AbGn-168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, figlotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

Non-limiting examples of additional therapeutic agents and/or regimens for treating autoimmune colitis include corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating iatrogenic autoimmune colitis include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis induced by one or more chemotherapeutics agents include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating colitis induced by treatment with adoptive cell therapy include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis associated with one or more alloimmune diseases include corticosteroids (e.g., budesonide, prednisone, prednisolone, beclometasone dipropionate), sulfasalazine, and eicopentaenoic acid.

Non-limiting examples of additional therapeutic agents and/or regimens for treating radaiation enteritis include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

Non-limiting examples of additional therapeutic agents and/or regimens for treating collagenous colitis include 6-mercaptopurine, azathaioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

Non-limiting examples of additional therapeutic agents and/or regimens for treating lyphocytic colitis include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

Non-limiting examples of additional therapeutic agents and/or regimens for treating microscopic colitis include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

Non-limiting examples of additional therapeutic agents and/or regimens for treating alloimmune disease include intrauterine platelet transfusions, intravenous immunoglobin, maternal steroids, abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating multiple sclerosis (MS) include alemtuzumab (Lemtrada®), ALKS 8700, amiloride, ATX-MS-1467, azathioprine, baclofen (Lioresal®), beta interferons (e.g., IFN-β-1a, IFN-β-1b), cladribine, corticosteroids (e.g., methylprednisolone), daclizumab, dimethyl fumarate (Tecfidera®), fingolimod (Gilenya®), fluoxetine, glatiramer acetate (Copaxone®), hydroxychloroquine, ibudilast, idebenone, laquinimod, lipoic acid, losartan, masitinib, MD1003 (biotin), mitoxantrone, montelukast, natalizumab (Tysabri®), NeuroVax™, ocrelizumab, ofatumumab, pioglitazone, and RPC1063.

Non-limiting examples of additional therapeutic agents and/or regimens for treating graft-vs-host disease include abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, imatinib, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating acute graft-vs-host disease include alemtuzumab, alpha-1 antitrypsin, antithymocyte globulin, basiliximab, brentuximab, corticosteroids (e.g., methylprednisone, prednisone), cyclosporine, dacilzumab, defribrotide, denileukin diftitox, ibrutinib, infliximab, itacitinib, LBH589, mycophenolate mofetil, natalizumab, neihulizumab, pentostatin, photopheresis, ruxolitinib, sirolimus, tacrolimus, and tocilizumab.

Non-limiting examples of additional therapeutic agents and/or regimens for treating chronic graft vs. host disease include abatacept, alemtuzumab, AMG592, antithymocyte globulin, basiliximab, bortezomib, corticosteroids (e.g., methylprednisone, prednisone), cyclosporine, dacilzumab, denileukin diftitox, glasdegib, ibrutinib, IL-2, imatinib, infliximab, mycophenolate mofetil, pentostatin, photobiomodulation, photopheresis, ruxolitinib, sirolimus, sonidegib, tacrolimus, tocilizumab, and vismodegib.

Non-limiting examples of additional therapeutic agents and/or regimens for treating celiac disease include AMG 714, AMY01, Aspergillus niger prolyl endoprotease, BL-7010, CALY-002, GBR 830, Hu-Mik-Beta-1, IMGX003, KumaMax, Larazotide Acetate, Nexvan2®, pancrelipase, TIMP-GLIA, vedolizumab, and ZED1227.

Non-limiting examples of additional therapeutic agents and/or regimens for treating psoriasis include topical corticosteroids, topical crisaborole/AN2728, topical SNA-120, topical SAN021, topical tapinarof, topical tocafinib, topical IDP-118, topical M518101, topical calcipotriene and betamethasone dipropionate (e.g., MC2-01 cream and Taclonex®), topical P-3073, topical LEO 90100 (Enstilar®), topical betamethasone dipropriate (Sernivo®), halobetasol propionate (Ultravate®), vitamin D analogues (e.g., calcipotriene (Dovonex®) and calcitriol (Vectical®)), anthralin (e.g., Dritho-scalp® and Dritho-creme®), topical retinoids (e.g., tazarotene (e.g., Tazorac® and Avage®)), calcineurin inhibitors (e.g., tacrolimus (Prograf®) and pimecrolimus (Elidel®)), salicylic acid, coal tar, moisturizers, phototherapy (e.g., exposure to sunlight, UVB phototherapy, narrow band UVB phototherapy, Goeckerman therapy, psoralen plus ultraviolet A (PUVA) therapy, and excimer laser), retinoids (e.g., acitretin (Soriatane®)), methotrexate (Trexall®, Otrexup®, Rasuvo®, Rheumatrex®), Apo805K1, baricitinib, FP187, KD025, prurisol, VTP-43742, XP23829, ZPL-389, CF101 (piclidenoson), LAS41008, VPD-737 (serlopitant), upadacitinib (ABT-494), aprmilast, tofacitibin, cyclosporine (Neoral®, Sandimmune®, Gengraf®), biologics (e.g., etanercept (Enbrel®), entanercept-szzs (Elrezi®), infliximab (Remicade®), adalimumab (Humira®), adalimumab-adbm (Cyltezo®), ustekinumab (Stelara®), golimumab (Simponi®), apremilast (Otezla®), secukinumab (Cosentyx®), certolixumab pegol, secukinumab, tildrakizumab-asmn, infliximab-dyyb, abatacept, ixekizumab (Taltz®), ABP 710, BCD-057, BI695501, bimekizumab (UCB4940), CHS-1420, GP2017, guselkumab (CNTO 1959), HD203, M923, MSB11022, Mirikizumab (LY3074828), PF-06410293, PF-06438179, risankizumab (BI655066), SB2, SB4, SB5, siliq (brodalumab), namilumab (MT203, tildrakizumab (MK-3222), and ixekizumab (Taltz®)), thioguanine, and hydroxyurea (e.g., Droxia® and Hydrea®).

Non-limiting examples of additional therapeutic agents and/or regimens for treating cutaneous T-cell lymphoma include phototherapy (e.g., exposure to sunlight, UVB phototherapy, narrow band UVB phototherapy, Goeckerman therapy, psoralen plus ultraviolet A (PUVA) therapy, and excimer laser), extracorporeal photopheresis, radiation therapy (e.g., spot radiation and total skin body electron beam therapy), stem cell transplant, corticosteroids, imiquimod, bexarotene gel, topical bis-chloroethyl-nitrourea, mechlorethamine gel, vorinostat (Zolinza®), romidepsin (Istodax®), pralatrexate (Folotyn®) biologics (e.g., alemtuzumab (Campath®), brentuximab vedotin (SGN-35), mogamulizumab, and IPH4102).

Non-limiting examples of additional therapeutic agents and/or regimens for treating uveitis include corticosteroids (e.g., intravitreal triamcinolone acetonide injectable suspensions), antibiotics, antivirals (e.g., acyclovir), dexamethasone, immunomodulators (e.g., tacrolimus, leflunomide, cyclophosphamide (Cytoxan®, Neosar®, Endoxan®), and cyclosporine (Neoral®, Sandimmune®, Gengraf®), chlorambucil, azathioprine, methotrexate, and mycophenolate mofetil), biologics (e.g., infliximab (Remicade®), adalimumab (Humira®), etanercept (Enbrel®), golimumab (Simponi®), certolizumab (Cimzia®), rituximab (Rituxan®), abatacept (Orencia®), basiliximab (Simulect®), anakinra (Kineret®), canakinumab (Ilaris®), gevokixumab (XOMA052), tocilizumab (Actemra®), alemtuzumab (Campath®), efalizumab (Raptiva®), LFG316, sirolimus (Santen®), abatacept, sarilumab (Kevzara®), and daclizumab (Zenapax®)), cytotoxic drugs, surgical implant (e.g., fluocinolone insert), and vitrectomy.

on-limiting examples of additional therapeutic agents and/or regimens for treating mucositis include AG013, SGX942 (dusquetide), amifostine (Ethyol®), cryotherapy, cepacol lonzenges, capsaicin lozenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, granules comprising Vaccinium myrtillus extract, Macleaya cordata alkaloids and Echinacea angustifolia extract (e.g., SAMITAL®), and gastrointestinal cocktail (an acid reducer such aluminum hydroxide and magnesium hydroxide (e.g., Maalox), an antifungal (e.g., nystatin), and an analgesic (e.g., hurricane liquid)). For example, non-limiting examples of treatments for oral mucositis include AG013, amifostine (Ethyol®), cryotherapy, cepacol lonzenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, and gastrointestinal cocktail (an acid reducer such aluminum hydroxide and magnesium hydroxide (e.g., Maalox), an antifungal (e.g., nystatin), and an analgesic (e.g., hurricane liquid)). As another example, non-limiting examples of treatments for esophageal mucositis include xylocaine (e.g., gel viscous Xylocaine 2%). As another example, treatments for intestinal mucositis, treatments to modify intestinal mucositis, and treatments for intestinal mucositis signs and symptoms include gastrointestinal cocktail (an acid reducer such aluminum hydroxide and magnesium hydroxide (e.g., Maalox), an antifungal (e.g., nystatin), and an analgesic (e.g., hurricane liquid)).

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of such treatment (e.g., by way of biopsy, endoscopy, or other conventional method known in the art). In certain embodiments, the STING protein can serve as a biomarker for certain types of cancer, e.g., colon cancer and prostate cancer. In other embodiments, identifying a subject can include assaying the patient's tumor microenvironment for the absence of T-cells and/or presence of exhausted T-cells, e.g., patients having one or more cold tumors. Such patients can include those that are resistant to treatment with checkpoint inhibitors. In certain embodiments, such patients can be treated with a chemical entity herein, e.g., to recruit T-cells into the tumor, and in some cases, further treated with one or more checkpoint inhibitors, e.g., once the T-cells become exhausted.

In some embodiments, the chemical entities, methods, and compositions described herein can be administered to certain treatment-resistant patient populations (e.g., patients resistant to checkpoint inhibitors; e.g., patients having one or more cold tumors, e.g., tumors lacking T-cells or exhausted T-cells).

Compound Preparation

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylamine, 1,8-diazabicycloundec-7-ene, 2,6-di-tert-butylpyridine, or tetrabutyiphosphazene).

The skilled artisan will recognize a variety of analytical methods that can be used to characterize the compounds described herein, including, for example, ne NMR, heteronuclear NMR, mass spectrometry, liquid chromatography, and infrared spectroscopy. The foregoing list is a subset of characterization methods available to a skilled artisan and is not intended to be limiting.

To further illustrate the foregoing, the following non-limiting, exemplary synthetic schemes are included. Variations of these examples within the scope of the claims are within the purview of one skilled in the art and are considered to fall within the scope of the invention as described, and claimed herein. The reader will recognize that the skilled artisan, provided with the present disclosure, and skill in the art is able to prepare and use the invention without exhaustive examples.

The following abbreviations have the indicated meanings:

Ac = acetyl
ACN = acetonitrile
AcOH = acetic acid
BINAP = 2,2′-bis(diphenylphosphino)-1,1′-binaphthalene
Bn = benzyl
Boc = t-butyloxy carbonyl
Boc₂O = di-tert-butyl dicarbonate
Bu = butyl
Bz = benzoyl
DAST = diethylaminosulphur trifluoride
DBU = 1,5-dizzabicyclo[5.4.0]undecen-5-ene
DCM = dichloromethane
DIEA = N,N-diisopropylethylamine
DMAP = dimethylaminopyridine
DMF = N,N-dimethylformamide
DMF-DMA = N,N-dimethylformamide dimethyl acetal
DMSO = dimethyl sulfoxide
DPPA = diphenylphosphoryl azide
Dppf = bis(diphenylphosphino)ferrocene
Et = ethyl
HATU = (1-[Bis(dimethylamino)methylene]-1H-1,2,3-triazolo[4,5-
b]pyridinium 3-oxide hexafluorophosphate
HMDS = hexamethyldisilazane
HPLC = high performance liquid chromatography
LC-MS = liquid chromatography mass spectrometry
Me = methyl
MeOH = methanol
Ms = methanesulfonyl
NMR = nuclear magnetic resonance
Ph = phenyl
RT = retention time
TBS = tertbutylaimethylsilyl chloride
TBS = tertbutyldimethylsilyl
TEA = triethylamine
TFA = trifluoroacetic acid
THF = tetrahydrofuran
Xphos = 2-(dicyclohexylphosphino)-2′,4′,6′-tri-i-propyl-1,1′-biphenyl

### EXAMPLES

Materials and Methods

The progress of reactions was often monitored by TLC or LC-MS. The identity of the products was often confirmed by LC-MS. The LC-MS was recorded using one of the following methods.

LCMS Method A: XBridge Shield RP18, 50*4.6 mm, 3.0 μL injection, 1.5 mL/min flowrate, 90-900 amu scan range, 254 nm UV detection. Mobile Phase A (MPA): Water/0.04% NH3.H₂O and Mobile Phase B (MPB): Acetonitrile. Elution 40% MPB to 70% in 2.80 min, up to 95% in 0.20 min, hold at 95% MPB for 0.5 min, 95% MPB to 10% in 0.05 min, then equilibration to 10% MPB for 0.25 min.

LCMS Method B: HALO C18, 30*3.0 mm, 0.1 μL injection, 1.2 mL/min flowrate, 90-900 amu scan range, 254 nm UV detection. Mobile Phase A (MPA): Water+0.05% TFA and Mobile Phase B (MPB): Acetonitrile+0.05% TFA. Elution 10% MPB to 100% in 1.30 min, hold at 100% MPB for 0.5 min, 100% MPB to 10% in 0.03 min, then equilibration to 5% MPB for 0.17 min.

LCMS Method C: XBridge BEH Shield RP, 50*3.0 mm, 0.5 μL injection, 1.2 mL/min flowrate, 30-2000 amu scan range, 254 nm UV detection. Mobile Phase A (MPA): Water+5 mM NH₄HCO₃ and Mobile Phase B (MPB): ACN. Elution 10% MPB to 95% in 1.29 min, hold at 95% MPB for 0.3 min, 95% MPB to 10% in 0.1 min, then equilibration to 5% MPB for 0.1 min.

LCMS Method D: XBridge BEH C18, 50*3 mm, 0.7 μL injection, 1.0 mL/min flowrate, 30-900 amu scan range, 254 nm UV detection. Mobile Phase A (MPA): Water+5 mmol NH₄HCO₃ and Mobile Phase B (MPB): Acetonitrile. Elution 5% MPB to 95% in 0.0.99 min, hold at 95% MPB for 0.7 min, 95% MPB to 5% in 0.10 min, then equilibration to 5% MPB for 0.2 min.

LCMS Method E: Shim-pack Scepter C18, 50*3 mm, 0.8 μL injection, 1.5 mL/min flowrate, 90-900 amu scan range, 254 nm UV detection. Mobile Phase A (MPA): Water/0.04% NH3.H₂O and Mobile Phase B (MPB): Acetonitrile. Elution 10% MPB to 95% in 2.00 min, hold at 95% MPB for 0.60 min, 95% MPB to 10% in 0.20 min, then equilibration to 10% MPB for 0.20 min.

LCMS Method F: Poroshell HPH-C18, 50*3 mm, 2.7 uL injection, 1.5 mL/min flowrate, 90-900 amu scan range, 254 nm UV detection. Mobile phase A: Water/0.04% NH3.H₂O and Mobile Phase B: Acetonitrile. 10% MPB to 95% in 1.0 min, hold at 95% MPB for 0.5 min, 95% MPB to 10% in 0.03 min, then equilibration to 10% MPB for 0.2 min.

LCMS Method G: Shim-pack XR-ODS, 50*3 mm, 3.0 μL injection, 1.2 mL/min flowrate, 90-900 amu scan range, 254 nm UV detection. Mobile Phase A (MPA): Water/0.05% TFA and Mobile Phase B (MPB): Acetonitrile/0.05% TFA. Elution 5% MPB to 100% in 2.00 min, hold at 100% MPB for 0.7 min, 100% MPB to 5% in 0.05 min, then equilibration to 5% MPB for 0.25 min.

LCMS Method H: EVO C18, 50*3 mm, 2.0 μL injection, 1.2 mL/min flowrate, 90-900 amu scan range, 254 nm UV detection. Mobile Phase A (MPA): Water/5 mM NH4HCO₃ and Mobile Phase B (MPB): Acetonitrile. Elution 10% MPB to 95% in 2.00 min, hold at 95% MPB for 0.6 min, 95% MPB to 10% in 0.05 min, then equilibration to 10% MPB for 0.25 min.

LCMS Method I: Titank C18, 50*3 mm, 2.0 μL injection, 1.2 mL/min flowrate, 90-900 amu scan range, 254 nm UV detection. Mobile Phase A (MPA): Water/5 mM NH₄HCO₃ and Mobile Phase B (MPB): Acetonitrile. Elution 40% MPB to 70% in 2.80 min, up to 95% in 0.20 min, hold at 95% MPB for 0.5 min, 95% MPB to 10% in 0.05 min, then equilibration to 10% MPB for 0.25 min.

LCMS Method J: Kinetex XB-C18 100A, 30*2.7 mm, 0.6 μL injection, 1.0 mL/min flowrate, 90-900 amu scan range, 254 nm UV detection. Mobile Phase A (MPA): Water/0.05% TFA and Mobile Phase B (MPB): Acetonitrile/0.05% TFA. Elution 5% MPB to 100% in 1.50 min, hold at 100% MPB for 0.8 min, 100% MPB to 5% in 0.03 min, then equilibration to 5% MPB for 0.17 min.

LCMS Method BA: Kinetex EVO C18 100A, 30*3 mm, 0.5 μL injection, 1.2 mL/min flowrate, 90-900 amu scan range, 254 nm UV detection. Mobile Phase A (MPA): Water/5 mM NH₄HCO₃ and Mobile Phase B (MPB): Acetonitrile. Elution 10% MPB to 95% in 2.0 min, hold at 95% MPB for 0.30 min, 95% MPB to 10% in 0.10 min.

LCMS Method BB: Xselect CSH C18, 50*3 mm, 1.0 μL injection, 1.2 mL/min flowrate, 90-900 amu scan range, 254 nm UV detection. Mobile Phase A (MPA): Water/0.1% FA and Mobile Phase B (MPB): Acetonitrile/0.1% FA. Elution 5% MPB to 100% in 2.00 min, hold at 100% MPB for 0.70 min, 100% MPB to 5% in 0.05 min, then equilibration to 5% MPB for 0.15 min.

LCMS Method BC: XBridge Shield RP18, 50*4.6 mm, 0.5 μL injection, 1.2 mL/min flowrate, 90-900 amu scan range, 254 nm UV detection. Mobile Phase A (MPA): Water/0.04% NH3.H₂O and Mobile Phase B (MPB): Acetonitrile. Elution 10% MPB to 95% in 2.00 min, hold at 95% MPB for 0.79 min, 95% MPB to 10% in 0.06 min, then equilibration to 10% MPB for 0.15 min.

LCMS Method BD: Shim-pack XR-ODS, 50*3 mm, 0.3 μL injection, 1.2 mL/min flowrate, 30-2000 amu scan range, 254 nm UV detection. Mobile Phase A (MPA): Water/0.05 TFA and Mobile Phase B (MPB): Acetonitrile/0.05% TFA. Elution 5% MPB to 100% in 1.10 min, hold at 100% MPB for 0.60 min, 100% MPB to 5% in 0.05 min, then equilibration to 5% MPB for 0.25 min.

LCMS Method BE: Kinetex 2.6 um EVO C18 100A, 50*3 mm, 0.6 μL injection, 1.2 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 1.20 min, hold at 95% MPB for 0.50 min, 95% MPB to 10% in 0.05 min, then equilibration to 10% MPB for 0.10 min.

LCMS Method BF: EVO C18, 50*3 mm, 0.1 μL injection, 1.2 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 2.00 min, hold at 95% MPB for 0.60 min, 95% MPB to 10% in 0.15 min, then equilibration to 10% MPB for 0.25 min.

LCMS Method BG: Titank C18, 50*3 mm, 0.5 μL injection, 1.5 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 1.80 min, hold at 95% MPB for 0.80 min, 95% MPB to 10% in 0.15 min, then equilibration to 10% MPB for 0.25 min.

LCMS Method BH: Poroshell HPH C18, 50*3 mm, 0.5 μL injection, 1.2 mL/min flowrate, 30-2000 amu scan range, 254 nm UV detection. Mobile Phase A (MPA): Water/5 mM NH₄HCO₃+5 mM NH₄OH and Mobile Phase B (MPB): Acetonitrile. Elution 10% MPB to 95% in 2.00 min, hold at 95% MPB for 0.70 min, 95% MPB to 5% in 0.05 min, then equilibration to 5% MPB for 0.25 min.

LCMS Method BI: HALOC18, 30*3 mm, 0.5 μL injection, 1.5 mL/min flowrate, 30-2000 amu scan range, 254 nm UV detection. Mobile Phase A (MPA): Water/0.05% TFA and Mobile Phase B (MPB): Acetonitrile/0.05% TFA. Elution 5% MPB to 100% in 1.20 min, hold at 100% MPB for 0.60 min, 100% MPB to 5% in 0.02 min, then equilibration to 5% MPB for 0.18 min.

LCMS Method BJ: HALOC18, 30*3 mm, 0.5 μL injection, 1.5 mL/min flowrate, 30-2000 amu scan range, 254 nm UV detection. Mobile Phase A (MPA): Water/0.1% FA and Mobile Phase B (MPB): Acetonitrile/0.1% FA. Elution 5% MPB to 100% in 1.20 min, hold at 100% MPB for 0.60 min, 100% MPB to 5% in 0.02 min, then equilibration to 5% MPB for 0.18 min.

NMR was recorded on BRUKER NMR 300.03 Mz, DUL-C-H, ULTRASHIELD™ 300, AVANCE 11 300 B-ACS™ 120 or BRUKER NMR 400.13 Mz, BBFO, ULTRASHIELD™ 400, AVANCE III 400, B-ACS™ 120.

### Preparative Examples

Scheme for the preparation of Exemplary Intermediates: Schemes below illustrate the preparation of exemplary intermediates.

### Synthesis of Intermediate 1 (1-(3-((tert-butoxycarbonyl)(methyl)amino)cyclobutyl)-5-fluoro-1H-pyrrolo[2,3-b]pyridine-3-carboxylic acid)

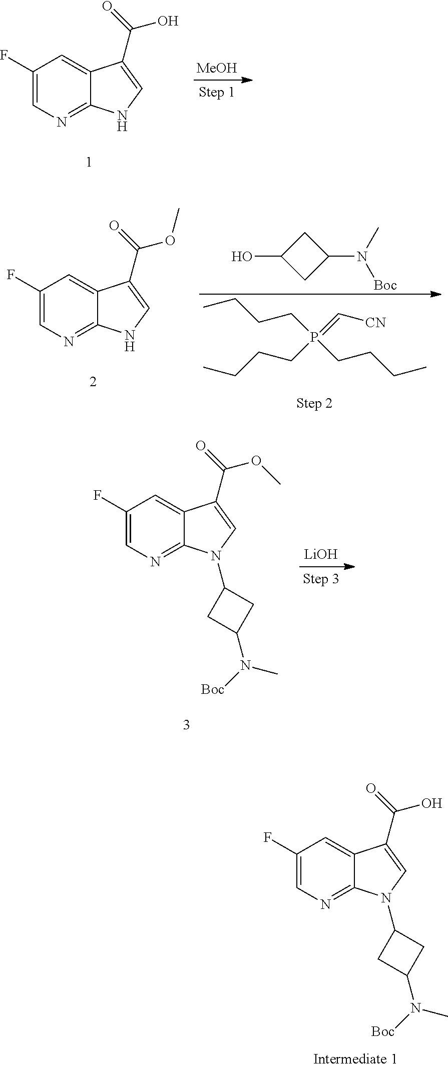

### Step 1: methyl 5-fluoro-1H-pyrrolo[2,3-b]pyridine-3-carboxylate

5-Fluoro-1H-pyrrolo[2,3-b]pyridine-3-carboxylic acid (3.0 g, 16.6 mmol, 1.0 eq.) was dissolved in THE (50 mL), then HATU (9.5 g, 25.0 mmol, 1.5 eq.), TEA (7.0 mL, 50.0 mmol, 3.0 eq.) and MeOH (2.0 mL, 50.0 mmol, 3.0 eq.) were added. The resulting solution was stirred for 6 hour at rt and concentrated in vacuo. The residue was purified by flash column chromatography on silica gel, eluting with DCM/methanol (100:1) to give methyl 5-fluoro-1H-pyrrolo[2,3-b]pyridine-3-carboxylate (1.8 g) as a white solid. LCMS Method A, MS-ESI: 195 [M+H]⁺.

### Step 2: tert-butyl (3-(5-fluoro-1H-pyrrolo[2,3-b]pyridin-1-yl)cyclobutyl)(methyl)carbamate

Methyl 5-fluoro-1H-pyrrolo[2,3-b]pyridine-3-carboxylate (500.0 mg, 2.6 mmol, 1.0 eq.) and tert-butyl N-(3-hydroxycyclobutyl)-N-methylcarbamate (777.4 mg, 3.9 mmol, 1.5 eq.) were dissolved in toluene (10 mL), then 2-(tributyl-λ5-phosphaneylidene)acetonitrile (2.5 g, 10.3 mmol, 4.0 eq.) was added in portions under nitrogen. The mixture was stirred for overnight at 115° C. and then concentrated in vacuo. The residue was purified by reverse flash chromatography with the following conditions: column, C18 silica gel; mobile phase, ACN/water, 0% to 100% gradient in 20 min; detector, UV 254 nm. This resulted in methyl 1-[3-[(tert-butoxycarbonyl)(methyl)amino]cyclobutyl]-5-fluoropyrrolo[2,3-b]pyridine-3-carboxylate (500 mg) as a yellow solid. LCMS Method C: [M+H]⁺=378.

### Step 3: 1-(3-((tert-butoxycarbonyl)(methyl)amino)cyclobutyl)-5-fluoro-1H-pyrrolo[2,3-b]pyridine-3-carboxylic acid

Methyl 1-[3-[(tert-butoxycarbonyl)(methyl)amino]cyclobutyl]-5-fluoropyrrolo[2,3-b]pyridine-3-carboxylate (400.0 mg, 1.1 mmol, 1.0 eq.) was dissolved 5 in methanol (4 mL) and water (4 mL), then LiOH (84.8 mg, 2.1 mmol, 2.0 eq.) was added. The solution was stirred for overnight at rt and then concentrated under vacuum. The result aqueous layer was adjusted to pH=5 by the dropwise addition of HCl (2N). The solution was extracted with DCM, dried over anhydrous Na₂SO₄ and concentrated under vacuum to give 1-[3-[(tert-butoxycarbonyl)(methyl)amino]cyclobutyl]-5-fluoropyrrolo[2,3-b]pyridine-3-carboxylic acid (380 mg) as a yellow solid. LCMS Method C: [M+H]⁺=364.

The intermediates in Table E1 were prepared using the same method described for Intermediate 1.

TABLE E1

| | Starting material | | |
|---|---|---|---|
| Intermediate | Used | Structure | LCMS data |
| Intermediate 2 | 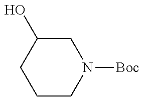 [SMILES: CC(C)(C)OC(=O)N1CCCC(O)C1] | 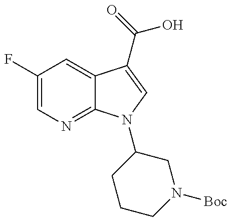 [SMILES: CC(C)(C)OC(=O)N1CCCC(n2cc(C(=O)O)c3cc(F)cnc32)C1] | Method D: MS- ESI: 364 [M + H]⁺ |
| Intermediate 3 | 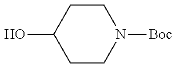 [SMILES: CC(C)(C)OC(=O)N1CCC(O)CC1] | 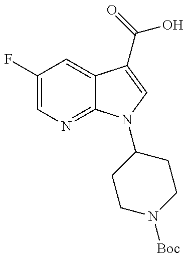 [SMILES: CC(C)(C)OC(=O)N1CCC(n2cc(C(=O)O)c3cc(F)cnc32)CC1] | Method D: MS- ESI: 364 [M + H]⁺ |

### Synthesis of Intermediate 4 (1-benzyl-5-fluoro-1H-pyrrolo[2,3-b]pyridine-3-carboxylic acid)

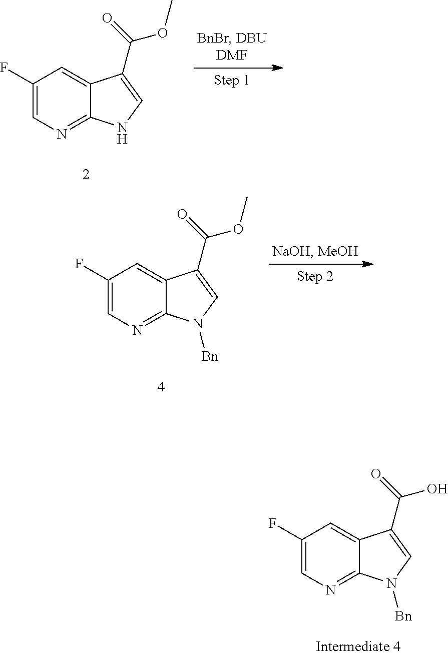 [SMILES: COC(=O)c1c[nH]c2ncc(F)cc12 ; COC(=O)c1cn(Cc2ccccc2)c2ncc(F)cc12 ; O=C(O)c1cn(Cc2ccccc2)c2ncc(F)cc12]

### Step 1: methyl 1-benzyl-5-fluoro-1H-pyrrolo[2,3-b]pyridine-3-carboxylate

Methyl 5-fluoro-1H-pyrrolo[2,3-b]pyridine-3-carboxylate (500.0 mg, 2.5 mmol, 1.0 eq.) was dissolved in DMF (40 mL), then DBU (411.6 mg, 2.7 mmol, 1.0 eq.) and benzyl bromide (440.4 mg, 2.5 mmol, 1.0 eq.) were added. The resulting solution was stirred for 2 hour at ambient temperature and quenched by the addition of water. The mixture was extracted with EtOAc and concentrated in vacuo. The residue was purified by flash column chromatography on silica gel, eluting with EtOAc/petroleum ether (1:7) to give methyl 1-benzyl-5-fluoropyrrolo[2,3-b]pyridine-3-carboxylate (420 mg) as a white solid. LCMS Method A: [M+H]⁺=285. ¹H NMR (400 MHz, DMSO-d₆): δ 8.57 (s, 1H), 8.41-8.39 (m, 1H), 8.10-8.07 (m, 1H), 7.35-7.25 (m, 5H), 5.54 (s, 2H), 3.83 (s, 3H).

### Step 2: 1-benzyl-5-fluoro-1H-pyrrolo[2,3-b]pyridine-3-carboxylic acid

Methyl 1-benzyl-5-fluoropyrrolo[2,3-b]pyridine-3-carboxylate (400.0 mg, 1.4 mmol, 1.0 eq.) was dissolved in MeOH (15 mL) and H₂O (5 mL), then NaOH (168.8 mg, 4.2 mmol, 3.0 eq.) was added. The resulting solution was stirred for 4 hour at 70° C. and cooled to rt. The solution was adjusted to pH=6.5 with HCl (2 mol/L) and the solids were collected by filtration to give 1-benzyl-5-fluoropyrrolo[2,3-b]pyridine-3-carboxylic acid (300 mg) as a white solid. LCMS Method A: [M+H]⁺=271. ¹H NMR (400 MHz, DMSO-d₆): δ 12.47 (s, 1H), 8.47 (s, 1H), 8.39-8.37 (m, 1H), 8.08-8.05 (m, 1H), 7.35-7.28 (m, 5H), 5.54 (s, 2H).

### Synthesis of Intermediate 5 (5-fluoro-1-methyl-1H-pyrrolo[2,3-b]pyridine-3-carboxylic acid)

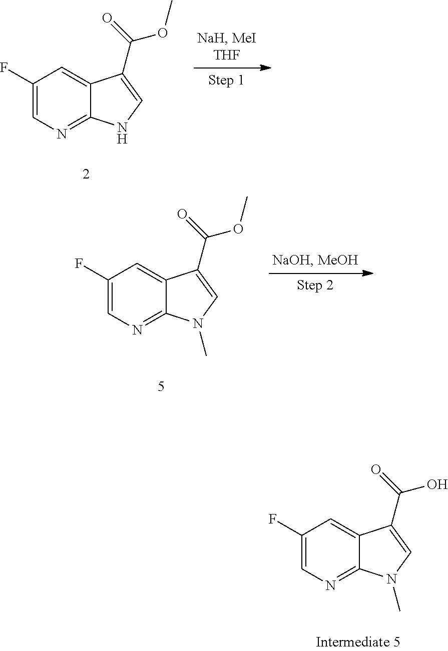

### Step 1: methyl 5-fluoro-1-methyl-1H-pyrrolo[2,3-b]pyridine-3-carboxylate

5-Fluoro-1H-pyrrolo[2,3-b]pyridine-3-carboxylate (200.0 mg, 1.0 mmol, 1.0 eq.) was dissolved in THF (10 mL), then NaH (60% wt., 40.0 mg, 1.0 mmol, 1.0 eq.) was added. After 30 min, Mel (219.3 mg, 1.5 mmol, 1.5 eq.) was added. The resulting solution was stirred 8 hour at ambient temperature and quenched with MeOH at 0° C. After concentration under vacuum, the residue was purified by reverse flash chromatography with the following conditions: column, C18; mobile phase A: water (10 MMOL/L NH₄HCO₃), mobile phase B: ACN; 10% B to 50% B gradient in 10 min; detector, UV 254 nm. This resulted in methyl 5-fluoro-1-methyl-1H-pyrrolo[2,3-b]pyridine-3-carboxylate (340.0 mg) as a yellow solid. LCMS Method A: [M+H]⁺=209.

### Step 2: 5-fluoro-1-methyl-1H-pyrrolo[2,3-b]pyridine-3-carboxylic acid

Methyl 5-fluoro-1-methyl-1H-pyrrolo[2,3-b]pyridine-3-carboxylate (300.0 mg, 1.4 mmol, 1.0 eq.) was dissolved in MeOH (6 mL), THE (6 mL) and H₂O (2 mL), then NaOH (288.2 mg, 7.2 mmol, 5.0 eq.) was added. The solution was stirred for overnight at 50° C. and cooled to ambient temperature, the resulting mixture was adjusted to pH 6˜7 with aq. NaOH (1 mol/L). The solution was extracted with EtOAc and concentrated under vacuum. The residue was purified by flash column chromatography on silica gel, eluting with petroleum ether/EtOAc (1:1) to give 5-fluoro-1-methyl-1H-pyrrolo[2,3-b]pyridine-3-carboxylic acid (240.0 mg) as a yellow solid. LCMS Method A: [M+H]⁺=195.

### Synthesis of Intermediate 6 (6-(4,4-difluorocyclohexyl)pyridin-3-amine)

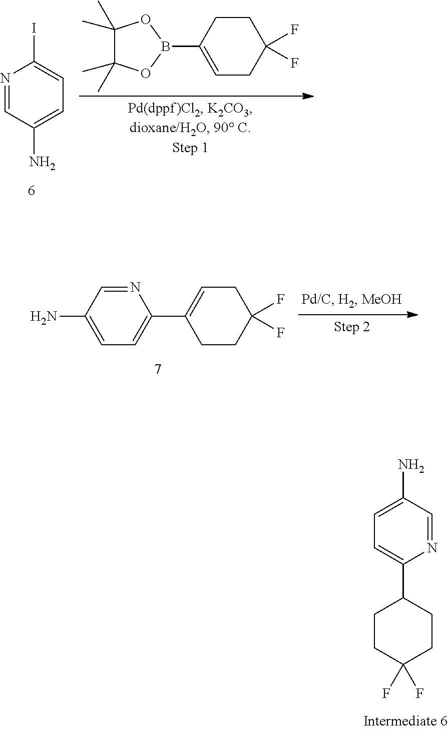

### Step 1: 6-(4,4-difluorocyclohex-1-en-1-yl)pyridin-3-amine

6-Iodopyridin-3-amine (5.0 g, 22.7 mmol, 1.0 eq.) was dissolved dioxane (80 mL) and H₂O (8 mL), then K₂CO₃ (9.4 g, 68.2 mmol, 3.0 eq.), 2-(4,4-difluorocyclohex-1-en-1-yl)-4,4,5,5-tetramethyl-1,3,2-dioxaborolane (9.5 g, 27.3 mmol, 1.2 eq.) and Pd(dppf)Cl₂ CH₂Cl₂ (185.6 mg, 0.2 mmol, 0.1 eq.) were added under nitrogen. The resulting solution was stirred for 12 hour at 90° C. and then concentrated under vacuum. The residue was purified by flash column chromatography on silica gel, eluting with ethyl acetate/petroleum ether (1:5) to give 6-(4,4-difluorocyclohex-1-en-1-yl)pyridin-3-amine (5.2 g) as a light yellow solid. LCMS Method H: [M+H]⁺=211.

### Step 2: 6-(4,4-difluorocyclohexyl)pyridin-3-amine

6-(4,4-difluorocyclohex-1-en-1-yl)pyridin-3-amine (5.2 g, 14.3 mmol, 1.0 eq.) was dissolved in MeOH (50 mL), then Pd/C (10% wt, 1.5 g, 1.4 mmol, 0.1 eq.) was added. The reaction vessel was evacuated then back filled with hydrogen three times, then stirred for 16 hour under an atmosphere of hydrogen. Filtration and concentration give 6-(4,4-difluorocyclohexyl)pyridin-3-amine (4.4 g) as a off-white solid. LCMS Method H: [M+H]⁺=213.

The intermediates in Table E2 were prepared using the same method described for Intermediate 6.

TABLE E2

| | Starting material | | | |
|---|---|---|---|---|
| Intermediate | Used | Bornic ester | Structure | LCMS data |
| Intermediate 7 | 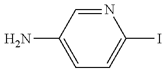 | 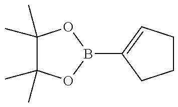 | 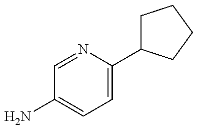 [SMILES: Nc1ccc(C2CCCC2)nc1] | Method E: MS-ESI: 163 [M + H]⁺ |
| Intermediate 8 | 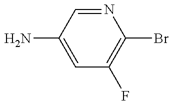 | 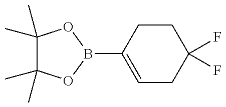 | 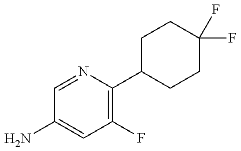 [SMILES: Nc1cnc(C2CCC(F)(F)CC2)c(F)c1] | Method E: MS-ESI: 231 [M + H]⁺ |
| Intermediate 11 | 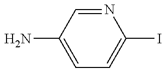 | 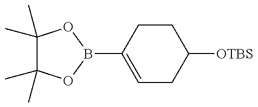 | 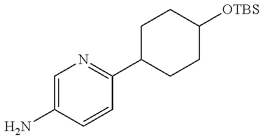 [SMILES: CC(C)(C)[Si](C)(C)OC1CCC(c2ccc(N)cn2)CC1] | Method E: MS-ESI: 307 [M + H]⁺ |

### Synthesis of Intermediate 9 (Intermediate B1) (5-chloro-6-(4,4-difluoropiperidin-1-yl)pyridin-3-amine)

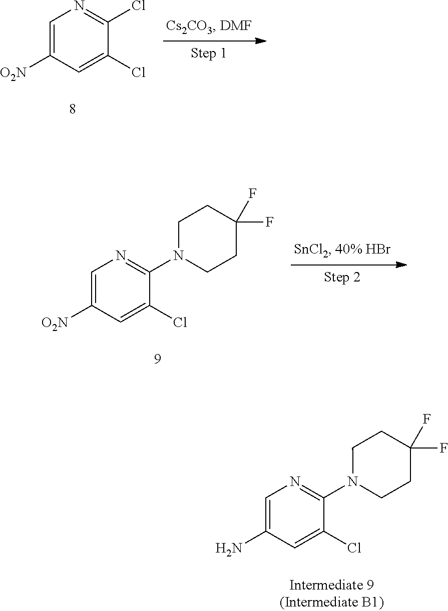 [SMILES: O=[N+]([O-])c1cnc(Cl)c(Cl)c1 ; O=[N+]([O-])c1cnc(N2CCC(F)(F)CC2)c(Cl)c1 ; Nc1cnc(N2CCC(F)(F)CC2)c(Cl)c1]

### Step 1: 3-chloro-2-(4,4-difluoropiperidin-1-yl)-5-nitropyridine

2,3-Dichloro-5-nitropyridine (5.0 g, 26.0 mmol, 1.0 eq.) and Cs₂CO₃ (42.2 g, 129.5 mmol, 5.0 eq.) were dissolved in DMF (200 mL), then 4,4-difluoropiperidine (3.8 g, 31.1 mmol, 1.2 eq.) was added. The resulting solution was stirred for 6 hour at 110° C. and then quenched by the addition of water. The resulting solution was extracted with ethyl acetate, washed with brine dried over anhydrous sodium sulfate and concentrated under vacuum. The residue was purified by reverse flash chromatography with following conditions: column, C18 silica gel; A: acetonitrile; B: water (0.05% NH₄OH), 30% B to 60% B in 30 min; detector, UV 254 nm. This resulted in 3-chloro-2-(4,4-difluoropiperidin-1-yl)-5-nitropyridine (4.3 g) as a yellow solid. LCMS Method F: [M+H]⁺=278.

### Step 2: 5-chloro-6-(4,4-difluoropiperidin-1-yl)pyridin-3-amine

3-Chloro-2-(4,4-difluoropiperidin-1-yl)-5-nitropyridine (2.0 g, 7.2 mmol, 1.0 eq.) was dissolved in HBr/H₂O (30 mL), then SnCl₂.2H₂O (3.2 g, 14.4 mmol, 2.0 eq.) was added. The resulting solution was stirred for 2 hour at ambient temperature. The solution was adjusted to pH 8 with aq. NaOH (2 mol/L). The resulting solution was extracted with ethyl acetate, dried over anhydrous sodium sulfate and concentrated under vacuum. The residue was purified by flash column chromatography on silica gel, eluting with ethyl acetate/petroleum ether (1:1) to give 5-chloro-6-(4,4-difluoropiperidin-1-yl)pyridin-3-amine (2.0 g) as a white solid. LCMS Method A: [M+H]⁺=248.

### Synthesis of Intermediate 10 (6-cyclobutoxy-5-fluoropyridin-3-amine)

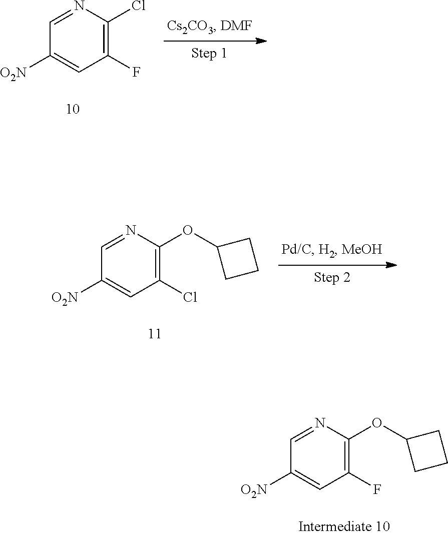 [SMILES: O=[N+]([O-])c1cnc(Cl)c(F)c1 ; O=[N+]([O-])c1cnc(OC2CCC2)c(Cl)c1 ; O=[N+]([O-])c1cnc(OC2CCC2)c(F)c1]

### Step 1: 2-cyclobutoxy-3-fluoro-5-nitropyridine

2-Chloro-3-fluoro-5-nitropyridine (500.0 mg, 2.8 mmol, 1.0 eq.) and cyclobutanol (306.3 mg, 4.2 mmol, 1.5 eq.) were dissolved in DMF (20 mL), then Cs₂CO₃ (1.8 g, 5.7 mmol, 2.0 eq.) was added. The resulting mixture was stirred for overnight at 120° C. and then concentrated under vacuum. The residue was purified by reverse flash chromatography with following conditions: column, C18 silica gel; mobile phase, A: water, B: MeOH, 10% B to 50% B in 30 min; detector, UV 254 nm. This resulted in 2-cyclobutoxy-3-fluoro-5-nitropyridine (500 mg) as yellow oil. LCMS Method B: [M+H]⁺=213.

### Step 2: 6-cyclobutoxy-5-fluoropyridin-3-amine

2-cyclobutoxy-3-fluoro-5-nitropyridine (700.0 mg, 3.3 mmol, 1.0 eq.) was dissolved MeOH (15 mL), then Pd/C (10% wt, 200 mg, 0.2 mmol, 0.1 eq.) was added. The reaction vessel was evacuated then back filled with hydrogen three times, then stirred for 8 h under an atmosphere of hydrogen. Filtration and concentration gave 6-cyclobutoxy-5-fluoropyridin-3-amine (120 mg) as a black solid, that was used without additional purification. LCMS Method A: [M+H]⁺=183.

### Synthesis of Intermediate 12 (1-(5-bromo-1H-pyrrolo[2,3-b]pyridin-3-yl)-3-(6-(4,4-difluorocyclohexyl)pyridin-3-yl)urea)

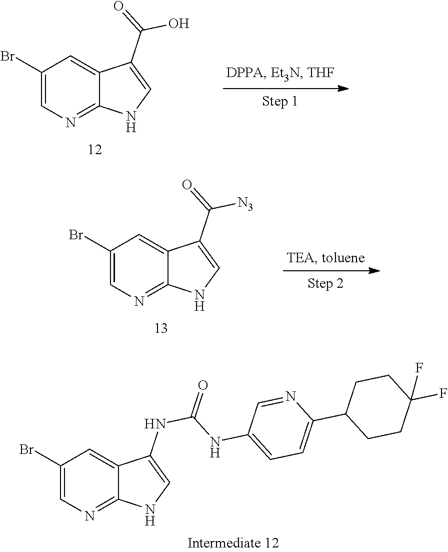 [SMILES: O=C(O)c1c[nH]c2ncc(Br)cc12 ; [N-]=[N+]=NC(=O)c1c[nH]c2ncc(Br)cc12 ; O=C(Nc1ccc(C2CCC(F)(F)CC2)nc1)Nc1c[nH]c2ncc(Br)cc12]

### Step 1: 5-bromo-1H-pyrrolo[2,3-b]pyridine-3-carbonyl azide

5-Bromo-1H-pyrrolo[2,3-b]pyridine-3-carboxylic acid (2.0 g, 8.3 mmol, 1.0 eq.) was dissolved in THE (10 mL), TEA (1.7 mL, 12.4 mmol, 1.5 eq.) and DPPA (2.7 mL, 12.4 mmol, 1.5 eq.) were added. The resulting solution was stirred for 2 hour at ambient temperature and then concentrated under vacuum. The residue was purified by flash column chromatography on silica gel, eluting with ethyl acetate/petroleum ether (1:3) to give 5-bromo-1H-pyrrolo[2,3-b]pyridine-3-carbonyl azide (1.5 g) as a white solid.

### Step 2: 1-(5-bromo-1H-pyrrolo[2,3-b]pyridin-3-yl)-3-(6-(4,4-difluorocyclohexyl)pyridin-3-yl)urea

5-Bromo-1H-pyrrolo[2,3-b]pyridine-3-carbonyl azide (500.0 mg, 1.9 mmol, 1.0 eq.) was dissolved in toluene (3 mL), then TEA (0.5 mL, 3.8 mmol, 2.0 eq.) and 6-(4,4-difluorocyclohexyl)pyridin-3-amine (478.6 mg, 2.3 mmol, 1.2 eq.) were added. The resulting solution was stirred for 2 hour at 90° C. and concentrated under vacuum. The residue was purified by flash column chromatography on silica gel, eluting with ethyl acetate/petroleum ether (1:1) to give 3-[5-bromo-1H-pyrrolo[2,3-b]pyridin-3-yl]-1-[6-(4,4-difluorocyclohexyl)pyridin-3-yl]urea (150 mg) as a white solid. LCMS Method B: [M+H]⁺=450.

### Synthesis of Intermediate B1 (5-chloro-6-(4,4-difluoropiperidin-1-yl)pyridin-3-amine)

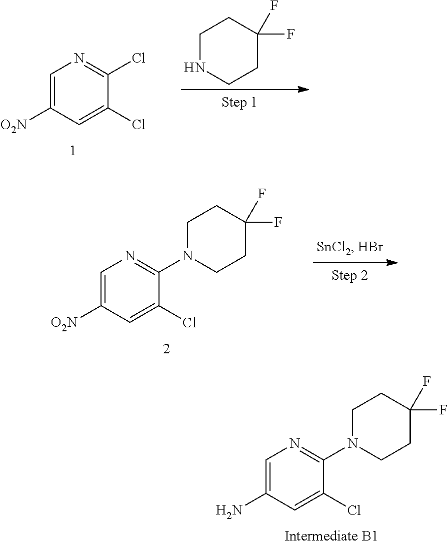 [SMILES: O=[N+]([O-])c1cnc(Cl)c(Cl)c1 ; FC1(F)CCNCC1 ; O=[N+]([O-])c1cnc(N2CCC(F)(F)CC2)c(Cl)c1 ; Nc1cnc(N2CCC(F)(F)CC2)c(Cl)c1]

### Step 1: 3-chloro-2-(4,4-difluoropiperidin-1-yl)-5-nitropyridine

2,3-Dichloro-5-nitropyridine (600.0 mg, 3.1 mmol, 1.0 equiv.) was dissolved in DMF (30 mL), Cs₂CO₃ (4.1 g, 12.4 mmol, 4.0 equiv.) and 4,4-difluoropiperidine (375.1 mg, 3.1 mmol, 1.0 equiv.) were added. The reaction mixture was stirred for 6 hours at 60° C. and then quenched by the addition of water. The resulting solution was extracted with ethyl acetate, washed with brine, dried over anhydrous sodium sulfate and concentrated under vacuum to give 3-chloro-2-(4,4-difluoropiperidin-1-yl)-5-nitropyridine (420 mg) as a yellow solid. LCMS Method BC: [M+H]⁺=278.

### Step 2: 5-chloro-6-(4,4-difluoropiperidin-1-yl)pyridin-3-amine

3-Chloro-2-(4,4-difluoropiperidin-1-yl)-5-nitropyridine (3.4 g, 12.2 mmol, 1.0 equiv.) was dissolved in 40% HBr (10.0 mL), then SnCl₂ (5.5 g, 29.0 mmol, 2.4 equiv.). The resulting solution was stirred for 2 hours at ambient temperature and adjusted to pH 8 with aqueous NaOH (1 mol/L). The mixture was extracted with ethyl acetate, dried over anhydrous Na₂SO₄ and concentrated under vacuum. The residue was purified by flash column chromatography on silica gel, eluting with DCM/MeOH (10:1) to give 5-chloro-6-(4,4-difluoropiperidin-1-yl)pyridin-3-amine (2.8 g) as a brown solid. LCMS Method BC: [M+H]⁺=248.

The intermediates in the following table were prepared using the method described for Intermediate B1.

| | Starting | Starting | | |
|---|---|---|---|---|
| Intermediate | material A | material B | Structure | LCMS data |
| Intermediate B2 | 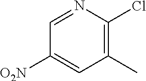 | 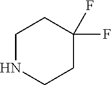 | 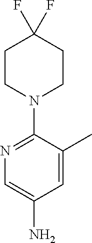 [SMILES: Cc1cc(N)cnc1N1CCC(F)(F)CC1] | Method BA: MS-ESI: 228 [M + H]⁺ |
| Intermediate B3 | 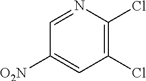 [SMILES: O=[N+]([O-])c1cnc(Cl)c(Cl)c1] | 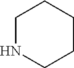 | 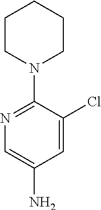 [SMILES: Nc1cnc(N2CCCCC2)c(Cl)c1] | Method BA: MS-ESI: 212 [M + H]+ |
| Intermediate B4 | 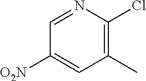 | 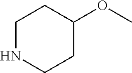 | 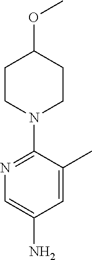 [SMILES: COC1CCN(c2ncc(N)cc2C)CC1] | Method BC: MS-ESI: 222 [M + H]+ |
| Intermediate B5 | 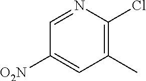 | 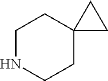 | 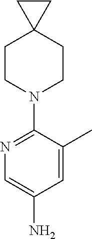 [SMILES: Cc1cc(N)cnc1N1CCC2(CC1)CC2] | Method BC: MS-ESI: 218 [M + H]+ |
| Intermediate B6 | 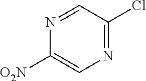 | 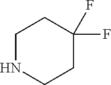 | 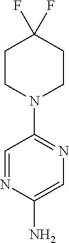 [SMILES: Nc1cnc(N2CCC(F)(F)CC2)cn1] | Method BC: MS-ESI: 215 [M + H]+ |
| Intermediate B7 | 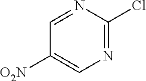 | 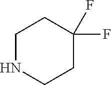 | 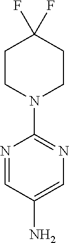 [SMILES: Nc1cnc(N2CCC(F)(F)CC2)nc1] | Method BA: MS-ESI: 215 [M + H]+ |
| Intermediate B8 | 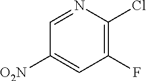 [SMILES: O=[N+]([O-])c1cnc(Cl)c(F)c1] | 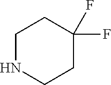 | 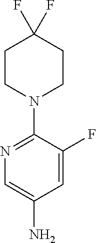 [SMILES: Nc1cnc(N2CCC(F)(F)CC2)c(F)c1] | Method BA: MS-ESI: 232 [M + H]+ |
| Intermediate B9 | 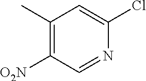 | 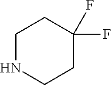 | 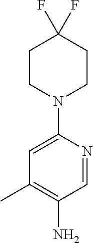 [SMILES: Cc1cc(N2CCC(F)(F)CC2)ncc1N] | Method BA: MS-ESI: 228 [M + H]+ |
| Intermediate B10 | 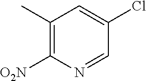 | 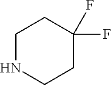 | 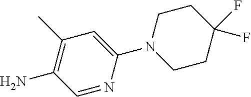 [SMILES: Cc1cc(N2CCC(F)(F)CC2)ncc1N] | Method BA: MS-ESI: 228 [M + H]+ |
| Intermediate B11 | 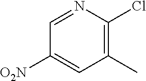 | 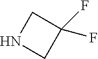 | 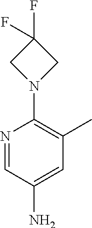 | Method BC: MS-ESI: 200 [M + H]+ |
| Intermediate B12 | 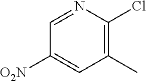 | 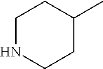 | 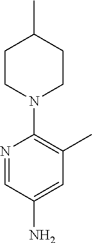 | Method BC: MS-ESI: 206 [M + H]+ |
| Intermediate B13 | 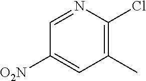 | 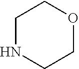 | 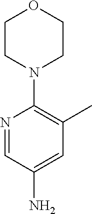 | Method BC: MS-ESI: 194 [M + H]+ |
| Intermediate B14 | 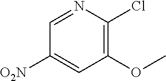 | 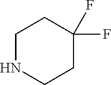 | 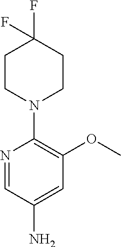 [SMILES: COc1cc(N)cnc1N1CCC(F)(F)CC1] | Method BA: MS-ESI: 244 [M + H]+ |
| Intermediate B15 | 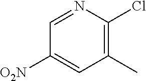 | 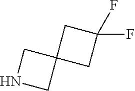 | 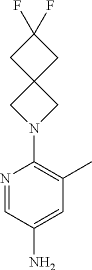 [SMILES: Cc1cc(N)cnc1N1CC2(C1)CC(F)(F)C2] | Method BA: MS-ESI: 240 [M + H]+ |
| Intermediate B16 | 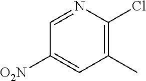 | 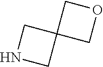 | 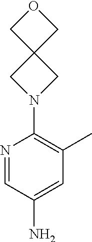 | Method BA: MS-ESI: 206 [M + H]+ |
| Intermediate B17 | 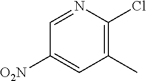 | 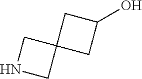 | 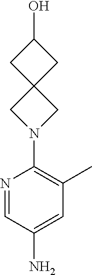 [SMILES: Cc1cc(N)cnc1N1CC2(CC(O)C2)C1] | Method BA: MS-ESI: 220 [M + H]+ |
| Intermediate B57 | 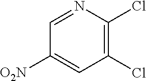 [SMILES: O=[N+]([O-])c1cnc(Cl)c(Cl)c1] | 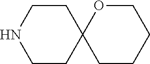 [SMILES: C1CCC2(CCNCC2)OC1] | 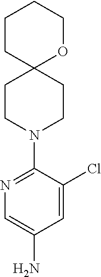 [SMILES: Nc1cnc(N2CCC3(CCCCO3)CC2)c(Cl)c1] | Method BA: MS-ESI: 282 [M + H]+ |
| Intermediate B58 | 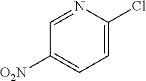 | 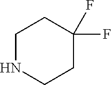 | 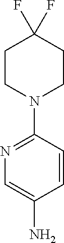 [SMILES: Nc1ccc(N2CCC(F)(F)CC2)nc1] | Method BA: MS-ESI: 214 [M + H]+ |
| Intermediate B59 | 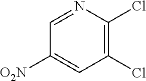 [SMILES: O=[N+]([O-])c1cnc(Cl)c(Cl)c1] | 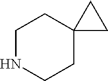 | 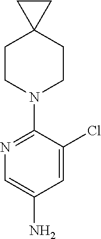 [SMILES: Nc1cnc(N2CCC3(CC2)CC3)c(Cl)c1] | Method BA: MS-ESI: 238 [M + H]+ |
| Intermediate B60 | 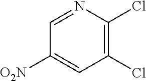 [SMILES: O=[N+]([O-])c1cnc(Cl)c(Cl)c1] | 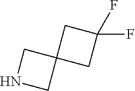 | 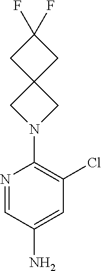 [SMILES: Nc1cnc(N2CC3(C2)CC(F)(F)C3)c(Cl)c1] | Method BC: MS-ESI: 260 [M + H]+ |
| Intermediate B61 | 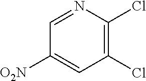 [SMILES: O=[N+]([O-])c1cnc(Cl)c(Cl)c1] | 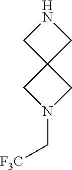 [SMILES: FC(F)(F)CN1CC2(CNC2)C1] | 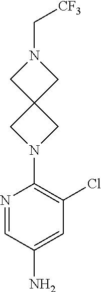 [SMILES: Nc1cnc(N2CC3(CN(CC(F)(F)F)C3)C2)c(Cl)c1] | Method BA: MS-ESI: 307 [M + H]+ |
| Intermediate B62 | 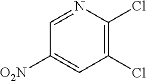 [SMILES: O=[N+]([O-])c1cnc(Cl)c(Cl)c1] | 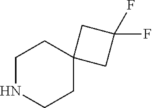 [SMILES: FC1(F)CC2(CCNCC2)C1] | 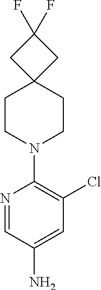 [SMILES: Nc1cnc(N2CCC3(CC2)CC(F)(F)C3)c(Cl)c1] | Method BA: MS-ESI: 288 [M + H]+ |
| Intermediate B63 | 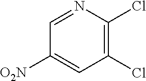 [SMILES: O=[N+]([O-])c1cnc(Cl)c(Cl)c1] | 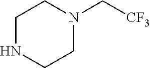 [SMILES: FC(F)(F)CN1CCNCC1] | 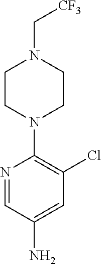 [SMILES: Nc1cnc(N2CCN(CC(F)(F)F)CC2)c(Cl)c1] | Method BC: MS-ESI: 295 [M + H]+ |
| Intermediate B64 | 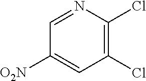 [SMILES: O=[N+]([O-])c1cnc(Cl)c(Cl)c1] | 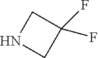 | 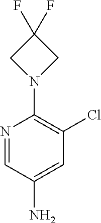 [SMILES: Nc1cnc(N2CC(F)(F)C2)c(Cl)c1] | Method BC: MS-ESI: 220 [M + H]+ |
| Intermediate B65 | 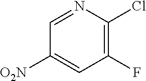 [SMILES: O=[N+]([O-])c1cnc(Cl)c(F)c1] | 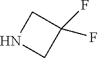 | 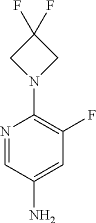 [SMILES: Nc1cnc(N2CC(F)(F)C2)c(F)c1] | Method BA: MS-ESI: 204 [M + H]+ |
| Intermediate B66 | 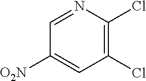 [SMILES: O=[N+]([O-])c1cnc(Cl)c(Cl)c1] | 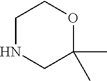 | 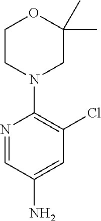 [SMILES: CC1(C)CN(c2ncc(N)cc2Cl)CCO1] | Method BA: MS-ESI: 242 [M + H]+ |

### Synthesis of Intermediate B18 (5-chloro-6-(4,4-difluorocyclohexyl)pyridin-3-amine)

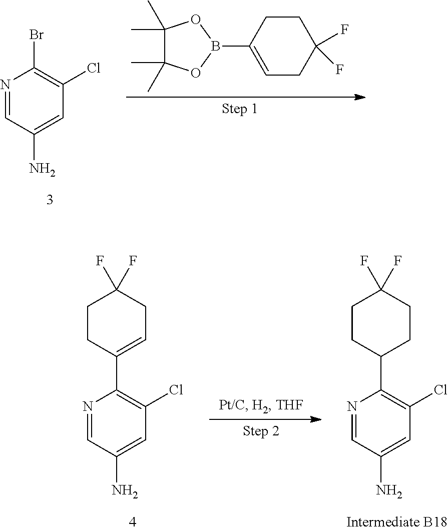

### Step 1: 5-chloro-6-(4,4-difluorocyclohex-1-en-1-yl)pyridin-3-amine

6-Bromo-5-chloropyridin-3-amine (2.0 g, 9.6 mmol, 1.0 equiv.) and 2-(4,4-difluorocyclohex-1-en-1-yl)-4,4,5,5-tetramethyl-1,3,2-dioxaborolane (2.8 g, 11.6 mmol, 1.2 equiv.) were dissolved in dioxane (35 mL) and water (7 mL), then K₂CO₃ (2.7 g, 19.3 mmol, 2.0 equiv.) and Pd(dppf)Cl₂ (705.4 mg, 1.0 mmol, 0.1 equiv.) were added nitrogen.

The reaction mixture was heated to 80° C. for 6 hours, then cooled to ambient temperature and concentrated under vacuum. The residue was purified by flash column chromatography on silica gel, eluting with ethyl acetate/petroleum ether (1:1) to give 5-chloro-6-(4,4-difluorocyclohex-1-en-1-yl)pyridin-3-amine (2.1 g) as an off-white solid. LCMS Method BA: [M+H]⁺=245.

### Step 2: 5-chloro-6-(4,4-difluorocyclohexyl)pyridin-3-amine

5-Chloro-6-(4,4-difluorocyclohex-1-en-1-yl)pyridin-3-amine (2.0 g, 8.2 mmol, 1.0 equiv.) was dissolved in THE (50 mL), then Pt/C (1.50 g, 3%) was added. The reaction mixture was solution was sparged with nitrogen, placed under an atmosphere of hydrogen gas (balloon), then heated to 80° C. for 36 hours. The solids were removed by filtration and the filtrate was concentrated under vacuum. The residue purified by flash column chromatography on silica gel, eluting with ethyl acetate/petroleum ether (1:1) to give 5-chloro-6-(4,4-difluorocyclohexyl)pyridin-3-amine (300 mg) as a pale yellow solid. LCMS Method BE: [M+H]⁺=247.

The intermediates in the following table were prepared using the method described for Intermediate B18.

| | Starting | Starting | | LCMS |
|---|---|---|---|---|
| Intermediate | material A | material B | Structure | data |
| Intermediate B19 | 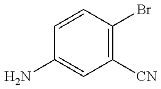 | 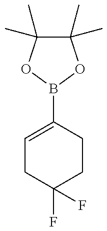 | 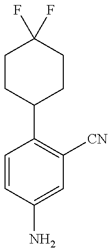 [SMILES: N#Cc1cc(N)ccc1C1CCC(F)(F)CC1] | Method BA: MS- ESI: 237 [M + H]⁺ |
| Intermediate B20 | 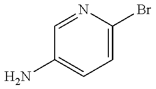 | 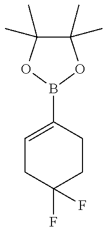 | 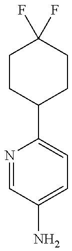 [SMILES: Nc1ccc(C2CCC(F)(F)CC2)nc1] | Method BC: MS- ESI: 213 [M + H]⁺ |
| Intermediate B21 | 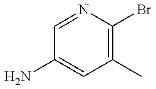 | 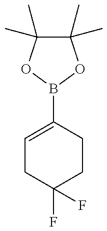 | 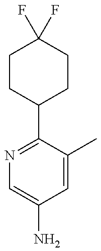 [SMILES: Cc1cc(N)cnc1C1CCC(F)(F)CC1] | Method BD: MS- ESI: 227 [M + H]⁺ |
| Intermediate B23 | 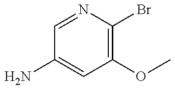 | 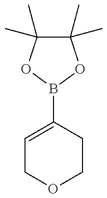 | 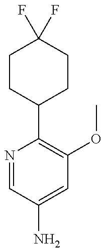 [SMILES: COc1cc(N)cnc1C1CCC(F)(F)CC1] | Method BA: MS- ESI: 243 [M + H]⁺ |
| Intermediate B54 | 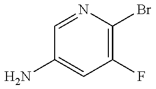 | 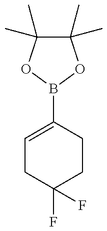 | 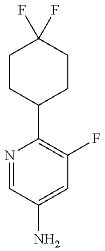 [SMILES: Nc1cnc(C2CCC(F)(F)CC2)c(F)c1] | Method BA: MS- ESI: 231 [M + H]+ |
| Intermediate B55 | 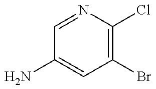 | 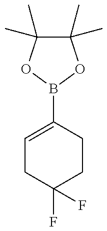 | 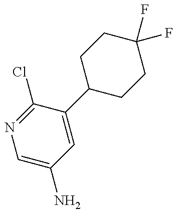 [SMILES: Nc1cnc(Cl)c(C2CCC(F)(F)CC2)c1] | Method BA: MS- ESI: 247 [M + H]+ |
| Intermediate B56 | 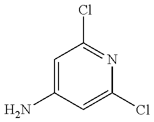 | 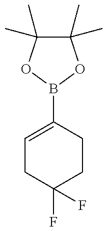 | 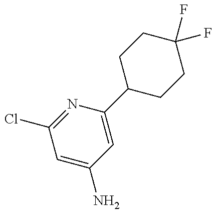 [SMILES: Nc1cc(Cl)nc(C2CCC(F)(F)CC2)c1] | Method BA: MS- ESI: 247 [M + H]+ |
| Intermediate B67 | 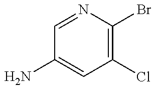 | 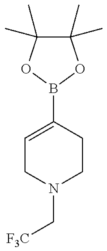 | 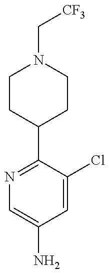 [SMILES: Nc1cnc(C2CCN(CC(F)(F)F)CC2)c(Cl)c1] | Method BC: MS- ESI: 294 [M + H]+ |

### Synthesis of intermediate B24 (4-(3,3-difluorocyclobutyl)-3-fluoroaniline)

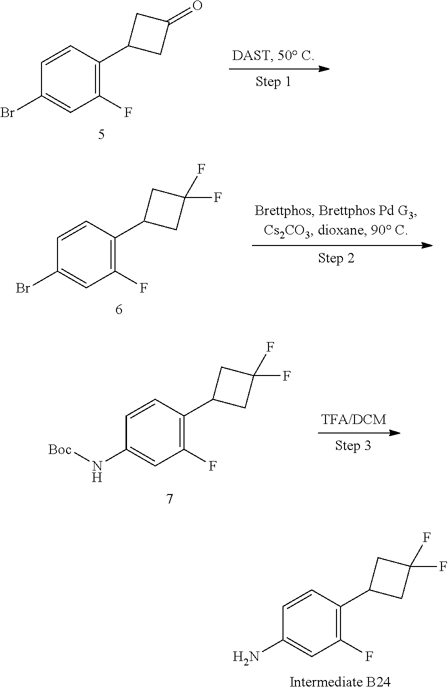 [SMILES: O=C1CC(c2ccc(Br)cc2F)C1 ; Fc1cc(Br)ccc1C1CC(F)(F)C1 ; CC(C)(C)OC(=O)Nc1ccc(C2CC(F)(F)C2)c(F)c1 ; Nc1ccc(C2CC(F)(F)C2)c(F)c1]

### Step 1: 4-bromo-1-(3,3-difluorocyclobutyl)-2-fluorobenzene

3-(4-Bromo-2-fluorophenyl)cyclobutan-1-one (1.3 g, 5.3 mmol, 1.0 equiv.) was dissolved in DAST (30 mL) at 0° C. under atmosphere of nitrogen. The resulting mixture was stirred overnight at ambient temperature, then cooled to 0° C. and quenched by the addition of aqueous NaHCO₃. The resulting mixture was extracted with DCM, dried over anhydrous Na₂SO₄ and concentrated under vacuum. The residue was purified by flash column chromatography on silica gel, eluting with ethyl acetate/petroleum ether (1:2) to give 4-bromo-1-(3,3-difluorocyclobutyl)-2-fluorobenzene (1.1 g) as a yellow oil. ¹H NMR (300 MHz, DMSO-d₄): δ 7.53-7.49 (m, 1H), 7.43-7.34 (m, 2H), 3.52-3.46 (m, 1H), 3.07-2.94 (m, 2H), 2.84-2.66 (m, 2H).

### Step 2: tert-butyl (4-(3,3-difluorocyclobutyl)-3-fluorophenyl)carbamate

4-Bromo-1-(3,3-difluorocyclobutyl)-2-fluorobenzene (1.1 g, 4.2 mmol, 1.0 equiv.) and BocNH₂ (2.4 g, 20.7 mmol, 5.0 equiv.) were dissolved in toluene (11 mL). Pd₂(dba)₃ (0.4 g, 0.4 mmol, 0.1 equiv.), XPhos (0.4 g, 0.8 mmol, 0.2 equiv.) and t-BuOK (2.3 g, 20.7 mmol, 5.0 equiv.) were added under atmosphere of nitrogen. The reaction mixture was heated to 100° C. overnight, then cooled to ambient temperature and quenched by the addition of water. The resulting solution was extracted with ethyl acetate, dried over anhydrous sodium sulfate and concentrated under vacuum. The residue was purified by flash column chromatography on silica gel, eluting with ethyl acetate/petroleum ether (1:8) to give tert-butyl [4-(3,3-difluorocyclobutyl)-3-fluorophenyl]carbamate (1.0 g) as a white solid. LCMS Method BA: [M+H]⁺=302.

### Step 3: 4-(3,3-difluorocyclobutyl)-3-fluoroaniline

tert-Butyl [4-(3,3-difluorocyclobutyl)-3-fluorophenyl]carbamate (1.2 g, 4.0 mmol, 1.0 equiv.) was dissolved in DCM (12 mL) and cooled to 0° C., then TFA (3 mL) was added dropwise, maintaining the solution at 0° C. The reaction mixture was stirred for 2 hours at ambient temperature and then concentrated under vacuum. The residue was dissolved in DCM, and adjusted to pH 8 with aqueous NaHCO₃. The resulting solution was extracted with DCM, washed with brine, dried over anhydrous sodium sulfate and concentrated under vacuum to give crude 4-(3,3-difluorocyclobutyl)-3-fluoroaniline (800 mg) as a red oil. LCMS Method BA: [M+H]⁺=202.

### Synthesis of Intermediate B25 (6-(4,4-difluoropiperidin-1-yl)pyridazin-3-amine)

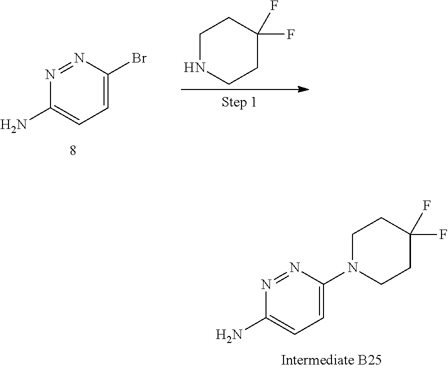 [SMILES: Nc1ccc(Br)nn1 ; FC1(F)CCNCC1 ; Nc1ccc(N2CCC(F)(F)CC2)nn1]

4,4-difluoropiperidine (1.0 g, 8.3 mmol, 1.0 equiv.) was dissolved in EtOH (10 mL), then 6-bromopyridazin-3-amine (1.4 g, 8.3 mmol, 1.0 equiv.) was added. The reaction mixture was heated to 80° C. overnight and concentrated under vacuum. The residue was purified by reverse flash chromatography with following conditions: column, C18 silica gel; mobile phase, ACN/water, 0% ACN increasing to 100% within 30 min; detector, UV 254 nm. This resulted in 6-(4,4-difluoropiperidin-1-yl)pyridazin-3-amine (410 mg) as a brown solid. LCMS Method BD: [M+H]⁺=215.

### Synthesis of Intermediate B27 (4-chloro-5-(4,4-difluoropiperidin-1-yl)pyridin-2-amine)

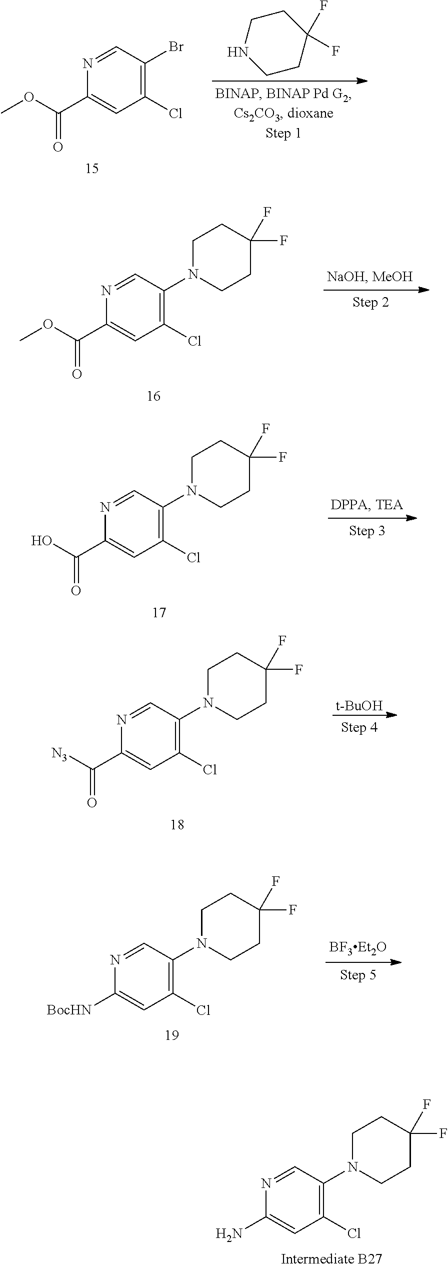

### Step 1: methyl 4-chloro-5-(4,4-difluoropiperidin-1-yl)picolinate

Methyl 5-bromo-4-chloropyridine-2-carboxylate (1.0 g, 3.9 mmol, 1.0 equiv.) was dissolved dioxane (10 mL), then Cs₂CO₃ (2.6 g, 7.9 mmol, 2.0 equiv.), BINAP (248.5 mg, 0.4 mmol, 0.1 equiv.), Binap Palladacycle Gen. 2 (0.3 mg, 0.1 equiv.) and 4,4-difluoropiperidine (967.2 mg, 7.9 mmol, 2.0 equiv.) were added under an atmosphere of nitrogen. The resulting solution was heated to 100° C. for 7 hours, then cooled to ambient temperature and concentrated under vacuum. The residue was purified by flash column chromatography on silica gel, eluting with ethyl acetate/petroleum ether (1:3) to give methyl 4-chloro-5-(4,4-difluoropiperidin-1-yl)pyridine-2-carboxylate (711.2 mg) as a yellow solid. LCMS Method BA: [M+H]⁺=291.

### Step 2: 4-chloro-5-(4,4-difluoropiperidin-1-yl)pyridine-2-carboxylic acid

Methyl 4-chloro-5-(4,4-difluoropiperidin-1-yl)pyridine-2-carboxylate (700.0 mg, 2.4 mmol, 1.0 equiv.) was dissolved MeOH (5 mL) and water (2 mL), then LiOH (288.3 mg, 12.0 mmol, 5.0 equiv.) was added. The reaction mixture was stirred for 3 hours at ambient temperature and then concentrated under vacuum. The residue was diluted with water, then the solution was adjusted to pH 5 with aqueous HCl (3 M). The solids were collected by filtration and dried to give 4-chloro-5-(4,4-difluoropiperidin-1-yl)pyridine-2-carboxylic acid (500.0 mg) as an off-white solid. LCMS Method BA: [M−H]⁻=275.

### Step 3: 4-chloro-5-(4,4-difluoropiperidin-1-yl)picolinoyl azide

4-Chloro-5-(4,4-difluoropiperidin-1-yl)pyridine-2-carboxylic acid (450.0 mg, 1.6 mmol, 1.0 equiv.) was dissolved THE (5 mL), then TEA (0.5 mL, 3.5 mmol, 2.2 equiv.), DPPA (671.4 mg, 2.4 mmol, 1.5 equiv.) were added. The resulting mixture was stirred for 6 hours at ambient temperature and then concentrated under vacuum. This resulted in 4-chloro-5-(4,4-difluoropiperidin-1-yl)picolinoyl azide (350.0 mg) as an off-white solid. LCMS Method BC: [M+H]⁺=302.

### Step 4: tert-butyl (4-chloro-5-(4,4-difluoropiperidin-1-yl)pyridin-2-yl)carbamate

4-Chloro-5-(4,4-difluoropiperidin-1-yl)pyridine-2-carbonyl azide (300.0 mg, 0.9 mmol, 1.0 equiv.) was dissolved t-BuOH (3 mL). The resulting solution was heated to 90° C. for 3 hours and then concentrated under vacuum. The residue was purified by flash column chromatography on silica gel, eluting with ethyl acetate/petroleum ether (1:3) to give tert-butyl (4-chloro-5-(4,4-difluoropiperidin-1-yl)pyridin-2-yl)carbamate (250.0 mg) of as an off-white solid. LCMS Method BC: [M+H]⁺=348.

### Step 5: 4-chloro-5-(4,4-difluoropiperidin-1-yl)pyridin-2-amine

tert-Butyl [4-chloro-5-(4,4-difluoropiperidin-1-yl)pyridin-2-yl]carbamate (250.0 mg, 0.7 mmol, 1.0 equiv.) was dissolved in BF₃.Et₂O (3.0 mL). The resulting solution was stirred for 3 hours at ambient temperature and then quenched by the addition of water. The resulting solution was adjusted to pH 7 with aqueous NaOH (3 M). The resulting solution was extracted with DCM, washed with brine, dried over anhydrous Na₂SO₄ and concentrated under vacuum to give 4-chloro-5-(4,4-difluoropiperidin-1-yl)pyridin-2-amine (180.0 mg) as an off-white solid. LCMS Method BC: [M+H]⁺=248.

The following intermediate was prepared using the method described for Intermediate B27.

| Intermediate | Starting material A | Starting material B | Structure | LCMS data |
|---|---|---|---|---|
| Intermediate B28 | 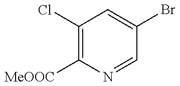 [SMILES: COC(=O)c1ncc(Br)cc1Cl] | 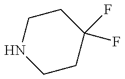 | 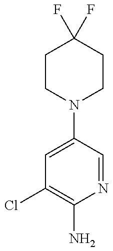 [SMILES: Nc1ncc(N2CCC(F)(F)CC2)cc1Cl] | Method BC: MS-ESI: 248 [M + H]⁺ |

### Synthesis of Intermediate B29 (4-chloro-6-(4,4-difluoropiperidin-1-yl)pyridin-3-amine)

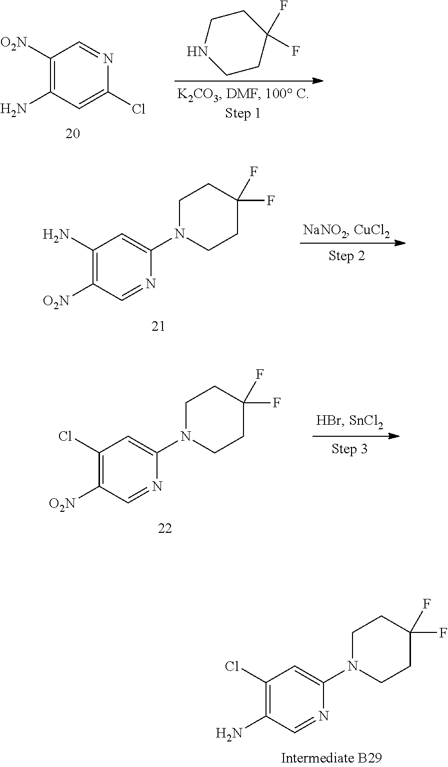 [SMILES: Nc1cc(Cl)ncc1[N+](=O)[O-] ; FC1(F)CCNCC1 ; Nc1cc(N2CCC(F)(F)CC2)ncc1[N+](=O)[O-] ; O=[N+]([O-])c1cnc(N2CCC(F)(F)CC2)cc1Cl ; Nc1cnc(N2CCC(F)(F)CC2)cc1Cl]

### Step 1: 2-(4,4-difluoropiperidin-1-yl)-5-nitropyridin-4-amine

2-Chloro-5-nitropyridin-4-amine (1.0 g, 5.8 mmol, 1.0 equiv.) and 4,4-difluoropiperidine (1.1 g, 9.1 mmol, 2.0 equiv.) were dissolved in DMF (50 mL), then K₂CO₃ (2.4 g, 17.4 mmol, 3.0 equiv.) was added. The reaction mixture was heated to 100° C. for 2 hours, then quenched by the addition of water. The resulting solution was extracted with ethyl acetate, washed with brine, dried over anhydrous Na₂SO₄ and concentrated under vacuum. The residue was purified by flash column chromatography on silica gel, eluting with ethyl acetate/petroleum ether (1:1) to give 2-(4,4-difluoropiperidin-1-yl)-5-nitropyridin-4-amine (949.2 mg) as an off-white solid. LCMS Method BA: [M+H]⁺=259.

### Step 2: 4-chloro-2-(4,4-difluoropiperidin-1-yl)-5-nitropyridine

2-(4,4-Difluoropiperidin-1-yl)-5-nitropyridin-4-amine (750.0 mg, 2.9 mmol, 1.0 equiv.) was dissolved in aqueous HCl (6M, 20 ml) and cooled to 0° C., then NaNO₂ (550.0 mg, 8.0 mmol, 2.7 equiv.) was added, maintaining the solution at 0° C. After 20 min at 0° C., CuCl₂ (781.5 mg, 5.8 mmol, 2.0 equiv.) was added. The reaction mixture was stirred for additional 1 hour at 0° C. and then quenched by the addition of water. The residue was purified by flash column chromatography on silica gel, eluting with ethyl acetate/petroleum ether (1:1) to give 4-chloro-2-(4,4-difluoropiperidin-1-yl)-5-nitropyridine (220.0 mg) as an off-white solid. LCMS Method BA: [M+H]⁺=278.

### Step 3: 4-chloro-6-(4,4-difluoropiperidin-1-yl)pyridin-3-amine

4-chloro-2-(4,4-difluoropiperidin-1-yl)-5-nitropyridine (200.0 mg, 0.7 mmol, 1.0 equiv.) was dissolved aqueous HBr (40%, 20 ml) and cooled to 0° C., then SnCl₂.2H₂O (914.0 mg, 4.1 mmol, 5.8 equiv.) was added, maintaining the solution at 0° C. The reaction mixture was stirred for 1 hour at ambient temperature and concentrated under vacuum. The residue was diluted with water and adjusted to pH 8 with aqueous NaOH (4 M). The resulting mixture was extracted with ethyl acetate, washed with brine and concentrated under vacuum. The residue was purified by flash column chromatography on silica gel, eluting with ethyl acetate/petroleum ether (1:3) to give 4-chloro-6-(4,4-difluoropiperidin-1-yl)pyridin-3-amine (126.4 mg) as an off-white soli. LCMS Method BC: [M+H]⁺=248.

### Synthesis of Intermediate B30 (5-(4,4-difluorocyclohexyl)-4-methoxypyridin-2-amine)

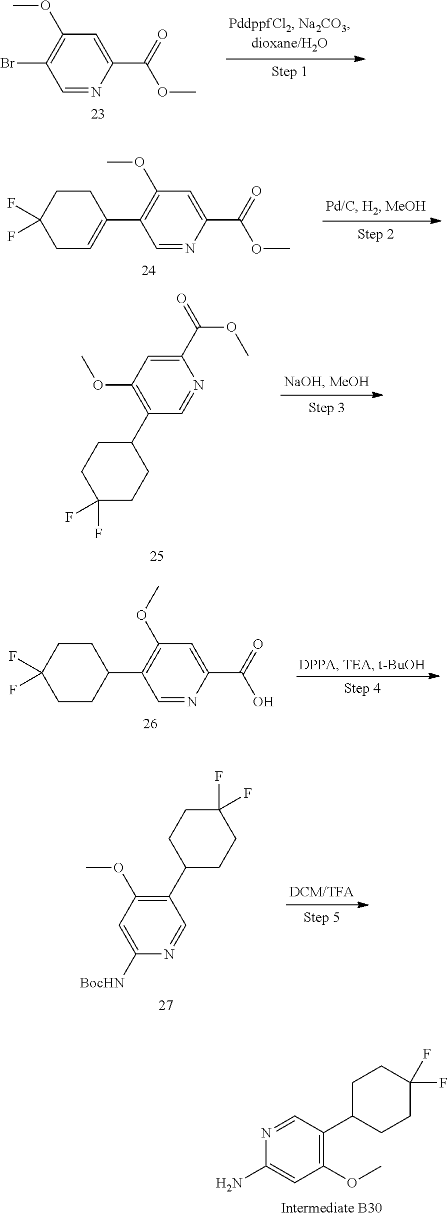

### Step 1: methyl 5-(4,4-difluorocyclohex-1-en-1-yl)-4-methoxypicolinate

Methyl 5-bromo-4-hydroxypyridine-2-carboxylate (1.5 g, 6.5 mmol, 1.0 equiv.) and 2-(4,4-difluorocyclohex-1-en-1-yl)-4,4,5,5-tetramethyl-1,3,2-dioxaborolane (4.7 g, 19.4 mmol, 3.0 equiv.) were dissolved in 1.4-dioxane (15 mL) and water (1.5 mL), then Pd(dppf)Cl₂ (0.5 g, 0.6 mmol, 0.1 equiv.) and Na₂CO₃ (2.1 g, 19.4 mmol, 3.0 equiv.) were added. The reaction mixture was heated 70° C. overnight, then quenched by the addition of water. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:1) to give methyl 5-(4,4-difluorocyclohex-1-en-1-yl)-4-hydroxypyridine-2-carboxylate (1.1 g) as a white solid. LCMS Method BD: [M+H]⁺=284.

### Step 2: methyl 5-(4,4-difluorocyclohexyl)-4-methoxypicolinate

Methyl 5-(4,4-difluorocyclohex-1-en-1-yl)-4-methoxypyridine-2-carboxylate (6.0 g, 21.2 mmol, 1.0 equiv.) was dissolved in ethyl acetate (60 mL), then Pd/C (10% wt., 1.2 g) was added. The reaction mixture was sparged with nitrogen, placed under an atmosphere of hydrogen gas (balloon), then stirred overnight at ambient temperature. The solids were removed by filtration and the filtrate was concentrated under vacuum to give methyl 5-(4,4-difluorocyclohexyl)-4-methoxypyridine-2-carboxylate (5.3 g) as an off-white solid. LCMS Method BD: [M+H]⁺=286.

### Step 3: 5-(4,4-difluorocyclohexyl)-4-methoxypicolinic acid

Methyl 5-(4,4-difluorocyclohexyl)-4-methoxypicolinate (800.0 mg, 2.8 mmol, 1.0 equiv.) was dissolved in MeOH (8 mL) and water (8 mL), then NaOH (449.0 mg, 11.2 mmol, 4.0 equiv.) was added. The reaction mixture was stirred overnight at ambient temperature and then concentrated under vacuum. The residue was diluted with water, and the solution was adjusted to pH 6 with aqueous HCl (6M). The resulting mixture was extracted with ethyl acetate, washed with brine, dried over anhydrous Na₂SO₄ and concentrated under vacuum. The resulting solid was washed with ACN and dried to give 5-(4,4-difluorocyclohexyl)-4-methoxypicolinic acid (450.0 mg) as a white solid. LCMS Method BA: [M+H]⁺=272.

### Step 4: tert-butyl (5-(4,4-difluorocyclohexyl)-4-methoxypyridin-2-yl)carbamate

5-(4,4-Difluorocyclohexyl)-4-methoxypicolinic acid (830.0 mg, 3.1 mmol, 1.0 equiv.) and TEA (0.5 mL, 3.7 mmol, 1.2 equiv.) were dissolved in t-BuOH (9 mL), then DPPA (1.0 mg, 3.7 mmol, 1.2 equiv.) was added in portions. The reaction mixture was heated to 90° C. overnight, then cooled to ambient temperature and quenched by the addition of water. The resulting mixture was extracted with ethyl acetate, washed with brine, dried over anhydrous Na₂SO₄ and concentrated under vacuum. The residue was purified by flash column chromatography on silica gel, eluting with DCM/MeOH (50:1) to give tert-butyl (5-(4,4-difluorocyclohexyl)-4-methoxypyridin-2-yl)carbamate (612.5 mg) as a white solid. LCMS Method BA: [M+H]⁺=343.

### Step 5: 5-(4,4-difluorocyclohexyl)-4-methoxypyridin-2-amine

tert-Butyl (5-(4,4-difluorocyclohexyl)-4-methoxypyridin-2-yl)carbamate (240.0 mg, 0.7 mmol, 1.0 equiv.) was dissolved in DCM (4 mL) and cooled to 0° C., then TFA (1 mL) was added dropwise, maintaining the solution at 0° C. The resulting mixture was stirred for 2 hours at ambient temperature and concentrated under vacuum. The residue was diluted with water, and then adjusted to pH 7 with saturated aqueous NaHCO₃. The resulting solution was extracted with ethyl acetate, washed with brine, dried over anhydrous Na₂SO₄ and concentrated under vacuum. The residue was purified by flash column chromatography on silica gel, eluting with DCM/MeOH (20:1) to give 5-(4,4-difluorocyclohexyl)-4-methoxypyridin-2-amine (152.5 mg) as a brown solid. LCMS Method BA: [M+H]⁺=243.

### Synthesis of Intermediate B31 (4-chloro-5-(4,4-difluorocyclohexyl)pyridin-2-amine)

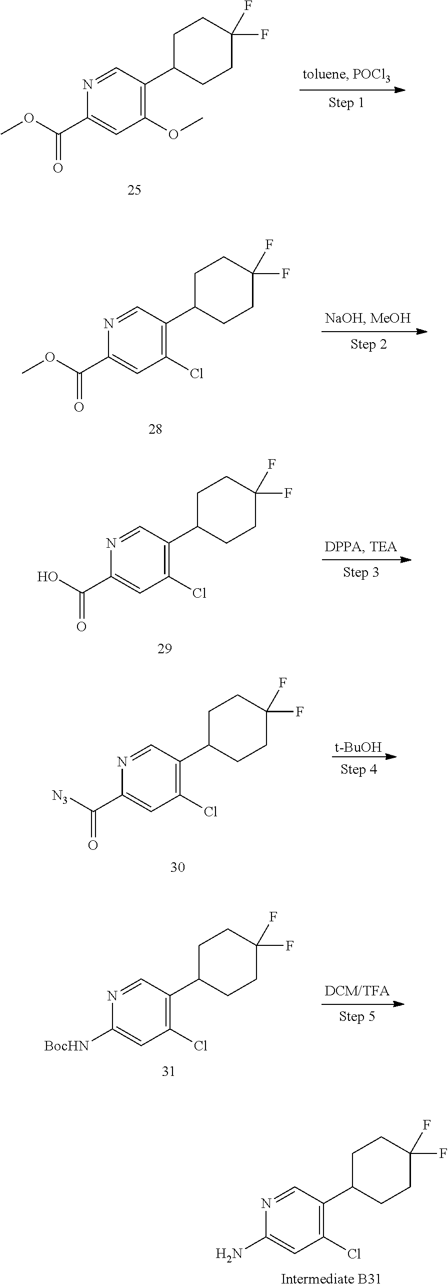

### Step 1: methyl 4-chloro-5-(4,4-difluorocyclohexyl)picolinate

Methyl 5-(4,4-difluorocyclohexyl)-4-methoxypyridine-2-carboxylate (0.8 g, 2.6 mmol, 1.0 equiv.) was dissolved in toluene (30 mL) and DMF (1 mL) and cooled to 0° C., then POCl₃ (1.1 mL, 13.1 mmol, 5.0 equiv.) was added dropwise, maintaining the temperature at 0° C. The reaction mixture was heated to 90° C. overnight, then cooled to 0° C. and quenched by the addition of ice-water. The mixture was adjusted to pH 8 with saturated aqueous NaHCO₃, then extracted with ethyl acetate, washed with brine, dried over anhydrous Na₂SO₄ and concentrated under vacuum. The residue was purified by flash column chromatography on silica gel, eluting with ethyl acetate/petroleum ether (1:3) to give methyl 4-chloro-5-(4,4-difluorocyclohexyl)pyridine-2-carboxylate (355.0 mg) as a white solid. LCMS Method BD: [M+H]⁺=290.

### Step 2: 4-chloro-5-(4,4-difluorocyclohexyl)picolinic acid

Methyl 4-chloro-5-(4,4-difluorocyclohexyl)pyridine-2-carboxylate (2.0 g, 6.9 mmol, 1.0 equiv.) was dissolved in MeOH (20 mL) and water (20 mL), then NaOH (1.1 g, 27.6 mmol, 4.0 equiv.) was added. The reaction mixture was stirred overnight at ambient temperature and concentrated under vacuum. The residue was diluted with water, then adjusted to pH 5 with aqueous HCl (6 M). The resulting solution was extracted with ethyl acetate, washed with brine, dried over anhydrous Na₂SO₄ and concentrated under vacuum to give 4-chloro-5-(4,4-difluorocyclohexyl)pyridine-2-carboxylic acid (705.1 mg) as a white solid. LCMS Method BB: [M−H]⁻=274.

### Step 3: 4-chloro-5-(4,4-difluorocyclohexyl)picolinoyl azide

4-Chloro-5-(4,4-difluorocyclohexyl)pyridine-2-carboxylic acid (430.0 mg, 1.6 mmol, 1.0 equiv.) and TEA (189 mg, 1.9 mmol, 1.2 equiv.) were dissolved in toluene (6 mL), then DPPA (515.0 mg, 1.9 mmol, 1.2 equiv.) was added. The reaction mixture was stirred overnight at ambient temperature and quenched by the addition of water. The resulting solution was extracted with ethyl acetate, washed with saturated aqueous NaHCO₃, dried over anhydrous Na₂SO₄ and concentrated under vacuum to give 4-chloro-5-(4,4-difluorocyclohexyl)pyridine-2-carbonyl azide (400.0 mg) as a light brown solid. LCMS Method BD: [M+H]⁺=301.

### Step 4: tert-butyl (4-chloro-5-(4,4-difluorocyclohexyl)pyridin-2-yl)carbamate

4-Chloro-5-(4,4-difluorocyclohexyl)pyridine-2-carbonyl azide (400.0 mg, 1.3 mmol, 1.0 equiv.) was dissolved in t-BuOH (4 mL). The solution was heated to 90° C. overnight. The precipitated solids were collected by filtration and washed with ethyl acetate to five tert-butyl N-[4-chloro-5-(4,4-difluorocyclohexyl)pyridin-2-yl]carbamate (380 mg) as a white solid. LCMS Method BD: [M+H]⁺=347.

### Step 5: 4-chloro-5-(4,4-difluorocyclohexyl)pyridin-2-amine

tert-Butyl N-[4-chloro-5-(4,4-difluorocyclohexyl)pyridin-2-yl]carbamate (190.0 mg, 0.5 mmol, 1.0 equiv.) was dissolved in DCM (2 mL) and TFA (0.5 mL). The reaction mixture was stirred for 2 hours at ambient temperature, and then concentrated under vacuum. The residue was dissolved in water and adjusted to pH=7 with saturated aqueous NaHCO₃. The resulting mixture was extracted with ethyl acetate, washed with brine, dried over anhydrous Na₂SO₄ and concentrated under vacuum. The residue was purified by flash column chromatography on silica gel, eluting with DCM/MeOH (20:1) to give 4-chloro-5-(4,4-difluorocyclohexyl)pyridin-2-amine (130 mg) as a light yellow solid. LCMS Method BD: [M+H]⁺=247.

### Synthesis of intermediate B32 (5-chloro-6-(3,3-difluorocyclobutyl)pyridin-3-amine)

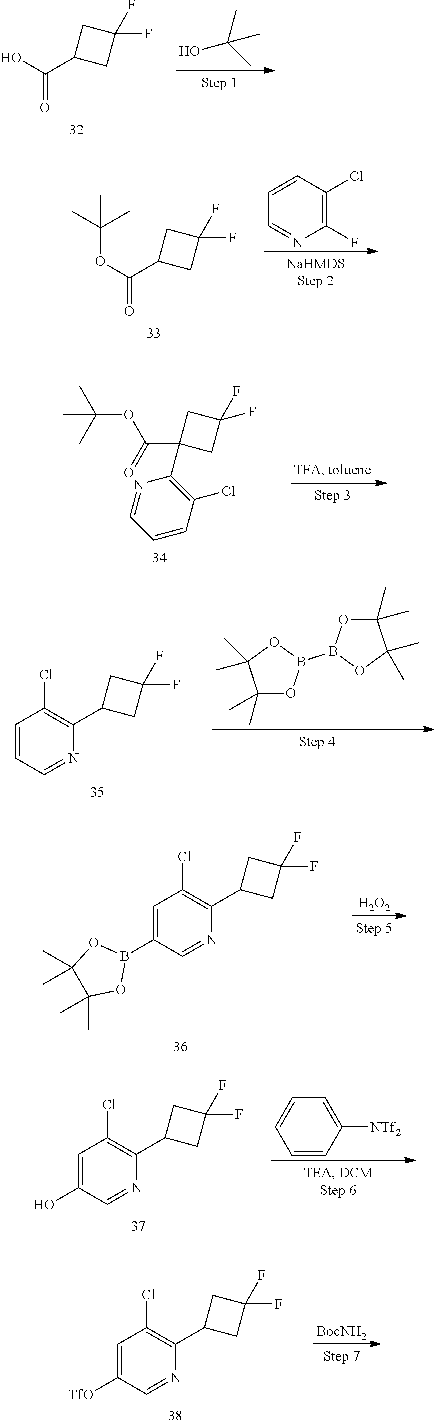

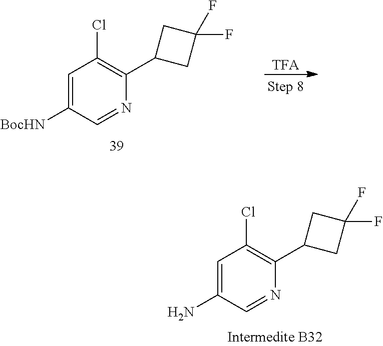 [SMILES: CC(C)(C)OC(=O)Nc1cnc(C2CC(F)(F)C2)c(Cl)c1 ; Nc1cnc(C2CC(F)(F)C2)c(Cl)c1 ; "TFA"]

### Step 1: tert-butyl 3,3-difluorocyclobutane-1-carboxylate

3,3-Difluorocyclobutanecarboxylic acid (1.0 g, 7.3 mmol, 1.0 equiv.) was dissolved in DCM (10 mL), N,N-dimethylpyridin-4-amine (92.0 mg, 0.7 mmol, 0.1 equiv.), 2-methylpropan-2-ol (1.1 g, 14.7 mmol, 2.0 equiv.) and N,N-dicyclohexylcarbodiimide (1.7 g, 8.1 mmol, 1.1 equiv.) were added at 10° C. The reaction mixture was warmed up to room temperature and stirred for 18 hours. The solid was removed by filtration and the filtrate was washed with aqueous HCl (2N), saturated aqueous NaHCO₃, brine, dried over anhydrous Na₂SO₄, and concentrated under vacuum to give crude tert-butyl 3,3-difluorocyclobutane-1-carboxylate (896.1 mg) as colorless oil. ¹H NMR (400 MHz, CDCl₃): δ 2.83-2.78 (m, 5H), 1.47 (s, 9H).

### Step 2: tert-butyl 1-(3-chloropyridin-2-yl)-3,3-difluorocyclobutane-1-carboxylate

3-Chloro-2-fluoropyridine (1.2 g, 10.4 mmol, 1.0 equiv.) and tert-butyl 3,3-difluorocyclobutane-1-carboxylate (2.0 g, 10.4 mmol, 1.0 equiv.) were dissolved in toluene (60 mL). This was followed by the addition of NaHMDS (2 M in THF, 6.2 ml, 12.4 mmol, 1.2 equiv.) dropwise with stirring at 0° C. in 10 min. The resulting solution was stirred for 2 hours at 0° C. and then quenched by the addition of saturated aqueous NH₄Cl. The resulting solution was extracted with ethyl acetate, dried over anhydrous sodium sulfate and concentrated under vacuum. The residue was purified by flash column chromatography on silica gel, eluting with ethyl acetate/petroleum ether (1:5) to give tert-butyl 1-(3-chloropyridin-2-yl)-3,3-difluorocyclobutane-1-carboxylate (1.6 g) as colorless oil. LCMS Method BD: [M+H]⁺=304.

### Step 3: 3-chloro-2-(3,3-difluorocyclobutyl)pyridine

tert-Butyl 1-(3-chloropyridin-2-yl)-3,3-difluorocyclobutane-1-carboxylate (1.5 g, 5.2 mmol, 1.0 equiv.) was dissolved in DCM (30 mL) and TFA (3 ml). The resulting solution was stirred for 10 hours at ambient temperature and then concentrated under vacuum. The residue was dissolved in toluene (30 mL) and stirred for 18 hours at 90° C. After cooling down to ambient temperature and quenching by addition of water, the pH value of the solution was adjusted to 7.5 with saturated aqueous Na₂CO₃. The solution was extracted with ethyl acetate, dried over anhydrous sodium sulfate and concentrated under vacuum. The residue was purified by flash column chromatography on silica gel, eluting with ethyl acetate/petroleum ether (1:7) to give 3-chloro-2-(3,3-difluorocyclobutyl)pyridine (700 mg) as colorless oil. LCMS Method BD: [M+H]⁺=204. ¹H NMR (400 MHz, DMSO-d₆): δ 8.45-8.43 (m, 1H), 7.69-7.67 (m, 1H), 7.40-7.38 (m, 1H), 3.72-3.70 (m, 1H), 3.02-2.85 (m, 4H).

### Step 4: 3-chloro-2-(3,3-difluorocyclobutyl)-5-(4,4,5,5-tetramethyl-1,3,2-dioxaborolan-2-yl)pyridine

3-chloro-2-(3,3-difluorocyclobutyl)pyridine (700.0 mg, 3.7 mmol, 1.0 equiv.) was dissolved in heptane (30 mL), bis(pinacolato)diboron (1.1 g, 4.4 mmol, 1.2 equiv.), 4,4-di-tert-butyl-2,2-dipyridyl (1.0 g, 3.7 mmol, 1.0 equiv.) and di-methanolatodiiridium(Ir—Ir)-cycloocta-1,5-diene (1:2) (495.8 mg, 0.7 mmol, 0.2 equiv.) were added under an atmosphere of nitrogen. The resulting solution was stirred for 18 hours at ambient temperature and then quenched by the addition of water. The resulting solution was extracted with ethyl acetate, dried over anhydrous sodium sulfate and concentrated under vacuum. The residue was purified by flash column chromatography on silica gel, eluting with ethyl acetate/petroleum ether (1:5) to give 3-chloro-2-(3,3-difluorocyclobutyl)-5-(4,4,5,5-tetramethyl-1,3,2-dioxaborolan-2-yl)pyridine (300 mg) as a white solid. LCMS Method BD: [M+H]⁺=330.

### Step 5: 5-chloro-6-(3,3-difluorocyclobutyl)pyridin-3-ol

3-chloro-2-(3,3-difluorocyclobutyl)-5-(4,4,5,5-tetramethyl-1,3,2-dioxaborolan-2-yl)pyridine (300.0 mg, 0.9 mmol, 1.0 equiv.) was dissolved in MeOH (10 mL) and H₂O (3 mL). Then H₂O₂ (30%, 0.14 ml, 1.4 mmol, 1.5 equiv.) was added. The resulting solution was stirred for 30 min at ambient temperature and then quenched by the addition of saturated aqueous Na₂S₂O₃. The resulting solution was extracted with ethyl acetate, dried over anhydrous sodium sulfate and concentrated under vacuum. The residue was purified by flash column chromatography on silica gel, eluting with ethyl acetate/petroleum ether (1:2) to give 5-chloro-6-(3,3-difluorocyclobutyl)pyridin-3-ol (160 mg) as a white solid. LCMS Method BD: [M+H]⁺=220. ¹H NMR (400 MHz, CD₃OD-d₄): δ 8.0 (s, 1H), 6.97-6.93 (m, 1H), 3.69-3.58 (m, 1H), 3.01-2.78 (m, 4H).

### Step 6: 5-chloro-6-(3,3-difluorocyclobutyl)pyridin-3-yl trifluoromethanesulfonate

5-chloro-6-(3,3-difluorocyclobutyl)pyridin-3-ol (160.0 mg, 0.7 mmol, 1.0 equiv.), was dissolved in DCM (20 mL), TEA (0.1 ml, 0.9 mmol, 1.2 equiv.) and 1,1,1-trifluoro-N-phenyl-N-trifluoromethanesulfonylmethanesulfonamide (309.4 mg, 0.8 mmol, 1.1 equiv.) were added. The resulting solution was stirred for 30 min at ambient temperature and then quenched by the addition of water. The solution was extracted with ethyl acetate, dried over anhydrous sodium sulfate and concentrated under vacuum. The residue was purified by flash column chromatography on silica gel, eluting with ethyl acetate/petroleum ether (1:8) to give 5-chloro-6-(3,3-difluorocyclobutyl)pyridin-3-yl trifluoromethanesulfonate (220 mg) as a white solid. LCMS Method BD: [M+H]⁺=352.

### Step 7: tert-butyl (5-chloro-6-(3,3-difluorocyclobutyl)pyridin-3-yl)carbamate

5-chloro-6-(3,3-difluorocyclobutyl)pyridin-3-yl trifluoromethanesulfonate (220.0 mg, 0.6 mmol, 1.0 equiv.) was dissolved in 1,4-dioxane (30 mL). Then NH₂Boc (230.3 mg, 1.9 mmol, 3.0 equiv.), 4,5-Bis(diphenylphosphino)-9,9-dimethylxanthene (75.8 mg, 0.1 mmol, 0.2 equiv.) and Pd₂(dba)₃ (120.1 mg, 0.1 mmol, 0.2 equiv.) were added under an atmosphere of nitrogen. The resulting solution was stirred for 3 hours at 90° C. under atmosphere of nitrogen and then concentrated under vacuum. The residue was purified by flash column chromatography on silica gel, eluting with ethyl acetate/petroleum ether (1:9) to give tert-butyl (5-chloro-6-(3,3-difluorocyclobutyl)pyridin-3-yl)carbamate (120 mg) as a white solid. LCMS Method BD: [M+H]⁺=319.

### Step 8: 5-chloro-6-(3,3-difluorocyclobutyl)pyridin-3-amine

tert-Butyl (5-chloro-6-(3,3-difluorocyclobutyl)pyridin-3-yl)carbamate (120.0 mg, 0.3 mmol, 1.0 equiv.) was dissolved in DCM (10 mL) and TFA (2 ml). The resulting solution was stirred for 30 min at ambient temperature and then diluted with water. The pH value of the solution was adjusted to 7.5 with saturated aqueous Na₂CO₃ and extracted with ethyl acetate. The organic layer was dried over anhydrous sodium sulfate and concentrated under vacuum. The residue was purified by flash column chromatography on silica gel, eluting with ethyl acetate/petroleum ether (1:3) to give 5-chloro-6-(3,3-difluorocyclobutyl)pyridin-3-amine (60 mg) as a white solid. LCMS Method BD: [M+H]⁺=219.

### Synthesis of Intermediate B33 (6-(3,3-difluorocyclobutyl)pyridin-3-amine)

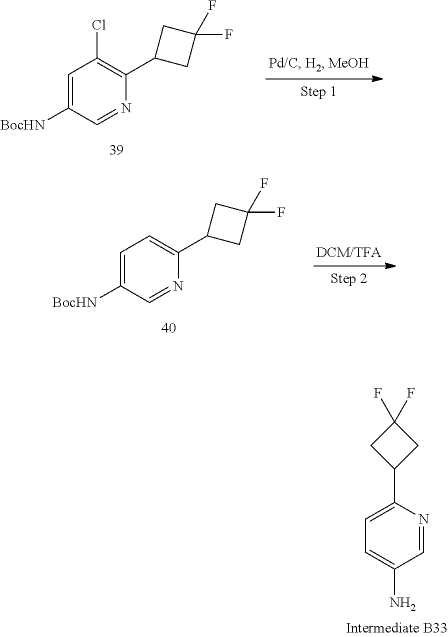 [SMILES: CC(C)(C)OC(=O)Nc1cnc(C2CC(F)(F)C2)c(Cl)c1 ; CC(C)(C)OC(=O)Nc1ccc(C2CC(F)(F)C2)nc1 ; Nc1ccc(C2CC(F)(F)C2)nc1]

### Step 1: tert-butyl (6-(3,3-difluorocyclobutyl)pyridin-3-yl)carbamate

tert-Butyl [5-chloro-6-(3,3-difluorocyclobutyl)pyridin-3-yl]carbamate (800.0 mg, 2.5 mmol, 1.0 equiv.) was dissolved MeOH (8 mL), then Pd/C (267.1 mg, wt 10%) was added under nitrogen. The mixture was sparged with nitrogen, placed under an atmosphere of hydrogen gas (balloon), then stirred overnight at ambient temperature. The solids were removed by filtration and the filtrate was concentrated under vacuum. The residue was purified by flash column chromatography on silica gel column, eluting with ethyl acetate/petroleum ether (1:1) to give tert-butyl (6-(3,3-difluorocyclobutyl)pyridin-3-yl)carbamate (500.0 mg) of as an off-white solid. LCMS Method BA: [M+H]⁺=285.

### Step 2: 6-(3,3-difluorocyclobutyl)pyridin-3-amine

tert-Butyl [6-(3,3-difluorocyclobutyl)pyridin-3-yl]carbamate (500.0 mg, 1.7 mmol, 1.0 equiv.) was dissolved DCM (5 mL) and cooled to 0° C., then TFA (1.0 mL) was added, maintaining the solution at 0° C. The reaction mixture was stirred for 3 hours at ambient temperature and concentrated under vacuum. The residue was diluted with water, and the solution was adjusted to pH 7 with aqueous NaOH (3 mol/L). The resulting solution was extracted with DCM, washed with brine, dried over anhydrous Na₂SO₄ and concentrated under vacuum to give 6-(3,3-difluorocyclobutyl)pyridin-3-amine (250.0 mg) as yellow oil. LCMS Method BC: [M+H]⁺=185.

### Synthesis of Intermediate B34 (6-(4,4-difluoropiperidin-1-yl)-5-ethylpyridin-3-amine

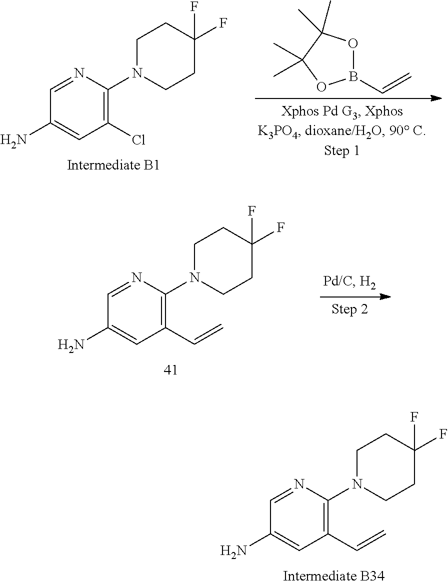 [SMILES: Nc1cnc(N2CCC(F)(F)CC2)c(Cl)c1 ; C=CB1OC(C)(C)C(C)(C)O1 ; C=Cc1cc(N)cnc1N1CCC(F)(F)CC1 ; C=Cc1cc(N)cnc1N1CCC(F)(F)CC1]

### Step 1: 6-(4,4-difluoropiperidin-1-yl)-5-ethenylpyridin-3-amine

5-Chloro-6-(4,4-difluoropiperidin-1-yl)pyridin-3-amine (3.0 g, 12.1 mmol, 1.0 equiv.) and K₃PO₄ (5.1 g, 24.2 mmol, 2.0 equiv.) were dissolved in 1,4-dioxane (60 mL) and water (6 mL), then Xphos Pd G3 (1.0 g, 1.2 mmol, 0.1 equiv.) and XPhos (577.4 mg, 1.2 mmol, 0.1 equiv.) were added under an atmosphere of nitrogen. The resulting mixture was heated to 90° C. overnight and then cooled to ambient temperature and quenched by the addition of water. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:1) to give 6-(4,4-difluoropiperidin-1-yl)-5-ethenylpyridin-3-amine (5.1 g) as a yellow solid. LCMS Method BD: [M+H]⁺=240. ¹H NMR (300 MHz, DMSO-d₆): δ 7.62 (d, 1H), 7.13 (d, 1H), 6.85-6.81 (m, 1H), 5.70-5.65 (m, 1H), 5.32-5.28 (m, 1H), 3.04-2.97 (m, 4H), 2.15-2.00 (m, 4H).

### Step 2: 6-(4,4-difluoropiperidin-1-yl)-5-ethylpyridin-3-amine

6-(4,4-difluoropiperidin-1-yl)-5-ethenylpyridin-3-amine (1.2 g, 2.5 mmol, 1.0 equiv.) was dissolved in THE (12 mL), then Pd/C (0.2 g, 2.5 mmol, 1.0 equiv.) was added. The mixture was sparged with nitrogen, placed under an atmosphere of hydrogen gas (balloon), then stirred overnight at ambient temperature. The solids were removed by filtration and the filtrate was concentrated under vacuum. The residue was purified by flash column chromatography on silica gel, eluting with ethyl acetate/petroleum ether (1:1) to give 6-(4,4-difluoropiperidin-1-yl)-5-ethylpyridin-3-amine (860 mg) as a dark yellow solid. LCMS Method BD: [M+H]⁺=242. ¹H NMR (300 MHz, DMSO-d₆): δ 7.52 (d, 1H), 6.84 (d, 1H), 2.96-2.91 (m, 5H), 2.56-2.54 (m, 2H), 2.07-2.01 (m, 4H), 1.14 (t, 3H).

### Synthesis of Intermediate B35 and intermediate B36 (2-(5-amino-2-(4,4-difluoropiperidin-1-yl)pyridin-3-yl)ethan-1-ol and 1-(5-amino-2-(4,4-difluoropiperidin-1-yl)pyridin-3-yl)ethan-1-ol)

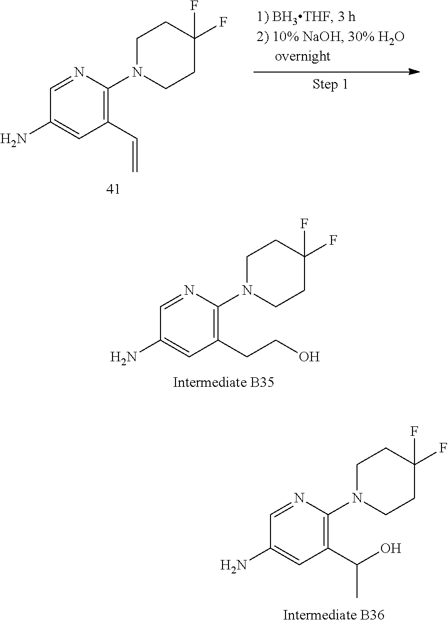 [SMILES: C=Cc1cc(N)cnc1N1CCC(F)(F)CC1 ; Nc1cnc(N2CCC(F)(F)CC2)c(CCO)c1 ; CC(O)c1cc(N)cnc1N1CCC(F)(F)CC1]

6-(4,4-difluoropiperidin-1-yl)-5-ethenylpyridin-3-amine (2.0 g, 8.4 mmol, 1.0 equiv.) was dissolved in THE (40 mL) and cooled to 0° C., then BH₃.THF (1M, 16.7 mL, 16.7 mmol, 2.0 equiv.) was added dropwise, maintaining the solution at 0° C. The resulting mixture was stirred for 3 hours at ambient temperature. To the above mixture was added NaOH (5.0 g, 12.5 mmol, 1.5 equiv.) and H₂O₂ (30%, 1.3 mL, 16.7 mmol, 2.0 equiv.). The resulting mixture was stirred for additional 4 hours at ambient temperature and quenched by the addition of water. The resulting mixture was extracted with ethyl acetate, washed with brine, dried over anhydrous Na₂SO₄ and concentrated under vacuum. The residue was purified by reverse flash chromatography with the following conditions: Column: C18; Mobile Phase A: Water/0.1% NH₃HCO₃, Mobile Phase B: ACN; Flow rate: 100 mL/min; Gradient: 5% B to 35% B in 30 min; 254 nm. This resulted in 2-[5-amino-2-(4,4-difluoropiperidin-1-yl)pyridin-3-yl]ethanol (front peak, 740 mg) as a yellow solid and 1-[5-amino-2-(4,4-difluoropiperidin-1-yl)pyridin-3-yl]ethanol (second peak, 540 mg) as a yellow solid.

Intermediate B35: LCMS Method BA: [M+H]⁺=258. ¹H NMR (400 MHz, DMSO-d₆): δ 7.53 (d, 1H), 6.85 (d, 1H), 4.92 (s, 2H), 4.67 (t, 1H), 3.65-3.60 (m, 2H), 2.94 (t, 4H), 2.66 (t, 2H), 2.08-2.03 (m, 4H).

Intermediate B36: LCMS Method BA: [M+H]⁺=258. ¹H NMR (400 MHz, DMSO-d₆): δ 7.56 (d, 1H), 7.08 (d, 1H), 5.01 (d, 2H), 5.00 (s, 1H), 4.98-4.89 (m, 1H), 3.07-2.80 (m, 4H), 2.13-1.98 (m, 4H), 1.28 (d, 3H).

### Synthesis of Intermediate B37 ((5-amino-2-(4,4-difluorocyclohexyl)pyridin-3-yl)methanol)

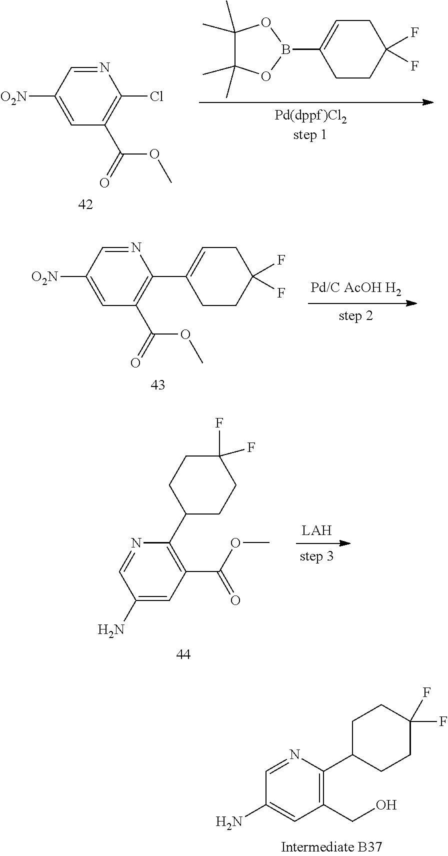 [SMILES: COC(=O)c1cc([N+](=O)[O-])cnc1Cl ; CC1(C)OB(C2=CCC(F)(F)CC2)OC1(C)C ; COC(=O)c1cc([N+](=O)[O-])cnc1C1=CCC(F)(F)CC1 ; COC(=O)c1cc(N)cnc1C1CCC(F)(F)CC1 ; Nc1cnc(C2CCC(F)(F)CC2)c(CO)c1]

### Step 1: methyl 2-(4,4-difluorocyclohex-1-en-1-yl)-5-nitronicotinate

2-Chloro-5-nitropyridine-3-carboxylate (1.0 g, 4.6 mmol, 1.0 equiv.) was dissolved in 1,4-dioxane (30 mL) and water (5 mL), then K₂CO₃ (1.0 g, 7.2 mmol, 1.5 equiv.), 2-(4,4-difluorocyclohex-1-en-1-yl)-4,4,5,5-tetramethyl-1,3,2-dioxaborolane (1.4 g, 5.7 mmol, 1.2 equiv.) and Pd(dppf)Cl₂ (0.7 g, 1.0 mmol, 0.2 equiv.) were added under an atmosphere of nitrogen. The resulting solution was heated to 90° C. for 2 hours and concentrated under vacuum. The residue was purified by flash column chromatography on silica gel, eluting with ethyl acetate/petroleum ether (1:6) to give methyl 2-(4,4-difluorocyclohex-1-en-1-yl)-5-nitropyridine-3-carboxylate (700 mg) as a white solid. LCMS Method BA: [M+H]⁺=299.

### Step 2: methyl 5-amino-2-(4,4-difluorocyclohexyl)nicotinate

2-(4,4-difluorocyclohex-1-en-1-yl)-5-nitropyridine-3-carboxylate (700.0 mg, 2.3 mmol, 1.0 equiv.) was dissolved in MeOH (20 mL), then Pd/C (70.0 mg, 0.7 mmol, 0.3 equiv.) and AcOH (28.2 mg, 0.5 mmol, 0.2 equiv.) were added. The mixture was sparged with nitrogen, placed under an atmosphere of hydrogen gas (balloon), then stirred for 3 days at ambient temperature. The solids were removed by filtration and the filtrate was concentrated under vacuum. The residue was purified by flash column chromatography on silica gel, eluting with ethyl acetate/petroleum ether (1:2) to give methyl 5-amino-2-(4,4-difluorocyclohexyl) pyridine-3-carboxylate (350 mg) as a white solid. LCMS Method BC: [M+H]⁺=271.

### Step 3: (5-amino-2-(4,4-difluorocyclohexyl)pyridin-3-yl)methanol

5-amino-2-(4,4-difluorocyclohexyl) pyridine-3-carboxylate (300.0 mg, 1.1 mmol, 1.0 equiv.) was dissolved in THE (20 mL) and cooled to 0° C., then LiAlH₄ (189.6 mg, 5.0 mmol, 4.5 equiv.) was added, maintaining the solution at 0° C. The resulting solution was stirred for 10 min at 0° C. and then quenched by the addition of aqueous HCl (1M). The solution was adjusted to pH 7 with aqueous Na₂CO₃. The resulting solution was extracted with DCM and concentrated under vacuum. The residue was purified by flash column chromatography on silica gel, eluting with ethyl acetate/petroleum ether (1:1) to give [5-amino-2-(4,4-difluorocyclohexyl) pyridin-3-yl] methanol (200 mg) as a white solid. LCMS Method BC: [M+H]⁺=243.

### Synthesis of Intermediate B38 (5-amino-2-(4,4-difluoropiperidin-1-yl)nicotinonitrile)

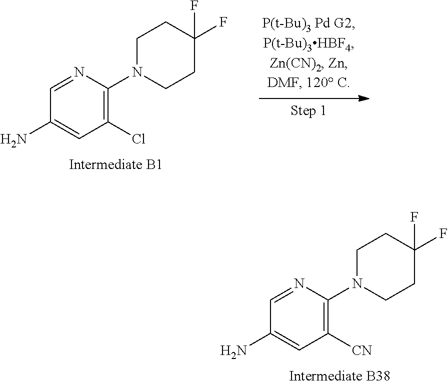 [SMILES: Nc1cnc(N2CCC(F)(F)CC2)c(Cl)c1 ; N#Cc1cc(N)cnc1N1CCC(F)(F)CC1]

5-chloro-6-(4,4-difluorocyclohexyl)pyridin-3-amine (300.0 mg, 1.2 mmol, 1.0 equiv.) was dissolved in DMF (20 mL), then P(t-Bu)₃ Palladacycle Gen. 3 (69.5 mg, 0.1 mmol, 0.1 equiv.), P(t-Bu)₃.HBF₄ (35.2 mg, 0.1 mmol, 0.1 equiv.), Zn(CN)₂ (285.6 mg, 2.4 mmol, 2.0 equiv.) and Zn (11.9 mg, 0.2 mmol, 0.2 equiv.) were added under an atmosphere of nitrogen. The resulting mixture was heated to 120° C. overnight and then quenched with NH₄OH. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:2) to give 5-amino-2-(4,4-difluorocyclohexyl)pyridine-3-carbonitrile (160 mg) as a colorless oil. LCMS Method BD: [M+H]⁺=239. ¹H NMR (300 MHz, Methanol-d₄): δ 8.15 (d, 1H), 7.25 (d, 1H), 3.21-3.05 (m, 1H), 2.26-1.80 (m, 8H).

The following intermediates were prepared using the method described for Intermediate 38.

| Intermediate | Starting material | Structure | LCMS data |
|---|---|---|---|
| Intermediate B39 | 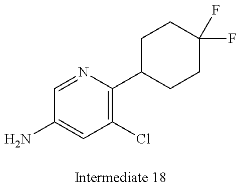 [SMILES: Nc1cnc(C2CCC(F)(F)CC2)c(Cl)c1] | 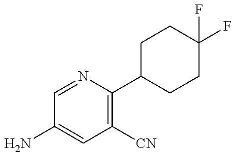 [SMILES: N#Cc1cc(N)cnc1C1CCC(F)(F)CC1] | Method BA: MS-ESI: 238 [M + H]⁺ |

### Synthesis of Intermediate B42 (1-acetyl-5-fluoro-1H-pyrrolo[2,3-b]pyridine-3-carboxylic acid)

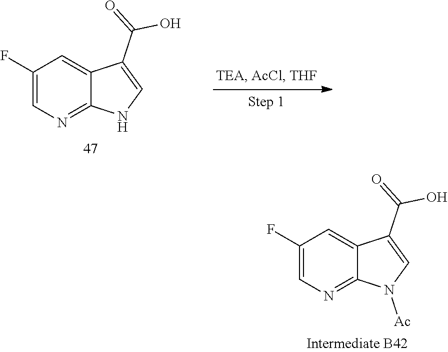 [SMILES: O=C(O)c1c[nH]c2ncc(F)cc12 ; CC(=O)n1cc(C(=O)O)c2cc(F)cnc21]

5-fluoro-1H-pyrrolo[2,3-b]pyridine-3-carboxylic acid (500.0 mg, 2.8 mmol, 1.0 equiv.) and TEA (1.2 mL, 8.3 mmol, 3.0 equiv.) were dissolved in THE (50 mL), then AcCl (0.6 mL, 8.3 mmol, 3.0 equiv.) was added. The reaction mixture was stirred for 16 hours at ambient temperature and then concentrated under vacuum. The residue was purified by flash column chromatography on silica gel, eluting with ethyl acetate/petroleum ether (1:10) to give 1-acetyl-5-fluoropyrrolo[2,3-b]pyridine-3-carboxylic acid (500.0 mg) as a yellow solid. LCMS Method BC: [M+H]⁺=223.

### Synthesis of Intermediate B43 (5-chloro-1H-pyrrolo[3,2-b]pyridin-3-amine)

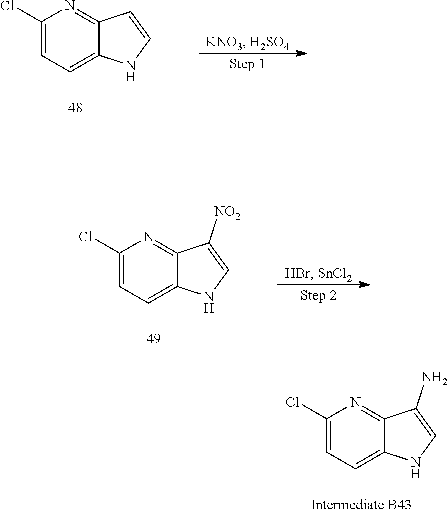

### Step 1: 5-chloro-3-nitro-1H-pyrrolo[3,2-b]pyridine

5-Chloro-1H-pyrrolo[3,2-b]pyridine (3.0 g, 19.7 mmol, 1.0 equiv.) was dissolved in concentrated H2SO₄ (5 mL) and cooled to 0° C., then KNO₃ (2.4 g, 23.8 mmol, 1.2 equiv.) was added in portions, maintaining the solution at 0° C. The reaction mixture was stirred for 2 hours at ambient temperature and then quenched by the addition of water. The resulting solution was adjusted to pH 9 with saturated aqueous NaHCO₃. The resulting mixture was extracted with ethyl acetate, washed with brine, dried over anhydrous Na₂SO₄ and concentrated under vacuum to 5-chloro-3-nitro-1H-pyrrolo[3,2-b]pyridine (3.1 g) as a yellow solid. LCMS Method BA: [M+H]⁺=198.

### Step 2: 5-chloro-1H-pyrrolo[3,2-b]pyridin-3-amine

5-Chloro-3-nitro-1H-pyrrolo[3,2-b]pyridine (1.0 g, 5.1 mmol, 1.0 equiv.) was dissolved in aqueous HBr (40%, 25 mL), then SnCl₂.2H₂O (6.7 g, 35.4 mmol, 7.0 equiv.) was added. The reaction mixture was stirred for 3 hours at ambient temperature, then adjusted to pH 9 with saturated aqueous NaHCO₃. The resulting solution was extracted with ethyl acetate, washed with brine, dried over anhydrous Na₂SO₄ and concentrated under vacuum to give 5-chloro-1H-pyrrolo[3,2-b]pyridin-3-amine (850.0 mg) as a dark green solid. LCMS Method BC: [M+H]⁺=168.

The following intermediates were prepared using the method described for Intermediate B43.

| Intermediate | Starting material A | Structure | LCMS data |
|---|---|---|---|
| Intermediate B44 | 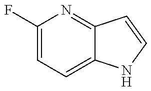 | 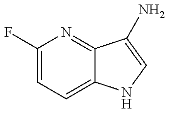 [SMILES: Nc1c[nH]c2ccc(F)nc12] | Method BA: MS-ESI: 152 [M + H]⁺ |
| Intermediate B45 | 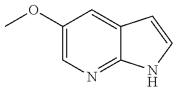 | 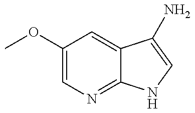 | Method BD: MS-ESI: 164 [M + H]⁺ |
| Intermediate B46 | 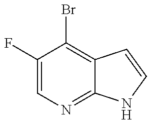 [SMILES: Fc1cnc2[nH]ccc2c1Br] | 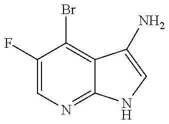 [SMILES: Nc1c[nH]c2ncc(F)c(Br)c12] | Method BC: MS-ESI: 230 [M + H]⁺ |
| Intermediate B47 | 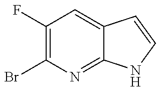 [SMILES: Fc1cc2cc[nH]c2nc1Br] | 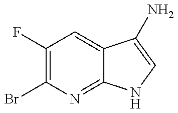 [SMILES: Nc1c[nH]c2nc(Br)c(F)cc12] | Method BC: MS-ESI: 230 [M + H]⁺ |
| Intermediate B48 | 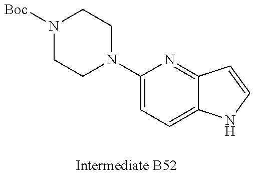 [SMILES: CC(C)(C)OC(=O)N1CCN(c2ccc3[nH]ccc3n2)CC1] | 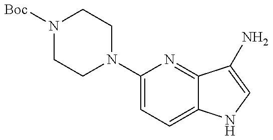 [SMILES: CC(C)(C)OC(=O)N1CCN(c2ccc3[nH]cc(N)c3n2)CC1] | Method BD: MS-ESI: 318 [M + H]⁺ |
| Intermediate B49 | 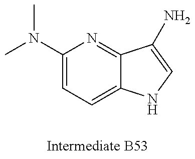 | 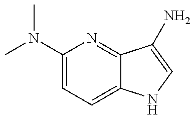 | Method BC: MS-ESI: 177 [M + H]⁺ |

### Synthesis of Intermediate B50 (5-(4-ethylpiperazin-1-yl)-1H-pyrrolo[3,2-b]pyridin-3-amine)

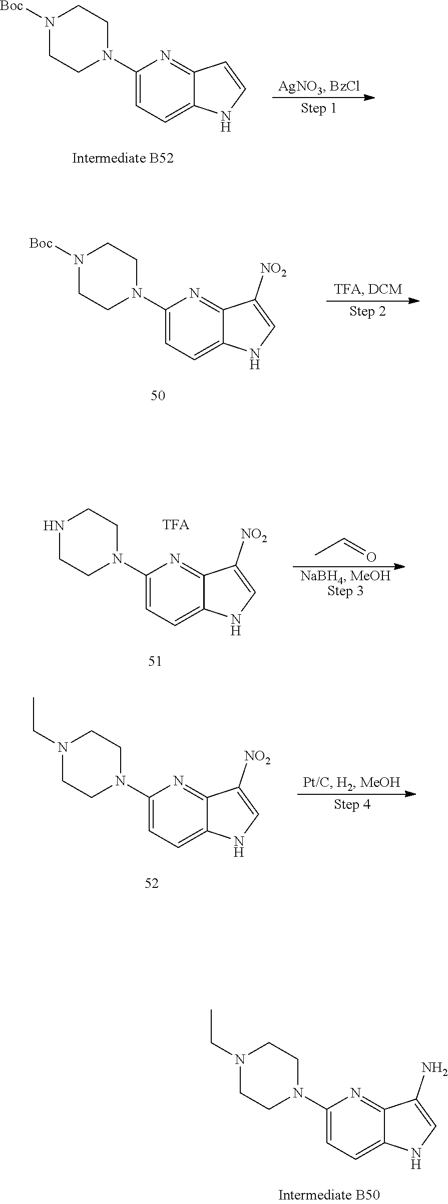

### Step 1: tert-butyl 4-[3-nitro-1H-pyrrolo[3,2-b]pyridin-5-yl]piperazine-1-carboxylate

tert-Butyl 4-[1H-pyrrolo[3,2-b]pyridin-5-yl]piperazine-1-carboxylate (500.0 mg, 1.7 mmol, 1.0 equiv.) was dissolved in ACN (30 ml), AgNO₃ (421.3 mg, 2.5 mmol, 1.5 equiv.) and benzoyl chloride (348.7 mg, 2.5 mmol, 1.5 equiv.) were added. The reaction mixture was stirred for 8 hours at ambient temperature and then quenched by the addition of water. The resulting solution was adjusted to pH 10 with aqueous Na₂CO₃ (2M). The resulting solution was extracted with ethyl acetate, washed with brine, dried over anhydrous sodium sulfate and concentrated under vacuum. The residue was purified by flash column chromatography on silica gel, eluting with ethyl acetate/petroleum ether (1:3) to give tert-butyl 4-[3-nitro-1H-pyrrolo[3,2-b]pyridin-5-yl]piperazine-1-carboxylate (311.2 mg) as a yellow solid. LCMS Method BF: [M+H]⁺=348.

### Step 2: 3-nitro-5-(piperazin-1-yl)-1H-pyrrolo[3,2-b]pyridine

tert-Butyl 4-[3-nitro-1H-pyrrolo[3,2-b]pyridin-5-yl]piperazine-1-carboxylate (700.0 mg, 2.0 mmol, 1.0 equiv.) was dissolved in DCM (30 mL), then TFA (0.8 mL, 10.1 mmol, 5.0 equiv.) was added. The reaction mixture was stirred for 5 hours at room temperature, and then concentrated under vacuum to give 3-nitro-5-(piperazin-1-yl)-1H-pyrrolo[3,2-b]pyridine TFA salt (521.5 mg) as a yellow solid. LCMS Method BF: [M+H]⁺=248.

### Step 3: 1-ethyl-4-[3-nitro-1H-pyrrolo[3,2-b]pyridin-5-yl]piperazine

3-Nitro-5-(piperazin-1-yl)-1H-pyrrolo[3,2-b]pyridine TFA salt (500.0 mg, 1.4 mmol, 1.0 equiv.) and acetaldehyde (95.7 mg, 2.2 mmol, 1.5 equiv.) were dissolved in MeOH (30 mL), then NaBH₄ (109.6 mg, 2.9 mmol, 2.0 equiv.) was added. The reaction mixture was stirred for 5 hours at room temperature and the concentrated under vacuum. The residue was purified by flash column chromatography on silica gel, eluting with ethyl acetate/petroleum ether (1:3) to give 1-ethyl-4-[3-nitro-1H-pyrrolo[3,2-b]pyridin-5-yl]piperazine (400 mg) as a yellow solid. LCMS Method BF: [M+H]⁺=276.

### Step 4: 5-(4-ethylpiperazin-1-yl)-1H-pyrrolo[3,2-b]pyridin-3-amine

1-Ethyl-4-[3-nitro-1H-pyrrolo[3,2-b]pyridin-5-yl]piperazine (400.0 mg, 1.5 mmol, 1.0 equiv.) was dissolved in MeOH (30 mL), then Pt/C (56.9 mg, 3%) was added under nitrogen. The mixture was sparged with nitrogen, placed under an atmosphere of hydrogen gas (balloon), then stirred for 5 hours at ambient temperature. The solids were removed by filtration and the filtrate was concentrated under vacuum. The residue was purified by flash column chromatography on silica gel column, eluting with ethyl acetate/petroleum ether (1:3) to give 5-(4-ethylpiperazin-1-yl)-1H-pyrrolo[3,2-b]pyridin-3-amine (255.2 mg) as a light yellow solid. LCMS Method BE: [M+H]⁺=246.

### Synthesis of Intermediate B51 (5-ethyl-1H-pyrrolo[3,2-b]pyridin-3-amine)

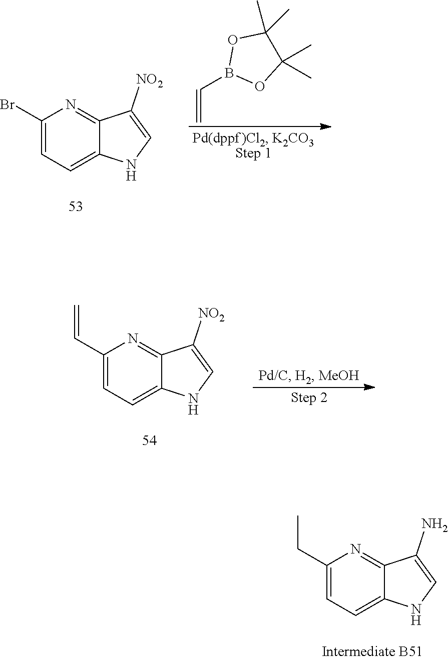

### Step 1: 5-ethenyl-3-nitro-1H-pyrrolo[3,2-b]pyridine

5-Bromo-3-nitro-1H-pyrrolo[3,2-b]pyridine (200.0 mg, 0.8 mmol, 1.0 equiv.) was dissolved in DMF (20 mL) and water (4 mL), then 2-ethenyl-4,4,5,5-tetramethyl-1,3,2-dioxaborolane (190.9 mg, 1.2 mmol, 1.5 equiv.), Pd(dppf)Cl₂ (60.5 mg, 0.1 mmol, 0.1 equiv.) and K₂CO₃ (228.4 mg, 1.7 mmol, 2.0 equiv.) were added under nitrogen. The reaction mixture was heated to 100° C. for 16 hours and then concentrated under vacuum. The residue was purified by flash column chromatography on silica gel column, eluting with ethyl acetate/petroleum ether (1:1) to give 5-ethenyl-3-nitro-1H-pyrrolo[3,2-b]pyridine (81.2 mg) as a yellow solid. LCMS Method BA: [M+H]⁺=190.

### Step 2: 5-ethyl-1H-pyrrolo[3,2-b]pyridin-3-amine

5-Ethenyl-3-nitro-1H-pyrrolo[3,2-b]pyridine (200.0 mg, 1.1 mmol, 1.0 equiv.) was dissolved in MeOH (10 mL), Pd/C (16.9 mg, 0.2 mmol, 0.2 equiv.) was added. The mixture was sparged with nitrogen, placed under an atmosphere of hydrogen gas (balloon), then stirred overnight at ambient temperature. The solids were removed by filtration and the filtrate was concentrated under vacuum to give crude 5-ethyl-1H-pyrrolo[3,2-b]pyridin-3-amine (110.1 mg) as a yellow solid. LCMS Method BA: [M+H]⁺=162.

### Synthesis of Intermediate B52 (tert-butyl 4-(1H-pyrrolo[3,2-b]pyridin-5-yl)piperazine-1-carboxylate)

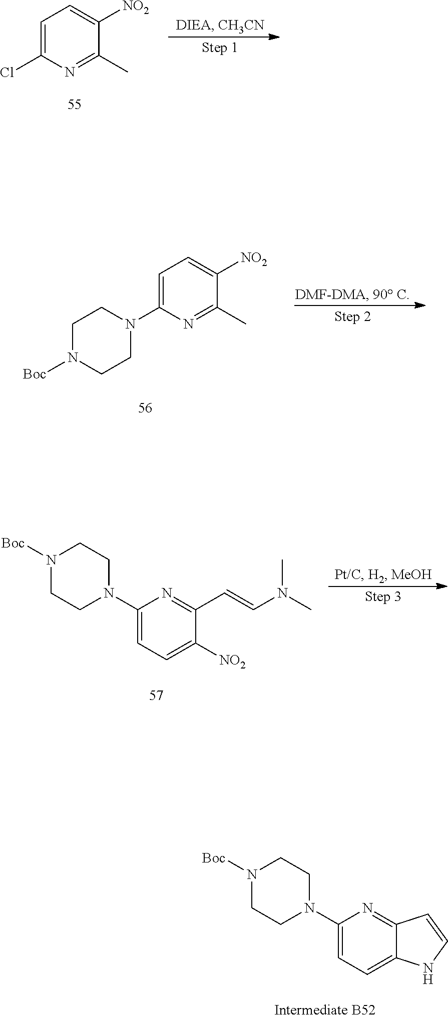

### Step 1: tert-butyl 4-(6-methyl-5-nitropyridin-2-yl)piperazine-1-carboxylate

6-Chloro-2-methyl-3-nitropyridine (500.0 mg, 2.9 mmol, 1.0 equiv.) was dissolved in ACN (30 mL), then tert-butyl piperazine-1-carboxylate (539.7 mg, 2.9 mmol, 1.0 equiv.) and DIEA (1.0 mL, 5.8 mmol, 2.0 equiv.) were added. The reaction mixture was heated to 50° C. for 16 hours and then quenched by the addition of water. The resulting solution was extracted with ethyl acetate, dried over anhydrous sodium sulfate and concentrated under vacuum. The residue was purified by flash column chromatography on silica gel column, eluting with ethyl acetate/petroleum ether (1:1) to give tert-butyl 4-(6-methyl-5-nitropyridin-2-yl)piperazine-1-carboxylate (552.3 mg) as a yellow solid. LCMS Method BF: [M+H]⁺=323.

### Step 2: tert-butyl 4-(6-methyl-5-nitropyridin-2-yl)piperazine-1-carboxylate

tert-Butyl 4-(6-methyl-5-nitropyridin-2-yl)piperazine-1-carboxylate (500.0 mg, 1.6 mmol, 1.0 equiv.) was dissolved in DMF (30 mL), then (dimethoxymethyl)dimethylamine (0.6 mL, 4.7 mmol, 3.0 equiv.) was added. The resulting solution was heated to 90° C. for 16 hours and then quenched by the addition of water. The resulting solution was extracted with DCM, washed with brine and concentrated under vacuum. The residue was purified by flash column chromatography on silica gel column, eluting with ethyl acetate/petroleum ether (1:2) to give tert-butyl 4-(6-methyl-5-nitropyridin-2-yl)piperazine-1-carboxylate (352.3 mg) as a red solid. LCMS Method BC: [M+H]⁺=378.

### Step 3: tert-butyl [5-cyclobutoxy-1H-pyrrolo[2,3-b]pyridin-3-yl]carbamate

tert-Butyl 4-(6-methyl-5-nitropyridin-2-yl)piperazine-1-carboxylate (400.0 mg, 1.1 mmol, 1.0 equiv.) was dissolved in MeOH (30 mL), then Pt/C (50.7 mg, 3%) was added under nitrogen. The mixture was sparged with nitrogen, placed under an atmosphere of hydrogen gas (balloon), then stirred for 5 hours at ambient temperature. The solids were removed by filtration and the filtrate was concentrated under vacuum. The residue was purified by flash column chromatography on silica gel column, eluting with ethyl acetate/petroleum ether (1:3) to give tert-butyl [5-cyclobutoxy-1H-pyrrolo[2,3-b]pyridin-3-yl]carbamate (298.5 mg) as a yellow solid. LCMS Method BA: [M+H]⁺=303.

The following intermediate was prepared using the method described for Intermediate B52.

| Intermediate | Structure | LCMS data |
|---|---|---|
| Intermediate B53 | 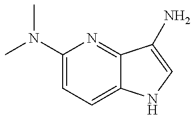 | Method BA: MS-ESI: 162 [M + H]⁺ |

### Synthesis of Intermediate B68 (1-(4,4-difluorocyclohexyl)-1H-pyrazol-4-amine)

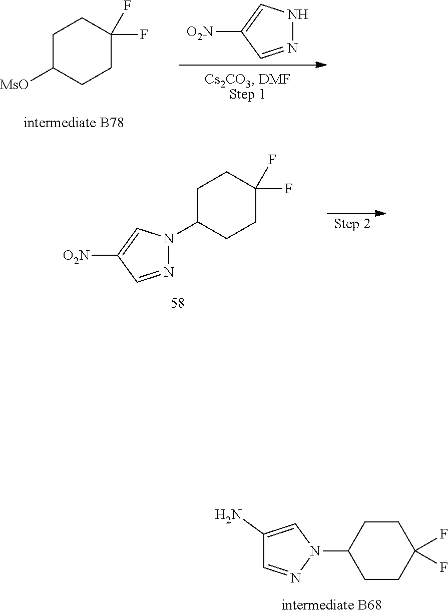

### Step 1: 1-(4,4-difluorocyclohexyl)-4-nitropyrazole

4,4-Difluorocyclohexyl methanesulfonate (500.0 mg, 2.3 mmol, 1.0 equiv.) was dissolved in DMF (10 mL), then 4-nitropyrazole (316.7 mg, 2.8 mmol, 1.2 equiv.), Cs₂CO₃ (1.5 g, 4.7 mmol, 2.0 equiv.) were added. The reaction mixture was heated to 90° C. for 12 hours, then cooled to ambient temperature and quenched by the addition of water. The resulting solution was extracted with ethyl acetate, washed with brine, dried over anhydrous sodium sulfate and concentrated under vacuum. The residue was purified by flash column chromatography on silica gel column, eluting with ethyl acetate/petroleum ether (1:10) to give 1-(4,4-difluorocyclohexyl)-4-nitropyrazole (420.0 mg) as an off-white solid. LCMS Method BC: [M+H]⁺=232.

### Step 2: 1-(4,4-difluorocyclohexyl)pyrazol-4-amine

1-(4,4-difluorocyclohexyl)-4-nitropyrazole (400.0 mg, 1.7 mmol, 1.0 equiv.) was dissolved in MeOH (10 mL), then Pd/C (184.1 mg, 10% wt.) was added. The reaction mixture was sparged with nitrogen, placed under an atmosphere of hydrogen gas (balloon), then stirred overnight at ambient temperature. The solids were removed by filtration and the filtrate was concentrated under vacuum to give 1-(4,4-difluorocyclohexyl)pyrazol-4-amine (243.1 mg) as a yellow solid. LCMS Method BC: [M+H]⁺=202.

The following intermediate was synthesized using the methods described for Intermediate B68, above.

| Intermediate | Starting material | Structure | LCMS data |
|---|---|---|---|
| Intermediate B69 | 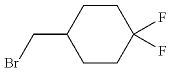 | 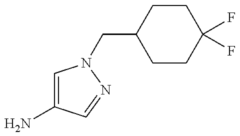 [SMILES: Nc1cnn(CC2CCC(F)(F)CC2)c1] | Method BC: MS-ESI: 216 [M + H]⁺ |

### Synthesis of Intermediate B70 (5-chloro-6-(3,3-difluoro-1-oxa-9-azaspiro[5.5]undecan-9-yl)pyridin-3-amine)

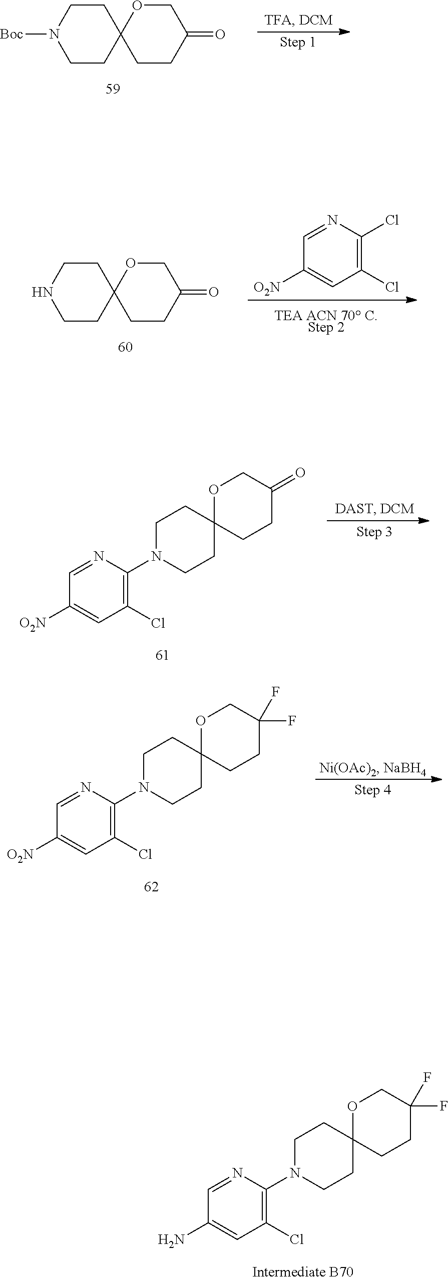

### Step 1: 1-oxa-9-azaspiro[5.5]undecan-3-one

tert-Butyl 3-oxo-1-oxa-9-azaspiro[5.5]undecane-9-carboxylate (1.0 g, 3.7 mmol, 1.0 equiv.) was dissolved in DCM (6 mL) and TFA (4 mL). The reaction mixture was stirred for 1 hour at ambient temperature and then quenched by the addition of water. The result solution was adjusted to pH 8 with aqueous Na₂CO₃, then extracted with ethyl acetate and concentrated under vacuum. The residue was purified by flash column chromatography on silica gel column, eluting with ethyl acetate/petroleum ether (1:3) to give 1-oxa-9-azaspiro[5.5]undecan-3-one (512 mg) as a white solid. LCMS Method BA: [M+H]⁺=170.

### Step 2: 9-(3-chloro-5-nitropyridin-2-yl)-1-oxa-9-azaspiro[5.5]undecan-3-one

1-Oxa-9-azaspiro[5.5]undecan-3-one (500.0 mg, 3.0 mmol, 1.0 equiv.) was dissolved in ACN (40 mL), then 2,3-dichloro-5-nitropyridine (570.2 mg, 3.0 mmol, 1.0 equiv.) and TEA (0.5 mL, 3.5 mmol, 1.2 equiv.) were added. The resulting solution was heated to 70° C. for 2 hours and then cooled to room temperature and quenched by the addition of water. The resulting solution was extracted with ethyl acetate and concentrated under vacuum. The residue was purified by flash column chromatography on silica gel column, eluting with ethyl acetate/petroleum ether (1:6) to give 9-(3-chloro-5-nitropyridin-2-yl)-1-oxa-9-azaspiro[5.5]undecan-3-one (580.2 mg) as a white solid. LCMS Method BD: [M+H]⁺=326.

### Step 3: 9-(3-chloro-5-nitropyridin-2-yl)-3,3-difluoro-1-oxa-9-azaspiro[5.5]undecane

9-(3-chloro-5-nitropyridin-2-yl)-1-oxa-9-azaspiro[5.5]undecan-3-one (500.0 mg, 1.5 mmol, 1.0 equiv.) was dissolved in DCM (12 mL) and cooled to −10° C., then DAST (272.2 mg, 1.7 mmol, 1.1 equiv.) was added. The reaction mixture was stirred for 30 min at −10° C. and then quenched by the addition of water. The result solution was adjusted to pH 7.5 with aqueous Na₂CO₃, extracted with ethyl acetate and concentrated under vacuum.

The residue was purified by flash column chromatography on silica gel column, eluting with ethyl acetate/petroleum ether (1:2) to give 9-(3-chloro-5-nitropyridin-2-yl)-3,3-difluoro-1-oxa-9-azaspiro[5.5]undecane (351.1 mg) as a white solid. LCMS Method BA: [M+H]⁺=348.

### Step 4: 5-chloro-6-[3,3-difluoro-1-oxa-9-azaspiro[5.5]undecan-9-yl]pyridin-3-amine

9-(3-chloro-5-nitropyridin-2-yl)-3,3-difluoro-1-oxa-9-azaspiro[5.5]undecane (350.0 mg, 1.0 mmol, 1.0 equiv.) was dissolved in THF/MeOH (10/10 mL) and cooled to 0° C., then Ni(OAc)₂ (177.9 mg, 1.0 mmol, 1.0 equiv.) was added. This was followed by the addition of NaBH₄ (38.1 mg, 1.0 mmol, 1.0 equiv.) at 0° C. The reaction mixture was stirred for 30 min at 0° C. and then quenched by the addition of water. The resulting solution was extracted with ethyl acetate, washed with brine and concentrated under vacuum. The residue was purified by flash column chromatography on silica gel column, eluting with ethyl acetate/petroleum ether (1:3) to give 5-chloro-6-[3,3-difluoro-1-oxa-9-azaspiro[5.5]undecan-9-yl]pyridin-3-amine (252.0 mg) as a white solid. LCMS Method BB: [M+H]⁺=318.

### Synthesis of Intermediate B72 (5-chloro-6-(1-(2,2,2-trifluoroethyl)pyrrolidin-3-yl)pyridin-3-amine)

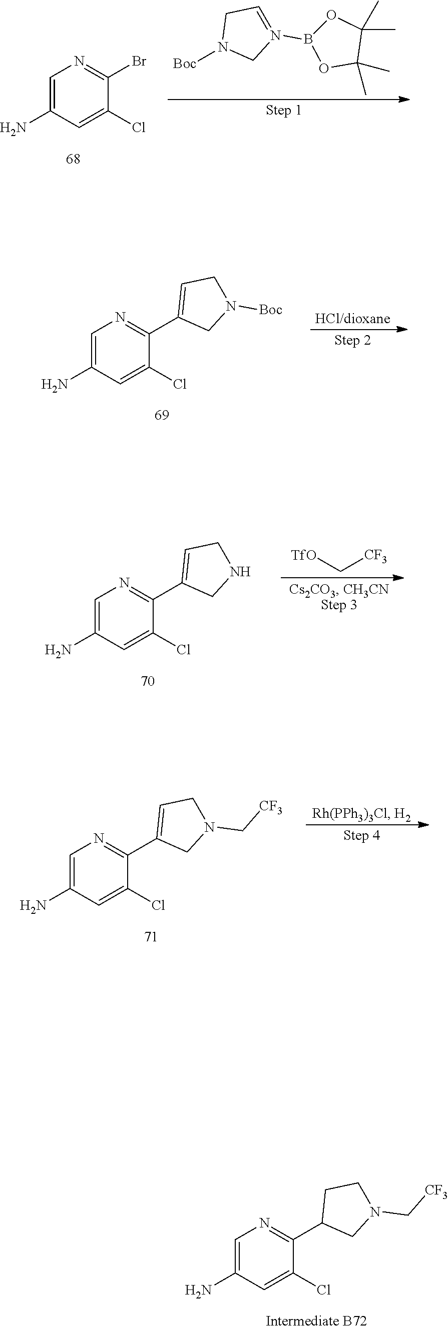

### Step 1: tert-butyl 3-(5-amino-3-chloropyridin-2-yl)-2,5-dihydropyrrole-1-carboxylate

6-Bromo-5-chloropyridin-3-amine (5.0 g, 24.1 mmol, 1.0 equiv.) and tert-butyl 3-(4,4,5,5-tetramethyl-1,3,2-dioxaborolan-2-yl)-2,5-dihydropyrrole-1-carboxylate (8.5 g, 28.9 mmol, 1.2 equiv.) were dissolved in 1,4-dioxane/water (25/5 mL), then Cs₂CO₃ (15.7 g, 48.2 mmol, 2.0 equiv.) and Pd(dppf)Cl₂ (1.8 g, 2.4 mmol, 0.1 equiv.) were added under an atmosphere of nitrogen. The reaction mixture was heated to 70° C. overnight under nitrogen, then cooled to ambient temperature and concentrated under vacuum. The residue was purified by flash column chromatography on silica gel column, eluting with ethyl acetate/petroleum ether (1:1) to give tert-butyl 3-(5-amino-3-chloropyridin-2-yl)-2,5-dihydropyrrole-1-carboxylate (2.6 g) as a yellow solid. LCMS Method BA: [M+H]⁺=296.

Step 2: 5-chloro-6-(2,5-dihydro-1H-pyrrol-3-yl)pyridin-3-amine

tert-Butyl 3-(5-amino-3-chloropyridin-2-yl)-2,5-dihydropyrrole-1-carboxylate (2.6 g, 8.8 mmol, 1.0 equiv.) was dissolved in HCl (4M in 1,4-dioxane, 10 mL). The resulting solution was stirred for 2 hours at ambient temperature and then concentrated under vacuum to give 5-chloro-6-(2,5-dihydro-1H-pyrrol-3-yl)pyridin-3-amine hydrochloride (420.0 mg) as a brown solid. LCMS Method BC: [M+H]⁺=196.

### Step 3: 5-chloro-6-(1-(2,2,2-trifluoroethyl)-2,5-dihydro-1H-pyrrol-3-yl)pyridin-3-amine

5-Chloro-6-(2,5-dihydro-1H-pyrrol-3-yl)pyridin-3-amine (600.0 mg, 3.1 mmol, 1.0 equiv.) was dissolved in ACN (10 mL), Cs₂CO₃ (4.0 g, 12.3 mmol, 4.0 equiv.) and 2,2,2-trifluoroethyl trifluoromethanesulfonate (1.1 g, 4.6 mmol, 1.5 equiv.) were added. The reaction mixture was heated to 50° C. for 12 hours, then cooled to ambient temperature, filtrated out the solid and the filtrate was concentrated under vacuum. The residue was purified by flash column chromatography on silica gel column, eluting with ethyl acetate/petroleum ether (1:1) to give 5-chloro-6-(1-(2,2,2-trifluoroethyl)-2,5-dihydro-1H-pyrrol-3-yl)pyridin-3-amine (310.2 mg) as a yellow solid. LCMS Method BD: [M+H]⁺=278.

### Step 4: 5-chloro-6-[1-(2,2,2-trifluoroethyl)pyrrolidin-3-yl]pyridin-3-amine

5-Chloro-6-[1-(2,2,2-trifluoroethyl)-2,5-dihydropyrrol-3-yl]pyridin-3-amine (300.0 mg, 1.1 mmol, 1.0 equiv.) was dissolved MeOH (10 mL), Rh(PPh₃)₃Cl (100.0 mg, 0.1 mmol, 0.1 equiv.) was added under nitrogen. The reaction mixture was stirred overnight at 50° C. under an atmosphere of hydrogen (30 atm.). The solids were removed by filtration and the filtrate was concentrated under vacuum. The residue was purified by flash column chromatography on silica gel column, eluting with ethyl acetate/petroleum ether (1:1) to give 5-chloro-6-[1-(2,2,2-trifluoroethyl)pyrrolidin-3-yl]pyridin-3-amine (150.0 mg) as a yellow solid. LCMS Method BA: [M+H]⁺=280.

### Synthesis of Intermediate B73 (3-chloro-4-(3,3-difluorocyclobutyl)aniline)

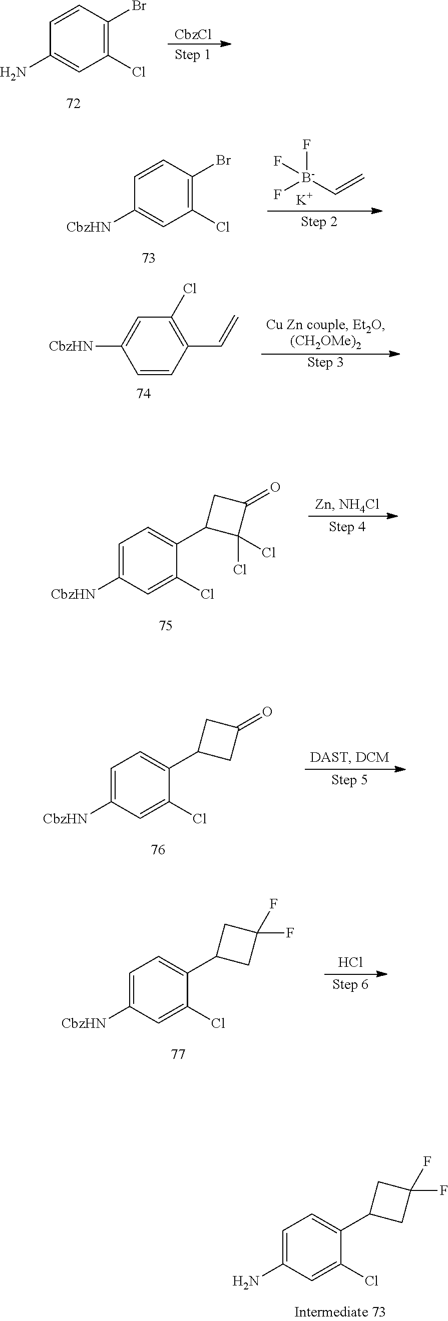

### Step 1: benzyl N-(4-bromo-3-chlorophenyl)carbamate

4-Bromo-3-chloroaniline (10.0 g, 48.4 mmol, 1.0 equiv.) was dissolved in THF (100 mL) and water (20 mL), then K₂CO₃ (13.4 g, 96.9 mmol, 2.0 equiv.) and CbzCl (12.4 g, 72.7 mmol, 1.5 equiv.) were added. The resulting solution was stirred for 12 hours at ambient temperature and quenched by the addition of water. The resulting solution was extracted with ethyl acetate, dried over anhydrous Na₂SO₄ and concentrated under vacuum to give benzyl N-(4-bromo-3-chlorophenyl)carbamate (15.2 g) as a white solid. LCMS Method BA: [M+H]⁺=340.

### Step 2: benzyl N-(3-chloro-4-ethenylphenyl)carbamate

Benzyl N-(4-bromo-3-chlorophenyl)carbamate (1.0 g, 2.9 mmol, 1.0 equiv.) were dissolved in 1,4-dioxane/water (20/4 mL), then Cs₂CO₃ (1.9 g, 5.9 mmol, 2.0 equiv.), potassium trifluoro(vinyl)borate (0.59 g, 4.4 mmol, 1.5 equiv.) and Pd(PPh₃)₄ (0.3 g, 0.3 mmol, 0.1 equiv.) were added under an atmosphere of nitrogen. The reaction mixture was heated to 90° C. for 12 hours, then cooled to ambient temperature and concentrated under vacuum. The residue was purified by flash column chromatography on silica gel column, eluting with ethyl acetate/petroleum ether (1:12) to give benzyl N-(3-chloro-4-ethenylphenyl)carbamate (0.6 g) as an off-white solid. LCMS Method BD: [M+H]⁺=288.

### Step 3: benzyl N-[3-chloro-4-(2,2-dichloro-3-oxocyclobutyl)phenyl]carbamate

Benzyl N-(3-chloro-4-ethenylphenyl)carbamate (35.0 g, 121.6 mmol, 1.0 equiv.) was dissolved in Et₂O (100 mL) and DME (20 mL), then trichloroacetyl chloride (33.2 g, 182.4 mmol, 1.5 equiv.) and, Zn—Cu (35.0 g, 271.3 mmol, 2.2 equiv.). The reaction was heated to 50° C. for 12 hours, then cooled to ambient temperature and quenched by the addition of water. After removing the solid by filtration, the filtrate was adjusted to pH 7 with NaOH aqueous (2N). The resulting solution was extracted with ethyl acetate, washed with brine and concentrated under vacuum. The residue was purified by flash column chromatography on silica gel column, eluting with ethyl acetate/petroleum ether (1:15) to give benzyl N-[3-chloro-4-(2,2-dichloro-3-oxocyclobutyl)phenyl]carbamate (10.3 g) as a yellow solid. LCMS Method BA: [M+H]⁺=398.

### Step 4: benzyl N-[3-chloro-4-(3-oxocyclobutyl)phenyl]carbamate

Benzyl N-[3-chloro-4-(2,2-dichloro-3-oxocyclobutyl)phenyl]carbamate (10.0 g, 25.1 mmol, 1.0 equiv.) was dissolved in THE (100 mL) and water (20 mL), then NH₄Cl (2.7 g, 50.2 mmol, 2.0 equiv.) and Zn (3.3 g, 50.5 mmol, 2.0 equiv.) were added. The reaction mixture was heated to 70° C. for 12 hours. After cooled to ambient temperature and filtration, the resulting solution was extracted with ethyl acetate, dried over anhydrous sodium sulfate and concentrated under vacuum to give benzyl N-[3-chloro-4-(3-oxocyclobutyl)phenyl]carbamate (6.1 g) as a white solid. LCMS Method BC: [M+H]⁺=330.

### Step 5: benzyl N-[3-chloro-4-(3,3-difluorocyclobutyl)phenyl]carbamate

Benzyl N-[3-chloro-4-(3-oxocyclobutyl)phenyl]carbamate (10.0 g, 30.3 mmol, 1.0 equiv.) was dissolved in DCM (100 mL) and cooled to 0° C., then DAST (9.8 g, 60.7 mmol, 2.0 equiv.) was added dropwise. The reaction mixture was stirred for 12 hours at 0° C. and then quenched by the addition of ice-water. The resulting solution was extracted with DCM, dried over anhydrous sodium sulfate and concentrated under vacuum. The residue was purified by flash column chromatography on silica gel column, eluting with ethyl acetate/petroleum ether (1:10) to give benzyl N-[3-chloro-4-(3,3-difluorocyclobutyl)phenyl]carbamate (4.2 g) as a yellow oil. LCMS Method BC: [M+H]⁺=352.

### Step 6: 3-chloro-4-(3,3-difluorocyclobutyl)aniline

Benzyl N-[3-chloro-4-(3,3-difluorocyclobutyl)phenyl]carbamate (1.0 g, 2.8 mmol, 1.0 equiv.) was dissolved in conc. HCl (10 mL). The resulting solution was heated to 70° C. for 12 hours, then cooled to ambient temperature and diluted with water. The solution was adjusted to pH 8 with NaOH aqueous (20%), extracted with ethyl acetate, washed with brine, dried over anhydrous Na₂SO₄ and concentrated under vacuum. The residue was purified by flash column chromatography on silica gel column, eluting with ethyl acetate/petroleum ether (1:10) to give 3-chloro-4-(3,3-difluorocyclobutyl)aniline (0.3 g) as a yellow solid. LCMS Method BA: [M+H]⁺=218.

### Synthesis of Intermediate B74 (5-chloro-6-(4-(3,3,3-trifluoropropyl)piperazin-1-yl)pyridin-3-amine

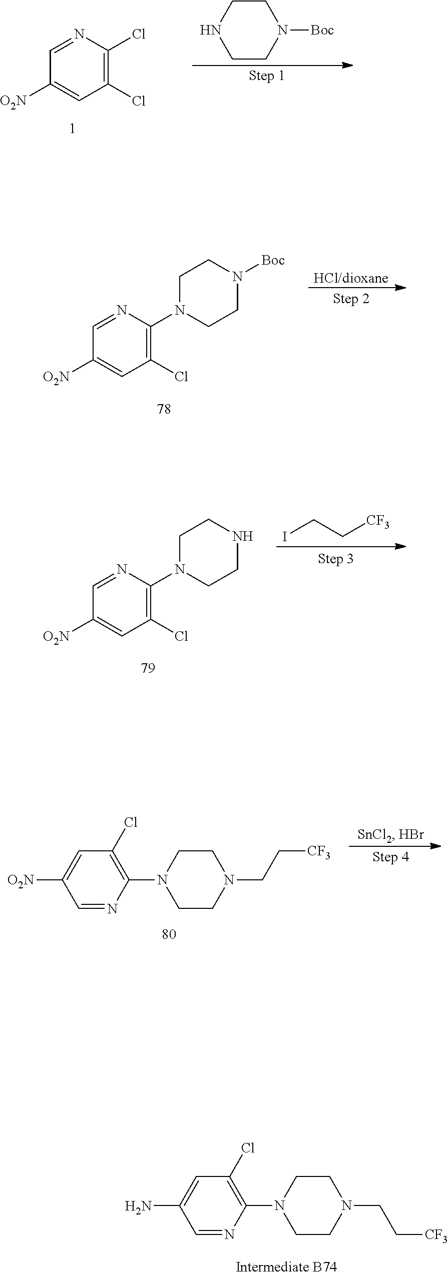

### Step 1: tert-butyl 4-(3-chloro-5-nitropyridin-2-yl)piperazine-1-carboxylate

2,3-Dichloro-5-nitropyridine (10.0 g, 51.8 mmol, 1.0 equiv.) and tert-butyl piperazine-1-carboxylate (9.7 g, 52.0 mmol, 1.0 equiv.) were dissolved in DMF (150 mL), then Cs₂CO₃ (33.8 g, 104.1 mmol, 2.0 equiv.) was added. The reaction mixture was heated to 70° C. for 12 hours, then cooled to ambient temperature and quenched by the addition of water. The resulting solution was extracted with ethyl acetate, washed with brine, dried over anhydrous Na₂SO₄ and concentrated under vacuum. The residue was purified by flash column chromatography on silica gel column, eluting with ethyl acetate/petroleum ether (1:1) to give tert-butyl 4-(3-chloro-5-nitropyridin-2-yl)piperazine-1-carboxylate (14.6 g) as a yellow solid. LCMS Method BA: [M+H]⁺=243.

### Step 2: 1-(3-chloro-5-nitropyridin-2-yl)piperazine

tert-Butyl 4-(3-chloro-5-nitropyridin-2-yl)piperazine-1-carboxylate (16.0 g, 46.7 mmol, 1.0 equiv.) was dissolved in HCl (4M in 1,4-dioxane, 100 mL). The resulting solution was stirred for 3 hours at ambient temperature and then concentrated under vacuum to give 1-(3-chloro-5-nitropyridin-2-yl)piperazine hydrochloride (18.1 g) as a yellow solid. LCMS Method BA: [M+H]⁺=243.

### Step 3: 1-(3-chloro-5-nitropyridin-2-yl)-4-(3,3,3-trifluoropropyl)piperazine

1-(3-chloro-5-nitropyridin-2-yl)piperazine (18.0 g, 74.2 mmol, 1.0 equiv.) and Cs₂CO₃ (96.7 g, 296.7 mmol, 4.0 equiv.) were dissolved in ACN (150 mL), then 1,1,1-trifluoro-3-iodopropane (66.5 g, 296.7 mmol, 4.0 equiv.) was added dropwise. The reaction mixture was heated to 50° C. overnight, then cooled to ambient temperature, filtrated and concentrated under vacuum. The residue was purified by flash column chromatography on silica gel column, eluting with ethyl acetate/petroleum ether (1:1) to give 1-(3-chloro-5-nitropyridin-2-yl)-4-(3,3,3-trifluoropropyl)piperazine (12.1 g) as a brown oil. LCMS Method BC: [M+H]⁺=339.

### Step 4: 5-chloro-6-[4-(3,3,3-trifluoropropyl)piperazin-1-yl]pyridin-3-amine

1-(3-chloro-5-nitropyridin-2-yl)-4-(3,3,3-trifluoropropyl)piperazine (10.0 g, 29.5 mmol, 1.0 equiv.) was dissolved in HBr (48% aqueous, 50 mL), then SnCl₂ (13.3 g, 59.0 mmol, 2.0 equiv.) was added. The reaction mixture was stirred for 4 hours at ambient temperature. Then the solution was adjusted to pH 9 with aqueous NaOH (25%), extracted with DCM, washed with brine, dried over anhydrous Na₂SO₄ and concentrated under vacuum. The residue was purified by flash column chromatography on silica gel column, eluting with DCM/MeOH (12:1) to give 5-chloro-6-[4-(3,3,3-trifluoropropyl)piperazin-1-yl]pyridin-3-amine (4.5 g) as a brown oil. LCMS Method BC: [M+H]⁺=309.

### Synthesis of Intermediate B77 (5-(2-methoxyethoxy)-1H-pyrrolo[3,2-b]pyridin-3-amine hydrochloride)

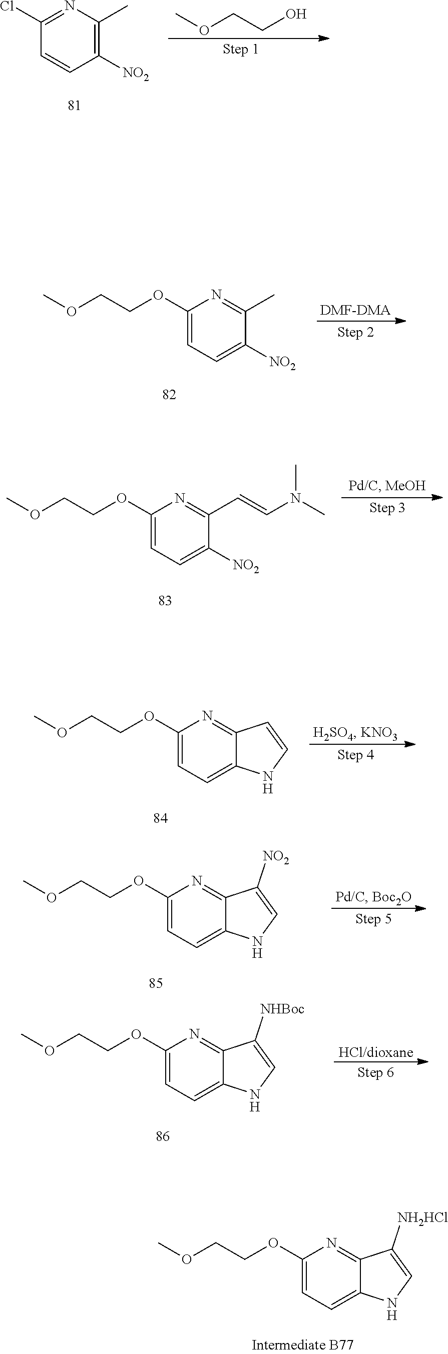

### Step 1: 6-(2-methoxyethoxy)-2-methyl-3-nitropyridine

6-Chloro-2-methyl-3-nitropyridine (10.0 g, 57.9 mmol, 1.0 equiv.) was dissolved in 2-methoxyethanol (100 mL) and cooled to 0° C., then NaH (60% wt., 3.5 g, 86.9 mmol, 1.5 equiv.) was added. The reaction mixture was stirred for 8 hours at ambient temperature and then quenched by the addition of ice-water. The resulting solution was extracted with ethyl acetate, washed with brine, dried over anhydrous Na₂SO₄ and concentrated under vacuum. The residue was purified by flash column chromatography on silica gel column, eluting with ethyl acetate/petroleum ether (1:5) to give 6-(2-methoxyethoxy)-2-methyl-3-nitropyridine (10.5 g) as an orange solid. LCMS Method BI: [M+H]⁺=213.

### Step 2: [(E)-2-[6-(2-methoxyethoxy)-3-nitropyridin-2-yl]ethenyl]dimethylamine

6-(2-Methoxyethoxy)-2-methyl-3-nitropyridine (10.5 g, 49.5 mmol, 1.0 equiv.) was dissolved in DMF (50 mL), then DMF-DMA (17.7 g, 148.4 mmol, 3.0 equiv.) was added. The resulting solution was heated to 110° C. for 8 hours, then cooled to ambient temperature and quenched by the addition of water. The resulting solution was extracted with ethyl acetate and concentrated under vacuum. The residue was purified by flash column chromatography on silica gel column, eluting with ethyl acetate/petroleum ether (1:1) to give [(E)-2-[6-(2-methoxyethoxy)-3-nitropyridin-2-yl]ethenyl]dimethylamine (14.1 g) as a red solid. LCMS Method BC: [.M+H]⁺=268.

### Step 3: 5-(2-methoxyethoxy)-1H-pyrrolo[3,2-b]pyridine

[(E)-2-[6-(2-methoxyethoxy)-3-nitropyridin-2-yl]ethenyl]dimethylamine (14.0 g, 52.4 mmol, 1.0 equiv.) was dissolved in MeOH (150 mL), then Pd/C (557.4 mg, 10%) was added under an atmosphere of nitrogen. The mixture was sparged with nitrogen, placed under an atmosphere of hydrogen gas (balloon), then stirred for 9 hours at ambient temperature. The solids were removed by filtration and the filtrate was concentrated under vacuum. The residue was purified by flash column chromatography on silica gel, eluting with dichloromethane/methanol (50:1) to give 5-(2-methoxyethoxy)-1H-pyrrolo[3,2-b]pyridine (7.2 g) as a yellow solid. LCMS Method BA: [M+H]⁺=193.

### Step 4: 5-(2-methoxyethoxy)-3-nitro-1H-pyrrolo[3,2-b]pyridine

5-(2-Methoxyethoxy)-1H-pyrrolo[3,2-b]pyridine (7.2 g, 37.2 mmol, 1.0 equiv.) was dissolved H2SO₄ (40. mL) and cooled to 0° C., then KNO₃ (4.9 g, 48.4 mmol, 1.3 equiv.) was added. The reaction mixture was stirred for 2 hours at ambient temperature and quenched by the addition of water/ice. The resulting solution was adjusted to pH 8 with NaOH aqueous (4 mol/L). The solids were collected by filtration and dried to give 5-(2-methoxyethoxy)-3-nitro-1H-pyrrolo[3,2-b]pyridine (8.1 g) as a light yellow solid. LCMS Method BB: [M+H]⁺=238.

### Step 5: tert-butyl N-[5-(2-methoxyethoxy)-1H-pyrrolo[3,2-b]pyridin-3-yl]carbamate

5-(2-Methoxyethoxy)-3-nitro-1H-pyrrolo[3,2-b]pyridine (8.0 g, 33.7 mmol, 1.0 equiv.) and (Boc)₂O (11.0 g, 50.6 mmol, 1.5 equiv.) were dissolved in MeOH (100 mL), then Pd/C (1.8 g, 10% wt.) was added. The reaction mixture was sparged with nitrogen, placed under an atmosphere of hydrogen gas (balloon), then stirred for 2 hours at ambient temperature. The solids were removed by filtration and the filtrate was concentrated under vacuum. The residue was purified by flash column chromatography on silica gel column, eluting with ethyl acetate/petroleum ether (1:2) to give tert-butyl N-[5-(2-methoxyethoxy)-1H-pyrrolo[3,2-b]pyridin-3-yl]carbamate (3.0 g) as a brown solid. LCMS Method BA: [M+H]⁺=308.

### Step 6: 5-(2-methoxyethoxy)-1H-pyrrolo[3,2-b]pyridin-3-amine hydrochloride

tert-Butyl N-[5-(2-methoxyethoxy)-1H-pyrrolo[3,2-b]pyridin-3-yl]carbamate (3.0 g, 9.8 mmol, 1.0 equiv.) was dissolved in HCl (4M in 1,4-dioxane, 30 mL). The resulting solution was stirred for 3 hours at ambient temperature and then concentrated under vacuum to give 5-(2-methoxyethoxy)-1H-pyrrolo[3,2-b]pyridin-3-amine hydrochloride (2.5 g) as a grey solid. LCMS Method BA: [M+H]⁺=208.

The following intermediates were prepared using the method described for Intermediate B77, above.

| Intermediate | Structure | LCMS data |
|---|---|---|
| Intermediate B75 | 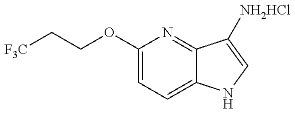 | Method BB: MS-ESI: 246 [M + H]⁺ |
| Intermediate B76 | 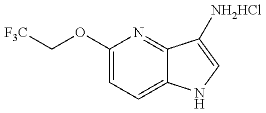 | Method BB: MS-ESI: 232 [M + H]⁺ |

### Synthesis of Intermediate B78 (4,4-difluorocyclohexyl methanesulfonate)

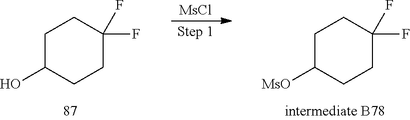 [SMILES: "MsCl" ; OC1CCC(F)(F)CC1 ; "Step 1" ; CS(=O)(=O)OC1CCC(F)(F)CC1]

4,4-Difluorocyclohexan-1-ol (2.5 g, 18.4 mmol, 1.0 equiv.) and TEA (7.6 mL, 55.1 mmol, 3.0 equiv.) were dissolved in DCM (80 mL) and cooled to 0° C., MsCl (2.8 mL, 36.7 mmol, 2.0 equiv.) was added dropwise under an atmosphere of nitrogen, maintaining the solution at 0° C. The reaction mixture was stirred for 1 hour at 0° C. and quenched by the addition of water. The organic layer was separated, washed with brine, dried over anhydrous Na₂SO₄ and concentrated under vacuum to give 4,4-difluorocyclohexyl methanesulfonate (3.8 g) as light yellow oil.

### Example 1: 1-(5-chloro-1H-pyrrolo[2,3-b]pyridin-3-yl)-3-(6-(4,4-difluorocyclohexyl)pyridin-3-yl)urea (Compound 108)

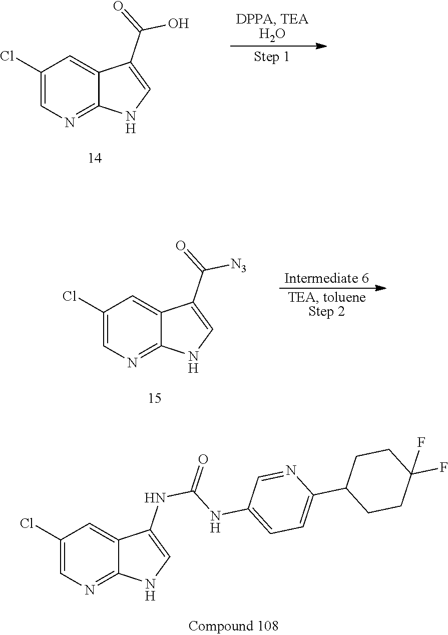

### Step 1: 5-chloro-1H-pyrrolo[2,3-b]pyridine-3-carbonyl azide

5-Chloro-1H-pyrrolo[2,3-b]pyridine-3-carboxylic acid (2.0 g, 10.2 mmol, 1.0 eq.) was dissolved in THE (20 mL), then TEA (1.7 mL, 12.3 mmol, 1.2 eq.) and DPPA (2.7 mL, 12.2 mmol, 1.2 eq.) were added. The resulting solution was stirred for 5 hour at ambient temperature and concentrated under vacuum. The residue was purified by flash column chromatography on silica gel, eluting with ethyl acetate/petroleum ether (1:5) to give 5-chloro-1H-pyrrolo[2,3-b]pyridine-3-carbonyl azide (1.2 g) as a brown yellow solid.

### Step 2: 3-[5-chloro-1H-pyrrolo[2,3-b]pyridin-3-yl]-1-[6-(4,4-difluorocyclohexyl) pyridin-3-yl]urea

5-Chloro-1H-pyrrolo[2,3-b]pyridine-3-carbonyl azide (200.0 mg, 0.9 mmol, 1.0 eq.) was dissolved in toluene (10 mL), then TEA (0.3 mL, 1.8 mmol, 2.0 eq.) and 6-(4,4-difluorocyclohexyl)pyridin-3-amine (191.6 mg, 0.9 mmol, 1.0 eq.) were added. The resulting solution was stirred for 2 hour at 90° C. and concentrated under vacuum. The crude product was purified by Prep-HPLC with following conditions: Column, YMC-Actus Triart C18, 30*250, Sum; mobile phase, Water (10 MMOL/L NH₄HCO₃+0.1% NH₃.H₂O) and ACN (45% Phase B up to 65% in 7 min); Detector, uv 254/220 nm. This resulted in 3-[5-chloro-1H-pyrrolo[2,3-b]pyridin-3-yl]-1-[6-(4,4-difluorocyclohexyl)pyridin-3-yl]urea as a white solid. LCMS Method G: [M+H]⁺=406. ¹H NMR (400 MHz, DMSO-d₆) δ 11.63 (s, 1H), 8.70 (d, 2H), 8.53 (dd, 1H), 8.22 (d, 1H), 8.01 (dd, 1H), 7.91 (dd, 1H), 7.62 (d, 1H), 7.22 (d, 1H), 2.81 (s, 1H), 2.13-1.97 (m, 2H), 1.93-1.91 (m, 4H), 1.77 (td, 2H).

The analogs prepared in Table E3 were prepared using the same method described for Example 1.

TABLE E3

| | | Starting materials | | LCMS |
|---|---|---|---|---|
| Example # | Compound # | Used | Structure | data |
| Example 2 | 106 | 5-methyl-1H- pyrrolo[2,3- b]pyridine-3- carboxylic acid; Intermediate 6 | 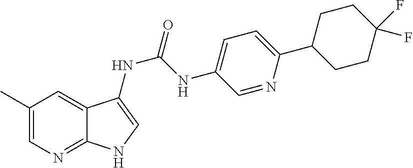 [SMILES: Cc1cnc2[nH]cc(NC(=O)Nc3ccc(C4CCC(F)(F)CC4)nc3)c2c1] 1-(6-(4,4- difluorocyclohexyl)pyridin-3-yl)- 3-(5-methyl-1H-pyrrolo[2,3- b]pyridin-3-yl)urea | Method H: MS-ESI: 386 [M + H]⁺. |
| Example 3 | 109 | 5-flouro-1H- pyrrolo[2,3- b]pyridine-3- carboxylic acid; Intermediate 7 | 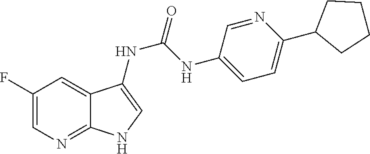 [SMILES: O=C(Nc1ccc(C2CCCC2)nc1)Nc1c[nH]c2ncc(F)cc12] 1-(6-cyclopentylpyridin-3-yl)-3- (5-fluoro-1H-pyrrolo[2,3- b]pyridin-3-yl)urea | Method I: MS-ESI: 340 [M + H]⁺. |
| Example 4 | 110 | 5-flouro-1H- pyrrolo[2,3- b]pyridine-3- carboxylic acid; Intermediate 9 | 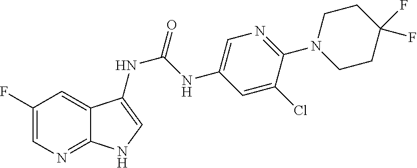 [SMILES: O=C(Nc1cnc(N2CCC(F)(F)CC2)c(Cl)c1)Nc1c[nH]c2ncc(F)cc12] 1-(5-chloro-6-(4,4- difluoropiperidin-1-yl)pyridin-3- yl)-3-(5-fluoro-1H-pyrrolo[2,3- b]pyridin-3-yl)urea | Method H: MS-ESI: 425 [M + H]⁺. |
| Example 5 | 111 | 5-flouro-1H- pyrrolo[2,3- b]pyridine-3- carboxylic acid; Intermediate 10 | 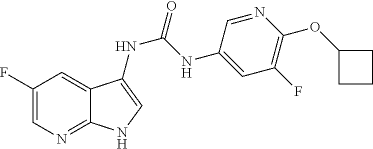 [SMILES: O=C(Nc1cnc(OC2CCC2)c(F)c1)Nc1c[nH]c2ncc(F)cc12] 1-(6-cyclobutoxy-5- fluoropyridin-3-yl)-3-(5-fluoro- 1H-pyrrolo[2,3-b]pyridin-3- yl)urea | Method G: MS-ESI: 360 [M + H]⁺. |
| Example 6 | 112 | 5-flouro-1H- pyrrolo[2,3- b]pyridine-3- carboxylic acid; N²- cyclobutylpyridine- 2,5-diamine | 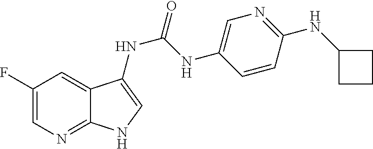 [SMILES: O=C(Nc1ccc(NC2CCC2)nc1)Nc1c[nH]c2ncc(F)cc12] 1-(6-(cyclobutylamino)pyridin-3- yl)-3-(5-fluoro-1H-pyrrolo[2,3- b]pyridin-3-yl)urea | Method A: MS-ESI: 341 [M − H]⁻. |
| Example 7 | 113 | 5-flouro-1H- pyrrolo[2,3- b]pyridine-3- carboxylic acid; Intermediate 8 | 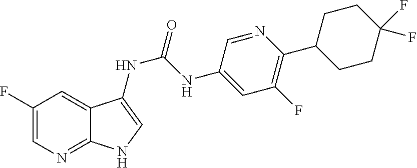 [SMILES: O=C(Nc1cnc(C2CCC(F)(F)CC2)c(F)c1)Nc1c[nH]c2ncc(F)cc12] 1-(6-(4,4-difluorocyclohexyl)-5- fluoropyridin-3-yl)-3-(5-fluoro- 1H-pyrrolo[2,3-b]pyridin-3- yl)urea | Method G: MS-ESI: 408 [M + H]⁺. |
| Example 8 | 115 | 5-flouro-1H- pyrrolo[2,3- b]pyridine-3- carboxylic acid; 1-(5-aminopyridin-2- yl)piperidin-4-ol | 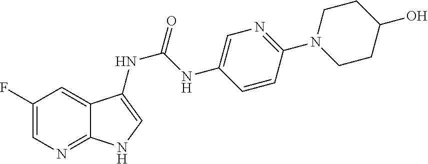 [SMILES: O=C(Nc1ccc(N2CCC(O)CC2)nc1)Nc1c[nH]c2ncc(F)cc12] 1-(5-fluoro-1H-pyrrolo[2,3- b]pyridin-3-yl)-3-(6-(4- hydroxypiperidin-1-yl)pyridin-3- yl)urea | Method G: MS-ESI: 371 [M + H]⁺. |
| Example 9 | 116 | 5-flouro-1H- pyrrolo[2,3- b]pyridine-3- carboxylic acid; Intermediate 6 | 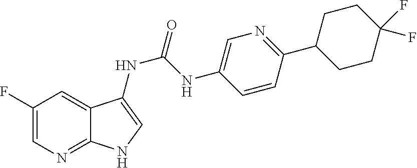 [SMILES: O=C(Nc1ccc(C2CCC(F)(F)CC2)nc1)Nc1c[nH]c2ncc(F)cc12] 1-(6-(4,4- difluorocyclohexyl)pyridin-3-yl)- 3-(5-fluoro-1H-pyrrolo[2,3- b]pyridin-3-yl)urea | Method G: MS-ESI: 390 [M + H]⁺. |
| Example 10 | 117 | 5-flouro-1H- pyrrolo [2,3- b]pyridine-3- carboxylic acid; 6- cyclobutoxypyridin- 3-amine | 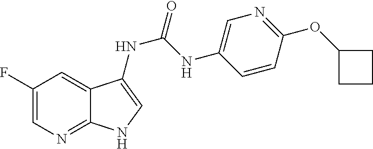 [SMILES: O=C(Nc1ccc(OC2CCC2)nc1)Nc1c[nH]c2ncc(F)cc12] 1-(6-cyclobutoxypyridin-3-yl)-3- (5-fluoro-1H-pyrrolo[2,3- b]pyridin-3-yl)urea | Method A: MS-ESI: 342 [M + H]⁺. |
| Example 11 | 118 | 5-flouro-1H- pyrrolo[2,3- b]pyridine-3- carboxylic acid; 6-(4,4- difluoropiperidin-1- yl)pyridin-3- amine•3HCl | 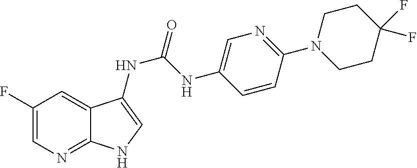 [SMILES: O=C(Nc1ccc(N2CCC(F)(F)CC2)nc1)Nc1c[nH]c2ncc(F)cc12] 1-(6-(4,4-difluoropiperidin-1- yl)pyridin-3-yl)-3-(5-fluoro-1H- pyrrolo[2,3-b]pyridin-3-yl)urea | Method G: MS-ESI: 391 [M + H]⁺. |
| Example 12 | 119 | 5-flouro-1H- pyrrolo [2,3- b]pyridine-3- carboxylic acid; 6- morpholinopyridin- 3-amine | 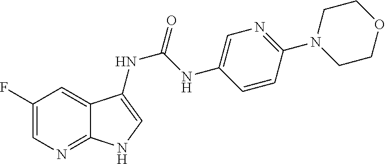 [SMILES: O=C(Nc1ccc(N2CCOCC2)nc1)Nc1c[nH]c2ncc(F)cc12] 1-(5-fluoro-1H-pyrrolo[2,3- b]pyridin-3-yl)-3-(6- morpholinopyridin-3-yl)urea | Method G: MS-ESI: 357 [M + H]⁺. |
| Example 13 | 104 | Intermediate 4 Intermediate 6 | 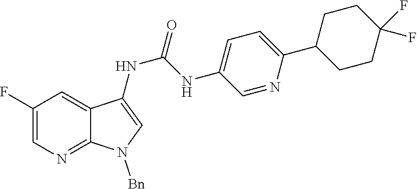 [SMILES: O=C(Nc1ccc(C2CCC(F)(F)CC2)nc1)Nc1cn(Cc2ccccc2)c2ncc(F)cc12] 1-(1-benzyl-5-fluoro-1H- pyrrolo[2,3-b]pyridin-3-yl)-3-(6- (4,4-difluorocyclohexyl)pyridin- 3-yl)urea | Method A: MS-ESI: 480 [M + H]⁺. |
| Example 14 | 127 | Intermediate 5 Intermediate 6 | 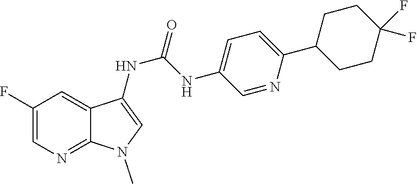 [SMILES: Cn1cc(NC(=O)Nc2ccc(C3CCC(F)(F)CC3)nc2)c2cc(F)cnc21] 1-(6-(4,4- difluorocyclohexyl)pyridin-3-yl)- 3-(5-fluoro-1-methyl-1H- pyrrolo[2,3-b]pyridin-3-yl)urea | Method G: MS-ESI: 404 [M − H]⁻. |

### Example 15: 1-(6-(4,4-difluorocyclohexyl)pyridin-3-yl)-3-(5-(1-isopropyl-1H-pyrazol-4-yl)-1H-pyrrolo[2,3-b]pyridin-3-yl)urea, (Compound 107)

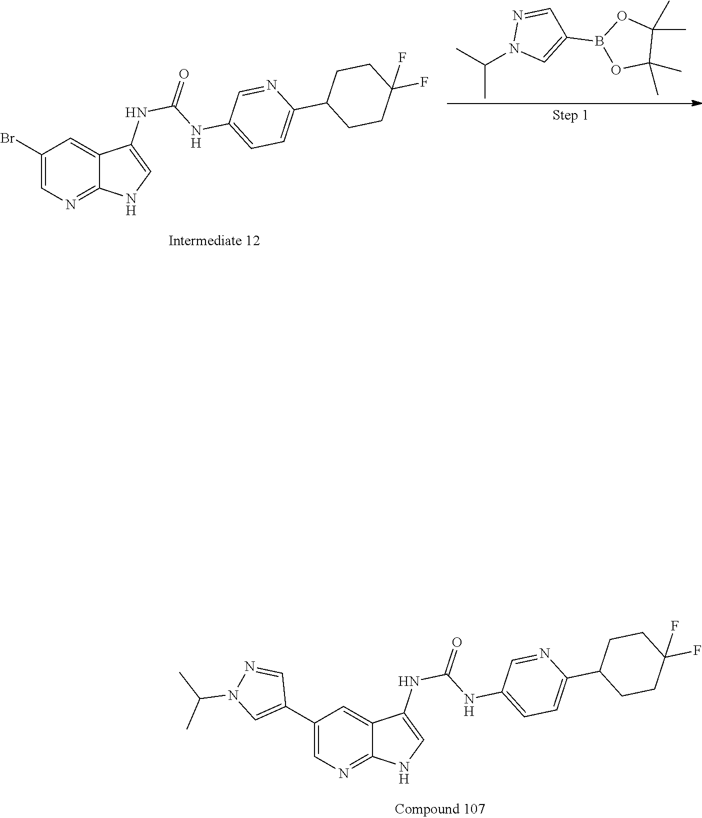

3-[5-Bromo-1H-pyrrolo[2,3-b]pyridin-3-yl]-1-[6-(4,4-difluorocyclohexyl)pyridin-3-yl]urea (350.0 mg, 0.8 mmol, 1.0 eq.) was dissolved dioxane (5 mL) and H₂O (1 mL), then K₃PO₄ (495.0 mg, 2.3 mmol, 3.0 eq.), XPhos Pd G3 (329.0 mg, 0.4 mmol, 0.5 eq.) and 1-isopropyl-4-(4,4,5,5-tetramethyl-1,3,2-dioxaborolan-2-yl)pyrazole (275.3 mg, 1.2 mmol, 1.5 eq.) were added under nitrogen. The resulting solution was stirred for 2 hour at 90° C. and concentrated under vacuum. The residue was purified by flash column chromatography on silica gel, eluting with ethyl acetate/petroleum ether (1:1). The crude product was further purified by Prep-HPLC with following conditions: Column, YMC-Actus Triart C18, 30*250, 5 um; mobile phase, Water (10 mM NH₄HCO₃) and ACN (10% Phase B up to 55% in 7 min); Detector, uv 254/220 nm. This resulted in 1-[6-(4,4-difluorocyclohexyl)pyridin-3-yl]-3-[5-(1-isopropylpyrazol-4-yl)-1H-pyrrolo[2,3-b]pyridin-3-yl]urea as a white solid. LCMS Method G: [M+H]⁺=480. ¹H NMR (400 MHz, DMSO-d₆) δ 11.31 (s, 1H), 8.73 (s, 1H), 8.62 (s, 1H), 8.52 (dd, 2H), 8.23 (s, 1H), 8.03 (d, 1H), 7.92 (dd, 1H), 7.85 (d, 1H), 7.52 (d, 1H), 7.22 (d, 1H), 4.53 (p, 1H), 2.81 (s, 1H), 2.11 (d, 2H), 1.83-1.69 (m, 2H), 1.47 (d, 6H).

The analogs prepared in Table E4 were prepared using the same method described for Example 15.

TABLE E4

| Example # | Boronic acid | Structure | LCMS data |
|---|---|---|---|
| Example 16 (Com- pound 105) | 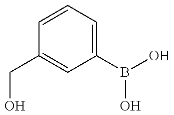 [SMILES: OCc1cccc(B(O)O)c1] | 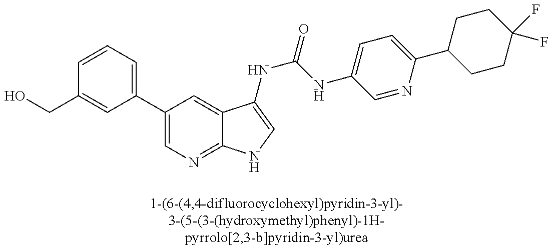 [SMILES: O=C(Nc1ccc(C2CCC(F)(F)CC2)nc1)Nc1c[nH]c2ncc(-c3cccc(CO)c3)cc12] | Method G: MS-ESI: 478 [M + H]⁺. |

### Example 17-18: tert-butyl 4-(3-(3-(6-(4,4-difluorocyclohexyl)pyridin-3-yl)ureido)-5-fluoro-1H-pyrrolo[2,3-b]pyridin-1-yl)piperidine-1-carboxylate (Compound 103) and 1-(6-(4,4-difluorocyclohexyl)pyridin-3-yl)-3-(5-fluoro-1-(piperidin-4-yl)-1H-pyrrolo[2,3-b]pyridin-3-yl)urea (Compound 128)

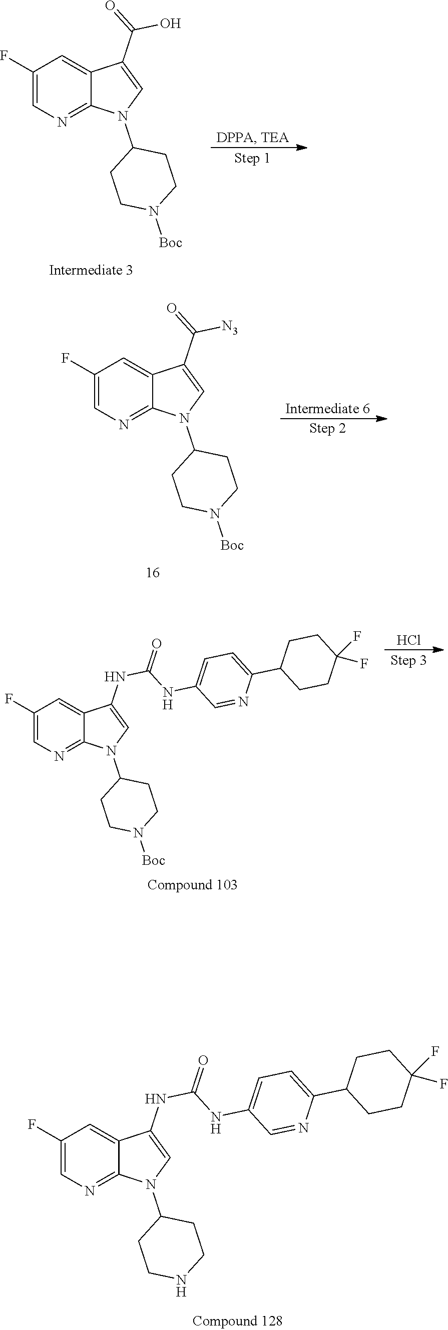

### Step 1: tert-butyl 4-(3-(azidocarbonyl)-5-fluoro-1H-pyrrolo[2,3-b]pyridin-1-yl)piperidine-1-carboxylate

1-[1-(tert-butoxycarbonyl)piperidin-4-yl]-5-fluoropyrrolo[2,3-b]pyridine-3-carboxylic acid (400.0 mg, 1.1 mmol, 1.0 eq.) and TEA (0.3 mL, 2.2 mmol, 2.0 eq.) were dissolved in THE (5 mL), then DPPA (0.5 mL, 2.2 mmol, 2.0 eq.) was added dropwise. The resulting mixture was stirred for overnight at ambient temperature and then concentrated under vacuum. The residue was purified by flash column chromatography on silica gel, eluting with petroleum ether/EtOAc (2:1) to give tert-butyl 4-[3-(azidocarbonyl)-5-fluoropyrrolo[2,3-b]pyridin-1-yl]piperidine-1-carboxylate as a yellow solid. LCMS Method D: [M+H]⁺=389.

### Step 2: tert-butyl 4-(3-(3-(6-(4,4-difluorocyclohexyl)pyridin-3-yl)ureido)-5-fluoro-1H-pyrrolo[2,3-b]pyridin-1-yl)piperidine-1-carboxylate

tert-butyl 4-[3-(azidocarbonyl)-5-fluoropyrrolo[2,3-b]pyridin-1-yl]piperidine-1-carboxylate (380.0 mg, 1.0 mmol, 1.0 eq.) and 6-(4,4-difluorocyclohexyl)pyridin-3-amine (207.7 mg, 1.0 mmol, 1.0 eq.) were dissolved in toluene (5 mL). The resulting mixture was stirred for 2 hour at 90° C. and concentrated under vacuum. The residue was purified by Prep-HPLC with the following conditions: Column: YMC-Actus Triart C18, 30*250.5 um; Mobile Phase A: Water (10 mM NH₄HCO₃), Mobile Phase B: ACN; Flow rate: 60 mL/min; Gradient: 57 B to 87 B in 7 min; 254 nm; RT1: 7.03. This resulted in tert-butyl 4-(3-(3-(6-(4,4-difluorocyclohexyl)pyridin-3-yl)ureido)-5-fluoro-1H-pyrrolo[2,3-b]pyridin-1-yl)piperidine-1-carboxylate as a white solid. LCMS Method B: [M+H]⁺=573. ¹H NMR (400 MHz, DMSO-d₆) δ 8.76 (s, 1H), 8.66 (s, 1H), 8.53 (d, 1H), 8.25 (t, 1H), 7.88 (d, 1H), 7.79 (t, 2H), 4.88 (t, 1H), 4.15 (d, 2H), 2.99 (s, 2H), 2.80 (t, 1H), 2.07-1.90 (m, 21H)

### Step 3: 1-(6-(4,4-difluorocyclohexyl)pyridin-3-yl)-3-(5-fluoro-1-(piperidin-4-yl)-1H-pyrrolo[2,3-b]pyridin-3-yl)urea

tert-Butyl4-(3-(3-(6-(4,4-difluorocyclohexyl)pyridin-3-yl)ureido)-5-fluoro-1H-pyrrolo[2,3-b]pyridin-1-yl)piperidine-1-carboxylate (100.0 mg, 0.2 mmol, 1.0 eq.) was dissolved in dioxane (2 mL), then a solution of HCl/dioxane (2 ml, 4 mol/L) was added. The resulting mixture was stirred for overnight at ambient temperature and concentrated under vacuum. The residue was purified by Prep-HPLC with the following conditions: Column: XBridge Prep OBD C18 Column, 30×150 mm Sum; Mobile Phase A: Water (10 MMOL/L NH₄HCO₃), Mobile Phase B: ACN; Flow rate: 60 mL/min; Gradient: 17 B to 47 B in 10 min; 254 nm; RT1: 9.63. This resulted in 1-[6-(4,4-difluorocyclohexyl)pyridin-3-yl]-3-[5-fluoro-1-(piperidin-4-yl)pyrrolo[2,3-b]pyridin-3-yl]urea as a white solid. LCMS Method B: [M+H]⁺=473. ¹H NMR (400 MHz, DMSO-d₆) δ 8.90 (d, 2H), 8.54 (s, 1H), 8.24 (s, 1H), 7.88 (d, 1H), 7.81 (d, 2H), 7.19 (d, 1H), 4.73 (t, 1H), 3.10 (d, 2H), 2.90 (t, 1H), 2.70 (t, 2H), 2.10-1.93 (m, 12H).

The analogs prepared in Table E5 were prepared using the same method described for Examples 17-18.

TABLE E5

| | | Starting | | |
|---|---|---|---|---|
| Example # | | materials | Structure | LCMS data |
| Example 19 | 101 | Intermediate 1 Intermediate 6 | 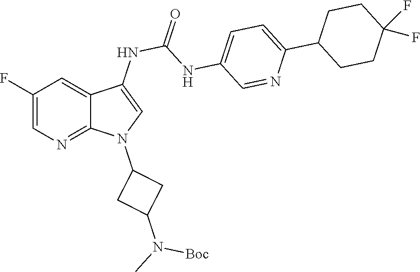 [SMILES: CN(C(=O)OC(C)(C)C)C1CC(n2cc(NC(=O)Nc3ccc(C4CCC(F)(F)CC4)nc3)c3cc(F)cnc32)C1] | Method C: MS-ESI: 573 [M + H]⁺. |
| | | | tert-butyl (3-(3-(3-(6-(4,4- | |
| | | | difluorocyclohexyl)pyridin-3- | |
| | | | yl)ureido)-5-fluoro-1H- | |
| | | | pyrrolo[2,3-b]pyridin-1- | |
| | | | yl)cyclobutyl)(methyl)carbamate | |
| Example 20 | 124 | Intermediate 1 Intermediate 6 | 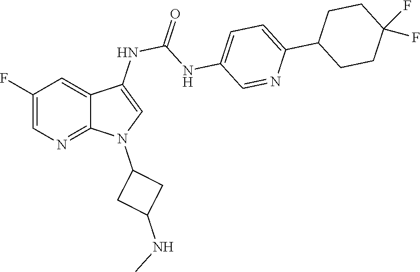 [SMILES: CNC1CC(n2cc(NC(=O)Nc3ccc(C4CCC(F)(F)CC4)nc3)c3cc(F)cnc32)C1] | Method B: MS-ESI: 473 [M + H]⁺. |
| | | | 1-(6-(4,4- | |
| | | | difluorocyclohexyl)pyridin-3-yl)- | |
| | | | 3-(5-fluoro-1-(3- | |
| | | | (methylamino)cyclobutyl)-1H- | |
| | | | pyrrolo[2,3-b]pyridin-3-yl)urea | |
| Example 21 | 102 | Intermediate 2 Intermediate 6 | 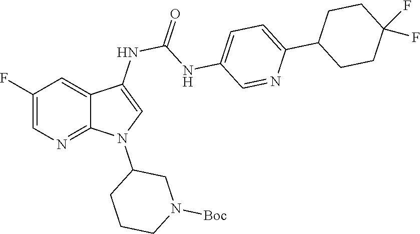 [SMILES: CC(C)(C)OC(=O)N1CCCC(n2cc(NC(=O)Nc3ccc(C4CCC(F)(F)CC4)nc3)c3cc(F)cnc32)C1] | Method E: MS-ESI: 573 [M + H]⁺. |
| | | | tert-butyl 3-(3-(3-(6-(4,4- | |
| | | | difluorocyclohexyl)pyridin-3- | |
| | | | yl)ureido)-5-fluoro-1H- | |
| | | | pyrrolo[2,3-b]pyridin-1- | |
| | | | yl)piperidine-1-carboxylate | |
| Example 22 | 123 | Intermediate 2 Intermediate 6 | 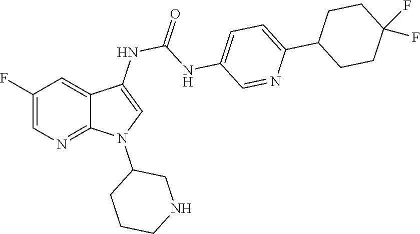 [SMILES: O=C(Nc1ccc(C2CCC(F)(F)CC2)nc1)Nc1cn(C2CCCNC2)c2ncc(F)cc12] | Method B: MS-ESI: 473 [M + H]⁺. |
| | | | 1-(6-(4,4- | |
| | | | difluorocyclohexyl)pyridin-3-yl)- | |
| | | | 3-(5-fluoro-1-(piperidin-3-yl)- | |
| | | | 1H-pyrrolo[2,3-b]pyridin-3- | |
| | | | yl)urea | |

### Example 23: 1-(5-fluoro-1H-pyrrolo[2,3-b]pyridin-3-yl)-3-(6-(4-hydroxycyclohexyl)pyridin-3-yl)urea (Compound 114)

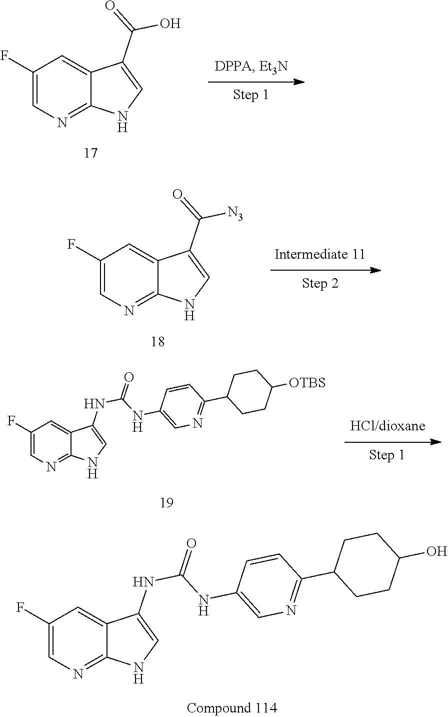 [SMILES: O=C(O)c1c[nH]c2ncc(F)cc12 ; [N-]=[N+]=NC(=O)c1c[nH]c2ncc(F)cc12 ; CC(C)(C)[Si](C)(C)OC1CCC(c2ccc(NC(=O)Nc3c[nH]c4ncc(F)cc34)cn2)CC1 ; O=C(Nc1ccc(C2CCC(O)CC2)nc1)Nc1c[nH]c2ncc(F)cc12]

### Step 1: 5-fluoro-1H-pyrrolo[2,3-b]pyridine-3-carbonyl azide

5-Fluoro-1H-pyrrolo[2,3-b]pyridine-3-carboxylic acid (2.0 g, 11.1 mmol, 1.0 eq.) was dissolved in THE (20 mL), then TEA (1.7 mL, 12.3 mmol, 1.1 eq.) and DPPA (2.7 mL, 12.2 mmol, 1.1 eq.) were added. The resulting solution was stirred for 5 hour at ambient temperature and concentrated under vacuum. The residue was purified by flash column chromatography on silica gel, eluting with ethyl acetate/petroleum ether (1:5) to give 5-fluoro-1H-pyrrolo[2,3-b]pyridine-3-carbonyl azide (1.2 g) as a brown yellow solid.

### Step 2: 1-(6-[4-[(tert-butyldimethylsilyl)oxy]cyclohexyl]pyridin-3-yl)-3-[5-fluoro-1H-pyrrolo[2,3-b]pyridin-3-yl]urea

5-fluoro-1H-pyrrolo[2,3-b]pyridine-3-carbonyl azide (133.9 mg, 0.7 mmol, 1.0 eq.) was dissolved in toluene (20 mL), then TEA (0.2 mL, 1.4 mmol, 2.0 eq.) and 6-[4-[(tert-butyldimethylsilyl)oxy]cyclohexyl]pyridin-3-amine (200.0 mg, 0.7 mmol, 1.0 eq.) were added. The resulting solution was stirred for 2 hour at 90° C. and concentrated under vacuum. The residue was purified by flash column chromatography on silica gel, eluting with ethyl acetate/petroleum ether (1:3) to give 1-(6-[4-[(tert-butyldimethylsilyl)oxy]cyclohexyl]pyridin-3-yl)-3-[5-fluoro-1H-pyrrolo[2,3-b]pyridin-3-yl]urea as a light yellow solid. LCMS: Method F, MS-ESI: 484 [M+H]⁺.

### Step 3: 5-fluoro-1H-pyrrolo[2,3-b]pyridine-3-carbonyl azide

1-(6-[4-[(tert-butyldimethylsilyl)oxy]cyclohexyl]pyridin-3-yl)-3-[5-fluoro-1H-pyrrolo[2,3-b]pyridin-3-yl]urea (60.0 mg, 0.1 mmol, 1.0 equiv) was dissolved in THE (15 mL), then a solution of HCl/1,4-dioxane (1 mL, 4 mol/L) was added. The resulting solution was stirred for 30 min at ambient temperature and concentrated under vacuum. The crude product was purified reverse flash chromatography with the following conditions: column, C18 silica gel; mobile phase, ACN/water, 0% to 40% gradient in 30 min; detector, UV 254 nm. The resulting product was further purified by Prep-HPLC with the following conditions: Column, XBridge Prep OBD C18 Column, 19*250 mm, Sum; mobile phase, Water (10 MMOL/L NH₄HCO₃) and MeOH (46% Phase B up to 60% in 7 min); Detector, uv 254 nm. This resulted in 3-[5-fluoro-1H-pyrrolo[2,3-b]pyridin-3-yl]-1-[6-(4-hydroxycyclohexyl)pyridin-3-yl]urea as a light yellow solid. LCMS: Method J, MS-ESI: 370 [M+H]+. ¹H NMR (400 MHz, DMSO-d₆) δ 11.53 (s, 1H), 8.66 (s, 1H), 8.60 (s, 1H), 8.50 (s, 1H), 8.21 (s, 1H), 7.88-7.86 (m, 1H), 7.76-7.73 (m, 1H), 7.62 (d, 1H), 7.17 (d, 1H), 4.31 (s, 1H), 3.90-3.86 (m, 1H), 2.61-2.58 (m, 1H), 1.96-1.90 (m, 2H), 1.73-1.70 (m, 2H), 1.58-1.52 (m, 4H).

### Example 24: 1-(5-chloro-6-(4,4-difluoropiperidin-1-yl)pyridin-3-yl)-3-(5-fluoro-1H-pyrrolo[2,3-b]pyridin-3-yl)urea (Compound 202)

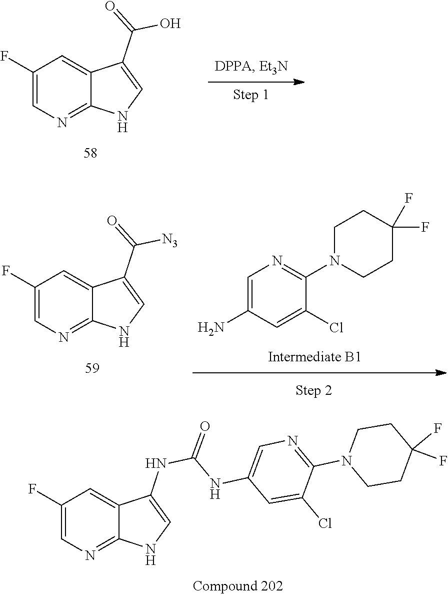 [SMILES: O=C(O)c1c[nH]c2ncc(F)cc12 ; [N-]=[N+]=NC(=O)c1c[nH]c2ncc(F)cc12 ; Nc1cnc(N2CCC(F)(F)CC2)c(Cl)c1 ; O=C(Nc1cnc(N2CCC(F)(F)CC2)c(Cl)c1)Nc1c[nH]c2ncc(F)cc12]

### Step 1: 5-fluoro-1H-pyrrolo[2,3-b]pyridine-3-carbonyl azide

5-fluoro-1H-pyrrolo[2,3-b]pyridine-3-carboxylic acid (1.0 g, 5.6 mmol, 1.0 equiv.) and TEA (1.5 mL, 11.1 mmol, 2.0 equiv.) were dissolved in THE (30 ml), then DPPA (2.3 g, 8.3 mmol, 1.5 equiv.) was added. The resulting mixture was stirred overnight at ambient temperature and then concentrated under vacuum. The precipitated solids were collected by filtration and washed with ethyl acetate to give 5-fluoro-1H-pyrrolo[2,3-b]pyridine-3-carbonyl azide as a yellow solid. LCMS Method BA: [M+H]⁺=206.

### Step 2: 1-(5-chloro-6-(4,4-difluoropiperidin-1-yl)pyridin-3-yl)-3-(5-fluoro-1H-pyrrolo[2,3-b]pyridin-3-yl)urea

5-Chloro-6-(4,4-difluoropiperidin-1-yl)pyridin-3-amine (150.0 mg, 0.6 mmol, 1.0 equiv.) and 5-fluoro-1H-pyrrolo[2,3-b]pyridine-3-carbonyl azide (248.5 mg, 1.2 mmol, 2.0 equiv.) was dissolved in toluene (40 mL), then DIEA (0.2 mL, 1.2 mmol, 2.0 equiv.) was added. The reaction mixture was heated to 90° C. for 4 hours, and then concentrated under vacuum. The residue was purified by flash column chromatography on silica gel column, eluting with ethyl acetate/petroleum ether (1:2) to give the crude product, which was further purified by Prep-HPLC with the following conditions Column: XBridge Prep OBD C18 Column 30×150 mm 5 m; Mobile Phase A: Water (10 mM NH₄HCO₃), Mobile Phase B: ACN; Flow rate: 60 mL/min; Gradient: 30% B to 60% B in 8 min; 254 nm. This resulted in 1-[5-chloro-6-(4,4-difluoropiperidin-1-yl)pyridin-3-yl]-3-[5-fluoro-1H-pyrrolo[2,3-b]pyridin-3-yl]urea as a white solid. LCMS Method BD: [M+H]⁺=425. ¹HNMR (400 MHz, DMSO-d₆): δ 11.56 (s, 1H), 8.78 (s, 1H), 8.68 (s, 1H), 8.26-8.21 (m, 2H), 8.13 (d, 1H), 7.77-7.74 (m, 1H), 7.62 (d, 1H), 3.30-3.27 (m, 4H), 2.16-2.06 (m, 4H).

The following compounds were prepared using the method described for Example 24.

| | | | LCMS |
|---|---|---|---|
| Compound | Starting Materials | Structure | data |
| Example 25 (Compound 201) | 5-fluoro-1H- pyrrolo[2,3- b]pyridine-3- carboxylic acid Intermediate B24 | 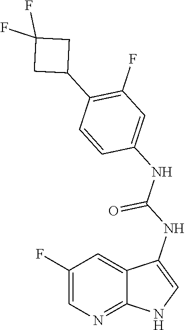 [SMILES: O=C(Nc1ccc(C2CC(F)(F)C2)c(F)c1)Nc1c[nH]c2ncc(F)cc12] | Method BD: MS- ESI: 379 [M + H]⁺. |
| | | 1-(4-(3,3-difluorocyclobutyl)-3- | |
| | | fluorophenyl)-3-(5-fluoro-1H- | |
| | | pyrrolo[2,3-b]pyridin-3-yl)urea | |
| Example 26 (Compound 200) | Intermediate 42 Intermediate B20 | 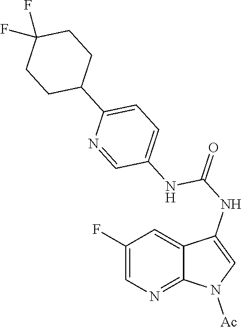 [SMILES: CC(=O)n1cc(NC(=O)Nc2ccc(C3CCC(F)(F)CC3)nc2)c2cc(F)cnc21] | Method BC: MS- ESI: 432 [M + H]⁺. |
| | | 1-(1-acetyl-5-fluoro-1H- | |
| | | pyrrolo[2,3-b]pyridin-3-yl)-3-(6- | |
| | | (4,4-difluorocyclohexyl)pyridin-3- | |
| | | yl)urea | |
| Example 27 (Compound 199) | 5-fluoro-1H- pyrrolo[2,3- b]pyridine-3- carboxylic acid Intermediate B18 | 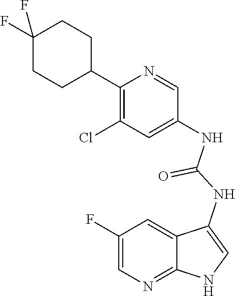 [SMILES: O=C(Nc1cnc(C2CCC(F)(F)CC2)c(Cl)c1)Nc1c[nH]c2ncc(F)cc12] | Method BD: MS- ESI: 424 [M + H]⁺. |
| | | 1-(5-chloro-6-(4,4- | |
| | | difluorocyclohexyl)pyridin-3-yl)- | |
| | | 3-(5-fluoro-1H-pyrrolo[2,3- | |
| | | b]pyridin-3-yl)urea | |
| Example 28 (Compound 198) | 5-fluoro-1H- pyrrolo[2,3- b]pyridine-3- carboxylic acid Intermediate B2 | 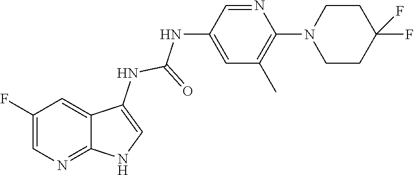 [SMILES: Cc1cc(NC(=O)Nc2c[nH]c3ncc(F)cc23)cnc1N1CCC(F)(F)CC1] | Method BD: MS- ESI: 405 [M + H]⁺. |
| | | 1-(6-(4,4-difluoropiperidin-1-yl)- | |
| | | 5-methylpyridin-3-yl)-3-(5-fluoro- | |
| | | 1H-pyrrolo[2,3-b]pyridin-3- | |
| | | yl)urea | |
| Example 29 (Compound 196) | 5-fluoro-1H- pyrrolo[2,3- b]pyridine-3- carboxylic acid Intermediate B3 | 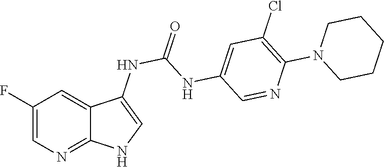 [SMILES: O=C(Nc1cnc(N2CCCCC2)c(Cl)c1)Nc1c[nH]c2ncc(F)cc12] | Method BF: MS- ESI: 389 [M + H]⁺. |
| | | 1-(5-chloro-6-(piperidin-1- | |
| | | yl)pyridin-3-yl)-3-(5-fluoro-1H- | |
| | | pyrrolo[2,3-b]pyridin-3-yl)urea | |
| Example 30 (Compound 195) | 5-fluoro-1H- pyrrolo[2,3- b]pyridine-3- carboxylic acid Intermediate B19 | 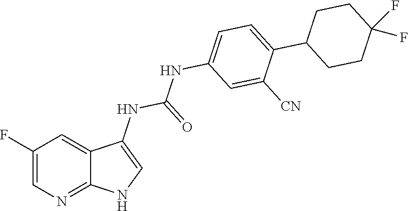 [SMILES: N#Cc1cc(NC(=O)Nc2c[nH]c3ncc(F)cc23)ccc1C1CCC(F)(F)CC1] | Method BG: MS- ESI: 414 [M + H]⁺. |
| | | 1-(3-cyano-4-(4,4- | |
| | | difluorocyclohexyl)phenyl)-3-(5- | |
| | | fluoro-1H-pyrrolo[2,3-b]pyridin- | |
| | | 3-yl)urea | |
| Example 31 (Compound 194) | 5-fluoro-1H- pyrrolo[2,3- b]pyridine-3- carboxylic acid Intermediate B4 | 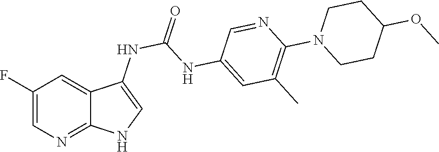 [SMILES: COC1CCN(c2ncc(NC(=O)Nc3c[nH]c4ncc(F)cc34)cc2C)CC1] | Method BD: MS- ESI: 399 [M + H]⁺. |
| | | 1-(5-fluoro-1H-pyrrolo[2,3- | |
| | | b]pyridin-3-yl)-3-(6-(4- | |
| | | methoxypiperidin-1-yl)-5- | |
| | | methylpyridin-3-yl)urea | |
| Example 32 (Compound 193) | 5-fluoro-1H- pyrrolo[2,3- b]pyridine-3- carboxylic acid Intermediate B25 | 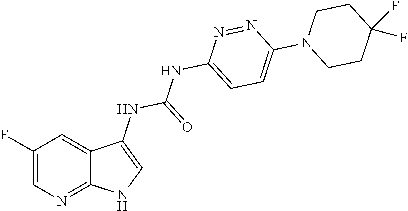 [SMILES: O=C(Nc1ccc(N2CCC(F)(F)CC2)nn1)Nc1c[nH]c2ncc(F)cc12] | Method BD MS- ESI: 392 [M + H]⁺. |
| | | 1-(6-(4,4-difluoropiperidin-1- | |
| | | yl)pyridazin-3-yl)-3-(5-fluoro-1H- | |
| | | pyrrolo[2,3-b]pyridin-3-yl)urea | |
| Example 33 (Compound 192) | 5-fluoro-1H- pyrrolo[2,3- b]pyridine-3- carboxylic acid Intermediate B5 | 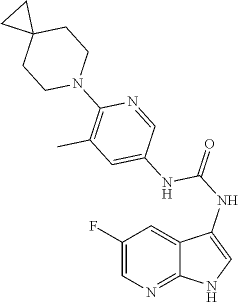 [SMILES: Cc1cc(NC(=O)Nc2c[nH]c3ncc(F)cc23)cnc1N1CCC2(CC1)CC2] | Method BD: MS- ESI: 395.1 [M + H]⁺. |
| | | 1-(5-fluoro-1H-pyrrolo[2,3- | |
| | | b]pyridin-3-yl)-3-(5-methyl-6-(6- | |
| | | azaspiro[2.5]octan-6-yl)pyridin-3- | |
| | | yl)urea | |
| Example 34 (Compound 191) | 5-fluoro-1H- pyrrolo[2,3- b]pyridine-3- carboxylic acid Intermediate B6 | 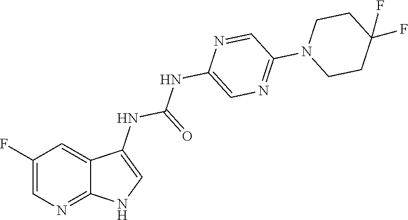 [SMILES: O=C(Nc1cnc(N2CCC(F)(F)CC2)cn1)Nc1c[nH]c2ncc(F)cc12] | Method BD: MS- ESI: 392 [M + H]⁺. |
| | | 1-(5-(4,4-difluoropiperidin-1- | |
| | | yl)pyrazin-2-yl)-3-(5-fluoro-1H- | |
| | | pyrrolo[2,3-b]pyridin-3-yl)urea | |
| Example 35 (Compound 190) | 5-fluoro-1H- pyrrolo[2,3- b]pyridine-3- carboxylic acid Intermediate B7 | 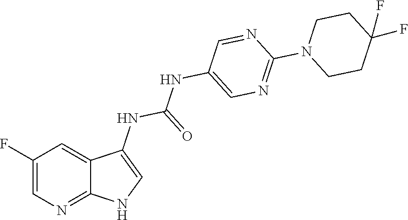 [SMILES: O=C(Nc1cnc(N2CCC(F)(F)CC2)nc1)Nc1c[nH]c2ncc(F)cc12] | Method BF: MS- ESI: 392 [M + H]⁺. |
| | | 1-(2-(4,4-difluoropiperidin-1- | |
| | | yl)pyrimidin-5-yl)-3-(5-fluoro-1H- | |
| | | pyrrolo[2,3-b]pyridin-3-yl)urea | |
| Example 36 (Compound 189) | 1H-pyrrolo[2,3- b]pyridine-3- carboxylic acid Intermediate B1 | 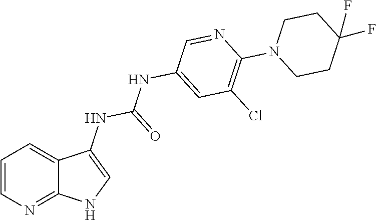 [SMILES: O=C(Nc1cnc(N2CCC(F)(F)CC2)c(Cl)c1)Nc1c[nH]c2ncccc12] | Method BD: MS- ESI: 407 [M + H]⁺. |
| | | 1-(5-chloro-6-(4,4- | |
| | | difluoropiperidin-1-yl)pyridin-3- | |
| | | yl)-3-(1H-pyrrolo[2,3-b]pyridin-3- | |
| | | yl)urea | |
| Example 37 (Compound 187) | 5-methoxy-1H- pyrrolo[3,2- b]pyridine-3- carboxylic acid Intermediate B8 | 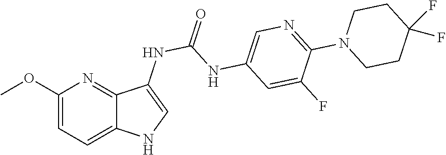 [SMILES: COc1ccc2[nH]cc(NC(=O)Nc3cnc(N4CCC(F)(F)CC4)c(F)c3)c2n1] | Method BD: MS- ESI: 421 [M + H]⁺. |
| | | 1-(6-(4,4-difluoropiperidin-1-yl)- | |
| | | 5-fluoropyridin-3-yl)-3-(5- | |
| | | methoxy-1H-pyrrolo[3,2- | |
| | | b]pyridin-3-yl)urea | |
| Example 38 (Compound 186) | 5-fluoro-1H- pyrrolo[2,3- b]pyridine-3- carboxylic acid Intermediate B27 | 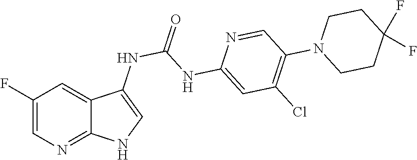 [SMILES: O=C(Nc1cc(Cl)c(N2CCC(F)(F)CC2)cn1)Nc1c[nH]c2ncc(F)cc12] | Method BD: MS- ESI: 425 [M + H]⁺. |
| | | 1-(4-chloro-5-(4,4- | |
| | | difluoropiperidin-1-yl)pyridin-2- | |
| | | yl)-3-(5-fluoro-1H-pyrrolo[2,3- | |
| | | b]pyridin-3-yl)urea | |
| Example 39 (Compound 185) | 5-fluoro-1H- pyrrolo[2,3- b]pyridine-3- carboxylic acid Intermediate B10 | 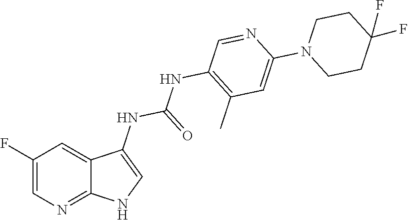 [SMILES: Cc1cc(N2CCC(F)(F)CC2)ncc1NC(=O)Nc1c[nH]c2ncc(F)cc12] | Method BD: MS- ESI: 405 [M + H]⁺. |
| | | 1-(6-(4,4-difluoropiperidin-1-yl)- | |
| | | 4-methylpyridin-3-yl)-3-(5-fluoro- | |
| | | 1H-pyrrolo[2,3-b]pyridin-3- | |
| | | yl)urea | |
| Example 40 (Compound 183) | 5-fluoro-1H- pyrrolo[2,3- b]pyridine-3- carboxylic acid Intermediate B29 | 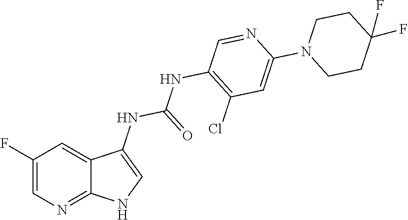 [SMILES: O=C(Nc1cnc(N2CCC(F)(F)CC2)cc1Cl)Nc1c[nH]c2ncc(F)cc12] | Method BF: MS- ESI: 425 [M + H]⁺. |
| | | 1-(4-chloro-6-(4,4- | |
| | | difluoropiperidin-1-yl)pyridin-3- | |
| | | yl)-3-(5-fluoro-1H-pyrrolo[2,3- | |
| | | b]pyridin-3-yl)urea | |
| Example 41 (Compound 179) | 4-methyl-1H- pyrrolo[2,3- b]pyridine-3- carboxylic acid Intermediate B10 | 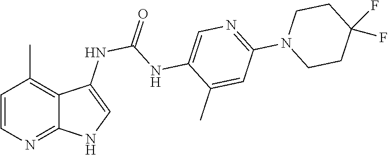 | Method BF: MS- ESI: 425 [M + H]⁺. |
| | | 1-(6-(4,4-difluoropiperidin-1-yl)- | |
| | | 4-methylpyridin-3-yl)-3-(4- | |
| | | methyl-1H-pyrrolo[2,3-b]pyridin- | |
| | | 3-yl)urea | |
| Example 42 (Compound 178) | 5-fluoro-1H- pyrrolo[2,3- b]pyridine-3- carboxylic acid Intermediate B28 | 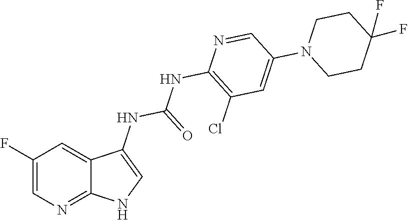 [SMILES: O=C(Nc1ncc(N2CCC(F)(F)CC2)cc1Cl)Nc1c[nH]c2ncc(F)cc12] | Method BF: MS- ESI: 425 [M + H]⁺. |
| | | 1-(3-chloro-5-(4,4- | |
| | | difluoropiperidin-1-yl)pyridin-2- | |
| | | yl)-3-(5-fluoro-1H-pyrrolo[2,3- | |
| | | b]pyridin-3-yl)urea | |
| Example 43 (Compound 177) | 5-fluoro-1H- pyrrolo[2,3- b]pyridine-3- carboxylic acid Intermediate B10 | 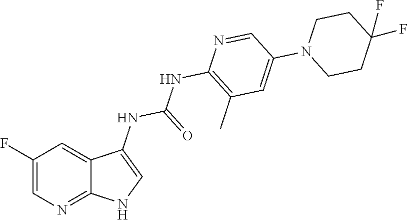 [SMILES: Cc1cc(N2CCC(F)(F)CC2)cnc1NC(=O)Nc1c[nH]c2ncc(F)cc12] | Method BD: MS- ESI: 405 [M + H]⁺. |
| | | 1-(5-(4,4-difluoropiperidin-1-yl)- | |
| | | 3-methylpyridin-2-yl)-3-(5-fluoro- | |
| | | 1H-pyrrolo[2,3-b]pyridin-3- | |
| | | yl)urea | |
| Example 44 (Compound 173) | 5-methoxy-1H- pyrrolo[3,2- b]pyridine-3- carboxylic acid Intermediate B2 | 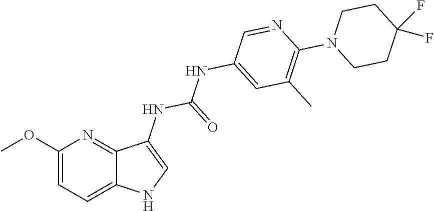 [SMILES: COc1ccc2[nH]cc(NC(=O)Nc3cnc(N4CCC(F)(F)CC4)c(C)c3)c2n1] | Method BD: MS- ESI: 417 [M + H]⁺. |
| | | 1-(6-(4,4-difluoropiperidin-1-yl)- | |
| | | 5-methylpyridin-3-yl)-3-(5- | |
| | | methoxy-1H-pyrrolo[3,2- | |
| | | b]pyridin-3-yl)urea | |
| Example 45 (Compound 172) | 7-fluoro-1H- pyrrolo[3,2- c]pyridine-3- carboxylic acid Intermediate B2 | 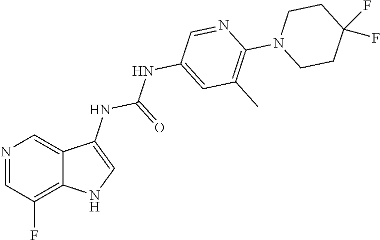 [SMILES: Cc1cc(NC(=O)Nc2c[nH]c3c(F)cncc23)cnc1N1CCC(F)(F)CC1] | Method BC: MS- ESI: 405 [M + H]⁺. |
| | | 1-(6-(4,4-difluoropiperidin-1-yl)- | |
| | | 5-methylpyridin-3-yl)-3-(7-fluoro- | |
| | | 1H-pyrrolo[3,2-c]pyridin-3- | |
| | | yl)urea | |
| Example 46 (Compound 171) | 5-fluoro-1H- pyrrolo[2,3- b]pyridine-3- carboxylic acid Intermediate B11 | 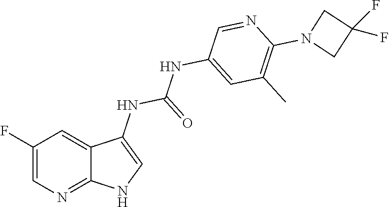 [SMILES: Cc1cc(NC(=O)Nc2c[nH]c3ncc(F)cc23)cnc1N1CC(F)(F)C1] | Method BD: MS- ESI: 377 [M + H]⁺. |
| | | 1-(6-(3,3-difluoroazetidin-1-yl)-5- | |
| | | methylpyridin-3-yl)-3-(5-fluoro- | |
| | | 1H-pyrrolo[2,3-b]pyridin-3- | |
| | | yl)urea | |
| Example 47 (Compound 170) | 5-fluoro-1H- pyrrolo[2,3- b]pyridine-3- carboxylic acid Intermediate B12 | 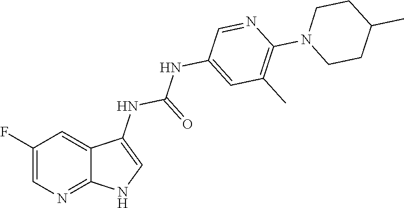 [SMILES: Cc1cc(NC(=O)Nc2c[nH]c3ncc(F)cc23)cnc1N1CCC(C)CC1] | Method BD: MS- ESI: 383 [M + H]⁺. |
| | | 1-(5-fluoro-1H-pyrrolo[2,3- | |
| | | b]pyridin-3-yl)-3-(5-methyl-6-(4- | |
| | | methylpiperidin-1-yl)pyridin-3- | |
| | | yl)urea | |
| Example 48 (Compound 169) | 5-methoxy-1H- pyrrolo[3,2- b]pyridine-3- carboxylic acid Intermediate B21 | 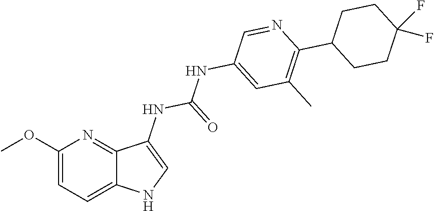 [SMILES: COc1ccc2[nH]cc(NC(=O)Nc3cnc(C4CCC(F)(F)CC4)c(C)c3)c2n1] | Method BD: MS- ESI: 416 [M + H]⁺. |
| | | 1-(6-(4,4-difluorocyclohexyl)-5- | |
| | | methylpyridin-3-yl)-3-(5- | |
| | | methoxy-1H-pyrrolo[3,2- | |
| | | b]pyridin-3-yl)urea | |
| Example 49 (Compound 167) | 5-fluoro-1H- pyrrolo[2,3- b]pyridine-3- carboxylic acid Intermediate B13 | 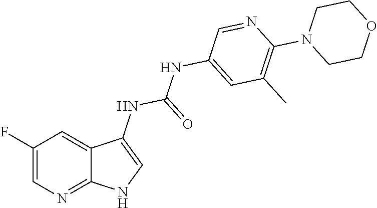 [SMILES: Cc1cc(NC(=O)Nc2c[nH]c3ncc(F)cc23)cnc1N1CCOCC1] | Method BD: MS- ESI: 371 [M + H]⁺. |
| | | 1-(5-fluoro-1H-pyrrolo[2,3- | |
| | | b]pyridin-3-yl)-3-(5-methyl-6- | |
| | | morpholinopyridin-3-yl)urea | |
| Example 50 (Compound 166) | 5-fluoro-1H- pyrrolo[2,3- b]pyridine-3- carboxylic acid Intermediate B32 | 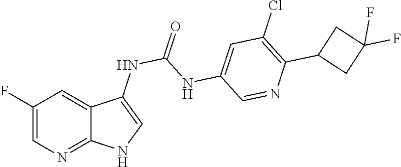 [SMILES: O=C(Nc1cnc(C2CC(F)(F)C2)c(Cl)c1)Nc1c[nH]c2ncc(F)cc12] | Method BD: MS- ESI: 396 [M + H]⁺. |
| | | 1-(5-chloro-6-(3,3- | |
| | | difluorocyclobutyl)pyridin-3-yl)- | |
| | | 3-(5-fluoro-1H-pyrrolo[2,3- | |
| | | b]pyridin-3-yl)urea | |
| Example 51 (Compound 165) | 5-fluoro-1H- pyrrolo[2,3- b]pyridine-3- carboxylic acid Intermediate B14 | 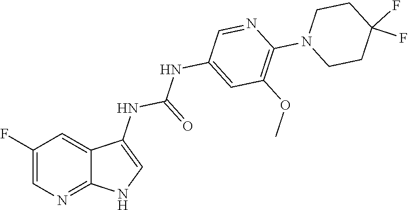 [SMILES: COc1cc(NC(=O)Nc2c[nH]c3ncc(F)cc23)cnc1N1CCC(F)(F)CC1] | Method BF: MS- ESI: 421 [M + H]⁺. |
| | | 1-(6-(4,4-difluoropiperidin-1-yl)- | |
| | | 5-methoxypyridin-3-yl)-3-(5- | |
| | | fluoro-1H-pyrrolo[2,3-b]pyridin- | |
| | | 3-yl)urea | |
| Example 52 (Compound 164) | 5-fluoro-1H- pyrrolo[2,3- b]pyridine-3- carboxylic acid Intermediate B23 | 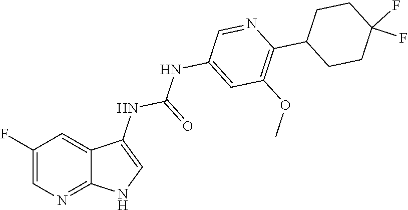 [SMILES: COc1cc(NC(=O)Nc2c[nH]c3ncc(F)cc23)cnc1C1CCC(F)(F)CC1] | Method BD: MS- ESI: 420 [M + H]⁺. |
| | | 1-(6-(4,4-difluorocyclohexyl)-5- | |
| | | methoxypyridin-3-yl)-3-(5-fluoro- | |
| | | 1H-pyrrolo[2,3-b]pyridin-3- | |
| | | yl)urea | |
| Example 53 (Compound 163) | 5-fluoro-1H- pyrrolo[2,3- b]pyridine-3- carboxylic acid Intermediate B34 | 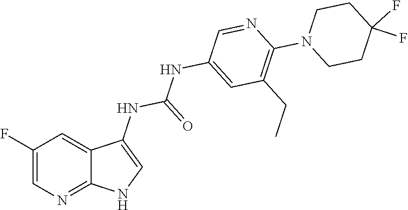 [SMILES: CCc1cc(NC(=O)Nc2c[nH]c3ncc(F)cc23)cnc1N1CCC(F)(F)CC1] | Method BC: MS- ESI: 419 [M + H]⁺. |
| | | 1-(6-(4,4-difluoropiperidin-1-yl)- | |
| | | 5-ethylpyridin-3-yl)-3-(5-fluoro- | |
| | | 1H-pyrrolo[2,3-b]pyridin-3- | |
| | | yl)urea | |
| Example 54 (Compound 162) | 5-fluoro-1H- pyrrolo[2,3- b]pyridine-3- carboxylic acid Intermediate B15 | 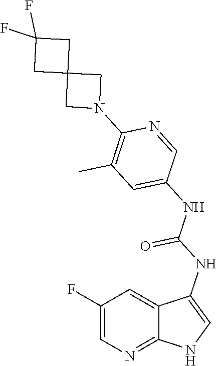 [SMILES: Cc1cc(NC(=O)Nc2c[nH]c3ncc(F)cc23)cnc1N1CC2(C1)CC(F)(F)C2] | Method BF: MS- ESI: 417 [M + H]⁺. |
| | | 1-(6-(6,6-difluoro-2- | |
| | | azaspiro[3.3]heptan-2-yl)-5- | |
| | | methylpyridin-3-yl)-3-(5-fluoro- | |
| | | 1H-pyrrolo[2,3-b]pyridin-3- | |
| | | yl)urea | |
| Example 55 (Compound 161) | 5-fluoro-1H- pyrrolo[2,3- b]pyridine-3- carboxylic acid Intermediate B33 | 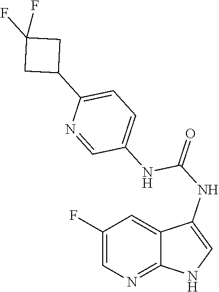 [SMILES: O=C(Nc1ccc(C2CC(F)(F)C2)nc1)Nc1c[nH]c2ncc(F)cc12] | Method BD: MS- ESI: 362 [M + H]⁺. |
| | | 1-(6-(3,3- | |
| | | difluorocyclobutyl)pyridin-3-yl)- | |
| | | 3-(5-fluoro-1H-pyrrolo[2,3- | |
| | | b]pyridin-3-yl)urea | |
| Example 56 (Compound 160) | 5-fluoro-1H- pyrrolo[2,3- b]pyridine-3- carboxylic acid Intermediate B30 | 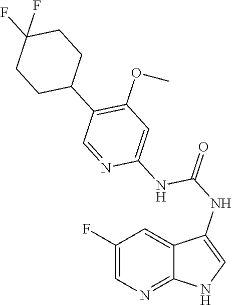 [SMILES: COc1cc(NC(=O)Nc2c[nH]c3ncc(F)cc23)ncc1C1CCC(F)(F)CC1] | Method BD: MS- ESI: 420 [M + H]⁺. |
| | | 1-(5-(4,4-difluorocyclohexyl)-4- | |
| | | methoxypyridin-2-yl)-3-(5-fluoro- | |
| | | 1H-pyrrolo[2,3-b]pyridin-3- | |
| | | yl)urea | |
| Example 57 (Compound 159) | 5-fluoro-1H- pyrrolo[2,3- b]pyridine-3- carboxylic acid Intermediate B16 | 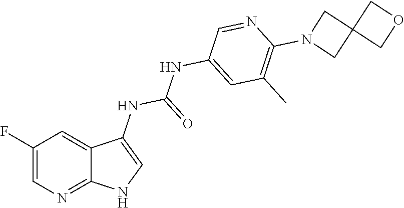 [SMILES: Cc1cc(NC(=O)Nc2c[nH]c3ncc(F)cc23)cnc1N1CC2(COC2)C1] | Method BF: MS- ESI: 383 [M + H]⁺. |
| | | 1-(5-fluoro-1H-pyrrolo[2,3- | |
| | | b]pyridin-3-yl)-3-(5-methyl-6-(2- | |
| | | oxa-6-azaspiro[3.3]heptan-6- | |
| | | yl)pyridin-3-yl)urea | |
| Example 58 (Compound 158) | 5-fluoro-1H- pyrrolo[2,3- b]pyridine-3- carboxylic acid Intermediate B37 | 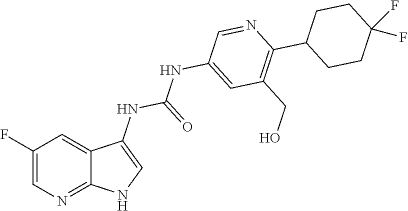 [SMILES: O=C(Nc1cnc(C2CCC(F)(F)CC2)c(CO)c1)Nc1c[nH]c2ncc(F)cc12] | Method BG: MS- ESI: 420 [M + H]⁺. |
| | | 1-(6-(4,4-difluorocyclohexyl)-5- | |
| | | (hydroxymethyl)pyridin-3-yl)-3- | |
| | | (5-fluoro-1H-pyrrolo[2,3- | |
| | | b]pyridin-3-yl)urea | |
| Example 59 (Compound 157) | 5-fluoro-1H- pyrrolo[2,3- b]pyridine-3- carboxylic acid Intermediate B38 | 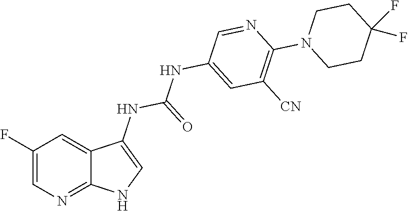 [SMILES: N#Cc1cc(NC(=O)Nc2c[nH]c3ncc(F)cc23)cnc1N1CCC(F)(F)CC1] | Method BE: MS- ESI: 416 [M + H]⁺. |
| | | 1-(5-cyano-6-(4,4- | |
| | | difluoropiperidin-1-yl)pyridin-3- | |
| | | yl)-3-(5-fluoro-1H-pyrrolo[2,3- | |
| | | b]pyridin-3-yl)urea | |
| Example 60 (Compound 156) | 5-fluoro-1H- pyrrolo[2,3- b]pyridine-3- carboxylic acid Intermediate B17 | 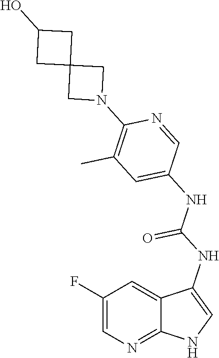 [SMILES: Cc1cc(NC(=O)Nc2c[nH]c3ncc(F)cc23)cnc1N1CC2(CC(O)C2)C1] | Method BF: MS- ESI: 397 [M + H]⁺. |
| | | 1-(5-fluoro-1H-pyrrolo[2,3- | |
| | | b]pyridin-3-yl)-3-(6-(6-hydroxy-2- | |
| | | azaspiro[3.3]heptan-2-yl)-5- | |
| | | methylpyridin-3-yl)urea | |
| Example 61 (Compound 155) | 5-fluoro-1H- pyrrolo[2,3- b]pyridine-3- carboxylic acid Intermediate B39 | 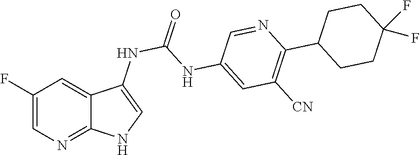 [SMILES: N#Cc1cc(NC(=O)Nc2c[nH]c3ncc(F)cc23)cnc1C1CCC(F)(F)CC1] | Method BD: MS- ESI: 415 [M + H]⁺. |
| | | 1-(5-cyano-6-(4,4- | |
| | | difluorocyclohexyl)pyridin-3-yl)- | |
| | | 3-(5-fluoro-1H-pyrrolo[2,3- | |
| | | b]pyridin-3-yl)urea | |
| Example 62 (Compound 154) | 5-methoxy-1H- pyrrolo[2,3- c]pyridine-3- carboxylic acid Intermediate B19 | 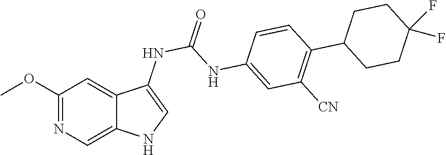 [SMILES: COc1cc2c(NC(=O)Nc3ccc(C4CCC(F)(F)CC4)c(C#N)c3)c[nH]c2cn1] | Method BD: MS- ESI: 426 [M + H]⁺. |
| | | 1-(3-cyano-4-(4,4- | |
| | | difluorocyclohexyl)phenyl)-3-(5- | |
| | | methoxy-1H-pyrrolo[2,3- | |
| | | c]pyridin-3-yl)urea | |
| Example 63 (Compound 152) | 5-fluoro-1H- pyrrolo[2,3- b]pyridine-3- carboxylic acid Intermediate B31 | 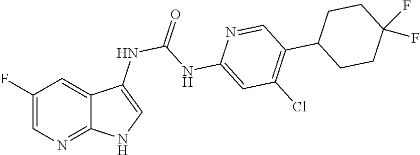 [SMILES: O=C(Nc1cc(Cl)c(C2CCC(F)(F)CC2)cn1)Nc1c[nH]c2ncc(F)cc12] | Method BD: MS- ESI: 424 [M + H]⁺. |
| | | 1-(4-chloro-5-(4,4- | |
| | | difluorocyclohexyl)pyridin-2-yl)- | |
| | | 3-(5-fluoro-1H-pyrrolo[2,3- | |
| | | b]pyridin-3-yl)urea | |
| Example 64 (Compound 151) | 5-chloro-1H- pyrrolo[2,3- b]pyridine-3- carboxylic acid Intermediate B39 | 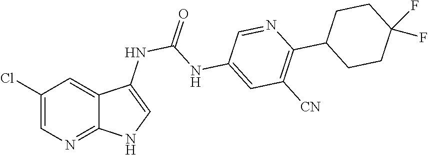 [SMILES: N#Cc1cc(NC(=O)Nc2c[nH]c3ncc(Cl)cc23)cnc1C1CCC(F)(F)CC1] | Method BD: MS- ESI: 431 [M + H]⁺. |
| | | 1-(5-chloro-1H-pyrrolo[2,3- | |
| | | b]pyridin-3-yl)-3-(5-cyano-6-(4,4- | |
| | | difluorocyclohexyl)pyridin-3- | |
| | | yl)urea | |
| Example 65 (Compound 150) | 5-methoxy-1H- pyrrolo[3,2- b]pyridine-3- carboxylic acid Intermediate B19 | 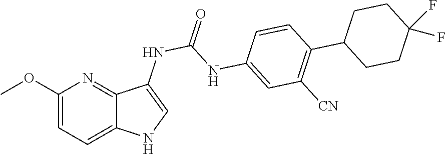 [SMILES: COc1ccc2[nH]cc(NC(=O)Nc3ccc(C4CCC(F)(F)CC4)c(C#N)c3)c2n1] | Method BD: MS- ESI: 426 [M + H]⁺. |
| | | 1-(3-cyano-4-(4,4- | |
| | | difluorocyclohexyl)phenyl)-3-(5- | |
| | | methoxy-1H-pyrrolo[3,2- | |
| | | b]pyridin-3-yl)urea | |
| Example 66 (Compound 149) | 7-fluoro-1H- pyrrolo[3,2- c]pyridine-3- carboxylic acid Intermediate B19 | 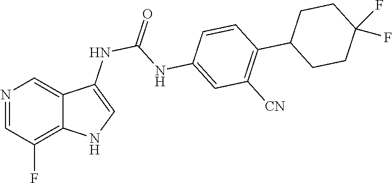 [SMILES: N#Cc1cc(NC(=O)Nc2c[nH]c3c(F)cncc23)ccc1C1CCC(F)(F)CC1] | Method BD: MS- ESI: 414 [M + H]⁺. |
| | | 1-(3-cyano-4-(4,4- | |
| | | difluorocyclohexyl)phenyl)-3-(7- | |
| | | fluoro-1H-pyrrolo[3,2-c]pyridin- | |
| | | 3-yl)urea | |
| Example 67 (Compound 148) | 1H-pyrrolo[3,2- b]pyridine-3- carboxylic acid Intermediate B19 | 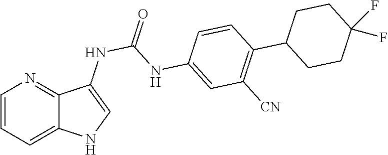 [SMILES: N#Cc1cc(NC(=O)Nc2c[nH]c3cccnc23)ccc1C1CCC(F)(F)CC1] | Method BG: MS- ESI: 396 [M + H]⁺. |
| | | 1-(3-cyano-4-(4,4- | |
| | | difluorocyclohexyl)phenyl)-3- | |
| | | (1H-pyrrolo[3,2-b]pyridin-3- | |
| | | yl)urea | |
| Example 68 (Compound 147) | 5-fluoro-1H- pyrrolo[2,3- b]pyridine-3- carboxylic acid Intermediate 35 | 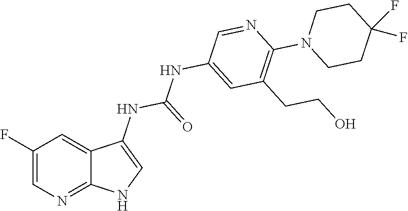 [SMILES: O=C(Nc1cnc(N2CCC(F)(F)CC2)c(CCO)c1)Nc1c[nH]c2ncc(F)cc12] | Method BC: MS- ESI: 435 [M + H]⁺. |
| | | 1-(6-(4,4-difluoropiperidin-1-yl)- | |
| | | 5-(2-hydroxyethyl)pyridin-3-yl)-3- | |
| | | (5-fluoro-1H-pyrrolo[2,3- | |
| | | b]pyridin-3-yl)urea | |
| Example 69 (Compound 146) | 1H-pyrrolo[3,2- b]pyridine-3- carboxylic acid Intermediate B2 | 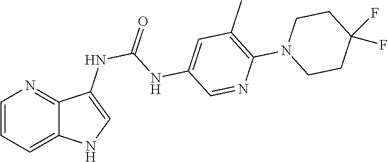 [SMILES: Cc1cc(NC(=O)Nc2c[nH]c3cccnc23)cnc1N1CCC(F)(F)CC1] | Method BD: MS- ESI: 387 [M + H]⁺. |
| | | 1-(6-(4,4-difluoropiperidin-1-yl)- | |
| | | 5-methylpyridin-3-yl)-3-(1H- | |
| | | pyrrolo[3,2-b]pyridin-3-yl)urea | |
| Example 70 (Compound 145) | 6-fluoro-1H- pyrrolo[3,2- b]pyridine-3- carboxylic acid Intermediate B1 | 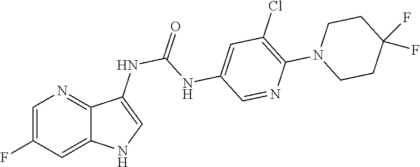 [SMILES: O=C(Nc1cnc(N2CCC(F)(F)CC2)c(Cl)c1)Nc1c[nH]c2cc(F)cnc12] | Method BD: MS- ESI: 425 [M + H]⁺. |
| | | 1-(5-chloro-6-(4,4- | |
| | | difluoropiperidin-1-yl)pyridin-3- | |
| | | yl)-3-(6-fluoro-1H-pyrrolo[3,2- | |
| | | b]pyridin-3-yl)urea | |
| Example 71 (Compound 143) | 1H-pyrrolo[3,2- b]pyridine-3- carboxylic acid Intermediate B32 | 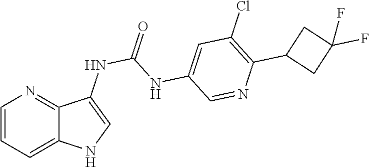 [SMILES: O=C(Nc1cnc(C2CC(F)(F)C2)c(Cl)c1)Nc1c[nH]c2cccnc12] | Method BD: MS- ESI: 378 [M + H]⁺. |
| | | 1-(5-chloro-6-(3,3- | |
| | | difluorocyclobutyl)pyridin-3-yl)- | |
| | | 3-(1H-pyrrolo[3,2-b]pyridin-3- | |
| | | yl)urea | |
| Example 72 (Compound 142) | 1H-pyrrolo[3,2- b]pyridine-3- carboxylic acid Intermediate B18 | 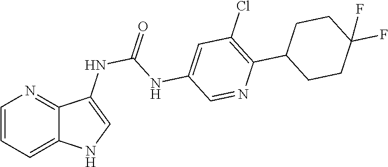 [SMILES: O=C(Nc1cnc(C2CCC(F)(F)CC2)c(Cl)c1)Nc1c[nH]c2cccnc12] | Method BD: MS- ESI: 406 [M + H]⁺. |
| | | 1-(5-chloro-6-(4,4- | |
| | | difluorocyclohexyl)pyridin-3-yl)- | |
| | | 3-(1H-pyrrolo[3,2-b]pyridin-3- | |
| | | yl)urea | |
| Example 73 (Compound 141) | 5-methoxy-1H- pyrrolo[3,2- b]pyridine-3- carboxylic acid Intermediate B1 | 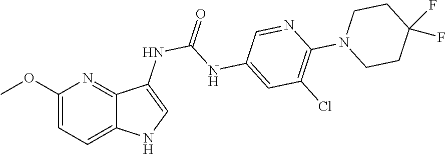 [SMILES: COc1ccc2[nH]cc(NC(=O)Nc3cnc(N4CCC(F)(F)CC4)c(Cl)c3)c2n1] | Method BD: MS- ESI: 437 [M + H]⁺. |
| | | 1-(5-chloro-6-(4,4- | |
| | | difluoropiperidin-1-yl)pyridin-3- | |
| | | yl)-3-(5-methoxy-1H-pyrrolo[3,2- | |
| | | b]pyridin-3-yl)urea | |
| Example 74 (Compound 140) | 5-methoxy-1H- pyrrolo[3,2- b]pyridine-3- carboxylic acid Intermediate B39 | 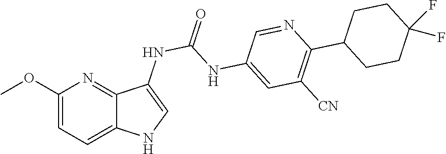 [SMILES: COc1ccc2[nH]cc(NC(=O)Nc3cnc(C4CCC(F)(F)CC4)c(C#N)c3)c2n1] | Method BC: MS- ESI: 427 [M + H]⁺. |
| | | 1-(5-cyano-6-(4,4- | |
| | | difluorocyclohexyl)pyridin-3-yl)- | |
| | | 3-(5-methoxy-1H-pyrrolo[3,2- | |
| | | b]pyridin-3-yl)urea | |
| Example 75 (Compound 138) | 5-fluoro-1H- pyrrolo[2,3- b]pyridine-3- carboxylic acid Intermediate B36 | 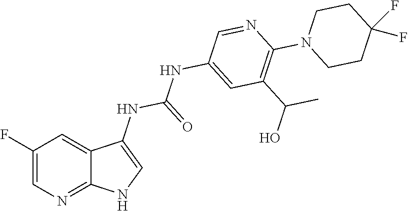 [SMILES: CC(O)c1cc(NC(=O)Nc2c[nH]c3ncc(F)cc23)cnc1N1CCC(F)(F)CC1] | Method BC: MS- ESI: 435 [M + H]⁺. |
| | | 1-(6-(4,4-difluoropiperidin-1-yl)- | |
| | | 5-(1-hydroxyethyl)pyridin-3-yl)-3- | |
| | | (5-fluoro-1H-pyrrolo[2,3- | |
| | | b]pyridin-3-yl)urea | |
| Example 76 (Compound 137) | 5-chloro-1H- pyrrolo[2,3- b]pyridine-3- carboxylic acid Intermediate B21 | 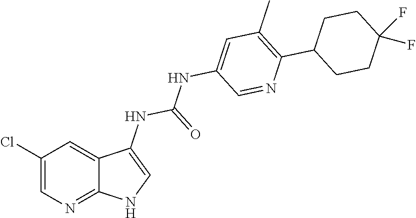 [SMILES: Cc1cc(NC(=O)Nc2c[nH]c3ncc(Cl)cc23)cnc1C1CCC(F)(F)CC1] | Method BD: MS- ESI: 420 [M + H]⁺. |
| | | 1-(5-chloro-1H-pyrrolo[2,3- | |
| | | b]pyridin-3-yl)-3-(6-(4,4- | |
| | | difluorocyclohexyl)-5- | |
| | | methylpyridin-3-yl)urea | |
| Example 77 (Compound 136) | 1H-pyrrolo[3,2- b]pyridine-3- carboxylic acid Intermediate B21 | 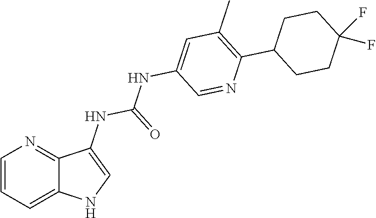 [SMILES: Cc1cc(NC(=O)Nc2c[nH]c3cccnc23)cnc1C1CCC(F)(F)CC1] | Method BD: MS- ESI: 386 [M + H]⁺. |
| | | 1-(6-(4,4-difluorocyclohexyl)-5- | |
| | | methylpyridin-3-yl)-3-(1H- | |
| | | pyrrolo[3,2-b]pyridin-3-yl)urea | |
| Example 78 (Compound 205) | 1H-pyrrolo[3,2- b]pyridine-3- carboxylic acid Intermediate B68 | 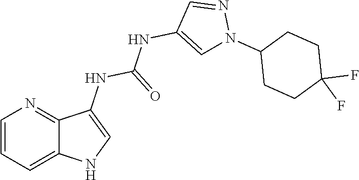 [SMILES: O=C(Nc1cnn(C2CCC(F)(F)CC2)c1)Nc1c[nH]c2cccnc12] | Method BD: MS- ESI: 361 [M + H]+. |
| | | 1-(1-(4,4-difluorocyclohexyl)-1H- | |
| | | pyrazol-4-yl)-3-(1H-pyrrolo[3,2- | |
| | | b]pyridin-3-yl)urea | |
| Example 79 (Compound 206) | 1H-pyrrolo[3,2- b]pyridine-3- carboxylic acid Intermediate B69 | 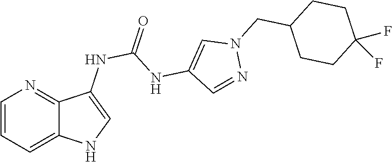 [SMILES: O=C(Nc1cnn(CC2CCC(F)(F)CC2)c1)Nc1c[nH]c2cccnc12] | Method BI: MS- ESI: 375 [M + H]+. |
| | | 1-(1-((4,4- | |
| | | difluorocyclohexyl)methyl)-1H- | |
| | | pyrazol-4-yl)-3-(1H-pyrrolo[3,2- | |
| | | b]pyridin-3-yl)urea | |
| Example 80 (Compound 207) | 1H-pyrrolo[3,2- b]pyridine-3- carboxylic acid Intermediate B70 | 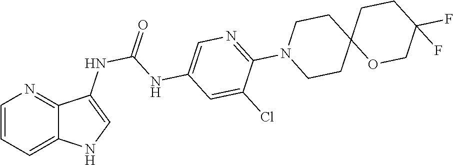 [SMILES: O=C(Nc1cnc(N2CCC3(CC2)CCC(F)(F)CO3)c(Cl)c1)Nc1c[nH]c2cccnc12] | Method BI: MS- ESI: 477 [M + H]+. |
| | | 1-(5-chloro-6-(3,3-difluoro-1-oxa- | |
| | | 9-azaspiro[5.5]undecan-9- | |
| | | yl)pyridin-3-yl)-3-(1H- | |
| | | pyrrolo[3,2-b]pyridin-3-yl)urea | |
| Example 81 (Compound 209) | 5-methoxy-1H- pyrrolo[3,2- b]pyridine-3- carboxylic acid Intermediate B68 | 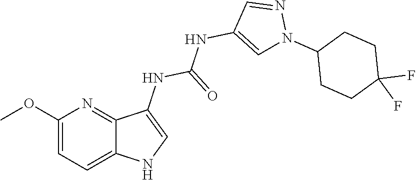 [SMILES: COc1ccc2[nH]cc(NC(=O)Nc3cnn(C4CCC(F)(F)CC4)c3)c2n1] | Method BD: MS- ESI: 391 [M + H]+. |
| | | 1-(1-(4,4-difluorocyclohexyl)-1H- | |
| | | pyrazol-4-yl)-3-(5-methoxy-1H- | |
| | | pyrrolo[3,2-b]pyridin-3-yl)urea | |
| Example 82 (Compound 213) | 5-methoxy-1H- pyrrolo[3,2- b]pyridine-3- carboxylic acid Intermediate B20 | 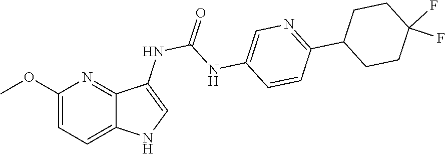 [SMILES: COc1ccc2[nH]cc(NC(=O)Nc3ccc(C4CCC(F)(F)CC4)nc3)c2n1] | Method BI: MS- ESI: 402 [M + H]+. |
| | | 1-(6-(4,4- | |
| | | difluorocyclohexyl)pyridin-3-yl)- | |
| | | 3-(5-methoxy-1H-pyrrolo[3,2- | |
| | | b]pyridin-3-yl)urea | |
| Example 83 (Compound 214) | 1H-pyrrolo[3,2- b]pyridine-3- carboxylic acid Intermediate B55 | 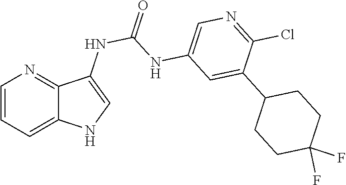 [SMILES: O=C(Nc1cnc(Cl)c(C2CCC(F)(F)CC2)c1)Nc1c[nH]c2cccnc12] | Method BI: MS- ESI: 406 [M + H]+. |
| | | 1-(6-chloro-5-(4,4- | |
| | | difluorocyclohexyl)pyridin-3-yl)- | |
| | | 3-(1H-pyrrolo[3,2-b]pyridin-3- | |
| | | yl)urea | |
| Example 85 (Compound 215) | 1H-pyrrolo[3,2- b]pyridine-3- carboxylic acid Intermediate B57 | 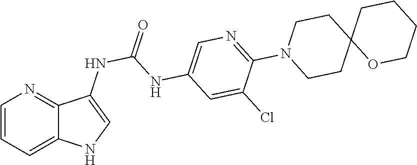 [SMILES: O=C(Nc1cnc(N2CCC3(CCCCO3)CC2)c(Cl)c1)Nc1c[nH]c2cccnc12] | Method BI: MS- ESI: 441 [M + H]+. |
| | | 1-(5-chloro-6-(1-oxa-9- | |
| | | azaspiro[5.5]undecan-9- | |
| | | yl)pyridin-3-yl)-3-(1H- | |
| | | pyrrolo[3,2-b]pyridin-3-yl)urea | |
| Example 86 (Compound 216) | 1H-pyrrolo[3,2- b]pyridine-3- carboxylic acid Intermediate B72 | 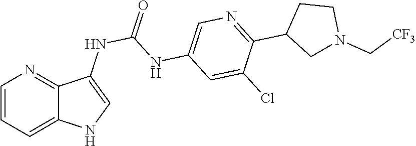 [SMILES: O=C(Nc1cnc(C2CCN(CC(F)(F)F)C2)c(Cl)c1)Nc1c[nH]c2cccnc12] | Method BD: MS- ESI: 439 [M + H]+. |
| | | 1-(5-chloro-6-(1-(2,2,2- | |
| | | trifluoroethyl)pyrrolidin-3- | |
| | | yl)pyridin-3-yl)-3-(1H- | |
| | | pyrrolo[3,2-b]pyridin-3-yl)urea | |
| Example 87 (Compound 217) | 1H-pyrrolo[3,2- b]pyridine-3- carboxylic acid Intermediate B56 | 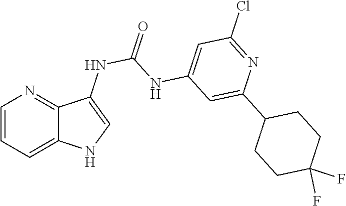 [SMILES: O=C(Nc1cc(Cl)nc(C2CCC(F)(F)CC2)c1)Nc1c[nH]c2cccnc12] | Method BD: MS- ESI: 406 [M + H]+. |
| | | 1-(2-chloro-6-(4,4- | |
| | | difluorocyclohexyl)pyridin-4-yl)- | |
| | | 3-(1H-pyrrolo[3,2-b]pyridin-3- | |
| | | yl)urea | |
| Example 88 (Compound 219) | 1H-pyrrolo[3,2- b]pyridine-3- carboxylic acid Intermediate B73 | 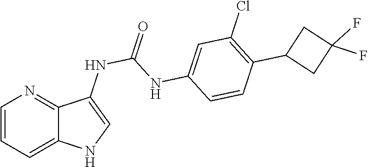 [SMILES: O=C(Nc1ccc(C2CC(F)(F)C2)c(Cl)c1)Nc1c[nH]c2cccnc12] | Method BD: MS- ESI: 377 [M + H]+. |
| | | 1-(3-chloro-4-(3,3- | |
| | | difluorocyclobutyl)phenyl)-3-(1H- | |
| | | pyrrolo[3,2-b]pyridin-3-yl)urea | |
| Example 89 (Compound 220) | 1H-pyrrolo[3,2- b]pyridine-3- carboxylic acid Intermediate B58 | 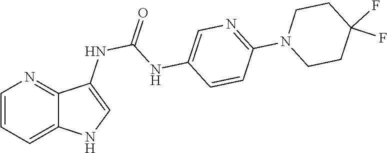 [SMILES: O=C(Nc1ccc(N2CCC(F)(F)CC2)nc1)Nc1c[nH]c2cccnc12] | Method BD: MS- ESI: 373 [M + H]+. |
| | | 1-(6-(4,4-difluoropiperidin-1- | |
| | | yl)pyridin-3-yl)-3-(1H- | |
| | | pyrrolo[3,2-b]pyridin-3-yl)urea | |
| Example 90 (Compound 221) | 1H-pyrrolo[3,2- b]pyridine-3- carboxylic acid Intermediate B59 | 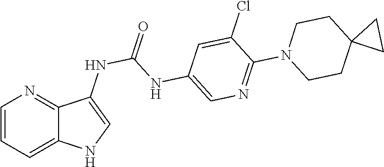 [SMILES: O=C(Nc1cnc(N2CCC3(CC2)CC3)c(Cl)c1)Nc1c[nH]c2cccnc12] | Method BD: MS- ESI: 397 [M + H]+. |
| | | 1-(5-chloro-6-(6- | |
| | | azaspiro[2.5]octan-6-yl)pyridin-3- | |
| | | yl)-3-(1H-pyrrolo[3,2-b]pyridin-3- | |
| | | yl)urea | |
| Example 91 (Compound 222) | 1H-pyrrolo[3,2- b]pyridine-3- carboxylic acid Intermediate B67 | 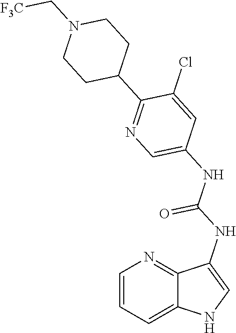 [SMILES: O=C(Nc1cnc(C2CCN(CC(F)(F)F)CC2)c(Cl)c1)Nc1c[nH]c2cccnc12] | Method BD: MS- ESI: 453 [M + H]+. |
| | | 1-(5-chloro-6-(1-(2,2,2- | |
| | | trifluoroethyl)piperidin-4- | |
| | | yl)pyridin-3-yl)-3-(1H- | |
| | | pyrrolo[3,2-b]pyridin-3-yl)urea | |
| Example 92 (Compound 223) | 1H-pyrrolo[3,2- b]pyridine-3- carboxylic acid Intermediate B60 | 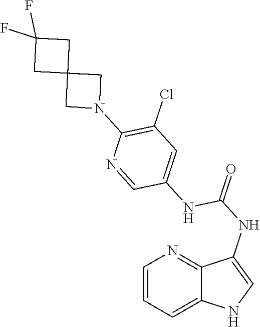 [SMILES: O=C(Nc1cnc(N2CC3(C2)CC(F)(F)C3)c(Cl)c1)Nc1c[nH]c2cccnc12] | Method BG: MS- ESI: 419 [M + H]+. |
| | | 1-(5-chloro-6-(6,6-difluoro-2- | |
| | | azaspiro[3.3]heptan-2-yl)pyridin- | |
| | | 3-yl)-3-(1H-pyrrolo[3,2-b]pyridin- | |
| | | 3-yl)urea | |
| Example 93 (Compound 224) | 1H-pyrrolo[3,2- b]pyridine-3- carboxylic acid Intermediate B61 | 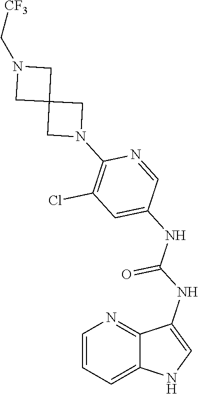 [SMILES: O=C(Nc1cnc(N2CC3(CN(CC(F)(F)F)C3)C2)c(Cl)c1)Nc1c[nH]c2cccnc12] | Method BD: MS- ESI: 466 [M + H]+. |
| | | 1-(5-chloro-6-(6-(2,2,2- | |
| | | trifluoroethyl)-2,6- | |
| | | diazaspiro[3.3]heptan-2- | |
| | | yl)pyridin-3-yl)-3-(1H- | |
| | | pyrrolo[3,2-b]pyridin-3-yl)urea | |
| Example 94 (Compound 225) | 1H-pyrrolo[3,2- b]pyridine-3- carboxylic acid Intermediate B74 | 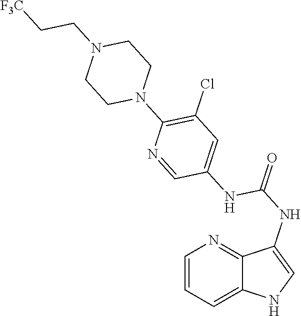 [SMILES: O=C(Nc1cnc(N2CCN(CCC(F)(F)F)CC2)c(Cl)c1)Nc1c[nH]c2cccnc12] | Method BD: MS- ESI: 468 [M + H]+. |
| | | 1-(5-chloro-6-(4-(3,3,3- | |
| | | trifluoropropyl)piperazin-1- | |
| | | yl)pyridin-3-yl)-3-(1H- | |
| | | pyrrolo[3,2-b]pyridin-3-yl)urea | |
| Example 95 (Compound 226) | 1H-pyrrolo[3,2- b]pyridine-3- carboxylic acid Intermediate B62 | 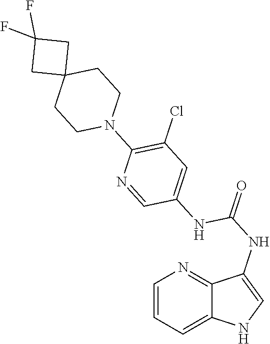 [SMILES: O=C(Nc1cnc(N2CCC3(CC2)CC(F)(F)C3)c(Cl)c1)Nc1c[nH]c2cccnc12] | Method BD: MS- ESI: 447 [M + H]+. |
| | | 1-(5-chloro-6-(2,2-difluoro-7- | |
| | | azaspiro[3.5]nonan-7-yl)pyridin- | |
| | | 3-yl)-3-(1H-pyrrolo[3,2-b]pyridin- | |
| | | 3-yl)urea | |
| Example 96 (Compound 227) | 1H-pyrrolo[3,2- b]pyridine-3- carboxylic acid Intermediate B63 | 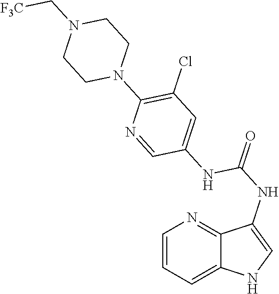 [SMILES: O=C(Nc1cnc(N2CCN(CC(F)(F)F)CC2)c(Cl)c1)Nc1c[nH]c2cccnc12] | Method BD: MS- ESI: 454 [M + H]+. |
| | | 1-(5-chloro-6-(4-(2,2,2- | |
| | | trifluoroethyl)piperazin-1- | |
| | | yl)pyridin-3-yl)-3-(1H- | |
| | | pyrrolo[3,2-b]pyridin-3-yl)urea | |
| Example 97 (Compound 203/228) | 5-methoxy-1H- pyrrolo[3,2- b]pyridine-3- carboxylic acid Intermediate B62 | 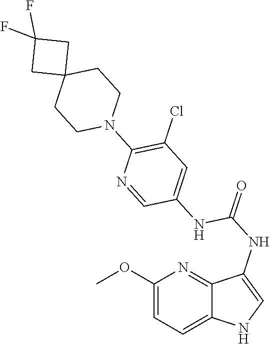 [SMILES: COc1ccc2[nH]cc(NC(=O)Nc3cnc(N4CCC5(CC4)CC(F)(F)C5)c(Cl)c3)c2n1] | Method BD: MS- ESI: 477 [M + H]+. |
| | | 1-(5-chloro-6-(2,2-difluoro-7- | |
| | | azaspiro[3.5]nonan-7-yl)pyridin- | |
| | | 3-yl)-3-(5-methoxy-1H- | |
| | | pyrrolo[3,2-b]pyridin-3-yl)urea | |
| Example 98 (Compound 229) | 5-methoxy-1H- pyrrolo[3,2- b]pyridine-3- carboxylic acid Intermediate B54 | 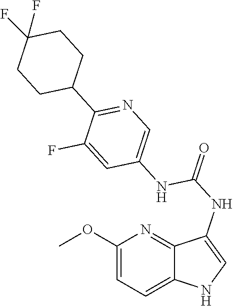 [SMILES: COc1ccc2[nH]cc(NC(=O)Nc3cnc(C4CCC(F)(F)CC4)c(F)c3)c2n1] | Method BD: MS- ESI: 420 [M + H]+. |
| | | 1-(6-(4,4-difluorocyclohexyl)-5- | |
| | | fluoropyridin-3-yl)-3-(5-methoxy- | |
| | | 1H-pyrrolo[3,2-b]pyridin-3- | |
| | | yl)urea | |
| Example 99 (Compound 230) | 1H-pyrrolo[3,2- b]pyridine-3- carboxylic acid Intermediate B8 | 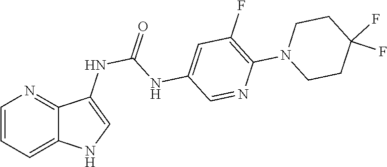 [SMILES: O=C(Nc1cnc(N2CCC(F)(F)CC2)c(F)c1)Nc1c[nH]c2cccnc12] | Method BE: MS- ESI: 391 [M + H]+. |
| | | 1-(6-(4,4-difluoropiperidin-1-yl)- | |
| | | 5-fluoropyridin-3-yl)-3-(1H- | |
| | | pyrrolo[3,2-b]pyridin-3-yl)urea | |
| Example 100 (Compound 231) | 5-methoxy-1H- pyrrolo[3,2- b]pyridine-3- carboxylic acid Intermediate B64 | 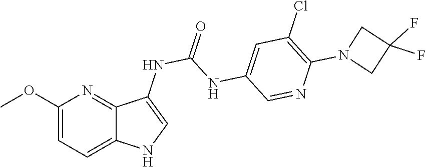 [SMILES: COc1ccc2[nH]cc(NC(=O)Nc3cnc(N4CC(F)(F)C4)c(Cl)c3)c2n1] | Method BD: MS- ESI: 409 [M + H]+. |
| | | 1-(5-chloro-6-(3,3- | |
| | | difluoroazetidin-1-yl)pyridin-3- | |
| | | yl)-3-(5-methoxy-1H-pyrrolo[3,2- | |
| | | b]pyridin-3-yl)urea | |
| Example 101 (Compound 232) | 5-methoxy-1H- pyrrolo[3,2- b]pyridine-3- carboxylic acid Intermediate B65 | 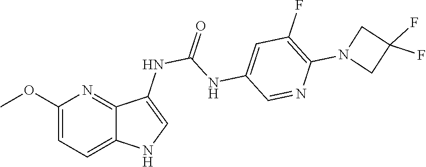 [SMILES: COc1ccc2[nH]cc(NC(=O)Nc3cnc(N4CC(F)(F)C4)c(F)c3)c2n1] | Method BD: MS- ESI: 393 [M + H]+. |
| | | 1-(6-(3,3-difluoroazetidin-1-yl)-5- | |
| | | fluoropyridin-3-yl)-3-(5-methoxy- | |
| | | 1H-pyrrolo[3,2-b]pyridin-3- | |
| | | yl)urea | |
| Example 102 (Compound 233) | 5-methoxy-1H- pyrrolo[3,2- b]pyridine-3- carboxylic acid Intermediate B18 | 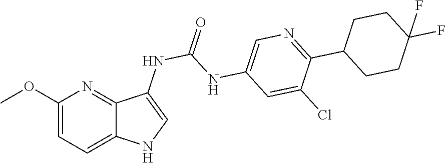 [SMILES: COc1ccc2[nH]cc(NC(=O)Nc3cnc(C4CCC(F)(F)CC4)c(Cl)c3)c2n1] | Method BD: MS- ESI: 436 [M + H]+. |
| | | 1-(5-chloro-6-(4,4- | |
| | | difluorocyclohexyl)pyridin-3-yl)- | |
| | | 3-(5-methoxy-1H-pyrrolo[3,2- | |
| | | b]pyridin-3-yl)urea | |
| Example 103 (Compound 234) | 1H-pyrrolo [3,2- b]pyridine-3- carboxylic acid Intermediate B54 | 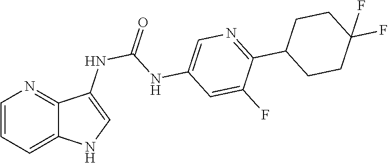 [SMILES: O=C(Nc1cnc(C2CCC(F)(F)CC2)c(F)c1)Nc1c[nH]c2cccnc12] | Method BD: MS- ESI: 390 [M + H]+. |
| | | 1-(6-(4,4-difluorocyclohexyl)-5- | |
| | | fluoropyridin-3-yl)-3-(1H- | |
| | | pyrrolo[3,2-b]pyridin-3-yl)urea | |
| Example 104 (Compound 235) | 1H-pyrrolo[3,2- b]pyridine-3- carboxylic acid Intermediate B27 | 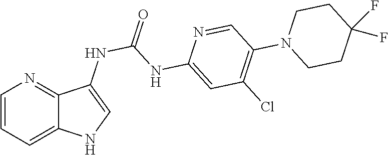 [SMILES: O=C(Nc1cc(Cl)c(N2CCC(F)(F)CC2)cn1)Nc1c[nH]c2cccnc12] | Method BF: MS- ESI: 407 [M + H]+. |
| | | 1-(4-chloro-5-(4,4- | |
| | | difluoropiperidin-1-yl)pyridin-2- | |
| | | yl)-3-(1H-pyrrolo[3,2-b]pyridin-3- | |
| | | yl)urea | |
| Example 105 (Compound 240) | 6-bromo-1H- pyrrolo[3,2- b]pyridine-3- carboxylic acid Intermediate B1 | 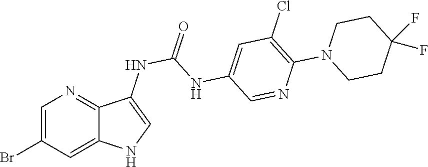 [SMILES: O=C(Nc1cnc(N2CCC(F)(F)CC2)c(Cl)c1)Nc1c[nH]c2cc(Br)cnc12] | Method BJ: MS- ESI: 485 [M + H]+. |
| | | 1-(6-bromo-1H-pyrrolo[3,2- | |
| | | b]pyridin-3-yl)-3-(5-chloro-6-(4,4- | |
| | | difluoropiperidin-1-yl)pyridin-3- | |
| | | yl)urea | |
| Example 106 (Compound 236) | 5-bromo-1H- pyrrolo[3,2- b]pyridine-3- carboxylic acid Intermediate B1 | 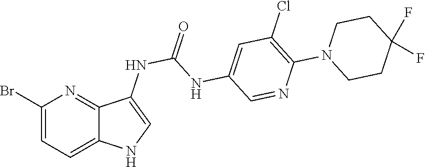 [SMILES: O=C(Nc1cnc(N2CCC(F)(F)CC2)c(Cl)c1)Nc1c[nH]c2ccc(Br)nc12] | Method BI: MS- ESI: 485 [M + H]+. |
| | | 1-(5-bromo-1H-pyrrolo[3,2- | |
| | | b]pyridin-3-yl)-3-(5-chloro-6-(4,4- | |
| | | difluoropiperidin-1-yl)pyridin-3- | |
| | | yl)urea | |
| Example 107 (Compound 237) | 1H-pyrrolo [3,2- b]pyridine-3- carboxylic acid Intermediate B39 | 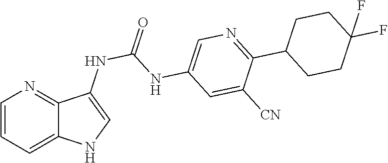 [SMILES: N#Cc1cc(NC(=O)Nc2c[nH]c3cccnc23)cnc1C1CCC(F)(F)CC1] | Method BC: MS- ESI: 397 [M + H]+. |
| | | 1-(5-cyano-6-(4,4- | |
| | | difluorocyclohexyl)pyridin-3-yl)- | |
| | | 3-(1H-pyrrolo[3,2-b]pyridin-3- | |
| | | yl)urea | |
| Example 108 (Compound 238) | 5-methoxy-1H- pyrrolo[3,2- b]pyridine-3- carboxylic acid Intermediate B24 | 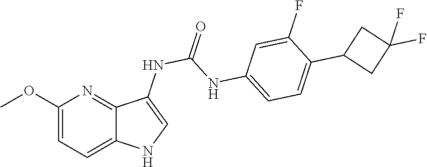 [SMILES: COc1ccc2[nH]cc(NC(=O)Nc3ccc(C4CC(F)(F)C4)c(F)c3)c2n1] | Method BD: MS- ESI: 391 [M + H]+. |
| | | 1-(4-(3,3-difluorocyclobutyl)-3- | |
| | | fluorophenyl)-3-(5-methoxy-1H- | |
| | | pyrrolo[3,2-b]pyridin-3-yl)urea | |
| Example 109 (Compound 239) | 1H-pyrrolo[3,2- b]pyridine-3- carboxylic acid Intermediate B24 | 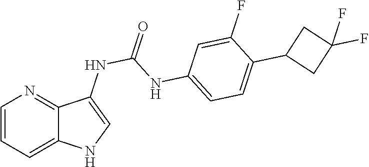 [SMILES: O=C(Nc1ccc(C2CC(F)(F)C2)c(F)c1)Nc1c[nH]c2cccnc12] | Method BD: MS- ESI: 361 [M + H]+. |
| | | 1-(4-(3,3-difluorocyclobutyl)-3- | |
| | | fluorophenyl)-3-(1H-pyrrolo[3,2- | |
| | | b]pyridin-3-yl)urea | |
| Example 110 (Compound 241) | 1H-pyrrolo[3,2- b]pyridine-3- carboxylic acid Intermediate B66 | 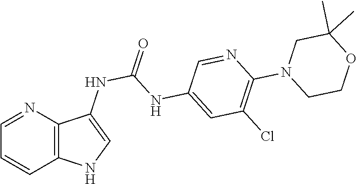 [SMILES: CC1(C)CN(c2ncc(NC(=O)Nc3c[nH]c4cccnc34)cc2Cl)CCO1] | Method BG: MS- ESI: 401 [M + H]+. |
| | | 1-(5-chloro-6-(2,2- | |
| | | dimethylmorpholino)pyridin-3- | |
| | | yl)-3-(1H-pyrrolo[3,2-b]pyridin-3- | |
| | | yl)urea | |
| Example 111 (Compound 242) | 1H-pyrrolo[3,2- b]pyridine-3- carboxylic acid Intermediate B64 | 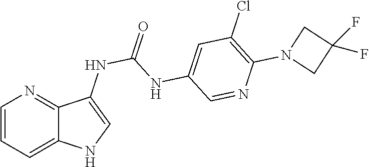 [SMILES: O=C(Nc1cnc(N2CC(F)(F)C2)c(Cl)c1)Nc1c[nH]c2cccnc12] | Method BD: MS- ESI: 379 [M + H]+. |
| | | 1-(5-chloro-6-(3,3- | |
| | | difluoroazetidin-1-yl)pyridin-3- | |
| | | yl)-3-(1H-pyrrolo[3,2-b]pyridin-3- | |
| | | yl)urea | |
| Example 112 (Compound 243) | 5-methoxy-1H- pyrrolo[3,2- b]pyridine-3- carboxylic acid Intermediate B32 | 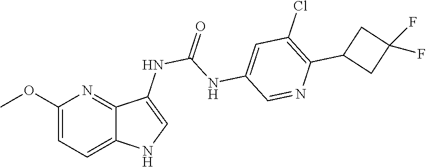 [SMILES: COc1ccc2[nH]cc(NC(=O)Nc3cnc(C4CC(F)(F)C4)c(Cl)c3)c2n1] | Method BD: MS- ESI: 408 [M + H]+. |
| | | 1-(5-chloro-6-(3,3- | |
| | | difluorocyclobutyl)pyridin-3-yl)- | |
| | | 3-(5-methoxy-1H-pyrrolo[3,2- | |
| | | b]pyridin-3-yl)urea | |

NMR Data for Compound 192 (Example 33): H NMR (300 MHz, DMSO-d₆) δ 11.48 (brs, 1H), 8.54 (s, 1H), 8.45 (s, 1H), 8.20-8.19 (m, 1H), 8.13 (d, J=2.7 Hz, 1H), 7.75-7.70 (m, 2H), 7.60 (d, J=2.1 Hz, 1H), 2.98 (t, J=5.1 Hz, 4H), 2.23 (s, 3H), 1.46-1.46 (m, 4H), 0.32 (s, 4H).

NMR Data for Compound 164 (Example 52): ¹H NMR (300 MHz, DMSO-d₆) δ 11.53 (brs, 1H), 8.76 (s, 1H), 8.63 (s, 1H), 8.22 (s, 1H), 8.08 (d, J=2.4 Hz, 1H), 7.76-7.73 (m, 1H), 7.72 (d, J=2.0 Hz, 1H), 7.64 (d, J=2.4 Hz, 1H), 3.81 (s, 3H), 3.15-3.11 (m, 1H), 2.13-2.06 (m, 2H), 2.01-1.85 (m, 2H), 1.82-1.76 (m, 4H).

NMR Data for Compound 143 (Example 71): ¹H NMR (300 MHz, DMSO-d₆) δ 10.96 (brs, 1H), 9.45 (s, 1H), 8.92 (s, 1H), 8.45 (d, J=2.1 Hz, 1H), 8.34-8.31 (m, 1H), 8.26 (d, J=2.1 Hz, 1H), 7.85-7.82 (m, 1H), 7.77-7.74 (m, 1H), 7.18-7.13 (m, 1H), 3.76-3.69 (m, 1H), 2.99-2.87 (m, 4H).

NMR data for Compound 141 (Example 73): ¹H NMR (400 MHz, DMSO-d₆) δ 10.80 (brs, 1H), 9.30 (s, 1H), 8.39 (s, 1H), 8.21-8.19 (m, 2H), 7.70-7.67 (m, 2H), 6.59 (d, J=8.8 Hz, 1H), 3.95 (s, 3H), 3.30-3.27 (m, 4H), 2.17-2.07 (m, 4H).

### Example 113: Synthesis of 1-(5-chloro-6-(4,4-difluoropiperidin-1-yl)pyridin-3-yl)-3-(1H-pyrrolo[3,2-b]pyridin-3-yl)urea (Compound 188)

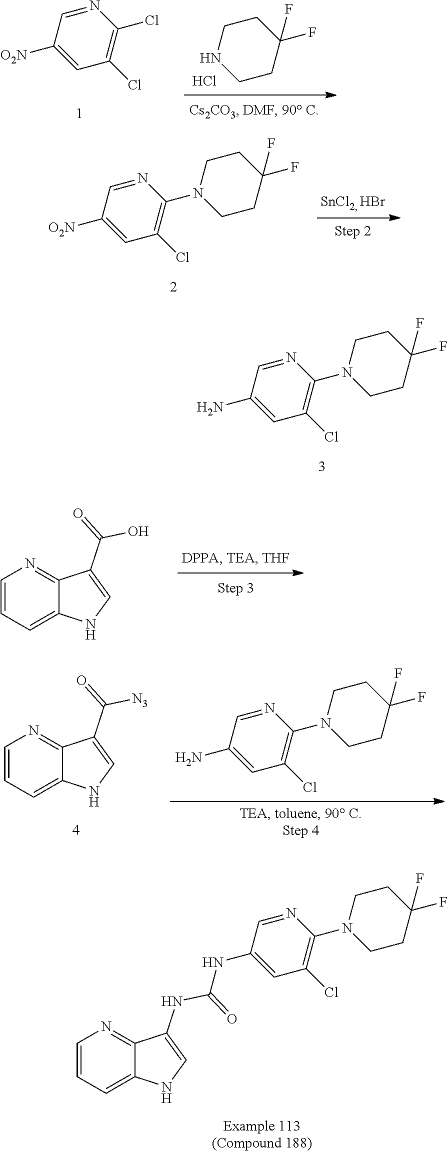

### Step 1: 3-chloro-2-(4,4-difluoropiperidin-1-yl)-5-nitropyridine

2,3-dichloro-5-nitropyridine (5.0 g, 26.1 mmol, 1.0 equiv.), 4,4-difluoropiperidine hydrochloride (4.5 g, 28.7 mmol, 1.1 equiv.) and Cs₂CO₃ (21.3 g, 65.3 mmol, 2.5 equiv.) were dissolved in DMF (70 mL). The reaction mixture was stirred overnight at 90° C. and then quenched by the addition of water. The resulting mixture was extracted with EtOAc, washed with brine, then dried over anhydrous Na₂SO₄ and concentrated under vacuum. This resulted in crude 3-chloro-2-(4,4-difluoropiperidin-1-yl)-5-nitropyridine (6.9 g) as a yellow solid. MS-ESI: 278 [M+H]+.

### Step 2: 5-chloro-6-(4,4-difluoropiperidin-1-yl)pyridin-3-amine

3-chloro-2-(4,4-difluoropiperidin-1-yl)-5-nitropyridine (6.9 g, 24.9 mmol, 1.0 equiv.) was dissolved in aq. HBr (40%, 40 mL), then SnCl₂ (14.2 g, 74.7 mmol, 3.0 equiv.) was added. The resulting mixture was heated to 70° C. for 2 h, then cooled to room temperature and quenched by the addition of water. The resulting mixture was extracted with EtOAc, washed with brine, then dried over anhydrous Na₂SO₄ and concentrated under vacuum. The residue was purified by flash column chromatography on silica gel, eluting with ethyl acetate/petroleum ether (1:3) to give 5-chloro-6-(4,4-difluoropiperidin-1-yl)pyridin-3-amine (5.5 g) as a dark green solid. MS-ESI: 248 [M+H]+.

### Step 3: 1H-pyrrolo[3,2-b]pyridine-3-carbonyl azide

1H-pyrrolo[3,2-b]pyridine-3-carboxylic acid (2.0 g, 12.3 mmol, 1.0 equiv.) was dissolved in THF (30 mL), then TEA (3.7 g, 36.9 mmol, 3.0 equiv.) and DPPA (10.1 g, 36.9 mmol, 3.0 equiv.) were added. The reaction mixture was stirred overnight at room temperature, then quenched by addition of ice/water. The desired product was precipitated and collected by filtration. This resulted in 1H-pyrrolo[3,2-b]pyridine-3-carbonyl azide (1.7 g) as an off-white solid. MS-ESI: 188 [M+H]⁺.

### Step 4: 1-(5-chloro-6-(4,4-difluoropiperidin-1-yl)pyridin-3-yl)-3-(1H-pyrrolo[3,2-b]pyridin-3-yl)urea

5-chloro-6-(4,4-difluoropiperidin-1-yl)pyridin-3-amine (1.0 g, 4.0 mmol, 1.0 equiv.) and 1H-pyrrolo[3,2-b]pyridine-3-carbonyl azide (897.6 mg, 4.8 mmol, 1.2 equiv.) were dissolved in toluene (20 mL), then TEA (808 mg, 8.0 mmol, 2.0 equiv.) was added. The reaction mixture was heated to 90° C. for 16 hours and then concentrated under vacuum. The crude product was purified by Prep-HPLC with the following conditions (Column: YMC-Actus Triant C18 Column, 30×250 mm 5 um; Mobile Phase A: Water (10 mM NH₄HCO₃+0.1% NH₄OH), Mobile Phase B: ACN; Flow rate: 60 mL/min; Gradient: 44 B to 53 B in 8 min, 254/220 nm; RT: 8.33 min) to afford 1-(5-chloro-6-(4,4-difluoropiperidin-1-yl)pyridin-3-yl)-3-(1H-pyrrolo[3,2-b]pyridin-3-yl)urea as a light yellow solid. MS-ESI: 407 [M+H]⁺.

¹H-NMR (400 MHz, DMSO-d₆) δ: 10.94 (d, J=2.0 Hz, 1H), 9.23 (s, 1H), 8.83 (s, 1H), 8.32 (dd, J=4.4, 1.2 Hz, 1H), 8.20-8.18 (m, 2H), 7.81 (dd, J=2.0, 1.2 Hz, 1H), 7.75 (dd, J=8.4, 1.2 Hz, 1H), 7.15 (dd, J=8.4, 4.4 Hz, 1H), 3.30-3.27 (m, 4H), 2.16-2.06 (m, 4H).

### Example 114: Synthesis of 1-(6-(4,4-difluorocyclohexyl)pyridin-3-yl)-3-(5-(3,3,3-trifluoropropoxy)-1H-pyrrolo[3,2-b]pyridin-3-yl)urea (Compound 204)

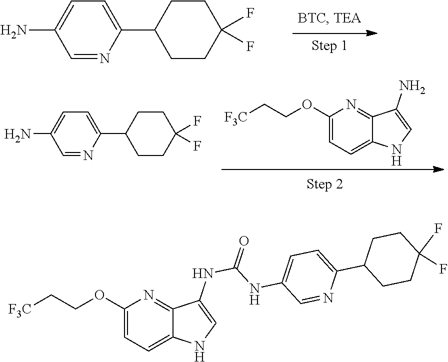 [SMILES: Nc1ccc(C2CCC(F)(F)CC2)nc1 ; Nc1ccc(C2CCC(F)(F)CC2)nc1 ; Nc1c[nH]c2ccc(OCCC(F)(F)F)nc12 ; O=C(Nc1ccc(C2CCC(F)(F)CC2)nc1)Nc1c[nH]c2ccc(OCCC(F)(F)F)nc12]

6-(4,4-Difluorocyclohexyl)pyridin-3-amine (250.0 mg, 1.2 mmol, 1.0 equiv.) and TEA (0.2 mL, 1.4 mmol, 1.2 equiv.) were dissolved in THE (5 mL), then triphosgene (430 mg, 1.5 mmol, 1.2 equiv.) was added. The reaction mixture was stirred overnight at room temperature. Then to above solution, 5-(3,3,3-trifluoropropoxy)-1H-pyrrolo[3,2-b]pyridin-3-amine (250.0 mg, 1.0 mmol, 0.8 equiv.) was added. The solution was stirred for additional 8 hours at room temperature and concentrated under vacuum. The residue was purified by Prep-HPLC with following conditions: Column: YMC-Actus Triart C18 ExRS, 30 mm×150 mm, Sum; Mobile Phase A: Water (10 mM NH₄HCO₃), Mobile Phase B: ACN; Flow rate: 60 mL/min; Gradient: 43 B to 70 B in 7 min; 254/220 nm; RT1: 7.12.

This resulted in 1-[6-(4,4-difluorocyclohexyl)pyridin-3-yl]-3-[5-(3,3,3-trifluoropropoxy)-1H-pyrrolo[3,2-b]pyridin-3-yl]urea as a white solid. LCMS Method A: [M+H]⁺=484. ¹HNMR (400 MHz, DMSO-d₆): δ 10.84 (brs, 1H), 9.19 (s, 1H), 8.51 (d, J=2.8 Hz, 1H), 8.37 (s, 1H), 7.96-7.93 (m, 1H), 7.73-7.70 (m, 2H), 7.22 (d, J=8.4 Hz, 1H), 6.60 (d, J=8.8 Hz, 2H), 4.58 (t, J=6.4 Hz, 1H), 2.89-2.81 (m, 3H), 2.13-1.91 (m, 6H), 1.85-1.78 (m, 2H).

The following compounds were prepared using the method described for Examples 24, 113, or 114.

| | Starting | | LCMS |
|---|---|---|---|
| Compound | Materials | Structure | data |
| Example 115 (Compound 184) | Intermediate B45 Intermediate B8 | 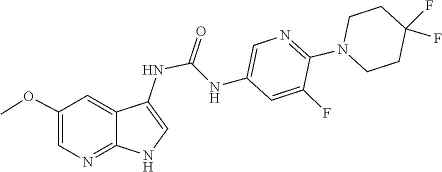 [SMILES: COc1cnc2[nH]cc(NC(=O)Nc3cnc(N4CCC(F)(F)CC4)c(F)c3)c2c1] 1-(6-(4,4-difluoropiperidin-1-yl)-5- fluoropyridin-3-yl)-3-(5-methoxy-1H- pyrrolo[2,3-b]pyridin-3-yl)urea | Method BD: MS- ESI: 421 [M + H]⁺. |
| Example 116 (Compound 182) | Intermediate B46 Intermediate B20 | 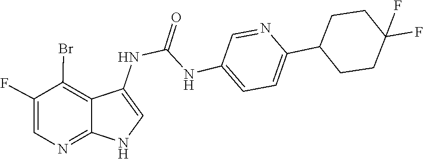 [SMILES: O=C(Nc1ccc(C2CCC(F)(F)CC2)nc1)Nc1c[nH]c2ncc(F)c(Br)c12] 1-(4-bromo-5-fluoro-1H-pyrrolo[2,3- b]pyridin-3-yl)-3-(6-(4,4- difluorocyclohexyl)pyridin-3-yl)urea | Method BG: MS- ESI: 468 [M + H]⁺. |
| Example 117 (Compound 181) | Intermediate B45 Intermediate B1 | 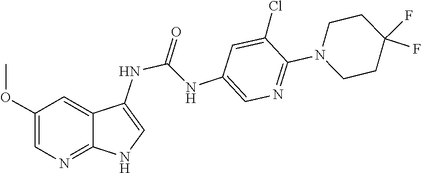 [SMILES: COc1cnc2[nH]cc(NC(=O)Nc3cnc(N4CCC(F)(F)CC4)c(Cl)c3)c2c1] 1-(5-chloro-6-(4,4-difluoropiperidin- 1-yl)pyridin-3-yl)-3-(5-methoxy-1H- pyrrolo[2,3-b]pyridin-3-yl)urea | Method BF: MS- ESI: 437 [M + H]⁺. |
| Example 118 (Compound 180) | Intermediate B47 Intermediate B20 | 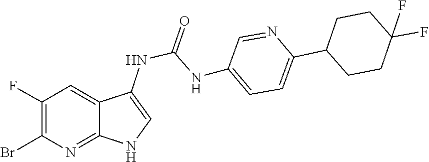 [SMILES: O=C(Nc1ccc(C2CCC(F)(F)CC2)nc1)Nc1c[nH]c2nc(Br)c(F)cc12] 1-(6-bromo-5-fluoro-1H-pyrrolo[2,3- b]pyridin-3-yl)-3-(6-(4,4- difluorocyclohexyl)pyridin-3-yl)urea | Method BD: MS- ESI: 468 [M + H]⁺. |
| Example 119 (Compound 175) | Intermediate B49 Intermediate B8 | 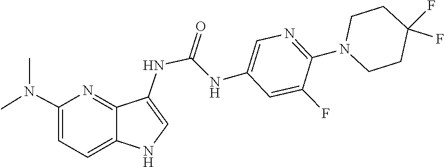 [SMILES: CN(C)c1ccc2[nH]cc(NC(=O)Nc3cnc(N4CCC(F)(F)CC4)c(F)c3)c2n1] 1-(6-(4,4-difluoropiperidin-1-yl)-5- fluoropyridin-3-yl)-3-(5- (dimethylamino)-1H-pyrrolo[3,2- b]pyridin-3-yl)urea | Method BD: MS- ESI: 434 [M + H]⁺. |
| Example 120 (Compound 174) | Intermediate B45 Intermediate B21 | 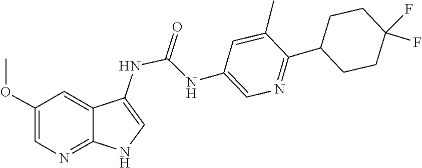 [SMILES: COc1cnc2[nH]cc(NC(=O)Nc3cnc(C4CCC(F)(F)CC4)c(C)c3)c2c1] 1-(6-(4,4-difluorocyclohexyl)-5- methylpyridin-3-yl)-3-(5-methoxy- 1H-pyrrolo[2,3-b]pyridin-3-yl)urea | Method BD: MS- ESI: 416 [M + H]⁺. |
| Example 121 (Compound 168) | Intermediate B45 Intermediate B2 | 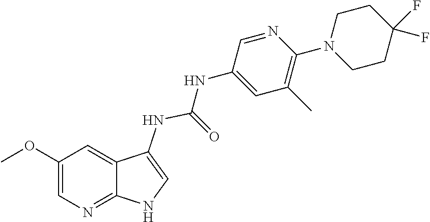 [SMILES: COc1cnc2[nH]cc(NC(=O)Nc3cnc(N4CCC(F)(F)CC4)c(C)c3)c2c1] 1-(6-(4,4-difluoropiperidin-1-yl)-5- methylpyridin-3-yl)-3-(5-methoxy- 1H-pyrrolo[2,3-b]pyridin-3-yl)urea | Method BD: MS- ESI: 417 [M + H]⁺. |
| Example 122 (Compound 144) | Intermediate B43 Intermediate B1 | 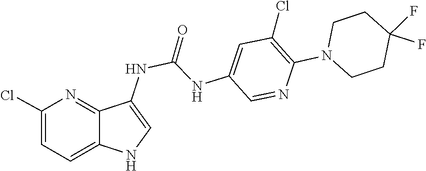 [SMILES: O=C(Nc1cnc(N2CCC(F)(F)CC2)c(Cl)c1)Nc1c[nH]c2ccc(Cl)nc12] 1-(5-chloro-1H-pyrrolo[3,2-b]pyridin- 3-yl)-3-(5-chloro-6-(4,4- difluoropiperidin-1-yl)pyridin-3- yl)urea | Method BD: MS- ESI: 441 [M + H]⁺. |
| Example 123 (Compound 139) | Intermediate B51 Intermediate B2 | 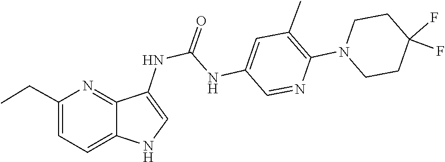 [SMILES: CCc1ccc2[nH]cc(NC(=O)Nc3cnc(N4CCC(F)(F)CC4)c(C)c3)c2n1] 1-(6-(4,4-difluoropiperidin-1-yl)-5- methylpyridin-3-yl)-3-(5-ethyl-1H- pyrrolo[3,2-b]pyridin-3-yl)urea | Method BG: MS- ESI: 415 [M + H]⁺. |
| Example 124 (Compound 208) | Intermediate B76 Intermediate B1 | 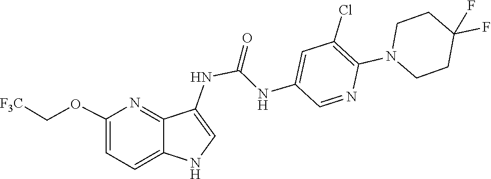 [SMILES: O=C(Nc1cnc(N2CCC(F)(F)CC2)c(Cl)c1)Nc1c[nH]c2ccc(OCC(F)(F)F)nc12] 1-(5-chloro-6-(4,4-difluoropiperidin- 1-yl)pyridin-3-yl)-3-(5-(2,2,2- trifluoroethoxy)-1H-pyrrolo[3,2- b]pyridin-3-yl)urea | Method BI: MS- ESI: 505 [M + H]+. |
| Example 125 (Compound 212) | Intermediate B77 Intermediate B20 | 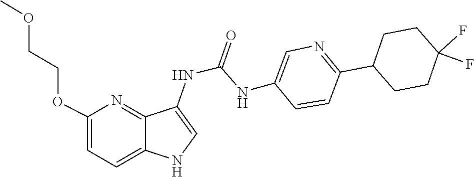 [SMILES: COCCOc1ccc2[nH]cc(NC(=O)Nc3ccc(C4CCC(F)(F)CC4)nc3)c2n1] 1-(6-(4,4-difluorocyclohexyl)pyridin- 3-yl)-3-(5-(2-methoxyethoxy)-1H- pyrrolo[3,2-b]pyridin-3-yl)urea | Method BE: MS- ESI: 446 [M + H]+. |
| Example 126 (Compound 211) | Intermediate B77 Intermediate B18 | 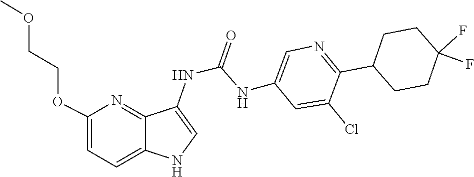 [SMILES: COCCOc1ccc2[nH]cc(NC(=O)Nc3cnc(C4CCC(F)(F)CC4)c(Cl)c3)c2n1] 1-(5-chloro-6-(4,4- difluorocyclohexyl)pyridin-3-yl)-3-(5- (2-methoxyethoxy)-1H-pyrrolo[3,2- b]pyridin-3-yl)urea | Method BE: MS- ESI: 480 [M + H]+. |
| Example 127 (Compound 139) | Intermediate B51 Intermediate B2 | 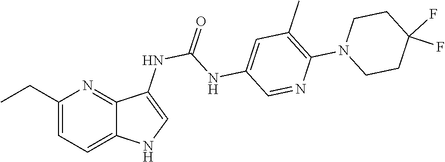 [SMILES: CCc1ccc2[nH]cc(NC(=O)Nc3cnc(N4CCC(F)(F)CC4)c(C)c3)c2n1] 1-(6-(4,4-difluoropiperidin-1-yl)-5- methylpyridin-3-yl)-3-(5-ethyl-1H- pyrrolo[3,2-b]pyridin-3-yl)urea | Method BE: MS- ESI: 415 [M + H]+. |
| Example 128 (Compound 218) | Intermediate B77 Intermediate B1 | 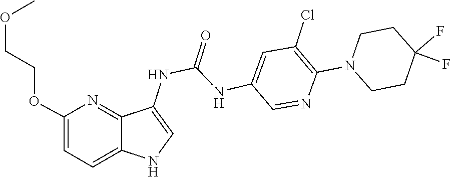 [SMILES: COCCOc1ccc2[nH]cc(NC(=O)Nc3cnc(N4CCC(F)(F)CC4)c(Cl)c3)c2n1] 1-(5-chloro-6-(4,4-difluoropiperidin- 1-yl)pyridin-3-yl)-3-(5-(2- methoxyethoxy)-1H-pyrrolo[3,2- b]pyridin-3-yl)urea | Method BE: MS- ESI: 481 [M + H]+. |

### Example 129: 1-(6-(4,4-difluorocyclohexyl)pyridin-3-yl)-3-(5-fluoro-1-(methylsulfonyl)-1H-pyrrolo[2,3-b]pyridin-3-yl)urea (Compound 129/197)

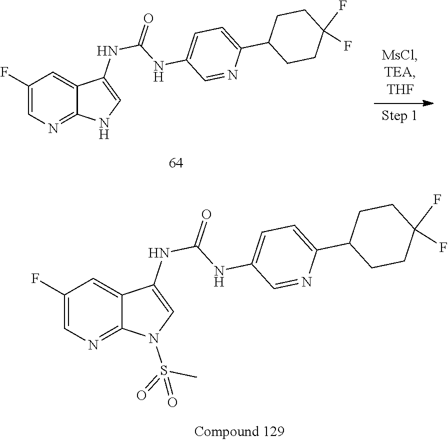 [SMILES: O=C(Nc1ccc(C2CCC(F)(F)CC2)nc1)Nc1c[nH]c2ncc(F)cc12 ; CS(=O)(=O)n1cc(NC(=O)Nc2ccc(C3CCC(F)(F)CC3)nc2)c2cc(F)cnc21]

Compound 64 was prepared using the method described for Example 24.

1-[6-(4,4-difluorocyclohexyl)pyridin-3-yl]-3-[5-fluoro-1H-pyrrolo[2,3-b]pyridin-3-yl]urea (200.0 mg, 0.5 mmol, 1.0 equiv.) and TEA (0.2 mL, 1.5 mmol, 3.0 equiv.) were dissolved in THE (35 mL), then MsCl (70.6 mg, 0.6 mmol, 1.2 equiv.) was added.

The resulting solution was stirred for 5 hours at ambient temperature and then quenched by the addition of water. The resulting mixture was extracted with ethyl acetate, dried over anhydrous sodium sulfate and concentrated under vacuum. The residue was purified by flash column chromatography on silica gel, eluting with dichloromethane/methanol (10:1) to give the crude product, which was further purified by Prep-HPLC with the following conditions: Column: YMC-Actus Triart C18, 30*250.5 um; Mobile Phase A: Water (10 mM NH₄HCO₃+0.1% NH₄OH), Mobile Phase B: ACN; Flow rate: 60 mL/min; Gradient: 40 B to 50 B in 7 min; 254/210 nm. This resulted in 1-[6-(4,4-difluorocyclohexyl)pyridin-3-yl]-3-[5-fluoro-1-methanesulfonylpyrrolo[2,3-b]pyridin-3-yl]urea as an off-white solid. LCMS Method BG: [M+H]⁺=468. ¹H NMR (400 MHz, DMSO-d₆) δ 9.54 (brs, 1H), 9.18 (brs, 1H), 8.62 (s, 1H), 8.51 (s, 1H), 8.08 (d, 1H), 7.93 (s, 1H), 7.92 (d, 1H), 7.24 (d, 1H), 3.66 (s, 3H), 2.85-2.82 (m, 1H), 2.10-2.04 (m, 2H), 1.94-1.91 (m, 4H), 1.78-1.72 (m, 2H).

The following compounds were synthesized using methods similar to those described herein.

| Compound # | Structure | LC/MS* |
|---|---|---|
| 120 | 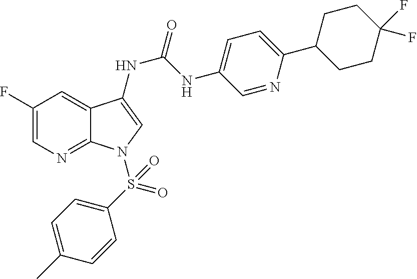 [SMILES: Cc1ccc(S(=O)(=O)n2cc(NC(=O)Nc3ccc(C4CCC(F)(F)CC4)nc3)c3cc(F)cnc32)cc1] 1-(6-(4,4-difluorocyclohexyl)pyridin-3-yl)-3-(5- fluoro-1-tosyl-1H-pyrrolo[2,3-b]pyridin-3-yl)urea | 544.3 |
| 121 | 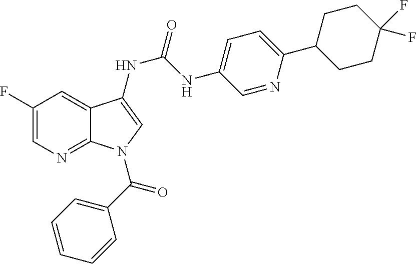 [SMILES: O=C(Nc1ccc(C2CCC(F)(F)CC2)nc1)Nc1cn(C(=O)c2ccccc2)c2ncc(F)cc12] 1-(1-benzoyl-5-fluoro-1H-pyrrolo[2,3-b]pyridin-3- yl)-3-(6-(4,4-difluorocyclohexyl)pyridin-3-yl)urea | 494.1 |
| 122 | 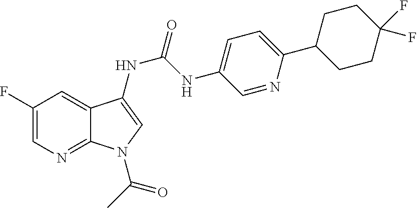 [SMILES: CC(=O)n1cc(NC(=O)Nc2ccc(C3CCC(F)(F)CC3)nc2)c2cc(F)cnc21] 1-(1-acetyl-5-fluoro-1H-pyrrolo[2,3-b]pyridin-3- yl)-3-(6-(4,4-difluorocyclohexyl)pyridin-3-yl)urea | 432.2 |
| 125 | 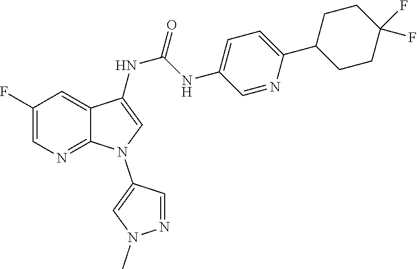 [SMILES: Cn1cc(-n2cc(NC(=O)Nc3ccc(C4CCC(F)(F)CC4)nc3)c3cc(F)cnc32)cn1] 1-(6-(4,4-difluorocyclohexyl)pyridin-3-yl)-3-(5- fluoro-1-(1-methyl-1H-pyrazol-4-yl)-1H- pyrrolo[2,3-b]pyridin-3-yl)urea | 470.2 |

*LCMS method: Shim-pack XR-ODS, C18, 3 × 50 mm, 2.5 um column, 1.0 uL injection, 1.5 mL/min flow rate, 90-900 amu scan range, 190-400 nm UV range, 5-100% (1.1 min), 100% (0.6 min) gradient with ACN (0.05% TFA) and water (0.05% TFA), with 2.0 minute as total run time.

### Biological Assays

STING pathway activation by the compounds described herein was measured using THP1-Dual™ cells (KO-IFNAR2).

THP1-Dual™ KO-IFNAR2 Cells (obtained from invivogen) were maintained in RPMI, 10% FCS, 5 ml P/S, 2 mM L-glut, 10 mM Hepes, and 1 mM sodium pyruvate. Compounds were spotted in empty 384 well tissue culture plates (Greiner 781182) by Echo for a final concentration of 0.0017-100 μM. Cells were plated into the TC plates at L per well, 2×10E6 cells/mL. For activation with STING ligand, 2′3′cGAMP (MW 718.38, obtained from Invivogen), was prepared in Optimem media.

The following solutions were prepared for each 1×384 plate:
Solution A: 2 mL Optimem with one of the following stimuli:
   60 uL of 10 mM 2′3′cGAMP->150 μM stock
Solution B: 2 mL Optimem with 60 μL Lipofectamine 2000->Incubate 5 min at RT

2 mL of solution A and 2 ml Solution B was mixed and incubated for 20 min at room temperature (RT). 20 uL of transfection solution (A+B) was added on top of the plated cells, with a final 2′3′cGAMP concentration of 15 μM. The plates were then centrifuged immediately at 340 g for 1 minute, after which they were incubated at 37° C., 5% CO₂, >98% humidity for 24 h. Luciferase reporter activity was then measured. EC₅₀ values were calculated by using standard methods known in the art.

Luciferase reporter assay: 10 μL of supernatant from the assay was transferred to white 384-plate with flat bottom and squared wells. One pouch of QUANTI-Luc™ Plus was dissolved in 25 mL of water. 100 μL of QLC Stabilizer per 25 mL of QUANTI-Luc™ Plus solution was added. 50 μL of QUANTI-Luc™ Plus/QLC solution per well was then added. Luminescence was measured on a Platereader (e.g., Spectramax I3X (Molecular Devices GF3637001)).

Luciferase reporter activity was then measured. EC₅₀ values were calculated by using standard methods known in the art.

Table BA shows the activity of compounds in STING reporter assay: <0.008 μM=“++++++”; ≥0.008 and <0.04 μM=“+++++”; ≥0.04 and <0.2 μM=“++++”; ≥0.2 and <1 μM=“+++”; ≥1 and <5 μM=“++”; ≥5 and <100 μM=“+”.

TABLE BA

| Compound | hSTING: | Compound | hSTING: |
|---|---|---|---|
| No. | EC₅₀ (μM) | No. | EC₅₀ (μM) |
| 101 | + | 158 | + |
| 102 | + | 159 | >30 |
| 103 | + | 160 | +++ |
| 104 | + | 161 | ++ |
| 105 | + | 162 | +++ |
| 106 | ++ | 163 | +++ |
| 107 | +++ | 164 | +++ |
| 108 | +++ | 165 | +++ |
| 109 | +++ | 166 | +++ |
| 110 | ++++ | 167 | >30 |
| 111 | +++ | 168 | +++ |
| 112 | ++ | 169 | +++ |
| 113 | ++++ | 170 | +++ |
| 114 | >100 | 171 | +++ |
| 116 | +++ | 172 | ++ |
| 118 | +++ | 173 | +++ |
| 121 | +++ | 174 | ++ |
| 122 | +++ | 175 | + |
| 123 | >30 | 177 | >30 |
| 124 | >30 | 178 | >30 |
| 125 | >30 | 179 | >30 |
| 127 | >30 | 180 | + |
| 128 | >30 | 181 | +++ |
| 136 | + | 182 | + |
| 137 | ++ | 183 | >30 |
| 138 | ++ | 184 | +++ |
| 139 | ++ | 185 | >30 |
| 140 | +++ | 186 | +++ |
| 141 | +++ | 187 | +++ |
| 142 | +++ | 188 | +++ |
| 143 | +++ | 189 | +++ |
| 144 | +++ | 190 | +++ |
| 145 | +++ | 191 | +++ |
| 146 | ++ | 192 | ++++ |
| 147 | + | 193 | + |
| 148 | +++ | 194 | ++ |
| 149 | +++ | 195 | ++++ |
| 150 | +++ | 196 | +++ |
| 151 | +++ | 197 | >30 |
| 152 | ++++ | 198 | ++++ |
| 154 | +++ | 199 | +++ |
| 155 | ++++ | 200 | +++ |
| 156 | >30 | 201 | +++ |
| 157 | +++ | 202 | ++++ |
| | | 203 | +++ |
| 204 | ++ | 225 | ++ |
| 205 | >30 | 226 | +++ |
| 206 | >30 | 227 | +++ |
| 207 | + | 228 | +++ |
| 208 | +++ | 229 | +++ |
| 209 | >30 | 230 | +++ |
| 210 | +++ | 231 | +++ |
| 211 | +++ | 232 | ++ |
| 212 | ++ | 233 | ++++ |
| 213 | +++ | 234 | +++ |
| 214 | + | 235 | ++ |
| 215 | ++ | 236 | ++++ |
| 216 | ++ | 237 | ++ |
| 217 | ++ | 238 | +++ |
| 218 | +++ | 239 | +++ |
| 219 | +++ | 240 | + |
| 220 | >30 | 241 | + |
| 221 | +++ | 242 | >10 |
| 222 | +++ | 243 | +++ |
| 223 | +++ | 244 | + |
| 224 | + | 245 | ++ |

### Numbered Clauses

The compounds, compositions, methods, and other subject matter described herein are further described in the following numbered clauses:

1. A compound of Formula I:

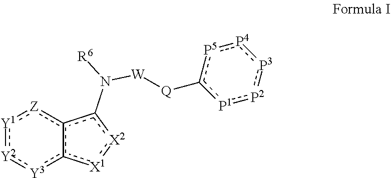

or a pharmaceutically acceptable salt thereof or a tautomer thereof,

each of Z, Y¹, Y², and Y³ is independently selected from the group consisting of CR¹, N, and NR², provided that 1-3 of Z, Y¹, Y², and Y³ is an independently selected N or NR²;

X¹ is selected from the group consisting of O, S, N, NR², and CR¹;

X² is selected from the group consisting of O, S, N, NR⁴, and CR⁵;

each [symbol] is independently a single bond or a double bond, provided that the five-membered ring comprising X¹ and X² is heteroaryl; the six-membered ring comprising Z, Y¹, Y², and Y³ is heteroaryl; and the ring comprising P¹, P², P³, P⁴, and P⁵ is aromatic;

W is selected from the group consisting of: (i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);

Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents the point of attachment to W;

P¹, P², P³, P⁴, and P⁵ are defined according to (AA) or (BB):
(AA)

each of P¹, P², P³, P⁴, and P⁵ is independently selected from the group consisting of: N, CH, CR⁷, and CR^{c}, provided that: 1-2 of P¹, P², P³, P⁴, and P⁵ is an independently selected CR⁷; or
(BB)

P¹ is absent, thereby providing a 5-membered ring,

each of P², P³, P⁴, and P⁵ is independently selected from the group consisting of O, S, N, NH, NR^{d}, NR⁷, CH, CR⁷, and CR^{c};

provided that 1-3 of P², P³, P⁴, and P⁵ is O, S, N, NH, NR^{d}, or NR⁷; and

1-2 of P², P³, P⁴, and P⁵ is an independently selected NR⁷ or CR⁷;

each R⁷ is independently selected from the group consisting of: —R⁸ and -L³-R⁹;

—R⁸ is selected from the group consisting of:

(a) C₃₋₁₂ cycloalkyl or C₃₋₁₂ cycloalkenyl, each of which is substituted with 1-4 independently selected R^{7′};

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′};

(c) C₃ cycloalkyl, C3 cycloalkenyl, C5 cycloalkyl, or C5 cycloalkenyl, each of which is optionally substituted with 1-4 independently selected C₁₋₄ alkyl;

(d) C₇₋₁₂ cycloalkyl or C₇₋₁₂ cycloalkenyl, each of which is optionally substituted with 1-4 independently selected C₁₋₄ alkyl;

(e) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl or heterocycloalkenyl ring is optionally substituted with 1-4 independently selected C₁₋₄ alkyl;

(f) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(g) C₆₋₁₀ aryl optionally substituted with 1-4 independently selected R^{7′};

-L³ is selected from the group consisting of —O—, —S—, —NH—, S(O)₁₋₂, —CH₂—, C(═O)NH, NHC(═O), C(═O)O, OC(═O), C(═O), NHS(O)₂, and S(O)₂NH;

—R⁹ is selected from the group consisting of:

(a) C₃₋₁₂ cycloalkyl or C₃₋₁₂ cycloalkenyl, each of which is optionally substituted with 1-4 independently selected R^{7′},

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and

(d) C₆₋₁₀ aryl optionally substituted with 1-4 independently selected R^{7′};

each occurrence of R^{7′} is independently selected from the group consisting of:

halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),

provided that when R⁷ is R⁸; and R⁸ is cycloalkyl, cycloalkenyl, heterocyclyl, or heterocycloalkenyl and substituted with 1-4 R^{7′}, then:

R⁸ cannot be monosubstituted with C₁₋₄ alkyl, and

when R⁸ is substituted with 2-4 R^{7′}, then at least one R^{7′} must be a substituent other than C₁₋₄ alkyl;

each occurrence of R¹ is independently selected from the group consisting of:

H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy optionally substituted with —OH, C₁₋₄ alkoxy, C₁₋₄ haloalkoxy, or —NR^{e}R^{f}; C₁₋₄ haloalkoxy; -L¹-L²-R^{h}; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);

each occurrence of R² is independently selected from the group consisting of:

(i) H;

(ii) C₁₋₆ alkyl optionally substituted with 1-3 independently selected R^{a};

(iii) —C(O)(C₁₋₆ alkyl) optionally substituted with 1-3 independently selected R^{a};

(iv) —C(O)O(C₁₋₄ alkyl) optionally substituted with 1-3 independently selected R^{a};

(v) —CON(R′)(R″);

(vi) —S(O)₁₋₂(NR′R″);

(vii) —S(O)₁₋₂(C₁₋₄ alkyl) optionally substituted with 1-3 independently selected R^{a};

(viii) —OH;

(ix) C₁₋₄ alkoxy; and

(x) -L⁴-L⁵-R;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂, wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

halo; cyano; C₁₋₁₀ alkyl optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};

R^{d} is selected from the group consisting of: C₁₋₆ alkyl optionally substituted with 1-3 substituents each independently selected from the group consisting of halo, C₁₋₄ alkoxy, and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or

R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene; -L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};

-L⁵- is selected from the group consisting of a bond and C₁₋₄ alkylene;

Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;

each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or

R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S; and

provided that the compound is other than:

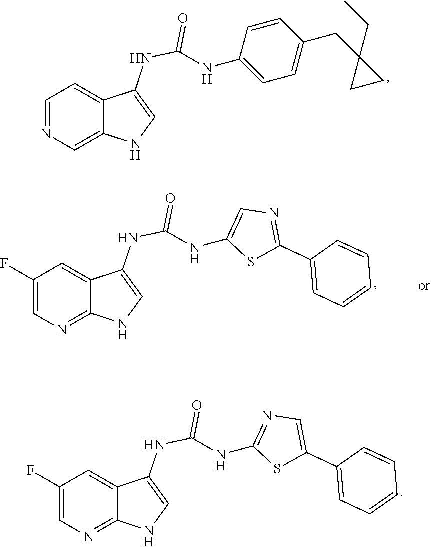 [SMILES: CCC1(Cc2ccc(NC(=O)Nc3c[nH]c4cnccc34)cc2)CC1 ; O=C(Nc1cnc(-c2ccccc2)s1)Nc1c[nH]c2ncc(F)cc12 ; O=C(Nc1ncc(-c2ccccc2)s1)Nc1c[nH]c2ncc(F)cc12]

2. The compound of clause 1, wherein P¹, P², P³, P⁴, and P⁵ are as defined according to (AA).

3. The compound of clause 2, wherein one or more, such as one, of P¹, P², P³, P⁴, and P⁵ is N.

4. The compound of clause 2, wherein each one of P¹, P², P³, P⁴, and P⁵ is independently selected from the group consisting of CH, CR⁷, and, CR^{c}.

5. The compound of any one of clauses 2-4, wherein one of P¹, P², P³, P⁴, and P⁵ is CR⁷.

6. The compound of any one of clauses 2-5, wherein P³ is CR⁷.

7. The compound of any one of clauses 2-3 or 5-6, wherein P⁴ is N.

8. The compound of any one of clauses 2-7, wherein each of P¹, P², and P⁵ is independently selected from the group consisting of CH and CR^{c}.

9. The compound of clause 2, wherein P³ is CR⁷; P⁴ is N; and each of P¹, P², and P⁵ is independently selected from the group consisting of CH and CR^{c}

10. The compound of clause 2, wherein P³ is CR⁷; and each of P¹, P², P⁴ and P⁵ is independently selected from the group consisting of CH and CR^{c}; or

wherein P³ is CR⁷; P¹ is N; and each of P², P⁴, and P⁵ is independently selected from the group consisting of CH and CR^{c}.

11. The compound of any one of clauses 2-5, wherein P⁴ is CR⁷.

12. The compound of clause 11, wherein each of P¹, P², P³, and P⁵ is independently selected from the group consisting of CH and CR^{c}.

13. The compound of clause 11, wherein one of P¹, P², P³, and P⁵ is N; and

each remaining of P¹, P², P³, and P⁵ is independently selected from the group consisting of CH and CR^{c}.

14. The compound of any one of clauses 1-2 or 9, wherein the

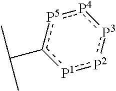

moiety has the formula:

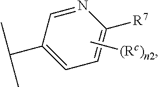

wherein n2 is 0, 1, or 2.

15. The compound of clause 14, wherein the

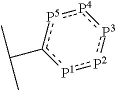

moiety has the formula.

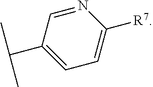

16. The compound of clause 14, wherein the

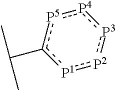

moiety has the formula:

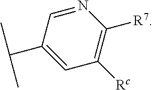

17. The compound of any one of clauses 1-2 or 10, wherein the

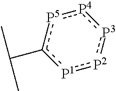

moiety has the formula:

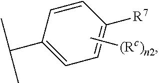

wherein n2 is 0, 1, or 2.

18. The compound of clause 17, wherein the

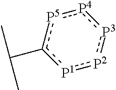

moiety has the formula:

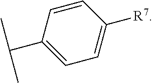

19. The compound of clause 17, wherein the

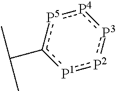

moiety has the formula:

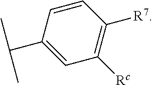

20. The compound of any one of clauses 1-19, wherein R⁷ is R⁸.

21. The compound of any one of clauses 1-20, wherein R⁸ is selected from the group consisting of:

(a) C₃₋₁₂ cycloalkyl or C₃₋₁₂ cycloalkenyl, each of which is substituted with 1-4 independently selected R^{7′}; and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

22. The compound of any one of clauses 1-21, wherein R⁸ is C₃₋₁₂ cycloalkyl or C₃₋₁₂ cycloalkenyl, each of which is substituted with 1-4 independently selected R^{7′}.

23. The compound of any one of clauses 1-22, wherein R⁸ is C₄₋₁₀ cycloalkyl or C₄₋₁₀ cycloalkenyl, each of which is substituted with 1-4 independently selected R^{7′}.

24. The compound of any one of clauses 1-23, wherein R⁸ is C₄₋₈ cycloalkyl or C₄₋₈ cycloalkenyl, each of which is substituted with 1-4 independently selected R^{7′} 25. The compound of any one of clauses 1-24, wherein R⁸ is C₄₋₈ cycloalkyl which is substituted with 1-4 independently selected R^{7′}.

26. The compound of any one of clauses 1-25, wherein R⁸ is C₄₋₈ cycloalkyl which is substituted with 2-4 independently selected R^{7′}.

27. The compound of clause 26, wherein R⁸ is cyclohexyl or cyclobutyl, each of which is substituted with 2-4 independently selected R^{7′}.

28. The compound of clause 27, wherein R⁸ is

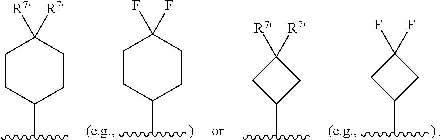

29. The compound of any one of clauses 1-21, wherein R⁸ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

30. The compound of any one of clauses 1-21 or 29, wherein R⁸ is heterocyclyl or heterocycloalkenyl of 4-10 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

31. The compound of any one of clauses 1-21 or 29-30, wherein R⁸ is heterocyclyl or heterocycloalkenyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

32. The compound of any one of clauses 1-21 or 29-31, wherein R⁸ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

33. The compound of any one of clauses 1-21 or 29-32, wherein R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 2-4 independently selected R^{7′}.

34. The compound of any one of clauses 1-21 or 29-33, wherein R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 2-4 (e.g., 2) independently selected R^{7′}; or

wherein R⁸ is selected from the group consisting of azetidinyl, pyrrolidinyl, and piperidinyl, each of which is substituted with 2-4 (e.g., 2) independently selected R^{7′}.

35. The compound of any one of clauses 1-21 or 29-34, wherein R⁸ is selected from the group consisting of:

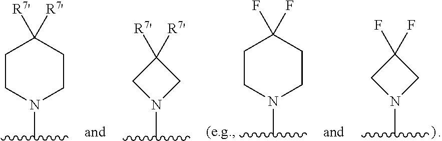

36. The compound of any one of clauses 1-21 or 29, wherein R⁸ is spirocyclic heterocyclyl of 6-12 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 2-4 independently selected R^{7′} (e.g., R⁸ is

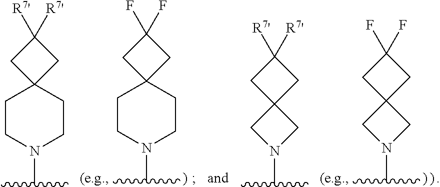

37. The compound of any one of clauses 1-20, wherein R⁸ is selected from the group consisting of:

(c) C₃ cycloalkyl, C3 cycloalkenyl, C5 cycloalkyl, or C5 cycloalkenyl, each of which is optionally substituted with 1-4 independently selected C₁₋₄ alkyl; and

(d) C₇₋₁₂ cycloalkyl or C₇₋₁₂ cycloalkenyl, each of which is optionally substituted with 1-4 independently selected C₁₋₄ alkyl.

38. The compound of any one of clauses 1-20 or 37, wherein R⁸ is selected from the group consisting of:

(c) C₃ cycloalkyl or C5 cycloalkyl, each of which is optionally substituted with 1-4 independently selected C₁₋₄ alkyl; and

(d) C₇₋₁₂ cycloalkyl which is optionally substituted with 1-4 independently selected C₁₋₄ alkyl.

39. The compound of any one of clauses 1-20 or 37-38, wherein R⁸ is cyclopentyl.

40. The compound of any one of clauses 1-20, wherein R⁸ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl or heterocycloalkenyl ring is optionally substituted with 1-4 independently selected C₁₋₄ alkyl.

41. The compound of any one of clauses 1-20 or 40, wherein R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl ring is optionally substituted with 1-4 independently selected C₁₋₄ alkyl.

42. The compound of clause 41, wherein R⁸ is selected from the group consisting of: azetidinyl, oxetanyl, pyrrolidinyl, tetrahydrofuranyl, piperidinyl, piperazinyl, morpholinyl, and azepinyl, each of which is optionally substituted with 1-4 independently selected C₁₋₄ alkyl.

43. The compound of any one of clauses 41-42, wherein R⁸ is azetidinyl, pyrrolidinyl, piperidinyl, piperazinyl, and morpholinyl, each of which is optionally substituted with 1-2 independently selected C₁₋₄ alkyl, such as: wherein R⁸ is morpholinyl, which is optionally substituted with 1-2 independently selected C₁₋₄ alkyl.

44. The compound of any one of clauses 1-20 or 40-41, wherein R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂, provided that R⁸ contains one ring N(R^{d}) group, and wherein one or more ring carbon atoms of the heterocyclyl ring is optionally substituted with 1-4 independently selected C₁₋₄ alkyl, such as wherein R⁸ is

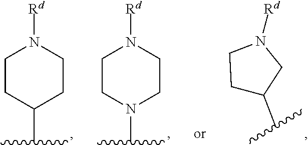

or optionally wherein R^{d} is C₁₋₆ alkyl optionally substituted with 1-3 substituents each independently selected from the group consisting of halo, C₁₋₄ alkoxy, and OH, such as wherein R^{d} is C₁₋₄ alkyl substituted with 1-3 independently selected halo.

45. The compound of any one of clauses 1-20 or 40, wherein R⁸ is bicyclic or polycyclic heterocyclyl or heterocycloalkenyl of 7-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected C₁₋₄ alkyl.

46. The compound of any one of clauses 1-20, 40, or 45, wherein R⁸ is bicyclic or polycyclic heterocyclyl of 7-12 ring atoms (e.g., spirocyclic heterocyclyl of 7-12 ring atoms), wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected C₁₋₄ alkyl.

47. The compound of clause 46, wherein R⁸ is

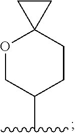 [SMILES: *C1CCC2(CC2)OC1]

or wherein R⁸ is

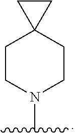 [SMILES: *N1CCC2(CC1)CC2]

48. The compound of any one of clauses 1-20, wherein R⁸ is heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}.

49. The compound of any one of clauses 1-20 or 48, wherein R⁸ is heteroaryl of 5-6 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R^{7′}.

50. The compound of any one of clauses 1-20, wherein R⁸ is bicyclic heteroaryl of 7-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R^{7′}.

51. The compound of clause 50, wherein R⁸ is

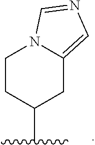 [SMILES: *C1CCn2cncc2C1]

52. The compound of any one of clauses 1-19, wherein R⁷ is -L³-R⁹.

53. The compound of any one of clauses 1-19 or 52, wherein -L³ is —O—.

54. The compound of any one of clauses 1-19 or 52, wherein -L³ is —NH—.

55. The compound of any one of clauses 1-19 or 52, wherein -L³ is —S— or S(O)₁₋₂.

56. The compound of any one of clauses 1-19 or 52, wherein -L³ is selected from the group consisting of: C(═O)NH, NHC(═O), C(═O)O, OC(═O), C(═O), NHS(O)₂, and S(O)₂NH.

57. The compound of any one of clauses 1-19 or 52-56, wherein R⁹ is selected from the group consisting of:

(a) C₃₋₁₂ cycloalkyl or C₃₋₁₂ cycloalkenyl, each of which is optionally substituted with 1-4 independently selected R^{7′}, and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′}.

58. The compound of any one of clauses 1-19 or 52-57, wherein R⁹ is C₃₋₁₂ cycloalkyl or C₃₋₁₂ cycloalkenyl, each of which is optionally substituted with 1-4 independently selected R^{7′}.

59. The compound of any one of clauses 1-19 or 52-58, wherein R⁹ is C₄₋₈ cycloalkyl which is optionally substituted with 1-2 independently selected R^{7′}.

60. The compound of clause 59, wherein R⁹ is cyclobutyl, cyclopentyl, or cyclohexyl, each of which is optionally substituted with 1-2 independently selected R^{7′} (e.g., unsubstituted).

61. The compound of any one of clauses 1-19 or 52-57, wherein R⁹ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′}.

62. The compound of any one of clauses 1-19, 52-57, or 61, wherein R⁹ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

63. The compound of clause 62, wherein R⁹ is selected from the group consisting of azetidinyl, oxetanyl, pyrrolidinyl, tetrahydrofuranyl, piperidinyl, piperazinyl, morpholinyl, and azepinyl, each of which is optionally substituted with 1-2 independently selected R^{7′} (e.g., unsubstituted).

64. The compound of any one of clauses 1-19, wherein R⁷ is L³-R⁹; L³ is —O— or —NH—; and R⁹ is selected from the group consisting:

C₄₋₈ cycloalkyl which is optionally substituted with 1-2 independently selected R^{7′}; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

65. The compound of clause 64, wherein R⁷ is L³-R⁹; L³ is —O— or —NH—; and R⁹ is selected from the group consisting of cyclobutyl, cyclopentyl, cyclohexyl, and oxetanyl, each of which is optionally substituted with 1-2 independently selected R^{7′} (e.g., unsubstituted).

66. The compound of any one of clauses 64-65, wherein L³ is —O—.

67. The compound of any one of clauses 64-66, wherein R⁷ is

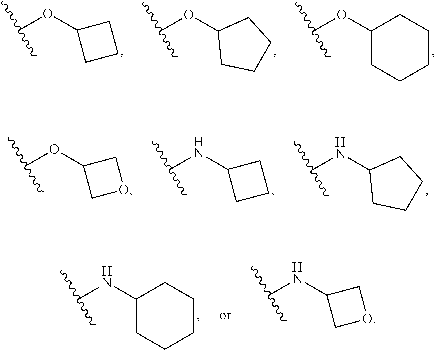

68. The compound of clause 1, wherein the

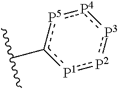

moiety has the formula:

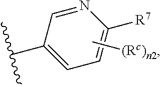

wherein n2 is 0, 1, or 2; and R⁷ is R⁸, wherein R⁸ is selected from the group consisting of:

C₄₋₈ cycloalkyl which is substituted with 1-4 independently selected R^{7′}; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

69. The compound of clause 68, wherein n2 is 0.

70. The compound of clause 68, wherein n2 is 1.

71. The compound of clause 70, wherein R^{c} is located ortho to R⁷.

72. The compound of any one of clauses 68-71, wherein R⁷ is R⁸; and R⁸ is C₄₋₈ cycloalkyl which is substituted with 2-4 independently selected R^{7′}.

73. The compound of clause 72, wherein R⁸ is cyclohexyl which is substituted with 2-4 independently selected R^{7′}, such as

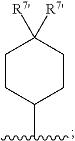

or wherein R⁸ is cyclobutyl which is substituted with 2-4 independently selected R^{7′}, such as

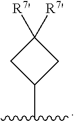

74. The compound of any one of clauses 68-71, wherein R⁷ is R⁸; and R⁸ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

75. The compound of clause 74, wherein R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 2-4 independently selected R^{7′}.

76. The compound of clause 75, wherein R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 2-4 (e.g., 2) independently selected R^{7′}.

77. The compound of clause 76, wherein R⁸ is selected from the group consisting of azetidinyl, pyrrolidinyl, and piperidinyl, each of which is substituted with 2-4 (e.g., 2) independently selected R^{7′}, such as

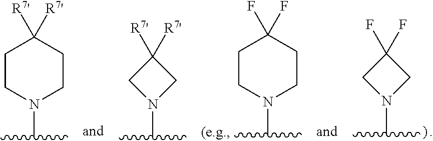

78. The compound of clause 1, wherein the

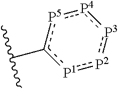

moiety has the formula:

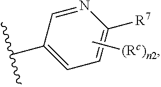

wherein n2 is 0, 1, or 2; and R⁷ is -L³-R⁹, wherein:

L³ is —NH— or —O—; and R⁹ is selected from the group consisting:

C₄₋₈ cycloalkyl which is optionally substituted with 1-2 independently selected R^{7′}; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

79. The compound of clause 78, wherein R⁷ is L³-R⁹; L³ is —O— or —NH—; and

R⁹ is selected from the group consisting of cyclobutyl, cyclopentyl, cyclohexyl, and oxetanyl, each of which is optionally substituted with 1-2 independently selected R^{7′} (e.g., unsubstituted).

80. The compound of any one of clauses 78-79, wherein L³ is —O—.

81. The compound of any one of clauses 78-80, wherein R⁷ is

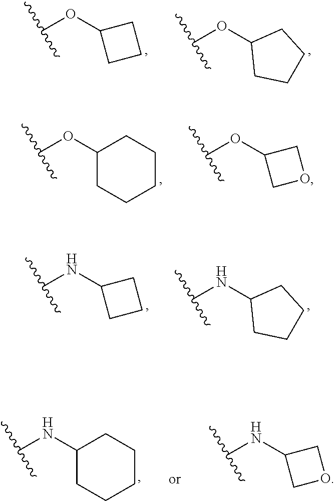

82. The compound of any one of clauses 1-81, wherein each R^{7′} when present is independently selected from the group consisting of: halo, —CN, —OH, —C₁₋₄ alkyl, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″), provided that when R⁷ is R⁸; and R⁸ is cycloalkyl, cycloalkenyl, heterocyclyl, or heterocycloalkenyl, then one or more occurrence of R^{7′} is other than —C₁₋₄ alkyl.

83. The compound of any one of clauses 1-82, wherein each R^{7′} when present is independently selected from the group consisting of: halo, —CN, —C₁₋₄ alkyl, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″), provided that when R⁷ is R⁸; and R⁸ is cycloalkyl, cycloalkenyl, heterocyclyl, or heterocycloalkenyl, then one or more occurrence of R^{7′} is other than —C₁₋₄ alkyl.

84. The compound of any one of clauses 1-83, wherein each R^{7′} when present is independently halo.

85. The compound of any one of clauses 1-84, wherein each R^{7′} when present is —F.

86. The compound of any one of clauses 1-85, wherein each R^{c} when present is independently selected from the group consisting of: halo; cyano; C₁₋₁₀ alkyl which is optionally substituted with 1-6 independently selected R^{a}; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; and —C(═O)N(R′)(R″).

87. The compound of any one of clauses 1-86, wherein each R^{c} when present is independently selected from the group consisting of: halo; cyano; C₁₋₁₀ alkyl optionally substituted with 1-6 independently selected —F, —Br, or —C₁; C₁₋₄ alkoxy; C₁₋₄ haloalkoxy; —S(O)₁₋₂(C₁₋₄ alkyl); and —C(═O)(C₁₋₁₀ alkyl), such as wherein each R^{c} is an independently selected halo (e.g., —F or —Cl), C₁₋₄ alkyl (e.g., CH₃), or CF₃ (e.g., each R^{c} is an independently selected halo (e.g., —F or —Cl)).

88. The compound of any one of clauses 1-87, wherein Q is NH.

89. The compound of any one of clauses 1-87, wherein Q is N(C₁₋₃ alkyl).

90. The compound of any one of clauses 1-89, wherein W is C(═O).

91. The compound of any one of clauses 1-89, wherein W is S(O)₂, C(═S), or C(═NR^{d}).

92. The compound of any one of clauses 1-91, wherein X¹ is NR².

93. The compound of any one of clauses 1-91, wherein X¹ is NH.

94. The compound of any one of clauses 1-93, wherein X² is CR⁵.

95. The compound of any one of clauses 1-93, wherein X² is CH.

96. The compound of any one of clauses 1-91, wherein X¹ is NR²; and X² is CR⁵.

97. The compound of any one of clauses 1-91, wherein X¹ is NH; and X² is CH.

98. The compound of any one of clauses 1-97, wherein Z is CR¹.

99. The compound of any one of clauses 1-98, wherein 1-2 of Y¹, Y², and Y³ is independently N or NR² (e.g., N); and each of the remaining of Y¹, Y², and Y³ is an independently selected CR¹.

100. The compound of any one of clauses 1-99, wherein one of Y¹, Y², and Y³ is independently N or NR²; and each of the remaining of Y¹, Y², and Y³ is an independently selected CR¹.

101. The compound of any one of clauses 1-100, wherein one of Y¹, Y², and Y³ is independently N; and each of the remaining of Y¹, Y², and Y³ is an independently selected CR¹.

102. The compound of any one of clauses 1-101, wherein the

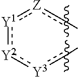

moiety is

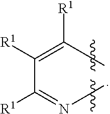

103. The compound of clause 102, wherein the

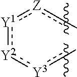

moiety is

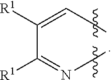

104. The compound of clause 102, wherein the

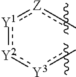

moiety is

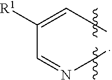

105. The compound of clause 102, wherein the

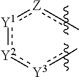

moiety is

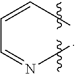 [SMILES: */C=C\C=N/*]

106. The compound of any one of clauses 1-101, wherein the

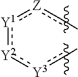

moiety is

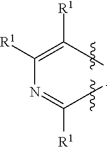

107. The compound of clause 106, wherein the

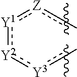

moiety is

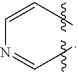 [SMILES: */C=C\N=C/*]

108. The compound of any one of clauses 1-101, wherein the

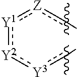

moiety is

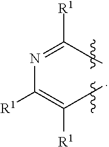

109. The compound of clause 108, wherein the

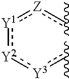

Y moiety is

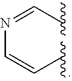 [SMILES: */C=C\N=C/*]

110. The compound of any one of clauses 1-97, wherein Z is N.

111. The compound of any one of clauses 1-97 or 110, wherein each of Y¹, Y², and Y³ is an independently selected CR¹.

112. The compound of any one of clauses 1-97 or 110-111, wherein the

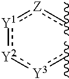

moiety is

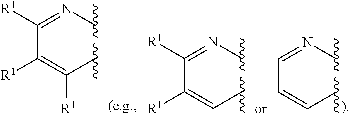

113. The compound of any one of clauses 1-97, wherein the compound is selected from a compound of the following formulae:

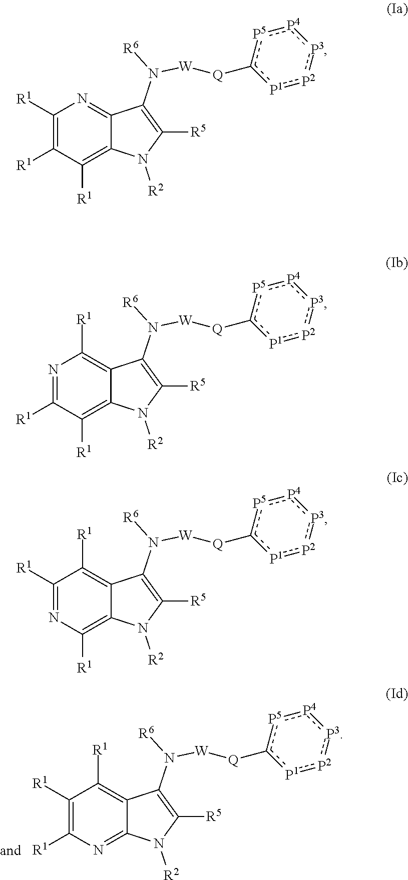

114. The compound of any one of clauses 1-97 or 113, wherein the compound has formula (I-a):

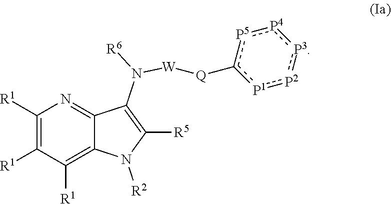

115. The compound of clause 114, wherein the compound has formula (I-a1), (I-a2), or (I-a3):

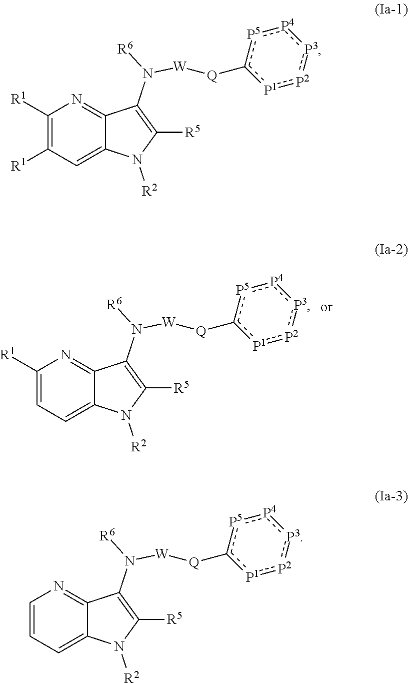

116. The compound of any one of clauses 1-97 or 113, wherein the compound has formula (I-b):

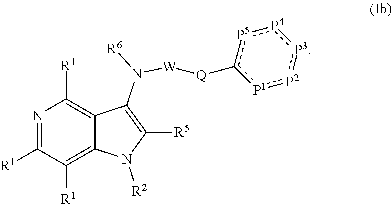

117. The compound of clause 116, wherein the compound has formula (Ib-1):

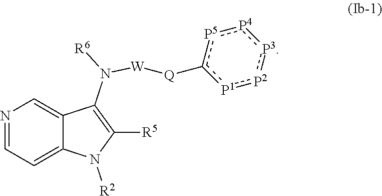

118. The compound of any one of clauses 1-97 or 113, wherein the compound has formula (I-c):

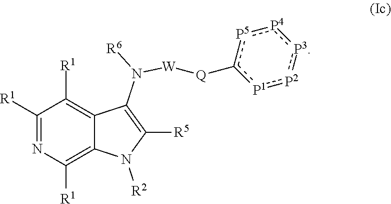

119. The compound of clause 118, wherein the compound has formula (Ic-1):

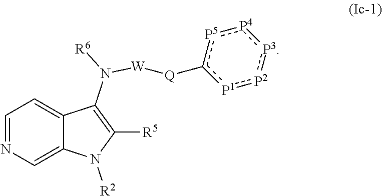

120. The compound of any one of clauses 1-97 or 113, wherein the compound has formula (I-d):

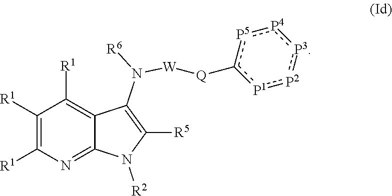

121. The compound of clause 120, wherein the compound has formula (Id-1):

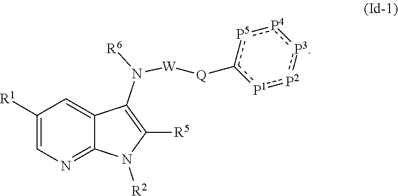

122. The compound of clause 120, wherein the compound has formula (Id-2):

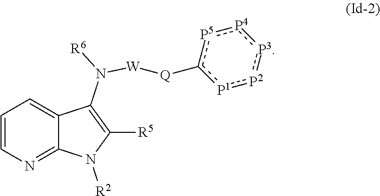

123. The compound of clause 120, wherein the compound has formula (Id-3):

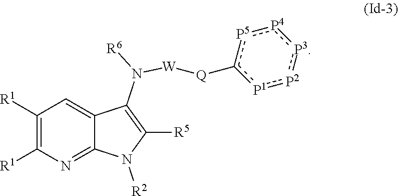

124. The compound of any one of clauses 1-123, wherein each occurrence of R¹ is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L¹-L²-R^{h}; —S(O)₁₋₂(C₁₋₄ alkyl); —S(O)(═NH)(C₁₋₄ alkyl); SF₅; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

125. The compound of any one of clauses 1-124, wherein 0-2 (e.g., 0, 1, or 2) occurrences of R¹ is other than H; and each of the remaining occurrences of R¹ is H.

126. The compound of any one of clauses 1-125, wherein each occurrence of R¹ is H.

127. The compound of any one of clauses 1-125, wherein 1-2 occurrences of R¹ is other than H.

128. The compound of clause 127, wherein one occurrence of R¹ is other than H.

129. The compound of any one of clauses 1-125 or 127-128, wherein one occurrence of R¹ is selected from the group consisting of: halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy optionally substituted with C₁₋₄ alkoxy; C₁₋₄ haloalkoxy; —S(O)₁₋₂(C₁₋₄ alkyl); —S(O)₁₋₂(NR′R″); —NO₂; -L¹-L²-R^{h}; —C(═O)(C₁₋₄ alkyl); —C(═O)O(C₁₋₄ alkyl); —C(═O)OH; and —C(═O)N(R′)(R″), such as, wherein:

one occurrence of R¹ is selected from the group consisting of: halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)₁₋₂(NR′R″); —NO₂; -L¹-L²-R^{h}; —C(═O)(C₁₋₄ alkyl); —C(═O)O(C₁₋₄ alkyl); —C(═O)OH; and —C(═O)N(R′)(R″).

130. The compound of any one of clauses 1-125 or 127-129, wherein one occurrence of R¹ is halo (e.g., F or Cl (e.g., F)); or wherein one occurrence of R¹ is C₁₋₃ alkyl, such as methyl or ethyl; or wherein one occurrence of R¹ is C₁₋₃ alkoxy such as methoxy; or wherein one occurrence of R¹ is C₁₋₄ haloalkoxy; or wherein one occurrence of R¹ is C₁₋₄ alkoxy substituted with C₁₋₄ alkoxy.

131. The compound of any one of clauses 1-125 or 127-129, wherein one occurrence of R¹ is -L¹-L²-R^{h}.

132. The compound of clause 131, wherein -L¹ is a bond.

133. The compound of any one of clauses 131-132, wherein -L² is a bond.

134. The compound of any one of clauses 131-133, wherein —R^{h} is selected from the group consisting of:
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

135. The compound of clause 134, wherein —R^{h} is selected from the group consisting of:
heteroaryl of 5-6 ring atoms (e.g., pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
phenyl, which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

136. The compound of clause 131, wherein one of R′ is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy

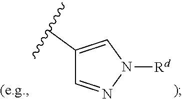

and
phenyl which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy

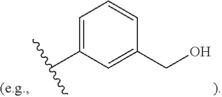 [SMILES: *c1cccc(CO)c1]

137. The compound of any one of clauses 1-136, wherein R² is H.

138. The compound of any one of clauses 1-136, wherein R² is selected from the group consisting of:

(iii) —C(O)(C₁₋₆ alkyl) optionally substituted with 1-3 independently selected R^{a};

(iv) —C(O)O(C₁₋₄ alkyl) optionally substituted with 1-3 independently selected R^{a};

(v) —CON(R′)(R″);

(vi) —S(O)₁₋₂(NR′R″); and

(vii) —S(O)₁₋₂(C₁₋₄ alkyl) optionally substituted with 1-3 independently selected R^{a}.

139. The compound of clause 138, wherein R² is —C(O)(C₁₋₆ alkyl) optionally substituted with 1-3 independently selected R^{a}.

140. The compound of clause 139, wherein each R^{a} substituent of R² is independently —F, —Cl, —OH, or —NR^{e}R^{f}.

141. The compound of any one of clauses 139-140, wherein R² is selected from the group consisting of: C(═O)Me,

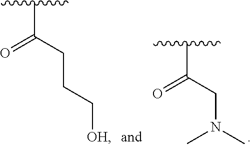

142. The compound of clause 138, wherein R² is —S(O)₁₋₂(C₁₋₄ alkyl) optionally substituted with 1-3 independently selected R^{a}, such as wherein R² is S(O)₂Me.

143. The compound of any one of clauses 1-136, wherein R² is -L⁴-L⁵-Rⁱ.

144. The compound of clause 143, wherein -L⁴ is a bond.

145. The compound of clause 143, wherein -L⁴ is C(═O).

146. The compound of clause 143, wherein -L⁴ is S(O)₂.

147. The compound of any one of clauses 143-146, wherein -L⁵ is a bond.

148. The compound of any one of clauses 143-146, wherein -L⁵ is C₁₋₄ alkylene (e.g., C₁₋₂ alkylene).

149. The compound of any one of clauses 143-148, wherein R′ is selected from the group consisting of: (a) C₃₋₈ cycloalkyl, optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is

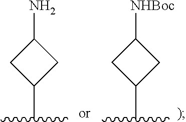 [SMILES: *C1CC(N)C1 ; *C1CC(NC(=O)OC(C)(C)C)C1]

and

(b) heterocyclyl, wherein the heterocyclyl has 3-8 ring atoms, wherein 1-3 ring atoms are heteroatoms, each independently selected from the group consisting of N, N(H), N(R^{d}), O, and S(O)₀₋₂, wherein the heterocyclyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is

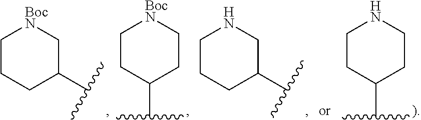

150. The compound of any one of clauses 143-148, wherein Rⁱ is selected from the group consisting of: (a) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(b) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

151. The compound of clause 143, wherein R² is -L⁴-L⁵-Rⁱ; L⁴ is a bond; L⁵ is a bond or C₁₋₄ alkylene; and Rⁱ is selected from the group consisting of:

(a) C₃₋₈ cycloalkyl, optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy

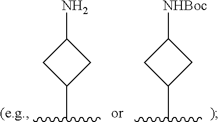 [SMILES: *C1CC(N)C1 ; *C1CC(NC(=O)OC(C)(C)C)C1]

(b) heterocyclyl, wherein the heterocyclyl has 3-8 ring atoms, wherein 1-3 ring atoms are heteroatoms, each independently selected from the group consisting of N, N(H), N(R^{d}), O, and S(O)₀₋₂, wherein the heterocyclyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy

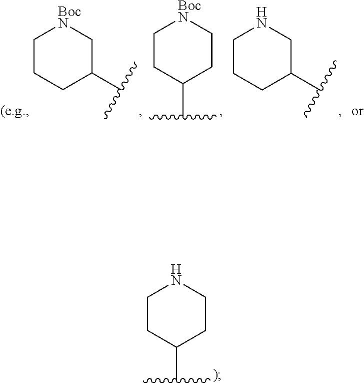

and

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

152. The compound of clause 143, wherein R² is -L⁴-L⁵-Rⁱ; L⁴ is C(═O) or S(O)₂; L⁵ is a bond or C₁₋₄ alkylene; and Rⁱ is selected from the group consisting of:

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

153. The compound of clause 152, wherein R² is selected from the group consisting of:

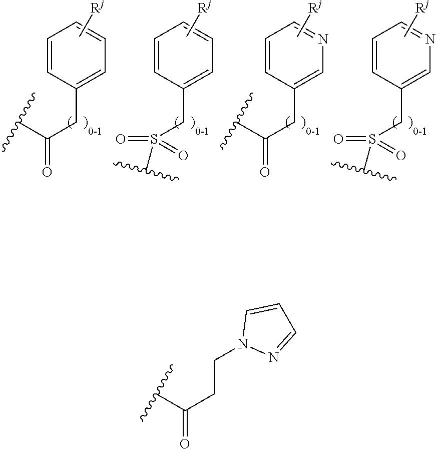

wherein Rⁱ is H; halo; C₁₋₄ alkyl; C₁₋₄ haloalkyl; cyano; C₁₋₄ alkoxy; or C₁₋₄ haloalkoxy.

154. The compound of any one of clauses 1-153, wherein R⁵ is H.

155. The compound of clause 1, wherein the compound is a compound of Formula (I-1a):

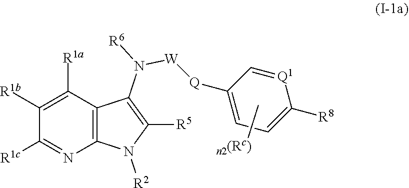

or a pharmaceutically acceptable salt thereof, wherein:

each of R^{1a}, R^{1b}, and R^{1c} is an independently selected R¹;

Q¹ is N or CH; and n2 is 0, 1, or 2.

156. The compound of clause 1, wherein the compound is a compound of Formula (I-1b):

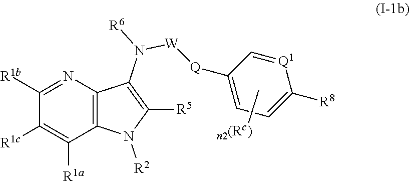

or a pharmaceutically acceptable salt thereof, wherein:

each of R^{1a}, R^{1b}, and R^{1c} is an independently selected R¹;

Q¹ is N or CH; and n2 is 0, 1, or 2.

157. The compound of clauses 155 or 156, wherein R⁸ is C₃₋₁₂ cycloalkyl or C₃₋₁₂ cycloalkenyl, each of which is substituted with 1-4 independently selected R^{7′}.

158. The compound of any one of clauses 155-157, wherein R⁸ is C₄₋₈ cycloalkyl which is substituted with 2-4 independently selected R^{7′}.

159. The compound of any one of clauses 155-158, wherein R⁸ is cyclohexyl which is substituted with 2-4 independently selected R^{7′}, such as: wherein R⁸ is

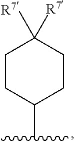

such as

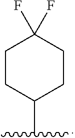 [SMILES: *C1CCC(F)(F)CC1]

160. The compound of any one of clauses 155-158, wherein R⁸ is cyclobutyl which is substituted with 2-4 independently selected R^{7′}, such as: wherein R⁸ is

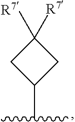

such as

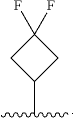 [SMILES: *C1CC(F)(F)C1]

161. The compound of clauses 155 or 156, wherein R⁸ is heterocyclyl or heterocycloalkenyl of 4-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

162. The compound of any one of clauses 155-156 or 161, wherein R⁸ is heterocyclyl of 4-8 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 2-4 independently selected R^{7′}.

163. The compound of clause 162, wherein R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 2-4 (e.g., 2) independently selected R^{7′}.

164. The compound of any one of clauses 162-163, wherein R⁸ is selected from the group consisting of azetidinyl, pyrrolidinyl, and piperidinyl, each of which is substituted with 2-4 (e.g., 2) independently selected R^{7′}.

165. The compound of any one of clauses 162-164, wherein R⁸ is selected from the group consisting of:

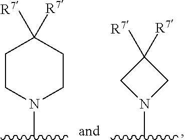

such as

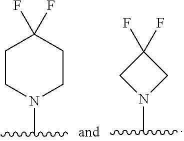 [SMILES: *N1CCC(F)(F)CC1 ; *N1CC(F)(F)C1]

166. The compound of clauses 155 or 156, wherein R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl ring is optionally substituted with 1-4 independently selected C₁₋₄ alkyl.

167. The compound of clause 166, wherein R⁸ is azetidinyl, oxetanyl, pyrrolidinyl, tetrahydrofuranyl, piperidinyl, piperazinyl, morpholinyl, and azepinyl, each of which is optionally substituted with 1-4 independently selected C₁₋₄ alkyl, such as wherein R⁸ is morpholinyl which is optionally substituted with 1-2 independently selected C₁₋₄ alkyl.

168. The compound of clauses 155 or 156, wherein R⁸ is selected from the group consisting of:

(c) C₃ cycloalkyl, or C5 cycloalkyl, each of which is optionally substituted with 1-4 independently selected C₁₋₄ alkyl; and

(d) C₇₋₁₂ cycloalkyl which is optionally substituted with 1-4 independently selected C₁₋₄ alkyl.

169. The compound of clause 168, wherein R⁸ is unsubstituted C₃, C₅, or C₇₋₁₂ cycloalkyl (such as cyclopentyl).

170. The compound of clause 1, wherein the compound is a compound of Formula (I-2):

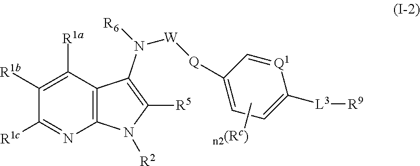

or a pharmaceutically acceptable salt thereof, wherein:

each of R^{1a}, R^{1b}, and R^{1c} is an independently selected R¹;

Q¹ is N or CH; and n2 is 0, 1, or 2.

171. The compound of clause 170, wherein L³ is —O—.

172. The compound of clause 170, wherein L³ is —NH—.

173. The compound of clause 170, wherein L³ is selected from the group consisting of: —S—, —S(O)₂—, C(═O)NH, NHC(═O), C(═O)O, OC(═O), C(═O), NHS(O)₂, and S(O)₂NH.

174. The compound of any one of clauses 170-173, wherein R⁹ is C₃₋₁₂ cycloalkyl or C₃₋₁₂ cycloalkenyl, each of which is optionally substituted with 1-4 independently selected R^{7′}.

175. The compound of any one of clauses 170-174, wherein R⁹ is C₄₋₈ cycloalkyl which is optionally substituted with 1-2 independently selected R^{7′}.

176. The compound of clause 175, wherein R⁹ is cyclobutyl, cyclopentyl, or cyclohexyl, each of which is optionally substituted with 1-2 independently selected R^{7′} (e.g., unsubstituted).

177. The compound of any one of clauses 170-173, wherein R⁹ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

178. The compound of clause 177, wherein R⁹ is selected from the group consisting of azetidinyl, oxetanyl, pyrrolidinyl, tetrahydrofuranyl, piperidinyl, piperazinyl, morpholinyl, and azepinyl, each of which is optionally substituted with 1-2 independently selected R^{7′} (e.g., unsubstituted).

179. The compound of clause 170, wherein -L³-R⁹ is selected from the group consisting of:

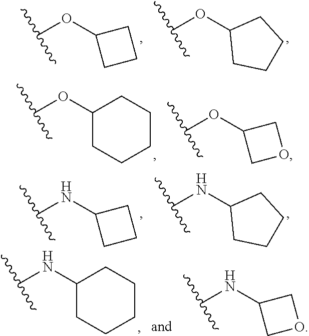

180. The compound of any one of clauses 155-178, wherein each R^{7′} when present is independently selected from the group consisting of: halo, —CN, —C₁₋₄ alkyl, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″), provided that when R^{7′} is a substituent of R⁸; and R⁸ is cycloalkyl, cycloalkenyl, heterocyclyl, or heterocycloalkenyl, then one or more occurrence of R^{7′} is other than —C₁₋₄ alkyl.

181. The compound of any one of clauses 155-180, wherein each R^{7′} when present is independently selected from the group consisting of halo, —CN, —C₁₋₄ alkyl, —C₁₋₄ haloalkyl, —C₁₋₆ alkoxy, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″).

182. The compound of any one of clauses 155-181, wherein each R^{7′} when present is independently halo.

183. The compound of clause 182, wherein each R^{7′} when present is —F.

184. The compound of any one of clauses 155-183, wherein n2 is 0.

185. The compound of any one of clauses 155-183, wherein n2 is 1.

186. The compound of any one of clauses 155-183 or 185, wherein each R^{c} when present is independently selected from the group consisting of: halo; cyano; C₁₋₁₀ alkyl; C₁₋₄ alkoxy; C₁₋₄ haloalkoxy; —S(O)₁₋₂(C₁₋₄ alkyl); and —C(═O)(C₁₋₁₀ alkyl).

187. The compound of any one of clauses 155-183 or 185-186, wherein each R^{c} when present is halo (e.g., —F, —Br, or —Cl) or cyano; or wherein each R^{c} when present is C₁₋₃ alkyl such as methyl or ethyl.

188. The compound of any one of clauses 155-187, wherein Q¹ is N.

189. The compound of any one of clauses 155-187, wherein Q¹ is CH.

190. The compound of any one of clauses 155-189, wherein Q is NH.

191. The compound of any one of clauses 155-190, wherein W is C(═O).

192. The compound of any one of clauses 155-190, wherein W is S(O)₂, C(═S), or C(═NR^{d}).

193. The compound of any one of clauses 155-192, wherein each of R^{1a}, R^{1b}, and R^{1c} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L¹-L²-R^{h}; —S(O)₁₋₂(C₁₋₄ alkyl); —S(O)(═NH)(C₁₋₄ alkyl); SF₅; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″).

194. The compound of any one of clauses 155-193, wherein R^{1a} is H.

195. The compound of any one of clauses 155-194, wherein R″ is H.

196. The compound of any one of clauses 155-195, wherein R^{1b} is H.

197. The compound of any one of clauses 155-195, wherein R^{1b} other than H, such as wherein R^{1b} is halo.

198. The compound of clause 197, wherein R^{1b} is —F.

199. The compound of clause 197, wherein R^{1b} is —Br or —Cl.

200. The compound of any one of clauses 155-195, wherein R^{1b} is C₁₋₃ alkoxy such as methoxy.

201. The compound of any one of clauses 155-195, wherein R^{1b} is L¹-L²-R^{h}.

202. The compound of clause 201, wherein -L¹ is a bond.

203. The compound of clauses 201 or 202, wherein -L² is a bond.

204. The compound of any one of clauses 201-203, wherein-R^{h} is selected from the group consisting of:
heteroaryl of 5-6 ring atoms (e.g., pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
phenyl which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy.

205. The compound of clause 201, wherein one of R^{1b} is selected from the group consisting of:
heteroaryl of 5-6 ring atoms (such as pyrazolyl), wherein 1-4 ring atoms are heteroatoms, each independently selected from the group consisting of N, N(H), N(R^{d}), O, and S(O)₀₋₂ and wherein the heteroaryl ring is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy N-R^{d}

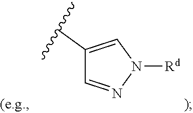

and
phenyl which is optionally substituted with 1-2 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy

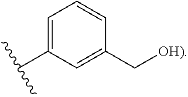

206. The compound of any one of clauses 155-205, wherein R⁵ is H.

207. The compound of any one of clauses 155-206, wherein R² is H.

208. The compound of any one of clauses 155-206, wherein R² is —C(O)(C₁₋₆ alkyl) optionally substituted with 1-3 independently selected R^{a}; or

—S(O)₁₋₂(C₁₋₄ alkyl) optionally substituted with 1-3 independently selected R^{a} (e.g., S(O)₂Me).

209. The compound of clause 208, wherein R² is selected from the group consisting of: C(═O)Me, S(O)₂Me,

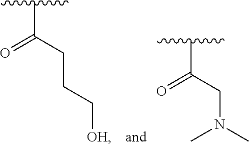

210. The compound of any one of clauses 155-206, wherein R² is -L⁴-L⁵-R; L⁴ is a bond; L⁵ is a bond or C₁₋₄ alkylene (e.g., CH₂); and Rⁱ is selected from the group consisting of:

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

211. The compound of any one of clauses 155-206, wherein R² is -L⁴-L⁵-R; L⁴ is C(═O) or S(O)₂; L⁵ is a bond or C₁₋₄ alkylene; and Rⁱ is selected from the group consisting of:

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

212. The compound of clause 211, wherein R² is selected from the group consisting of:

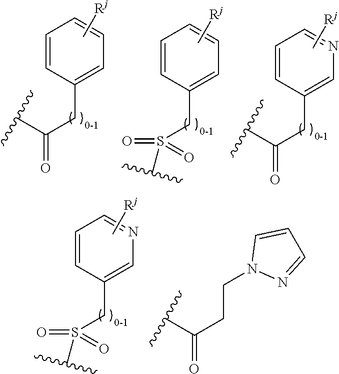

wherein R^{j} is H; halo; C₁₋₄ alkyl; C₁₋₄ haloalkyl; cyano; C₁₋₄ alkoxy; or C₁₋₄ haloalkoxy.

213. The compound of any one of clauses 1-212, wherein R⁶ is hydrogen.

214. The compound of clause 1, wherein the compound is a compound of Formula (I-3a) or (I-3b)

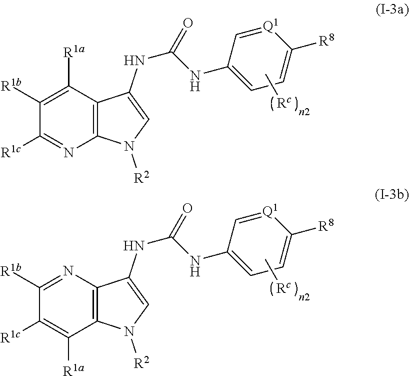

or a pharmaceutically acceptable salt thereof, wherein:

each of R^{1a}, R^{1b}, and R^{1c} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

Q¹ is N or CH;

R⁸ is selected from the group consisting of:

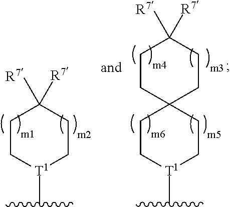

n2 is 0, 1, or 2;

each R^{c} when present is independently selected from the group consisting of: halo, cyano, C₁₋₃ alkyl, and C₁₋₃ alkoxy;

m1 and m2 are independently 0, 1, or 2; m3, m4, m5, and m6 are independently 0 or 1; and

T¹ is CH or N.

215. The compound of clause 214, wherein R² is H.

216. The compound of clauses 214 or 215, wherein R^{1a} and R^{1c} are H.

217. The compound of any one of clauses 214-216, wherein R^{1b} is H; or wherein R^{1b} is halo, such as —F; or wherein R^{1b} is C₁₋₃ alkoxy.

218. The compound of any one of clauses 214-217, wherein n2 is 1, optionally wherein R^{c} is ortho to R⁸.

219. The compound of any one of clauses 214-218, wherein R⁸ is selected from the group consisting of:

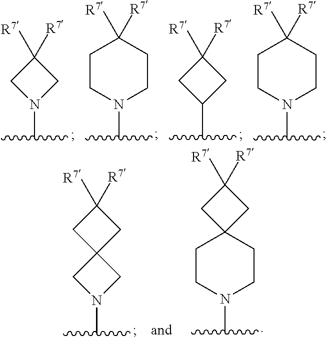

220. The compound of any one of clauses 214-219, wherein each R^{7′} is halo, such as —F.

221. The compound of clause 1, wherein the compound is selected from the group consisting of the compounds delineated in Table C₁, or a pharmaceutically acceptable salt thereof.

222. The compound of clause 1, wherein the compound is selected from the group consisting of the following:

| Compound # | Name |
|---|---|
| 108 | 1-(5-chloro-1H-pyrrolo[2,3-b]pyridin-3-yl)-3-(6-(4,4- |
| | difluorocyclohexyl)pyridin-3-yl)urea |
| 106 | 1-(6-(4,4-difluorocyclohexyl)pyridin-3-yl)-3-(5-methyl-1H-pyrrolo[2,3- |
| | b]pyridin-3-yl)urea |
| 109 | 1-(6-cyclopentylpyridin-3-yl)-3-(5-fluoro-1H-pyrrolo[2,3-b]pyridin-3- |
| | yl)urea |
| 110 | 1-(5-chloro-6-(4,4-difluoropiperidin-1-yl)pyridin-3-yl)-3-(5-fluoro-1H- |
| | pyrrolo[2,3-b]pyridin-3-yl)urea |
| 111 | 1-(6-cyclobutoxy-5-fluoropyridin-3-yl)-3-(5-fluoro-1H-pyrrolo[2,3- |
| | b]pyridin-3-yl)urea |
| 112 | 1-(6-(cyclobutylamino)pyridin-3-yl)-3-(5-fluoro-1H-pyrrolo[2,3-b]pyridin- |
| | 3-yl)urea |
| 113 | 1-(6-(4,4-difluorocyclohexyl)-5-fluoropyridin-3-yl)-3-(5-fluoro-1H- |
| | pyrrolo[2,3-b]pyridin-3-yl)urea |
| 115 | 1-(5-fluoro-1H-pyrrolo[2,3-b]pyridin-3-yl)-3-(6-(4-hydroxypiperidin-1- |
| | yl)pyridin-3-yl)urea |
| 116 | 1-(6-(4,4-difluorocyclohexyl)pyridin-3-yl)-3-(5-fluoro-1H-pyrrolo[2,3- |
| | b]pyridin-3-yl)urea |
| 117 | 1-(6-cyclobutoxypyridin-3-yl)-3-(5-fluoro-1H-pyrrolo[2,3-b]pyridin-3- |
| | yl)urea |
| 118 | 1-(6-(4,4-difluoropiperidin-1-yl)pyridin-3-yl)-3-(5-fluoro-1H-pyrrolo[2,3- |
| | b]pyridin-3-yl)urea |
| 119 | 1-(5-fluoro-1H-pyrrolo[2,3-b]pyridin-3-yl)-3-(6-morpholinopyridin-3- |
| | yl)urea |
| 104 | 1-(1-benzyl-5-fluoro-1H-pyrrolo[2,3-b]pyridin-3-yl)-3-(6-(4,4- |
| | difluorocyclohexyl)pyridin-3-yl)urea |
| 127 | 1-(6-(4,4-difluorocyclohexyl)pyridin-3-yl)-3-(5-fluoro-1-methyl-1H- |
| | pyrrolo[2,3-b]pyridin-3-yl)urea |
| 107 | 1-(6-(4,4-difluorocyclohexyl)pyridin-3-yl)-3-(5-(1-isopropyl-1H-pyrazol-4- |
| | yl)-1H-pyrrolo[2,3-b]pyridin-3-yl)urea |
| 105 | 1-(6-(4,4-difluorocyclohexyl)pyridin-3-yl)-3-(5-(3-(hydroxymethyl)phenyl)- |
| | 1H-pyrrolo[2,3-b]pyridin-3-yl)urea |
| 103 | tert-butyl 4-(3-(3-(6-(4,4-difluorocyclohexyl)pyridin-3-yl)ureido)-5-fluoro- |
| | 1H-pyrrolo[2,3-b]pyridin-1-yl)piperidine-1-carboxylate |
| 128 | 1-(6-(4,4-difluorocyclohexyl)pyridin-3-yl)-3-(5-fluoro-1-(piperidin-4-yl)- |
| | 1H-pyrrolo[2,3-b]pyridin-3-yl)urea |
| 101 | tert-butyl(3-(3-(3-(6-(4,4-difluorocyclohexyl)pyridin-3-yl)ureido)-5-fluoro- |
| | 1H-pyrrolo[2,3-b]pyridin-1-yl)cyclobutyl)(methyl)carbamate |
| 124 | 1-(6-(4,4-difluorocyclohexyl)pyridin-3-yl)-3-(5-fluoro-1-(3- |
| | (methylamino)cyclobutyl)-1H-pyrrolo[2,3-b]pyridin-3-yl)urea |
| 102 | tert-butyl 3-(3-(3-(6-(4,4-difluorocyclohexyl)pyridin-3-yl)ureido)-5-fluoro- |
| | 1H-pyrrolo[2,3-b]pyridin-1-yl)piperidine-1-carboxylate |
| 123 | 1-(6-(4,4-difluorocyclohexyl)pyridin-3-yl)-3-(5-fluoro-1-(piperidin-3-yl)- |
| | 1H-pyrrolo[2,3-b]pyridin-3-yl)urea |
| 114 | 1-(5-fluoro-1H-pyrrolo[2,3-b]pyridin-3-yl)-3-(6-(4- |
| | hydroxycyclohexyl)pyridin-3-yl)urea |
| 202 | 1-(5-chloro-6-(4,4-difluoropiperidin-1-yl)pyridin-3-yl)-3-(5-fluoro-1H- |
| | pyrrolo[2.3-b]pyridin-3-yl)urea |
| 201 | 1-(4-(3,3-difluorocyclobutyl)-3-fluorophenyl)-3-(5-fluoro-1H-pyrrolo[2,3- |
| | b]pyridin-3-yl)urea |
| 200 | 1-(1-acetyl-5-fluoro-1H-pyrrolo[2,3-b]pyridin-3-yl)-3-(6-(4,4- |
| | difluorocyclohexyl)pyridin-3-yl)urea |
| 199 | 1-(5-chloro-6-(4,4-difluorocyclohexyl)pyridin-3-yl)-3-(5-fluoro-1H- |
| | pyrrolo[2,3-b]pyridin-3-yl)urea |
| 198 | 1-(6-(4,4-difluoropiperidin-1-yl)-5-methylpyridin-3-yl)-3-(5-fluoro-1H- |
| | pyrrolo[2,3-b]pyridin-3-yl)urea |
| 196 | 1-(5-chloro-6-(piperidin-1-yl)pyridin-3-yl)-3-(5-fluoro-1H-pyrrolo[2,3- |
| | b]pyridin-3-yl)urea |
| 195 | 1-(3-cyano-4-(4,4-difluorocyclohexyl)phenyl)-3-(5-fluoro-1H-pyrrolo[2,3- |
| | b]pyridin-3-yl)urea |
| 194 | 1-(5-fluoro-1H-pyrrolo[2,3-b]pyridin-3-yl)-3-(6-(4-methoxypiperidin-1-yl)- |
| | 5-methylpyridin-3-yl)urea |
| 193 | 1-(6-(4,4-difluoropiperidin-1-yl)pyridazin-3-yl)-3-(5-fluoro-1H-pyrrolo[2,3- |
| | b]pyridin-3-yl)urea |
| 192 | 1-(5-fluoro-1H-pyrrolo[2,3-b]pyridin-3-yl)-3-(5-methyl-6-(6-azaspiro[2.5]octan-6- |
| | yl)pyridin-3-yl)urea |
| 191 | 1-(5-(4,4-difluoropiperidin-1-yl)pyrazin-2-yl)-3-(5-fluoro-1H-pyrrolo[2,3- |
| | b]pyridin-3-yl)urea |
| 190 | 1-(2-(4,4-difluoropiperidin-1-yl)pyrimidin-5-yl)-3-(5-fluoro-1H-pyrrolo[2,3- |
| | b]pyridin-3-yl)urea |
| 189 | 1-(5-chloro-6-(4,4-difluoropiperidin-1-yl)pyridin-3-yl)-3-(1H-pyrrolo[2,3- |
| | b]pyridin-3-yl)urea |
| 187 | 1-(6-(4,4-difluoropiperidin-1-yl)-5-fluoropyridin-3-yl)-3-(5-methoxy-1H- |
| | pyrrolo[3,2-b]pyridin-3-yl)urea |
| 186 | 1-(4-chloro-5-(4,4-difluoropiperidin-1-yl)pyridin-2-yl)-3-(5-fluoro-1H- |
| | pyrrolo[2,3-b]pyridin-3-yl)urea |
| 185 | 1-(6-(4,4-difluoropiperidin-1-yl)-4-methylpyridin-3-yl)-3-(5-fluoro-1H- |
| | pyrrolo[2,3-b]pyridin-3-yl)urea |
| 183 | 1-(4-chloro-6-(4,4-difluoropiperidin-1-yl)pyridin-3-yl)-3-(5-fluoro-1H- |
| | pyrrolo[2,3-b]pyridin-3-yl)urea |
| 179 | 1-(6-(4,4-difluoropiperidin-1-yl)-4-methylpyridin-3-yl)-3-(4-methyl-1H- |
| | pyrrolo[2,3-b]pyridin-3-yl)urea |
| 178 | 1-(3-chloro-5-(4,4-difluoropiperidin-1-yl)pyridin-2-yl)-3-(5-fluoro-1H- |
| | pyrrolo[2,3-b]pyridin-3-yl)urea |
| 177 | 1-(5-(4,4-difluoropiperidin-1-yl)-3-methylpyridin-2-yl)-3-(5-fluoro-1H- |
| | pyrrolo[2,3-b]pyridin-3-yl)urea |
| 173 | 1-(6-(4,4-difluoropiperidin-1-yl)-5-methylpyridin-3-yl)-3-(5-methoxy-1H- |
| | pyrrolo[3,2-b]pyridin-3-yl)urea |
| 172 | 1-(6-(4,4-difluoropiperidin-1-yl)-5-methylpyridin-3-yl)-3-(7-fluoro-1H- |
| | pyrrolo[3,2-c]pyridin-3-yl)urea |
| 171 | 1-(6-(3,3-difluoroazetidin-1-yl)-5-methylpyridin-3-yl)-3-(5-fluoro-1H- |
| | pyrrolo[2,3-b]pyridin-3-yl)urea |
| 170 | 1-(5-fluoro-1H-pyrrolo[2,3-b]pyridin-3-yl)-3-(5-methyl-6-(4- |
| | methylpiperidin-1-yl)pyridin-3-yl)urea |
| 169 | 1-(6-(4,4-difluorocyclohexyl)-5-methylpyridin-3-yl)-3-(5-methoxy-1H- |
| | pyrrolo[3,2-b]pyridin-3-yl)urea |
| 167 | 1-(5-fluoro-1H-pyrrolo[2,3-b]pyridin-3-yl)-3-(5-methyl-6- |
| | morpholinopyridin-3-yl)urea |
| 166 | 1-(5-chloro-6-(3,3-difluorocyclobutyl)pyridin-3-yl)-3-(5-fluoro-1H- |
| | pyrrolo[2,3-b]pyridin-3-yl)urea |
| 165 | 1-(6-(4,4-difluoropiperidin-1-yl)-5-methoxypyridin-3-yl)-3-(5-fluoro-1H- |
| | pyrrolo[2,3-b]pyridin-3-yl)urea |
| 164 | 1-(6-(4,4-difluorocyclohexyl)-5-methoxypyridin-3-yl)-3-(5-fluoro-1H- |
| | pyrrolo[2,3-b]pyridin-3-yl)urea |
| 163 | 1-(6-(4,4-difluoropiperidin-1-yl)-5-ethylpyridin-3-yl)-3-(5-fluoro-1H- |
| | pyrrolo[2,3-b]pyridin-3-yl)urea |
| 162 | 1-(6-(6,6-difluoro-2-azaspiro[3.3]heptan-2-yl)-5-methylpyridin-3-yl)-3-(5- |
| | fluoro-1H-pyrrolo[2,3-b]pyridin-3-yl)urea |
| 161 | 1-(6-(3,3-difluorocyclobutyl)pyridin-3-yl)-3-(5-fluoro-1H-pyrrolo[2,3- |
| | b]pyridin-3-yl)urea |
| 160 | 1-(5-(4,4-difluorocyclohexyl)-4-methoxypyridin-2-yl)-3-(5-fluoro-1H- |
| | pyrrolo[2,3-b]pyridin-3-yl)urea |
| 159 | 1-(5-fluoro-1H-pyrrolo[2,3-b]pyridin-3-yl)-3-(5-methyl-6-(2-oxa-6- |
| | azaspiro[3.3]heptan-6-yl)pyridin-3-yl)urea |
| 158 | 1-(6-(4,4-difluorocyclohexyl)-5-(hydroxymethyl)pyridin-3-yl)-3-(5-fluoro- |
| | 1H-pyrrolo[2,3-b]pyridin-3-yl)urea |
| 157 | 1-(5-cyano-6-(4,4-difluoropiperidin-1-yl)pyridin-3-yl)-3-(5-fluoro-1H- |
| | pyrrolo[2,3-b]pyridin-3-yl)urea |
| 156 | 1-(5-fluoro-1H-pyrrolo[2,3-b]pyridin-3-yl)-3-(6-(6-hydroxy-2- |
| | azaspiro[3.3]heptan-2-yl)-5-methylpyridin-3-yl)urea |
| 155 | 1-(5-cyano-6-(4,4-difluorocyclohexyl)pyridin-3-yl)-3-(5-fluoro-1H- |
| | pyrrolo[2,3-b]pyridin-3-yl)urea |
| 154 | 1-(3-cyano-4-(4,4-difluorocyclohexyl)phenyl)-3-(5-methoxy-1H- |
| | pyrrolo[2,3-c]pyridin-3-yl)urea |
| 152 | 1-(4-chloro-5-(4,4-difluorocyclohexyl)pyridin-2-yl)-3-(5-fluoro-1H- |
| | pyrrolo[2,3-b]pyridin-3-yl)urea |
| 151 | 1-(5-chloro-1H-pyrrolo[2,3-b]pyridin-3-yl)-3-(5-cyano-6-(4,4- |
| | difluorocyclohexyl)pyridin-3-yl)urea |
| 150 | 1-(3-cyano-4-(4,4-difluorocyclohexyl)phenyl)-3-(5-methoxy-1H- |
| | pyrrolo[3,2-b]pyridin-3-yl)urea |
| 149 | 1-(3-cyano-4-(4,4-difluorocyclohexyl)phenyl)-3-(7-fluoro-1H-pyrrolo[3,2- |
| | c]pyridin-3-yl)urea |
| 148 | 1-(3-cyano-4-(4,4-difluorocyclohexyl)phenyl)-3-(1H-pyrrolo[3,2-b]pyridin- |
| | 3-yl)urea |
| 147 | 1-(6-(4,4-difluoropiperidin-1-yl)-5-(2-hydroxyethyl)pyridin-3-yl)-3-(5- |
| | fluoro-1H-pyrrolo[2,3-b]pyridin-3-yl)urea |
| 146 | 1-(6-(4,4-difluoropiperidin-1-yl)-5-methylpyridin-3-yl)-3-(1H-pyrrolo[3,2- |
| | b]pyridin-3-yl)urea |
| 145 | 1-(5-chloro-6-(4,4-difluoropiperidin-1-yl)pyridin-3-yl)-3-(6-fluoro-1H- |
| | pyrrolo[3,2-b]pyridin-3-yl)urea |
| 143 | 1-(5-chloro-6-(3,3-difluorocyclobutyl)pyridin-3-yl)-3-(1H-pyrrolo[3,2- |
| | b]pyridin-3-yl)urea |
| 142 | 1-(5-chloro-6-(4,4-difluorocyclohexyl)pyridin-3-yl)-3-(1H-pyrrolo[3,2- |
| | b]pyridin-3-yl)urea |
| 141 | 1-(5-chloro-6-(4,4-difluoropiperidin-1-yl)pyridin-3-yl)-3-(5-methoxy-1H- |
| | pyrrolo[3,2-b]pyridin-3-yl)urea |
| 140 | 1-(5-cyano-6-(4,4-difluorocyclohexyl)pyridin-3-yl)-3-(5-methoxy-1H- |
| | pyrrolo[3,2-b]pyridin-3-yl)urea |
| 138 | 1-(6-(4,4-difluoropiperidin-1-yl)-5-(1-hydroxyethyl)pyridin-3-yl)-3-(5- |
| | fluoro-1H-pyrrolo[2,3-b]pyridin-3-yl)urea |
| 137 | 1-(5-chloro-1H-pyrrolo[2,3-b]pyridin-3-yl)-3-(6-(4,4-difluorocyclohexyl)-5- |
| | methylpyridin-3-yl)urea |
| 136 | 1-(6-(4,4-difluorocyclohexyl)-5-methylpyridin-3-yl)-3-(1H-pyrrolo[3,2- |
| | b]pyridin-3-yl)urea |
| 205 | 1-(1-(4,4-difluorocyclohexyl)-1H-pyrazol-4-yl)-3-(1H-pyrrolo[3,2- |
| | b]pyridin-3-yl)urea |
| 206 | 1-(1-((4,4-difluorocyclohexyl)methyl)-1H-pyrazol-4-yl)-3-(1H-pyrrolo[3,2- |
| | b]pyridin-3-yl)urea |
| 207 | 1-(5-chloro-6-(3,3-difluoro-1-oxa-9-azaspiro[5.5]undecan-9-yl)pyridin-3- |
| | yl)-3-(1H-pyrrolo[3,2-b]pyridin-3-yl)urea |
| 209 | 1-(1-(4,4-difluorocyclohexyl)-1H-pyrazol-4-yl)-3-(5-methoxy-1H- |
| | pyrrolo[3,2-b]pyridin-3-yl)urea |
| 213 | 1-(6-(4,4-difluorocyclohexyl)pyridin-3-yl)-3-(5-methoxy-1H-pyrrolo[3,2- |
| | b]pyridin-3-yl)urea |
| 214 | 1-(6-chloro-5-(4,4-difluorocyclohexyl)pyridin-3-yl)-3-(1H-pyrrolo[3,2- |
| | b]pyridin-3-yl)urea |
| 215 | 1-(5-chloro-6-(1-oxa-9-azaspiro[5.5]undecan-9-yl)pyridin-3-yl)-3-(1H- |
| | pyrrolo[3,2-b]pyridin-3-yl)urea |
| 216 | 1-(5-chloro-6-(1-(2,2,2-trifluoroethyl)pyrrolidin-3-yl)pyridin-3-yl)-3-(1H- |
| | pyrrolo[3,2-b]pyridin-3-yl)urea |
| 217 | 1-(2-chloro-6-(4,4-difluorocyclohexyl)pyridin-4-yl)-3-(1H-pyrrolo[3,2- |
| | b]pyridin-3-yl)urea |
| 219 | 1-(3-chloro-4-(3,3-difluorocyclobutyl)phenyl)-3-(1H-pyrrolo[3,2-b]pyridin- |
| | 3-yl)urea |
| 220 | 1-(6-(4,4-difluoropiperidin-1-yl)pyridin-3-yl)-3-(1H-pyrrolo[3,2-b]pyridin- |
| | 3-yl)urea |
| 221 | 1-(5-chloro-6-(6-azaspiro[2.5]octan-6-yl)pyridin-3-yl)-3-(1H-pyrrolo[3,2- |
| | b]pyridin-3-yl)urea |
| 222 | 1-(5-chloro-6-(1-(2,2,2-trifluoroethyl)piperidin-4-yl)pyridin-3-yl)-3-(1H- |
| | pyrrolo[3,2-b]pyridin-3-yl)urea |
| 223 | 1-(5-chloro-6-(6,6-difluoro-2-azaspiro[3.3]heptan-2-yl)pyridin-3-yl)-3-(1H- |
| | pyrrolo[3,2-b]pyridin-3-yl)urea |
| 224 | 1-(5-chloro-6-(6-(2,2,2-trifluoroethyl)-2,6-diazaspiro[3.3]heptan-2- |
| | yl)pyridin-3-yl)-3-(1H-pyrrolo[3,2-b]pyridin-3-yl)urea |
| 225 | 1-(5-chloro-6-(4-(3,3,3-trifluoropropyl)piperazin-1-yl)pyridin-3-yl)-3-(1H- |
| | pyrrolo[3,2-b]pyridin-3-yl)urea |
| 226 | 1-(5-chloro-6-(2,2-difluoro-7-azaspiro[3.5]nonan-7-yl)pyridin-3-yl)-3-(1H- |
| | pyrrolo[3,2-b]pyridin-3-yl)urea |
| 227 | 1-(5-chloro-6-(4-(2,2,2-trifluoroethyl)piperazin-1-yl)pyridin-3-yl)-3-(1H- |
| | pyrrolo[3,2-b]pyridin-3-yl)urea |
| 203/228 | 1-(5-chloro-6-(2,2-difluoro-7-azaspiro[3.5]nonan-7-yl)pyridin-3-yl)-3-(5- |
| | methoxy-1H-pyrrolo[3,2-b]pyridin-3-yl)urea |
| 229 | 1-(6-(4,4-difluorocyclohexyl)-5-fluoropyridin-3-yl)-3-(5-methoxy-1H- |
| | pyrrolo[3,2-b]pyridin-3-yl)urea |
| 230 | 1-(6-(4,4-difluoropiperidin-1-yl)-5-fluoropyridin-3-yl)-3-(1H-pyrrolo[3,2- |
| | b]pyridin-3-yl)urea |
| 231 | 1-(5-chloro-6-(3,3-difluoroazetidin-1-yl)pyridin-3-yl)-3-(5-methoxy-1H- |
| | pyrrolo[3,2-b]pyridin-3-yl)urea |
| 232 | 1-(6-(3,3-difluoroazetidin-1-yl)-5-fluoropyridin-3-yl)-3-(5-methoxy-1H- |
| | pyrrolo[3,2-b]pyridin-3-yl)urea |
| 233 | 1-(5-chloro-6-(4,4-difluorocyclohexyl)pyridin-3-yl)-3-(5-methoxy-1H- |
| | pyrrolo[3,2-b]pyridin-3-yl)urea |
| 234 | 1-(6-(4,4-difluorocyclohexyl)-5-fluoropyridin-3-yl)-3-(1H-pyrrolo[3,2- |
| | b]pyridin-3-yl)urea |
| 235 | 1-(4-chloro-5-(4,4-difluoropiperidin-1-yl)pyridin-2-yl)-3-(1H-pyrrolo[3,2- |
| | b]pyridin-3-yl)urea |
| 240 | 1-(6-bromo-1H-pyrrolo[3,2-b]pyridin-3-yl)-3-(5-chloro-6-(4,4- |
| | difluoropiperidin-1-yl)pyridin-3-yl)urea |
| 236 | 1-(5-bromo-1H-pyrrolo\|3,2-b]pyridin-3-yl)-3-(5-chloro-6-(4,4- |
| | difluoropiperidin-1-yl)pyridin-3-yl)urea |
| 237 | 1-(5-cyano-6-(4,4-difluorocyclohexyl)pyridin-3-yl)-3-(1H-pyrrolo[3,2- |
| | b]pyridin-3-yl)urea |
| 238 | 1-(4-(3,3-difluorocyclobutyl)-3-fluorophenyl)-3-(5-methoxy-1H- |
| | pyrrolo[3,2-b]pyridin-3-yl)urea |
| 239 | 1-(4-(3,3-difluorocyclobutyl)-3-fluorophenyl)-3-(1H-pyrrolo[3,2-b]pyridin- |
| | 3-yl)urea |
| 241 | 1-(5-chloro-6-(2,2-dimethylmorpholino)pyridin-3-yl)-3-(1H-pyrrolo[3,2- |
| | b]pyridin-3-yl)urea |
| 242 | 1-(5-chloro-6-(3,3-difluoroazetidin-1-yl)pyridin-3-yl)-3-(1H-pyrrolo[3,2- |
| | b]pyridin-3-yl)urea |
| 243 | 1-(5-chloro-6-(3,3-difluorocyclobutyl)pyridin-3-yl)-3-(5-methoxy-1H- |
| | pyrrolo[3,2-b]pyridin-3-yl)urea |
| 188 | 1-(5-chloro-6-(4,4-difluoropiperidin-1-yl)pyridin-3-yl)-3-(1H-pyrrolo[3,2- |
| | b]pyridin-3-yl)urea |
| 204 | 1-(6-(4,4-difluorocyclohexyl)pyridin-3-yl)-3-(5-(3,3,3-trifluoropropoxy)- |
| | 1H-pyrrolo[3,2-b]pyridin-3-yl)urea |
| 184 | 1-(6-(4,4-difluoropiperidin-1-yl)-5-fluoropyridin-3-yl)-3-(5-methoxy-1H- |
| | pyrrolo[2,3-b]pyridin-3-yl)urea |
| 182 | 1-(4-bromo-5-fluoro-1H-pyrrolo[2,3-b]pyridin-3-yl)-3-(6-(4,4- |
| | difluorocyclohexyl)pyridin-3-yl)urea |
| 181 | 1-(5-chloro-6-(4,4-difluoropiperidin-1-yl)pyridin-3-yl)-3-(5-methoxy-1H- |
| | pyrrolo[2,3-b]pyridin-3-yl)urea |
| 180 | 1-(6-bromo-5-fluoro-1H-pyrrolo[2,3-b]pyridin-3-yl)-3-(6-(4,4- |
| | difluorocyclohexyl)pyridin-3-yl)urea |
| 175 | 1-(6-(4,4-difluoropiperidin-1-yl)-5-fluoropyridin-3-yl)-3-(5- |
| | (dimethylamino)-1H-pyrrolo[3,2-b]pyridin-3-yl)urea |
| 174 | 1-(6-(4,4-difluorocyclohexyl)-5-methylpyridin-3-yl)-3-(5-methoxy-1H- |
| | pyrrolo[2,3-b]pyridin-3-yl)urea |
| 168 | 1-(6-(4,4-difluoropiperidin-1-yl)-5-methylpyridin-3-yl)-3-(5-methoxy-1H- |
| | pyrrolo[2,3-b]pyridin-3-yl)urea |
| 144 | 1-(5-chloro-1H-pyrrolo[3,2-b]pyridin-3-yl)-3-(5-chloro-6-(4,4- |
| | difluoropiperidin-1-yl)pyridin-3-yl)urea |
| 139 | 1-(6-(4,4-difluoropiperidin-1-yl)-5-methylpyridin-3-yl)-3-(5-ethyl-1H- |
| | pyrrolo[3,2-b]pyridin-3-yl)urea |
| 208 | 1-(5-chloro-6-(4,4-difluoropiperidin-1-yl)pyridin-3-yl)-3-(5-(2,2,2- |
| | trifluoroethoxy)-1H-pyrrolo[3,2-b]pyridin-3-yl)urea |
| 212 | 1-(6-(4,4-difluorocyclohexyl)pyridin-3-yl)-3-(5-(2-methoxyethoxy)-1H- |
| | pyrrolo[3,2-b]pyridin-3-yl)urea |
| 211 | 1-(5-chloro-6-(4,4-difluorocyclohexyl)pyridin-3-yl)-3-(5-(2- |
| | methoxyethoxy)-1H-pyrrolo[3,2-b]pyridin-3-yl)urea |
| 218 | 1-(5-chloro-6-(4,4-difluoropiperidin-1-yl)pyridin-3-yl)-3-(5-(2- |
| | methoxyethoxy)-1H-pyrrolo[3,2-b]pyridin-3-yl)urea |
| 129 | 1-(6-(4,4-difluorocyclohexyl)pyridin-3-yl)-3-(5-fluoro-1-(methylsulfonyl)- |
| | 1H-pyrrolo\|2,3-b]pyridin-3-yl)urea |
| 120 | 1-(6-(4,4-difluorocyclohexyl)pyridin-3-yl)-3-(5-fluoro-1-tosyl-1H- |
| | pyrrolo[2,3-b]pyridin-3-yl)urea |
| 121 | 1-(1-benzoyl-5-fluoro-1H-pyrrolo[2,3-b]pyridin-3-yl)-3-(6-(4,4- |
| | difluorocyclohexyl)pyridin-3-yl)urea |
| 122 | 1-(1-acetyl-5-fluoro-1H-pyrrolo[2,3-b]pyridin-3-yl)-3-(6-(4,4- |
| | difluorocyclohexyl)pyridin-3-yl)urea |
| 125 | 1-(6-(4,4-difluorocyclohexyl)pyridin-3-yl)-3-(5-fluoro-1-(1-methyl-1H- |
| | pyrazol-4-yl)-1H-pyrrolo[2,3-b]pyridin-3-yl)urea. |

223. The compound of clause 1, wherein the compound is selected from the group consisting of the following:

| Compound # | Name |
|---|---|
| 130 | 1-(6-(4,4-difluorocyclohexyl)pyridin-3-yl)-3-(5-fluoro-1-(phenylsulfonyl)- |
| | 1H-pyrrolo[2,3-b]pyridin-3-yl)urea |
| 131 | 1-(1-(benzylsulfonyl)-5-fluoro-1H-pyrrolo[2,3-b]pyridin-3-yl)-3-(6-(4,4- |
| | difluorocyclohexyl)pyridin-3-yl)urea |
| 132 | 1-(6-(4,4-difluorocyclohexyl)pyridin-3-yl)-3-(5-fluoro-1-((pyridin-3- |
| | ylmethyl)sulfonyl)-1H-pyrrolo[2,3-b]pyridin-3-yl)urea |
| 133 | 1-(1-(3-(1H-pyrazol-1-yl)propanoyl)-5-fluoro-1H-pyrrolo[2,3-b]pyridin-3- |
| | yl)-3-(6-(4,4-difluorocyclohexyl)pyridin-3-yl)urea |
| 134 | 1-(6-(4,4-difluorocyclohexyl)pyridin-3-yl)-3-(1-(dimethylglycyl)-5-fluoro- |
| | 1H-pyrrolo[2,3-b]pyridin-3-yl)urea |
| 135 | 1-(6-(4,4-difluorocyclohexyl)pyridin-3-yl)-3-(5-fluoro-1-(4- |
| | hydroxybutanoyl)-1H-pyrrolo[2,3-b]pyridin-3-yl)urea |
| 210 | 1-(6-(4,4-difluorocyclohexyl)-5-fluoropyridin-3-yl)-3-(5-(2- |
| | methoxyethoxy)-1H-pyrrolo[3,2-b]pyridin-3-yl)urea. |

224. A pharmaceutical composition comprising a compound of clauses 1-223 and one or more pharmaceutically acceptable excipients.

225. A method for inhibiting STING activity, the method comprising contacting STING with a compound as defined in any one of clauses 1-223.

226. The method of clause 225, wherein the inhibiting comprises antagonizing STING.

227. The method of any one of clauses 225-226, which is carried out in vitro.

228. The method of clause 227, wherein the method comprises contacting a sample comprising one or more cells comprising STING with the compound.

229. The method of clause 227 or 228, wherein the one or more cells are one or more cancer cells.

230. The method of clause 228 or 229 wherein the sample further comprises one or more cancer cells (e.g., wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma).

231. The method of clause 225 or 226, which is carried out in vivo.

232. The method of clause 231, wherein the method comprises administering the compound to a subject having a disease in which increased (e.g., excessive) STING signaling contributes to the pathology and/or symptoms and/or progression of the disease.

233. The method of clause 232, wherein the subject is a human.

234. The method of clause 233, wherein the disease is cancer.

235. The method of clause 234, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

236. The method of clause 234 or 235, wherein the cancer is a refractory cancer.

237. The method of clause 232, wherein the compound is administered in combination with one or more additional cancer therapies.

238. The method of clause 237, wherein the one or more additional cancer therapies comprises surgery, radiotherapy, chemotherapy, toxin therapy, immunotherapy, cryotherapy or gene therapy, or a combination thereof.

239. The method of clause 238, wherein chemotherapy comprises administering one or more additional chemotherapeutic agents.

240. The method of clause 239, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

241. The method of any one of clauses 232-240, wherein the compound is administered intratumorally.

242. A method of treating cancer, comprising administering to a subject in need of such treatment an effective amount of a compound as defined in any one of clauses 1-223, or a pharmaceutical composition as defined in clause 224.

243. The method of clause 242, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

244. The method of clause 242 or 243, wherein the cancer is a refractory cancer.

245. The method of clause 242, wherein the compound is administered in combination with one or more additional cancer therapies.

246. The method of clause 245, wherein the one or more additional cancer therapies comprises surgery, radiotherapy, chemotherapy, toxin therapy, immunotherapy, cryotherapy or gene therapy, or a combination thereof.

247. The method of clause 246, wherein chemotherapy comprises administering one or more additional chemotherapeutic agents.

248. The method of clause 246, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

249. The method of any one of clauses 242-248, wherein the compound is administered intratumorally.

250. A method of inducing an immune response in a subject in need thereof, the method comprising administering to the subject an effective amount of a compound as defined in any one of clauses 1-223, or a pharmaceutical composition as defined in clause 224.

251. The method of clause 250, wherein the subject has cancer.

252. The method of clause 251, wherein the subject has undergone and/or is undergoing and/or will undergo one or more cancer therapies.

253. The method of clause 251, wherein the cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

254. The method of clause any one of clauses 251-253, wherein the cancer is a refractory cancer.

255. The method of clause 250, wherein the immune response is an innate immune response.

256. The method of clause 255, wherein the at least one or more cancer therapies comprises surgery, radiotherapy, chemotherapy, toxin therapy, immunotherapy, cryotherapy or gene therapy, or a combination thereof.

257. The method of clause 256, wherein chemotherapy comprises administering one or more additional chemotherapeutic agents.

258. The method of clause 257, wherein the one or more additional chemotherapeutic agents is selected from alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

259. A method of treatment of a disease in which increased (e.g., excessive) STING signaling contributes to the pathology and/or symptoms and/or progression of the disease, comprising administering to a subject in need of such treatment an effective amount of a compound as defined in any one of clauses 1-223, or a pharmaceutical composition as defined in clause 224.

260. A method of treatment comprising administering to a subject having a disease in which increased (e.g., excessive) STING signaling contributes to the pathology and/or symptoms and/or progression of the disease an effective amount of a compound as defined in any one of clauses 1-223, or a pharmaceutical composition as defined in clause 224.

261. A method of treatment comprising administering to a subject a compound as defined in any one of clauses 1-223, or a pharmaceutical composition as defined in clause 224, wherein the compound or composition is administered in an amount effective to treat a disease in which increased (e.g., excessive) STING signaling contributes to the pathology and/or symptoms and/or progression of the disease, thereby treating the disease.

262. The method of any one of clauses 259-261, wherein the disease is cancer.

263. The method of clause 262, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

264. The method of clause 262 or 263, wherein the cancer is a refractory cancer.

265. The method of any one of clauses 262-264, wherein the compound is administered in combination with one or more additional cancer therapies.

266. The method of clause 265, wherein the one or more additional cancer therapies comprises surgery, radiotherapy, chemotherapy, toxin therapy, immunotherapy, cryotherapy or gene therapy, or a combination thereof.

267. The method of clause 264, wherein chemotherapy comprises administering one or more additional chemotherapeutic agents.

268. The method of clause 267, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

269. The method of any one of clauses 259-268, wherein the compound is administered intratumorally.

270. A method of treatment of a disease, disorder, or condition associated with STING, comprising administering to a subject in need of such treatment an effective amount of a compound as defined in any one of clauses 1-223, or a pharmaceutical composition as defined in clause 224.

271. The method of clause 270, wherein the disease, disorder, or condition is selected from type I interferonopathies, Aicardi-Goutieres Syndrome (AGS), genetic forms of lupus, inflammation-associated disorders, and rheumatoid arthritis.

272. The method of clause 270, wherein the disease, disorder, or condition is a type I interferonopathy (e.g., STING-associated vasculopathy with onset in infancy (SAVI)).

273. The method of clause 272, wherein the type I interferonopathy is STING-associated vasculopathy with onset in infancy (SAVI)).

274. The method of clause 271, wherein the disease, disorder, or condition is Aicardi-Goutieres Syndrome (AGS).

275. The method of clause 271, wherein the disease, disorder, or condition is a genetic form of lupus.

276. The method of clause 271, wherein the disease, disorder, or condition is inflammation-associated disorder.

277. The method of clause 276, wherein the inflammation-associated disorder is systemic lupus erythematosus.

278. The method of any one of clauses 225-277, wherein the method further comprises identifying the subject.

279. A combination comprising a compounds defined in any one of clauses 1-223 or a pharmaceutically acceptable salt or tautomer thereof, and one or more therapeutically active agents.

280. A compound defined in any one of clauses 1-223 or a pharmaceutically acceptable salt or tautomer thereof, or a pharmaceutical composition defined in clause 224, for use as a medicament.

281. A compound defined in any one of clauses 1-223 or a pharmaceutically acceptable salt or tautomer thereof, or a pharmaceutical composition defined in clause 224, for use in the treatment of a disease, condition or disorder modulated by STING inhibition.

282. A compound defined in any one of clauses 1-223 or a pharmaceutically acceptable salt or tautomer thereof, or the pharmaceutical composition defined in clause 224, for use in the treatment of a disease mentioned in any one of clauses 225 to 278.

283. Use of a compound defined in any one of clauses 1-223 or a pharmaceutically acceptable salt or tautomer thereof, or a pharmaceutical composition defined in clause 224, in the manufacture of a medicament for the treatment of a disease mentioned in in any one of clauses 225 to 278.

What is claimed is:

1. A compound of Formula I: 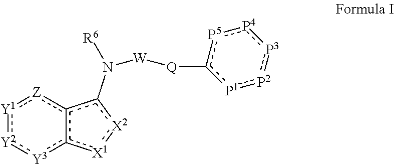
or a pharmaceutically acceptable salt thereof or a tautomer thereof, wherein:
each of Z, Y¹, Y², and Y³ is independently selected from the group consisting of CR¹, N, and NR², provided that 1-3 of Z, Y¹, Y², and Y³ is an independently selected N or NR²;
X¹ is selected from the group consisting of O, S, N, NR², and CR¹;
X² is selected from the group consisting of O, S, N, NR⁴, and CR⁵;
each [symbol] is independently a single bond or a double bond, provided that the five-membered ring comprising X¹ and X² is heteroaryl; the six-membered ring comprising Z, Y¹, Y², and Y³ is heteroaryl; and the ring comprising P¹, P², P³, P⁴, and P⁵ is aromatic;
W is selected from the group consisting of: (i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);
Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents the point of attachment to W;
P¹, P², P³, P⁴, and P⁵ are defined according to (AA) or (BB):
(AA)
each of P¹, P², P³, P⁴, and P⁵ is independently selected from the group consisting of: N, CH, CR⁷, and CR^{c}, provided that: 1-2 of P¹, P², P³, P⁴, and P⁵ is an independently selected CR⁷; or
(BB)
P¹ is absent, thereby providing a 5-membered ring,
each of P², P³, P⁴, and P⁵ is independently selected from the group consisting of O, S, N, NH, NR^{d}, NR⁷, CH, CR⁷, and CR^{c};
provided that 1-3 of P², P³, P⁴, and P⁵ is O, S, N, NH, NR^{d}, or NR⁷; and
1-2 of P², P³, P⁴, and P⁵ is an independently selected NR⁷ or CR⁷;
each R⁷ is independently selected from the group consisting of: —R⁸ and -L³-R⁹;
—R⁸ is selected from the group consisting of:
(a) C₃₋₁₂ cycloalkyl or C₃₋₁₂ cycloalkenyl, each of which is substituted with 1-4 independently selected R^{7′};
(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′};
(c) C₃ cycloalkyl, C3 cycloalkenyl, C5 cycloalkyl, or C5 cycloalkenyl, each of which is optionally substituted with 1-4 independently selected C₁₋₄ alkyl;
(d) C₇₋₁₂ cycloalkyl or C₇₋₁₂ cycloalkenyl, each of which is optionally substituted with 1-4 independently selected C₁₋₄ alkyl;
(e) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl or heterocycloalkenyl ring is optionally substituted with 1-4 independently selected C₁₋₄ alkyl;
(f) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and
(g) C₆₋₁₀ aryl optionally substituted with 1-4 independently selected R^{7′};
-L³ is selected from the group consisting of —O—, —S—, —NH—, S(O)₁₋₂, —CH₂—, C(═O)NH, NHC(═O), C(═O)O, OC(═O), C(═O), NHS(O)₂, and S(O)₂NH;
—R⁹ is selected from the group consisting of:
(a) C₃₋₁₂ cycloalkyl or C₃₋₁₂ cycloalkenyl, each of which is optionally substituted with 1-4 independently selected R^{7′},
(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};
(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and
(d) C₆₋₁₀ aryl optionally substituted with 1-4 independently selected R^{7′};
each occurrence of R^{7′} is independently selected from the group consisting of:
halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),
provided that when R⁷ is R⁸; and R⁸ is cycloalkyl, cycloalkenyl, heterocyclyl, or heterocycloalkenyl and substituted with 1-4 R^{7′}, then:
R⁸ cannot be monosubstituted with C₁₋₄ alkyl, and
when R⁸ is substituted with 2-4 R^{7′}, then at least one R^{7′} must be a substituent other than C₁₋₄ alkyl;
each occurrence of R¹ is independently selected from the group consisting of:
H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy optionally substituted with —OH, C₁₋₄ alkoxy, C₁₋₄ haloalkoxy, or —NR^{e}R^{f}; C₁₋₄ haloalkoxy; -L¹-L²-R^{h}; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);
each occurrence of R² is independently selected from the group consisting of:
(i) H;
(ii) C₁₋₆ alkyl optionally substituted with 1-3 independently selected R^{a};
(iii) —C(O)(C₁₋₆ alkyl) optionally substituted with 1-3 independently selected R^{a};
(iv) —C(O)O(C₁₋₄ alkyl) optionally substituted with 1-3 independently selected R^{a};
(v) —CON(R′)(R″);
(vi) —S(O)₁₋₂(NR′R″);
(vii) —S(O)₁₋₂(C₁₋₄ alkyl) optionally substituted with 1-3 independently selected R^{a};
(viii) —OH;
(ix) C₁₋₄ alkoxy; and
(x) -L⁴-L⁵-R;
R⁴ is selected from the group consisting of H and C₁₋₆ alkyl optionally substituted with 1-3 independently selected R^{a};
R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂, wherein the heteroaryl ring is optionally substituted with 1-4 independently selected C₁₋₄ alkyl;
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
each occurrence of R^{b} is independently selected from the group consisting of: C₁₋₁₀ alkyl optionally substituted with 1-6 independently selected R^{a}; C₁₋₄ haloalkyl; —OH;
oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and
-L¹-L²-R^{h};
each occurrence of R^{c} is independently selected from the group consisting of:
halo; cyano; C₁₋₁₀ alkyl optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};
R^{d} is selected from the group consisting of: C₁₋₆ alkyl optionally substituted with 1-3 substituents each independently selected from the group consisting of halo, C₁₋₄ alkoxy, and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or
R^{e} and R together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl;
and (b) 0-3 ring heteroatoms (in addition to the nitrogen atom attached to R^{e} and R^{f}), which are each independently selected from the group consisting of N(R^{d}), NH, O, and S;
-L¹ is a bond or C₁₋₃ alkylene; -L² is —O—, —N(H)—, —S(O)₀₋₂—, or a bond;
R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};
-L⁵- is selected from the group consisting of a bond and C₁₋₄ alkylene;
Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy;
each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or
R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S; and
provided that the compound is other than:

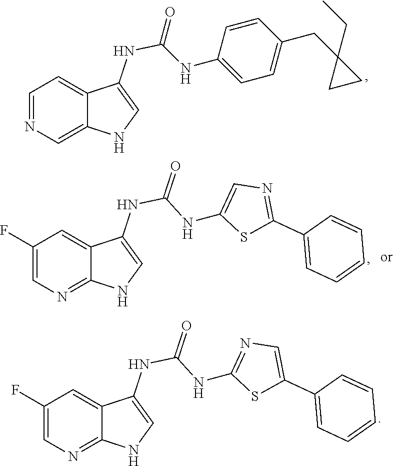 [SMILES: CCC1(Cc2ccc(NC(=O)Nc3c[nH]c4cnccc34)cc2)CC1 ; O=C(Nc1cnc(-c2ccccc2)s1)Nc1c[nH]c2ncc(F)cc12 ; O=C(Nc1ncc(-c2ccccc2)s1)Nc1c[nH]c2ncc(F)cc12]

2. The compound of claim 1, wherein P¹, P², P³, P⁴, and P⁵ are as defined according to (AA.

3. The compound of claim 2, wherein one or more of P¹, P², P³, P⁴, and P⁵ is N, or one of P¹, P², P³, P⁴, and P⁵ is N.

4. The compound of claim 2 or 3, wherein the 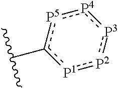
moiety has the formula: 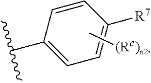
wherein n2 is 0, 1, or 2.

5. The compound of any one of claims 2 to 4, wherein the 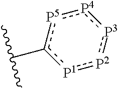
moiety has the formula

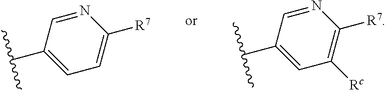

6. The compound of claim 2, wherein each of P¹, P², P³, P⁴, and P⁵ is independently selected from the group consisting of CH, CR⁷, and CR^{c}.

7. The compound of claim 2 or 6, wherein the 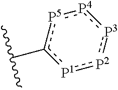
moiety has the formula: 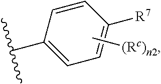
wherein n2 is 0, 1, or 2.

8. The compound of claims 2, 6, or 7, wherein the 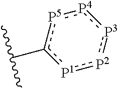
moiety has the formula:

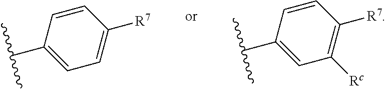

9. The compound of any one of claims 1-8, wherein R⁷ is R⁸.

10. The compound of any one of claims 1-9, wherein R⁸ is
i) C₃₋₁₂ cycloalkyl or C₃₋₁₂ cycloalkenyl, each of which is substituted with 1-4 independently selected R^{7′};
ii) C₄₋₈ cycloalkyl which is substituted with 1-4 independently selected R^{7′};
iii) cyclohexyl or cyclobutyl, each of which is substituted with 1-4 independently selected R^{7′};
iv) 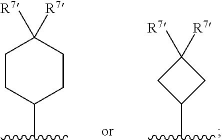
wherein each R^{7′} is independently halo;
or
v) an R⁸ of any one of i) to iv) wherein each R^{7′} is independently halo.

11. The compound of any one of claims 1-9, wherein R⁸ is
i) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};
ii) heterocyclyl of 4-6 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′};
iii) heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 2-4 independently selected R^{7′};
iv) 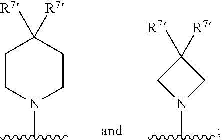
v) spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R^{7′};
vi) 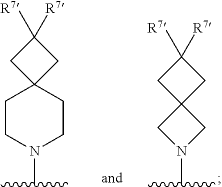
or
vii) an R⁸ of any one of i) to vi) wherein each R^{7′} is independently halo.

12. The compound of any one of claims 1-11, wherein each occurrence of R^{c} is an independently selected halo.

13. The compound of any one of claims 1-12, wherein Q is NH; and W is C(═O).

14. The compound of any one of claims 1-13, wherein one of Y¹, Y², and Y³ is independently N; and each of the remaining Y¹, Y², and Y³ is an independently selected CR¹; or
Z is N; and each of Y¹, Y², and Y³ is an independently selected CR¹.

15. The compound of any one of claims 1-14, wherein the 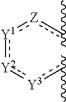
moiety is selected from the group consisting of:

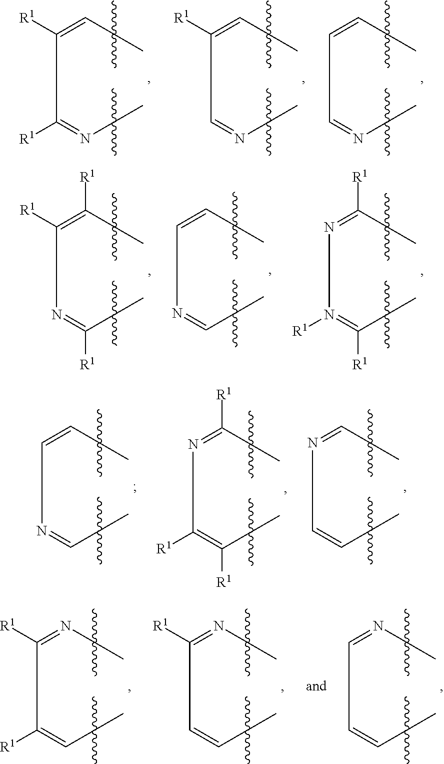

16. The compound of any one of claims 1-15, wherein X¹ is NR²; and X² is CR⁵, or wherein X¹ is NH; and X² is CH.

17. The compound of any one of claims 1-16, wherein the compound is a compound of Formula (Ia): 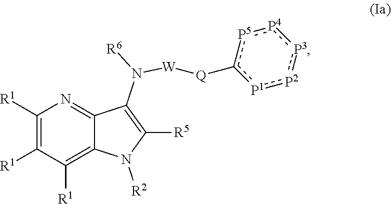
or a pharmaceutically acceptable salt thereof,
or wherein the compound is a compound of Formula (Id): 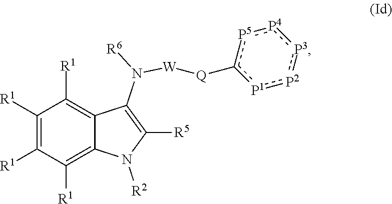
or a pharmaceutically acceptable salt thereof,

18. The compound of any one of claims 1-16, wherein the compound is
i) a compound of Formula (Ia-1), (Ia-2), or (Ia-3): 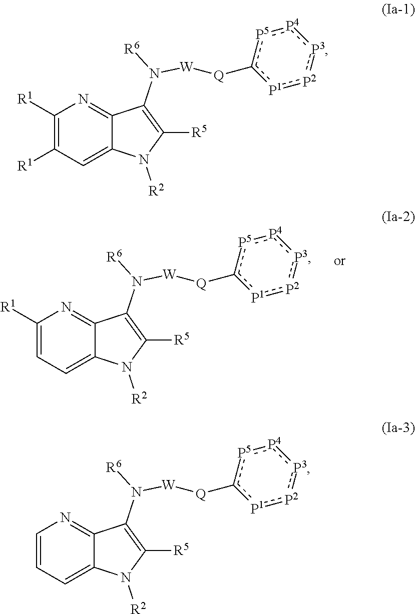
or a pharmaceutically acceptable salt thereof,
ii) a compound of Formula (Id-1), (Id-2), or (Id-3): 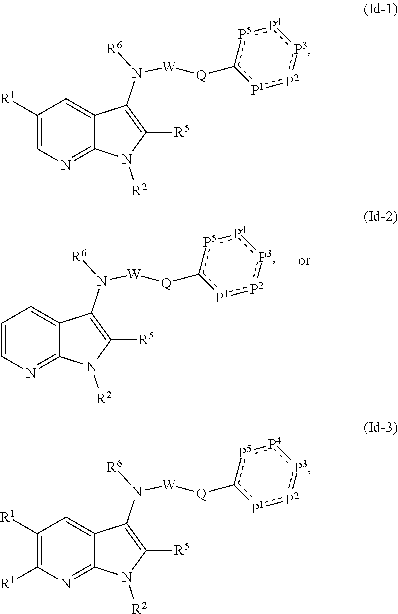
or a pharmaceutically acceptable salt thereof; or
iii) a compound of any one of i) or ii) wherein R² is H; and R⁵ is H.

19. The compound of any one of claims 1-18, wherein
i) each occurrence of R¹ is H;
ii) 1-2 occurrences of R¹ is other than H;
iii) 1 occurrence of Rⁱ is other than H;
or
iv) one occurrence of R¹ is selected from the group consisting of: halo; cyano;
C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)₁₋₂(NR′R″); —NO₂; -L¹-L²-R^{h}; —C(═O)(C₁₋₄ alkyl); —C(═O)O(C₁₋₄ alkyl); —C(═O)OH; and C(═O)N(R′)(R″).

20. The compound of claim 1, wherein the compound is a compound of Formula (I-3a) or (I-3b): 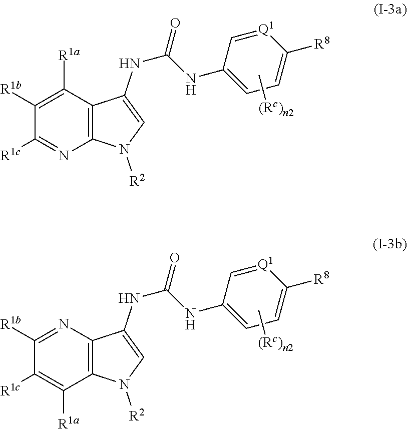
or a pharmaceutically acceptable salt thereof, wherein:
each of R^{1a}, R^{1b}, and R^{1c} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;
Q¹ is N or CH;
R⁸ is selected from the group consisting of: 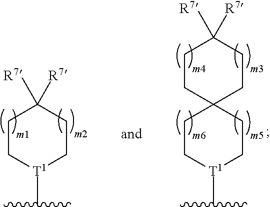
n2 is 0, 1, or 2;
each R^{c} when present is independently selected from the group consisting of: halo, cyano, C₁₋₃ alkyl, and C₁₋₃ alkoxy;
m1 and m2 are independently 0, 1, or 2; m3, m4, m5, and m6 are independently 0 or 1; and
T¹ is CH or N.

21. The compound of claim 20, wherein R² is H; n2 is 1; R^{c} is ortho to R⁸; and each R^{7′} is independently halo.

22. The compound of claim 20 or 21, wherein R⁸ is selected from the group consisting of:

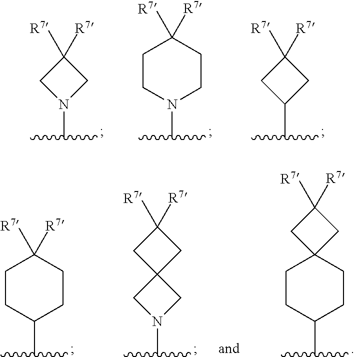

23. The compound of claim 1, wherein the compound is selected from the group consisting of the compounds delineated in Table C1, or a pharmaceutically acceptable salt thereof.

24. A pharmaceutical composition comprising a compound of claims 1-23 and one or more pharmaceutically acceptable excipients.

25. A method for inhibiting STING activity, the method comprising contacting STING with a compound as claimed in any one of claims 1-23, or a pharmaceutically acceptable salt thereof; or a pharmaceutical composition as claimed in claim 24.

26. A method of inducing an immune response in a subject in need thereof, the method comprising administering to the subject an effective amount of a compound as claimed in any one of claims 1-23, or a pharmaceutically acceptable salt thereof; or a pharmaceutical composition as claimed in claim 24.

27. A method of treatment of disease, disorder, or condition associated with STING, such as a disease, disorder, or condition, in which increased STING signaling, such as excessive STING signaling, contributes to the pathology and/or symptoms and/or progression of the disease comprising administering to a subject in need of such treatment an effective amount of a compound as claimed in any one of claims 1-23, or a pharmaceutically acceptable salt thereof, or a pharmaceutical composition as claimed in claim 24.
